(19) United States
(12) Patent Application Publication
(10) Pub. No.: US 2021/0105962 A1
(43) Pub. Date: Apr. 15, 2021

(54) METHODS AND COMPOSITIONS RELATING TO MAINTAINER LINES

(71) Applicant: ELSOMS DEVELOPMENTS LTD, Lincolnshire (GB)

(72) Inventors: Anthony Gordon KEELING, Lincolnshire (GB); Matthew John MILNER, Cambridgeshire (GB)

(73) Assignee: ELSOMS DEVELOPMENTS LTD, Lincolnshire (GB)

(21) Appl. No.: 16/967,439

(22) PCT Filed: Feb. 22, 2019
(86) PCT No.: PCT/US2019/019139

§ 371 Date: Aug. 5, 2020

Related U.S. Application Data
(60) Provisional application No. 62/633,668, filed on Feb. 22, 2018.
(60) Provisional application No. 62/664,340, filed on Apr. 30, 2018.

(51) Int. Cl. A01H 1/08; A01H 6/46; A01H 5/10; A01H 1/02

(52) U.S. Cl. CPC A01H 1/08; A01H 1/02; A01H 5/10; A01H 6/46

(57) ABSTRACT

The methods and compositions described herein relate to maintainer lines (e.g, male-fertile lines) for producing or propogation of plants with a male-sterile phenotype.

### CROSS-REFERENCE TO RELATED APPLICATIONS

This application claims benefit under 35 U.S.C. § 119(e) of U.S. Provisional Application Nos. 62/633,668 filed Feb. 22, 2018 and 62/664,340 filed Apr. 30, 2018, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 21, 2019, is named 077524-090370WOPT_SL.txt and is 273,002 bytes in size.

### TECHNICAL FIELD

The technology described herein relates to engineered plants, e.g., maintainer lines and/or non-transgenic plants with co-segregating constructs.

### BACKGROUND

Male-sterile lines, particularly recessive male-steriles which can be pollinated by wild-type pollen which restores fertility to the progeny, are of significant value in plant breeding operations, allowing certainty in the production of hybrids and avoiding costly manual procedures. However, a male-sterile line obviously cannot propagate itself. Instead, the male-sterile line is propogated via the use of a maintainer line whose pollen carries the same male-sterile alleles as the cognate male-sterile plant. The genetics of maintainer lines vary, but the general concept is that the line is arranged in such a way that the pollen produced can cross with a cognate male-sterile plant to produce a next generation of male-sterile plants. The maintainer line is further arranged such that at least a proportion of self-pollination propogates the same maintainer line genotype of the parent plant.

However, maintainer lines for recessive male-sterility lines have traditionally necessitated transgenic and/or GMO approaches. Typical approaches that are incorporated into maintainer lines include expression cassettes or transgenes to “rescue” the male-sterility, selection markers for “purified” propogation of the maintainer line, or cassettes designed to induce death or ineffectiveness of pollen or ovules of the undesired genotypes. In view of current worldwide agricultural regulatory approaches, such maintainer lines can be difficult and expensive to bring to bear.

### SUMMARY

Described herein is an approach to engineering a maintainer line without the need for exogenous genetic sequences and/or transgenic/GMO constructs. The nature of this novel approach to maintainer line construction also means that the maintainer line is suitable for use with cognate lines that relate to multi-gene phenotypes and that the maintainer line can reduce or avoid the need for seed or plant selection/deselection during propagation.

In one aspect of any of the embodiments, described herein is a male-fertile maintainer plant for a male-sterile polyploid plant, the maintainer plant comprising: in the first chromosome of a homologous pair in a first genome:
a. an endogenous, wild-type functional allele of a male-fertility gene (Mf gene);
b. an engineered knock-out modification at the allele of a pollen-grain-vital gene (PV gene);
c. an endogenous, wild-type functional allele of an ovule-vital gene (OV gene); and
d. at least one engineered modification comprising a deletion of endogenous intervening sequence between the Mf; PV; and/or OV loci;
   in the second chromosome of the same homologous pair in the first genome:
e. an engineered knock-out modification at the allele of the Mf gene;
f. an endogenous, wild-type functional allele of the PV gene; and
g. an engineered knock-out modification at the allele of the OV gene;
h. at least one engineered modification comprising a deletion of endogenous intervening sequence between the Mf; PV; and/or OV loci;
   in a second and any subsequent genomes:
i. an engineered knock-out modification at each allele of the Mf gene;
j. an engineered knock-out modification at each allele of the PV gene;
k. an engineered knock-out modification at each allele of the OV gene;
   whereby the pollen grains produced by the male-fertile maintainer plant comprise the second chromosome of the first genome and do not comprise the first chromosome of the first genome (hereinafter referred to as the pollen construct); and the ovules produced by the male-fertile maintainer plant comprise the first chromosome of the first genome and do not comprise the second chromosome of the first genome (hereinafter referred to as the ovule construct). In some embodiments of any of the aspects, the first and second chromosomes of the first genome comprise at least one engineered modification comprising a deletion of endogenous intervening sequence between any two of the Mf; PV; and OV loci. In some embodiments of any of the aspects, the first and second chromosomes of the first genome comprise two engineered modifications comprising deletions of endogenous intervening sequence between the Mf; PV; and OV loci.

In one aspect of any of the embodiments, described herein is a male-fertile maintainer plant for a male-sterile polyploid plant, the maintainer plant comprising

a. an engineered knock-out modification at each allele of the Mf gene in every genome;

b. an engineered knock-out modification at each allele of the PV gene in every genome;

c. an engineered knock-out modification at each allele of the OV gene in every genome; and

d. a modification in a first genome comprising:
i. at a loci on a first member of a homologous pair of chromosomes, an engineered insertion or knock-in of the PV gene; and
ii. at a loci on a second member of the homologous pair of chromosomes, an engineered insertion or knock-in of the Mf and OV genes;
wherein the loci of i and ii are homolgous, intra-genic, or inter-genic regions and not coextensive with the alleles of a, b, or c.
   The foregoing modifications result in viable, germinating, pollen grains produced by the male-fertile maintainer plant comprising all the knockouts of a, b and c above and the knock-in of the PV gene (the other 50% of pollen grains without the PV gene will not be viable). (This is hereinafter referred to as the knock-in pollen construct); and the viable ovules produced by the male-fertile maintainer plant comprising all the knockouts of a, b and c above and the knock-in of the Mf and OV genes (the other 50% of ovules without the OV gene will not be viable). (This is hereinafter referred to as the knock-in ovule construct.) In some embodiments of any of the aspects, the chromosomes of d are different from the chromosomes comprising the alleles of a, b, and c. In some embodiments of any of the aspects, the alleles of a, b, and c are found on the same chromosome. In some embodiments of any of the aspects, two alleles of a, b, and c are found on the same chromosome, and the third allele is found a different chromosome. In some embodiments of any of the aspects, the alleles of a, b, and c are each found on a different chromosome, e.g., each allele of a, b, and c is found on a chromosome not comprising the other two alleles. It is noted that insertion of a gene from the same (or a crossable) plant species—cis-genesis—as proposed in certain embodiments herein, is a gene transfer technique which is not regulated as GM in at least the United States and so can be useful in certain embodiments of the instant compositions and methods.

In one aspect of any of the embodiments, described herein is a method of producing a male-fertile maintainer plant as described herein, wherein the method comprises:
a. engineering the knock-out modifications in each allele of Mf, OV, and PV in the second and any subsequent genomes, resulting in a fertile plant;
b. engineering the modifications in the first chromosome of the first genome; and
c. engineering the modifications in the second chromosome of the first genome.
   In some embodiments of any of the aspects, the modifications in the first chromosome of the first genome are engineered in a first plant; the modifications in the second chromosome of the first genome are engineered in a second plant; the resulting plants are crossed; and the F2 progeny which comprise the engineered first and second chromosomes of the first genome are selected.

In one aspect of any of the embodiments, described herein is a method of producing a male-fertile maintainer plant as described herein, wherein the method comprises:

engineering the pollen construct and/or ovule construct in a first plant;

transferring the pollen construct and/or ovule construct to a second, wild-type cultivar plant by:
a) crossing pollen comprising the pollen construct from the first plant onto the second plant;
b) selfing the F1 generation
c) in the F2 generation, selecting plants homozygous for the pollen construct and crossing the pollen from the selected homozygous F2 plants onto third, wild-type cultivar plant of the same cultivar as the second plant; and
d) repeating this process until the crossed plants are substantially isogenic with the wild-type cultivar with the exception of the pollen construct; and
e) crossing pollen from a fourth wildtype cultivar plant onto a plant comprising the ovule construct;
f) selfing the F1 generation
g) in the F2 generation, selecting plants homozygous for the ovule construct and crossing pollen from a fifth, wild-type cultivar plant of the same cultivar as the fourth plant onto the selected homozygous F2 plants; and
h) repeating this process until the crossed plants are substantially isogenic with the wild-type cultivar with the exception of the ovule construct; and
i) crossing the pollen from the plant obtained from step (d) onto the plant obtained from step (h); and
j) selfing the F1 generation, whereby the resulting progeny will have a heterozygous genotype and produce pollen with the pollen construct only and ovules with the ovule construct only.
   In some embodiments of any of the aspects, steps a-d and e-h are performed concurrently.

In some embodiments of any of the aspects, the plant is hexaploid and the male-sterile plant comprises an engineered knock-out modification at each of the six alleles of the male-fertility gene. In some embodiments of any of the aspects, the plant is tetraploid and the male-sterile plant comprises an engineered knock-out modification at each of the four alleles of the male-fertility gene.

In some embodiments of any of the aspects, the maintainer plant is substantially isogenic with the male-sterile plant with the exception of the engineered modifications. In some embodiments of any of the aspects, the male sterile plant comprises engineered knock-out modifications at each allele of the Mf gene.

In some embodiments of any of the aspects, at least one copy of any of the engineered modifications is engineered by using a site-specific guided nuclease. In some embodiments of any of the aspects, the site-specific guided nuclease is a form of CRISPR-Cas (such as CRISPR-Cas9). In some embodiments of any of the aspects, a multi-guide construct is used, e.g., to engineer the deletions. In some embodiments of any of the aspects, engineering one or more modifications comprises a single step of contacting a plant cell with a Cas enzyme and one or more multi-guide constructs that direct each modification, e.g., target each allele of Mf, OV, and PV in the second and subsequent genomes.

In some embodiments of any of the aspects, the endogenous Mf, PV, and OV genes are located on the same arms of the same homologous pair of chromosomes.

In some embodiments of any of the aspects, the plant is wheat. In some embodiments of any of the aspects, the plant is hexaploid wheat, tetraploid wheat, Triticum aestivum, or Triticum durum. In some embodiments of any of the aspects, the plant is triticale, oat, canola/oilseed rape or indian mustard.

In some embodiments of any of the aspects, the PV gene has homology to a gene demonstrated to be vital for post-meiosis events such as pollen-grain development, germination, or pollen tube extension in a plant. In some embodiments of any of the aspects, the PV gene is selected from the genes of Table 1. In some embodiments of any of the aspects, the PV gene displays the same type of activity and shares at least 80%, at least 85%, at least 90%, at least 95%, or greater sequence identity with a PV gene of Table 1.

In some embodiments of any of the aspects, the OV gene has homology to a gene demonstrated to be vital for post-meiosis events such as cell division of the initial archesporial haploid cell, differentiation into an egg cell, a central cell, two synergid cells and three antipodal cells or synthesis and export of pollen-tube attractant compounds in a plant. In some embodiments of any of the aspects, the OV gene is selected from the genes of Table 2. In some embodiments of any of the aspects, the OV gene displays the same type of activity and shares at least 80%, at least 85%, at least 90%, at least 95%, or greater sequence identity with a OV gene of Table 2.

In some embodiments of any of the aspects, the plant does not comprise any genetic sequences which are exogenous to that plant species.

In one aspect of any of the embodiments, described herein is a method of selecting a chromosome arm of a cultivar genome as the site of production of a co-segregating construct, wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequence between the Mf; PV; and/or OV loci;
   the method comprising:
a. selecting one of a Mf gene, PV gene, or OV gene;
b. identifying one of each of the two genes not selected in step a which map to the same arm of the same chromosome as the gene selected in step a;
c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
d. selecting the chromosome arm of a cultivar genome as an appropriate site of production if target sequences of step (c) can be identified.
   In some embodiments of any of the aspects, identifying the two genes not selected in step a comprises first identifying the genes in a reference genome and then searching the cultivar genome to identify any translocations or mutations that would affect the two genes. In some embodiments of any of the aspects, the Mf gene is a gene which has been identified to produce a male-sterile phenotype when modified to a knock-out allele.

In one aspect of any of the embodiments, described herein is a system for selecting a chromosome arm of a cultivar genome as the site of production of a co-segregating construct;

wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequence between the Mf; PV; and/or OV loci;
   the system comprising:
i. a memory having processor-readable instructions stored therein; and
ii. a processor configured to access the memory and execute the processor-readable instructions, which, when executed by the processor configures the processor to preform a method, the method comprising:
   A. receiving initial data relating to a co-segregating construct, the initial data including one of a Mf gene, PV gene, or OV gene and a reference genome;
   B. processing, using the processor, the initial data to identify one each of the two genes not provided in step A which map to the same arm of the same chromosome as the gene provided in step A;
   C. processing, using the processor, the data obtained from step B) to identify, for each set of a Mf gene, a PV gene, and an OV gene, at least one target sequences for a site-specific guided nuclease guide, with one target sequence identified from each of:
      the sequence distal of regulatory elements regulating expression of the start or end of the open reading frame on the distal side of the proximal gene of any subset of two genes identified in step A; and
      the sequence proximal of regulatory elements proximal of the start or end of the open reading frame on the proximal side of the distal gene of any subset of two genes identified in step A;
   D. creating a library of sets of Mf, PV, and OV genes and associated target sequences;
   E. selecting, using the processor, a set of Mf, PV, and OV genes and associated target sequences with the minimal distance from the target sequences to the border of the open reading frame of the nearest gene from the library of sets.

In one aspect of any of the embodiments, described herein is a method of producing a co-segregating construct in a chromosome arm of a cultivar genome; wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequence between the Mf; PV; and/or OV loci;
   the method comprising:
a. selecting one of a Mf gene, PV gene, or OV gene;
b. identifying one of each of the two genes not selected in step a which map to the same arm of the same chromosome as the gene selected in step a;
c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
d. selecting the chromosome arm of a cultivar genome as an appropriate site of production if target sequences of step (c) can be identified; and
e. engineering the at least one modification comprising a deletion of endogenous intervening sequence between the Mf; PV; and/or OV loci by contacting a cell comprising the chromosome with a site-specific guided nuclease and the guides which hybridize to the target sequences identified in step (c).

In one aspect of any of the embodiments, described herein is a plant or plant cell comprising a deactivating modification of at least one OV gene. In some embodiments of any of the aspects, the plant or cell further comprises a deactivating modification of at least one PV or Mf gene. In one aspect of any of the embodiments, described herein is a plant or plant cell comprising a deactivating modification of at least one PV gene. In some embodiments of any of the aspects, the plant or cell further comprises a deactivating modification of at least one OV or Mf gene. In some embodiments of any of the aspects, the plant permits seed segregation of its progeny. In some embodiments of any of the aspects, the plant or cell further comprises deactivating modifications of each of the copy of the gene(s). In some embodiments of any of the aspects, the deactivating modification is identical across each genome of the plant. In some embodiments of any of the aspects, each genome of the plant comprises a different deactivating modification. In some embodiments of any of the aspects, the gene(s) is selected from the genes of Tables 1-3. In some embodiments of any of the aspects, the gene(s) has at least 60%, at least 90%, or at least 95% identity with any of the genes of Tables 1-3. In some embodiments of any of the aspects, the gene(s) has the same activity and at least 60%, at least 90%, or at least 95% identity with any of the genes of Tables 1-3.

In some embodiments of any of the aspects, the deactivating modification is a site-directed mutagenic event resulting from the activity of a site-specific nuclease; or the at least one gene is deactivated by site-directed mutagenesis resulting from the activity of a site-specific nuclease. In some embodiments of any of the aspects, the site-specific nuclease is CRISPR-Cas. In some embodiments of any of the aspects, the deactivating modification is excision of at least part of a coding or regulatory sequence; or the at least one gene is deactivated by excision of at least part of a coding or regulatory sequence. In some embodiments of any of the aspects, the deactivating modification is insertion of RNAi-encoding sequences; or the at least one gene is deactivated by inhibition by expression of RNAi. In some embodiments of any of the aspects, the deactivating modification is non-transgenic mutagenesis; or the at least gene is deactivated by non-transgenic mutagenesis.

In one aspect of any of the embodiments, described herein is a plant or plant cell comprising a modification comprising the deletion of endogenous sequence between a first and second gene, whereby the co-segregation of the first and second genes is increased. In some embodiments of any of the aspects, the plant or cell further comprises the deletion of a second endogenous sequence between the second gene and a third gene, whereby the co-segregation of the first, second, and third genes is increased. In some embodiments of any of the aspects, the first, second, or third gene is a Mf, OV, or PV gene. In some embodiments of any of the aspects, the at least one deletion is present on a first chromosome or genome, and the plant further comprises a deactivating modification of copies of the first, second, and/or third genes on another chromosome or genome.

### BRIEF DESCRIPTION OF THE DRAWINGS

FIGS. 1A-1D depict diagrams of exemplary chromosomes comprising modifications according to certain aspects described herein. Chromosomes from each of three genomes (e.g., as in a wheat plant) are depicted. FIG. 1A depicts three exemplary genomes of wheat chromosome 7 in the wild-type, before any of the edits or modifications described herein. FIG. 1B depicts three exemplary genomes of wheat chromosome 7, reflecting multiplex editing of all three genes of interest. FIG. 1C depicts three exemplary genomes of wheat chromosome 7, reflecting the intergenic deletions. FIG. 1D depicts three exemplary genomes of wheat chromosome 7, reflecting the final product maintainer genotype.

FIG. 2 depicts a diagram of exemplary chromosomes comprising modifications according to certain aspects described herein, e.g., the exemplary modifications described in Example 3. Chromosomes from each of three genomes (e.g., as in a wheat plant) are depicted.

FIG. 3 depicts a diagram of exemplary chromosomes comprising modifications according to certain aspects described herein. Chromosomes from each of three genomes (e.g., as in a wheat plant) are depicted.

### DETAILED DESCRIPTION

The methods and compositions described herein relate to polyploidal maintainer plants in which a first genome is engineered, without introducing exogenous sequences, to allow two or more genes to cosegregate. The first genome comprises functional or wild-type, endogenous copies of genes controlling a trait of interest are present. The second or further genomes can comprise the mutated or recessive alleles of those genes which give rise to a phenotype of interest when the plant is homozygous in that respect. For example, when male-sterility is the trait of interest, the first genome comprises at least one allele that confers male-fertility. In the further genomes, alleles are present which confer the phenotype of interest. Stated another way, the first genome comprises at least one dominant allele, while the further genomes comprise recessive alleles which confer the phenotype of interest.

In the first genome, the two or more genes are caused to cosegregate by engineering one or more deletions of endogenous sequence between the two or more such genes, thereby increasing their genetic linkage. This approach avoids introducing exogenous sequences and any loss of genetic information can be compensated for by the second or further genomes in which the relevant intergenic sequences are not modified.

It is noted that the approach of increasing genetic linkage of multiple gene(s) (whether recessive or dominant alleles) in a first genome is applicable to any phenotype of interest and any gene(s) of interest. Embodiments relating to male-fertile maintainer plants for a male-sterile polyploid plant are provided herein as a non-limiting exemplar. It is contemplated that such an approach would also be suitable for use with, e.g., disease resistance genes, drought tolerance genes, or any other desired phenotype. For example, if two disease resistance genes are found on the same chromosome arm in a first cultivar, the cultivar can be engineered to remove endogenous intergenic sequence and the two genes will be more closely linked. The engineered cultivar can be successfully used to cross the two disease resistance genes into a second cultivar or a new hybrid cultivar by traditional crossing approaches. Such an approach avoids transgenic/GMO approaches while also providing a large increase in the efficiency of introgression.

Accordingly, in one aspect, described herein is a plant or plant cell comprising a modification comprising the deletion of endogenous sequence between a first and second gene, whereby the co-segregation of the first and second genes is increased. In some embodiments of any of the aspects, the plant or plant cell further comprises the deletion of a second endogenous sequence between the second gene and a third gene, whereby the co-segregation of the first, second, and third genes is increased. In some embodiments of any of the aspects, the first, second, or third gene is a Mf, OV, or PV gene (defined below). In some embodiments of any of the aspects, the at least one deletion is present on a first chromosome or genome, and the plant further comprises a deactivating modification of copies of the first, second, and/or third genes on the second chromosome of that genome, or on one or more chromosome(s) of further genomes. Within the term ‘plants’ in this specification is included seeds and seedlings.

With regard to maintainer lines for male-sterile plants, in one aspect of any of the embodiments, described herein is a male-fertile maintainer plant for a male-sterile polyploid plant. The male-sterile polyploid plant comprises only knock-out and/or non-functional alleles of a male-fertility gene (Mf gene) across all genomes. The maintainer plant comprises in the first chromosome of a homologous pair in a first genome:
a. an endogenous, wild-type functional allele of a male-fertility gene which functions largely before meiosis (Mf gene);
b. an engineered knock-out modification at the allele of a pollen-grain-vital gene which functions after meiosis (PV gene);
c. an endogenous, wild-type functional allele of an ovule-vital gene (OV gene); and
d. at least one modification comprising a deletion of endogenous intervening sequences between the Mf; PV; and/or OV loci;
   in the second chromosome of the same homologous pair in the first genome:
e. an engineered knock-out modification at the allele of the Mf gene;
f. an endogenous, wild-type functional allele of the PV gene; and
g. an engineered knock-out modification at the allele of the OV gene;
h. at least one engineered modification comprising a deletion of endogenous intervening sequences between the Mf; PV; and/or OV loci
   in a second and any subsequent genomes:
i. an engineered knock-out modification at each allele of the Mf gene;
j. an engineered knock-out modification at each allele of the PV gene;
k. an engineered knock-out modification at each allele of the OV gene.
   In some embodiments of any of the aspects, the first and second chromosomes of the first genome can comprise additional engineered modifications comprising deletions of endogenous intervening sequences between the three genes, or in alternative embodiments two of the genes can be adjacent and/or in have a high enough genetic linkage at deletions of the intergenic sequence are not made. In some embodiments of any of the aspects, the first and second chromosomes of the first genome can comprise two engineered modifications comprising deletions of endogenous intervening sequences between the Mf, PV, and OV loci.

The foregoing plant therefore will produce viable pollen grains which comprise the second chromosome of the first genome and never the first chromosome of the first genome as the latter will comprise pollen-grains with the knocked-out PV gene and will not be viable. Similarly, the foregoing plant therefore will only produce ovules which comprise the first chromosome of the first genome and not the second chromosome of the first genome as the latter will comprise ovules with the knocked-out OV gene and will not be viable. Elements a.-d. on the first chromosome of the first genome are referred to collectively herein as the ovule construct. Elements e.-h. on the second chromosome of the first genome are referred to collectively herein as the pollen construct.

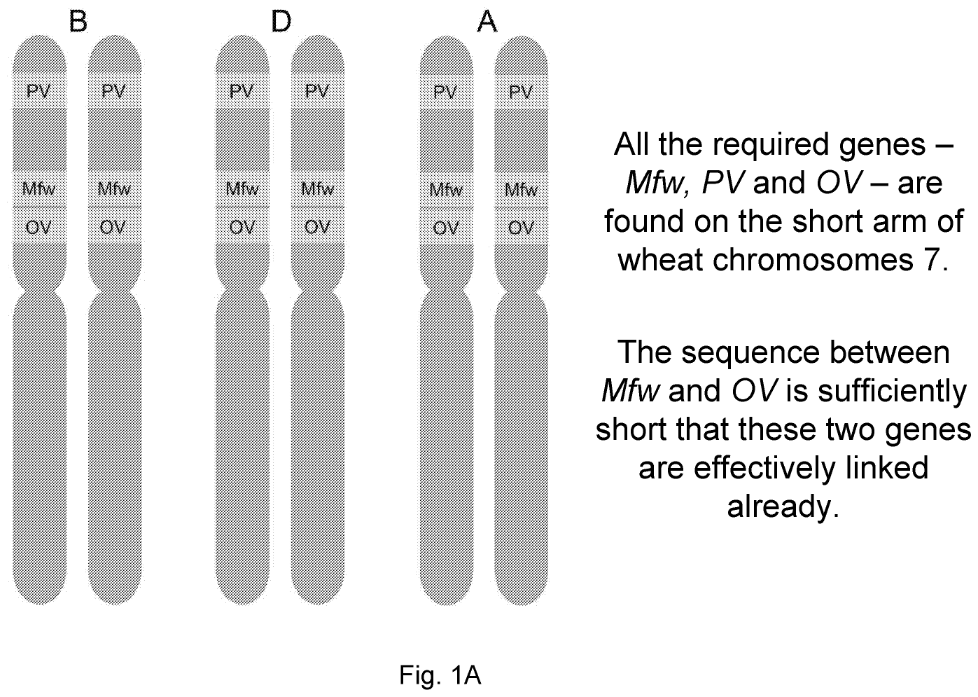

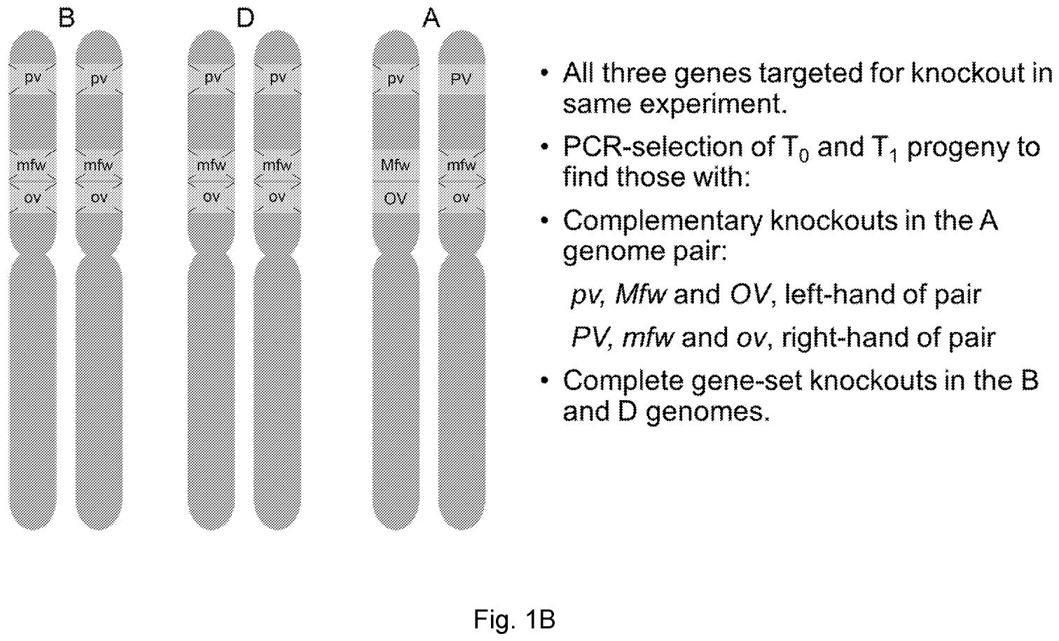

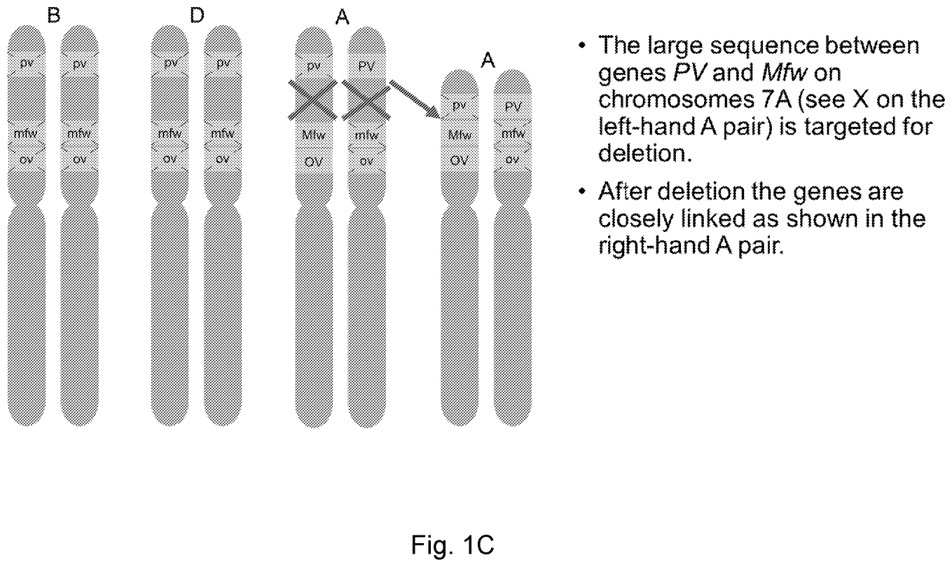

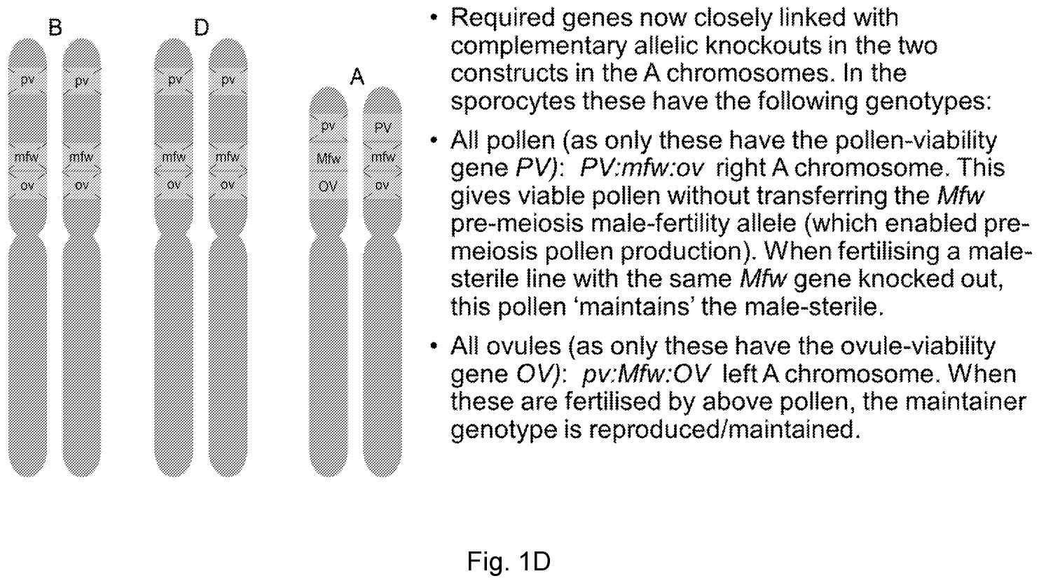

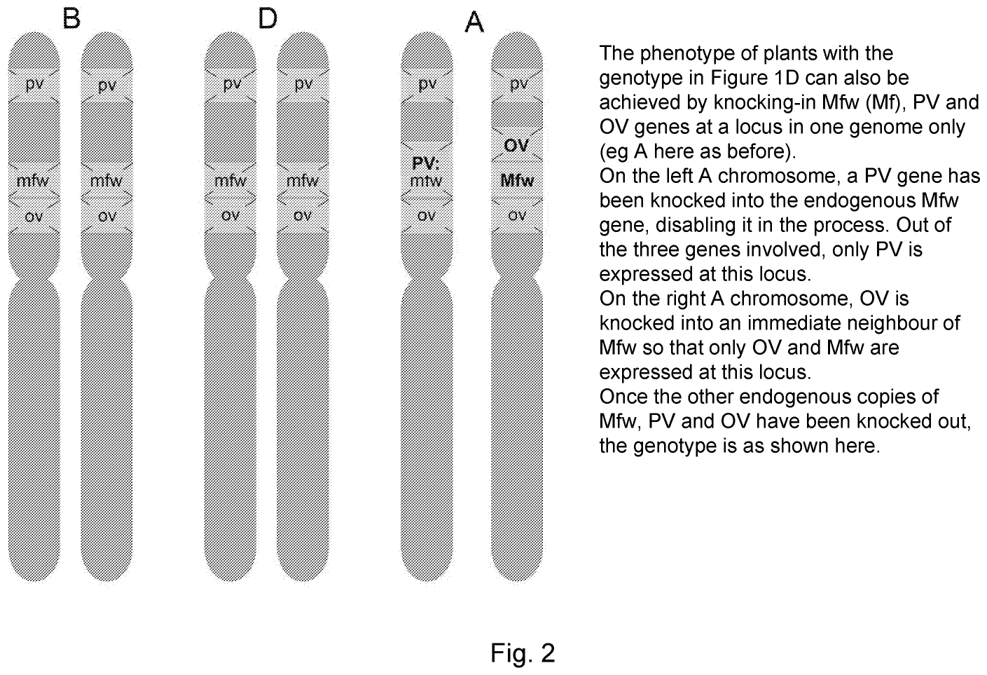

For illustrative purposes, FIG. 1 provides a schematic of the modifications described herein. As described below, Mf genes function largely pre-meiosis and therefore, the presence of the single Mf allele in the maintainer line's diploid, pre-meiosis reproductive cells will provide reproductive functionality for the Mf gene's activity, so the Mf allele carried by an individual pollen grain post-meiosis is not determinative of its viability. However, the PV gene (as described below) is post-meiosis in function, so each pollen grain carrying a pv allele will be non-viable. Thus, as shown the schematic, the pollen grains with a PV allele will be viable, while those with a pv allele are not viable. Due to the tight genetic linkage between the PV allele and the mf alleles in the first genome, the viable pollen grains also necessarily comprise a mf allele (e.g., all viable pollen is mf:PV:ov in the first genome). In the case of ovules, ovules with an OV construct will be viable (e.g., viable ovules are Mf:pv:OV). This means that self-fertilization will create progeny with the same genotype as the parent maintainer plant. If the maintainer plant is crossed with the cognate male-sterile plant, the resulting progeny will be more cognate male-sterile plants.

As used herein, “cognate” with respect to the maintainer line and it's phenotypic relative (e.g., a male-sterile line), refers to the two plants carrying recessive alleles of the same phenotype-controlling gene(s) of interest according to the schemes described herein. For example, a male-sterile plant which comprises only recessive non-functional alleles of a first Mf gene is not cognate with a maintainer line which carries recessive non-functional alleles of a second Mf gene. It is noted that the recessive alleles need not be identical in sequence in order for a maintainer and the phenotypic relative to be cognate.

It is noted that the Mf, PV, and OV loci may be in any 5′ to 3′ order and any recitation of the genes provided herein is not meant to limit the embodiments to a particular 5′ to 3′ order.

Further provided herein are male-fertile maintainer plants that do not require deletion of intergenic sequences, but still provide maintainer line technology without the introduction of exogenous sequences. In one aspect of any of the embodiments, described herein is a male-fertile maintainer plant for a male-sterile polyploid plant, the maintainer plant comprising:

a. an engineered knock-out modification at each allele of the Mf gene in every genome;

b. an engineered knock-out modification at each allele of the PV gene in every genome;

c. an engineered knock-out modification at each allele of the OV gene in every genome; and

d. an engineered modification in a first genome comprising:
i. at a loci on a first member of a homologous pair of chromosomes, an engineered insertion or knock-in of the PV gene; and
ii. at a loci on a second member of the homologous pair of chromosomes, an engineered insertion or knock-in of the Mf and OV genes;
wherein the loci of i and ii are homolgous, inter-genic regions and not coextensive with the alleles of a, b, or c.
   In one embodiment of any of the aspects, a maintainer plant can be provided without knocking-out a Mf gene, for example, described herein is a male-fertile maintainer plant for a male-sterile polyploid plant, the maintainer plant comprising:
   in the first chromosome of a homologous pair in a first genome:
a. an engineered knock-out modification at the allele of a pollen-grain-vital gene (PV gene);
b. an endogenous, wild-type functional allele of an ovule-vital gene (OV gene); and
c. at least one engineered modification comprising a deletion of endogenous intervening sequence between the PV; and OV loci;
   in the second chromosome of the same homologous pair in the first genome:
d. an endogenous, wild-type functional allele of the PV gene; and
e. an engineered knock-out modification at the allele of the OV gene;
f. at least one engineered modification comprising a deletion of endogenous intervening sequence between the PV; and OV loci;
   in a second and any subsequent genomes:
g. an engineered knock-out modification at each allele of the PV gene;
h. an engineered knock-out modification at each allele of the OV gene;
   whereby the pollen grains produced by the male-fertile maintainer plant comprise the second chromosome of the first genome and do not comprise the first chromosome of the first genome (hereinafter referred to as the pollen construct); and the ovules produced by the male-fertile maintainer plant comprise the first chromosome of the first genome and do not comprise the second chromosome of the first genome (hereinafter referred to as the minimal ovule construct). The foregoing modifications result in viable, germinating, pollen grains produced by the male-fertile maintainer plant comprising all the knockouts of PV, OV, and in some embodiments, Mf above and the knock-in of the PV gene (the other 50% of pollen grains without the PV gene will not be viable). (This is hereinafter referred to as the knock-in pollen construct); and the viable ovules produced by the male-fertile maintainer plant comprising all the knockouts of PV, OV, and in some embodiments, Mf above and the knock-in of the OV and in some embodiments, Mf genes (the other 50% of ovules without the OV gene will not be viable). (This, whether knocking-in the Mf and OV genes or the OV gene only, is hereinafter referred to as the knock-in ovule construct. When only the OV gene is knocked-in, the construct can be referred to as a “minimal ovule construct”. When both the OV and Mf gene are knocked-in, the construct can be referred to as a “two-gene ovule construct.”) In some embodiments of any of the aspects, the chromosomes of the homologous pair of chromosomes are different from the chromosomes comprising the endogenous/wild-type PV, OV, and in some embodiments, Mf alleles. In some embodiments of any of the aspects, the chromosomes comprising the knock-in modifications are the same as the chromosomes comprising the endogenous/wild-type PV, OV, and in some embodiments, Mf alleles. In some embodiments of any of the aspects, the endogenous/wild-type PV, OV, and in some embodiments, Mf alleles are found on the same chromosome. In some embodiments of any of the aspects, two alleles of the endogenous/wild-type PV, OV, and Mf alleles are found on the same chromosome, and the third allele is found on a different chromosome. In some embodiments of any of the aspects, those relating to knock-in constructs, the endogenous/wild-type PV, OV, and in some embodiments, Mf alleles are each found on a different chromosome, e.g., the alleles of endogenous/wild-type PV, OV, and in some embodiments, Mf are each found on a chromosome not comprising the other two alleles.

It is contemplated herein that the knock-out modifications knock-out the endogenous Mfw, OV, and/or PV allele. The knock-out modification can further comprise, or be followed by or preceded by, a knock-in of an engineered insertion, engineered construct, endogenous or exogenous allele. For example, a construct can be inserted into an endogenous wild-type Mfw allele using Cas-CRISPR technology, thereby knocking-out the endogenous wild-type Mfw allele and knocking in the construct (e.g. a construct comprising a wild-type PV or OV gene).

Further provided herein are other male-fertile maintainer plants that do not require deletion of intergenic sequences, but still provide maintainer line technology without the introduction of exogenous and/or foreign sequences. In such aspects of any of the embodiments, described herein is a male-fertile maintainer plant for a male-sterile polyploid plant comprising a first and further genomes, the maintainer plant comprising:
a. an engineered modification in the first genome comprising:
   i. at a loci on a first member of a homologous pair of chromosomes, an engineered insertion or knock-in of the PV gene; and
   ii. at a loci on a second member of the homologous pair of chromosomes, an engineered insertion or knock-in of the OV gene;
   wherein the loci of a. i and a. ii are homolgous, intra-genic or inter-genic regions and optionally, not coextensive with the alleles of c. or d. below,
b. an engineered knock-out modification at each allele of the endogenous PV gene in every genome; and
c. an engineered knock-out modification at each allele of the endogenous OV gene in every genome.
   In one aspect of any of the embodiments, described herein is a male-fertile maintainer plant for a male-sterile polyploid plant comprising a first and one or more further genomes, and modifications of a first and second gene, wherein the first and second genes are selected, in any order, from the group consisting of a PV gene, and an OV gene,

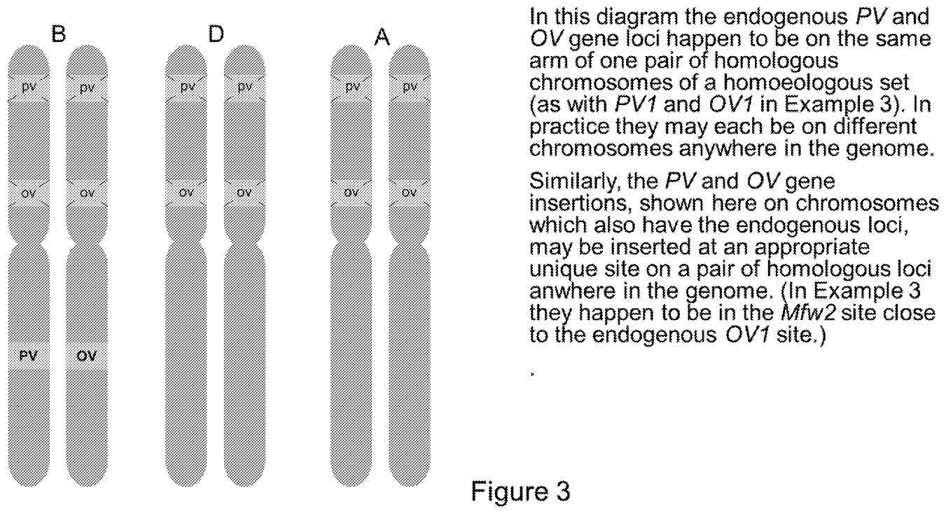

the modifications comprising:
a. an engineered knock-out modification at each allele of a first gene in the further genomes;
b. an engineered knock-out modification at each allele of a second gene in every genome; and
c. engineered modifications in the first genome comprising:
   i. an engineered knock-out modification of at least one allele of the first gene;
   ii. at a loci on a first member of a homologous pair of chromosomes, an engineered insertion or knock-in of the second gene;
      wherein at least one functional copy of the first gene is present in the first genome. The foregoing knock-in modifications can simultaneously comprise an engineered knock-out modification at each allele of one homologous pair only of a given gene (e.g., a Mf gene) in oe genome only (if an intra-genic loci, such as Mfw2 is used, it not being knocked out in the other genomes, the other copies of the polyploid's homoeologues will still express the relevant gene). The foregoing modifications result in viable, germinating, pollen grains produced by the male-fertile maintainer plant comprising all the knockouts of the PV and OV genes and the knock-in of the PV gene (the 50% of pollen grains without the PV gene will not be viable); and the viable ovules produced by the male-fertile maintainer plant comprising all the knockouts of the PV and OV genes and the knock-in of the OV gene (the 50% of ovules without the OV gene will not be viable). In some embodiments of any of the aspects, the alleles and/or loci of a, b, and c are found on the same chromosome. It is contemplated herein that alleles of the knockouts of the PV and OV genes may each be effected on any homoeologous set of chromosomes, alleles of the knockin inserts may be located at any location in the genome, e.g, in any one genome with an appropriately unique target site (see, e.g, FIG. 3). In some embodiments, the first genome comprises an engineered knock-out modification of both alleles of the first gene in the first genome and at a loci on a second member of the homologous pair of chromosomes an engineered insertion or knock-in of the first gene. In some embodiments, in the first genome the loci on the first member of a homologous pair of chromosomes is the loci of the first gene and the wild-type functional allele of the first gene is not modified on the second member of the homologous pair of chromosomes.

Approaches which do not require intergenic sequence deletion can also be applied to embodiments relating to plants comprising Mf, PV, and OV gene modifications. For example, in one aspect of any of the embodiments, described herein is a male-fertile maintainer plant for a male-sterile polyploid plant comprising:

a first and one or more further genomes, and modifications of a first, second, and third gene, wherein the first and second, and third genes are selected, in any order, from the group consisting of a Mf gene, a PV gene, and an OV gene, the modifications comprising:
a. an engineered knock-out modification at each allele of the first gene in the further genomes;
b. an engineered knock-out modification at each allele of the second gene in every genome;
c. an engineered knock-out modification at each allele of the third gene in every genome; and
d. engineered modifications in the first genome comprising:
   i. an engineered knock-out modification of at least one allele of the first gene;
   ii. at a loci on a first member of a homologous pair of chromosomes, an engineered insertion or knock-in of the second gene; and
   iii. at a loci on a second member of the homologous pair of chromosomes (which may be the same loci as in d.ii above), an engineered insertion or knock-in of the third gene;
      wherein at least one functional copy of the first gene is present in the first genome and in the first genome the one member of the homologous pair of chromosomes comprises a functional copy of the Mf and OV genes and the other member of the homologous pair of chromosomes comprises a functional copy of the PV gene. The knock-in modifications can comprise (e.g, simultaneously be, or create by their insertion), one or more of the knock-out modifications, e.g, the engineered insertion or knock-in of the second or third gene also comprises a knock-out modification of the first gene. Accordingly, one or more of the loci of the knock-in modifications can be the loci of the first gene, e.g, the knock-in modification is made at the intragenic sequence of one of the genes (e.g., the first gene). In some embodiments of any of the aspects, where an endogenous wild-type copy of the first gene is to be retained, rather than inserting a functional copy in a construct, the loci of d.iii. is located within the intergenic space separating the loci of the first gene from the adjacent genes or within one of the genes adjacent to the first gene.

In some embodiments of any of the aspects, the first gene and third genes are, in either order, the Mf and OV genes, the engineered modifications of d. comprise:
i. at the loci of the first gene on a first member of a homologous pair of chromosomes, an engineered insertion or knock-in of the second gene and an engineered knock-out of the first gene; and
ii. at the loci of the first gene, within the intergenic space separating the loci of the first gene from the adjacent genes, or within one of the adjacent genes, on a second member of the homologous pair of chromosomes, an engineered insertion or knock-in of the third gene and either:
   1. no modification of the first gene itself; or
   2. a knockout modification of the endogenous loci of the first gene and a knock-in or insertion of the first gene.
      In some embodiments of any of the aspects, wherein the first gene is the PV gene, the engineered modifications of d. comprise:
i. at the loci of the first gene on a first member of a homologous pair of chromosomes, an engineered insertion or knock-in of the second and third genes and an engineered knock-out of the first gene; and
ii. at the loci of the first gene, within the intergenic space separating the loci of the first gene from the adjacent genes, or within one of the adjacent genes, on a second member of the homologous pair of chromosomes either:
   1. no modification of the first gene itself; or
   2. a knockout modification of the endogenous loci of the first gene and a knock-in or insertion of the first gene.
      In some embodiments of any of the aspects, the plant comprises an engineered knock-out modification at each allele of the first gene in every genome and the engineered modifications of d. comprise:
i. at a loci on one member of a homologous pair of chromosomes, an engineered insertion or knock-in of the second gene; and
ii. at a loci on the other member of the homologous pair of chromosomes, an engineered insertion or knock-in of the third gene.

In one aspect of any of the embodiments, described herein is a male-fertile maintainer plant for a male-sterile polyploid plant comprising a first and one or more further genomes, the maintainer plant comprising:

a. an engineered knock-out modification at each allele of a PV gene in every genome;

b. an engineered knock-out modification at each allele of an OV gene in every genome; and

c. engineered modifications in the first genome comprising:
i. at a loci on a first member of a homologous pair of chromosomes, an engineered insertion or knock-in of the PV gene; and
ii. at a loci on a second member of the homologous pair of chromosomes, an engineered insertion or knock-in of the OV gene.
   In some embodiments, the plant further comprises an engineered knock-out modification at each allele of a Mf gene in every genome. In some embodiments, the modification of c.ii. father comprises an engineered insertion or knock-in of the OV gene and Mf gene.

In one aspect of any of the embodiments, described herein is a male-fertile maintainer plant for a male-sterile polyploid plant comprising a first and one or more further genomes, the maintainer plant comprising:

a. an engineered knock-out modification at each allele of a Mf gene in the further genomes;

b. an engineered knock-out modification at each allele of a PV gene in every genome;

c. an engineered knock-out modification at each allele of an OV gene in every genome; and

d. engineered modifications in the first genome comprising:
i. an engineered knock-out modification of at least one allele of the Mf gene;
ii. at a loci on a first member of a homologous pair of chromosomes, an engineered insertion or knock-in of the PV gene; and
iii. at a loci on a second member of the homologous pair of chromosomes, an engineered insertion or knock-in of the OV;
   wherein at least one functional copy of the Mf gene is present in the first genome. In some embodiments, the engineered insertion or knock-in of the PV gene also comprises a knock-out modification of the Mf gene. In some embodiments, the loci on the first member of the pair of chromosomes is located within the intergenic space separating the Mf loci from the adjacent genes or within one of the adjacent genes. In some embodiments, the engineered modifications of d. comprise:
i. at the Mf loci on a first member of a homologous pair of chromosomes, an engineered insertion or knock-in of the PV gene and an engineered knock-out of the Mf gene; and
ii. at the Mf loci, within the intergenic space separating the Mf loci from the adjacent genes, or within one of the adjacent genes, on a second member of the homologous pair of chromosomes, an engineered insertion or knock-in of the OV gene and either:
   1. no modification of the Mf gene itself; or
   2. a knockout modification of the endogenous Mf loci and a knock-in or insertion of the Mf gene.
      In some embodiments, the plant comprises an engineered knock-out modification at each allele of the Mf gene in every genome and the engineered modifications of d. comprise:
i. at a loci on a first member of a homologous pair of chromosomes, an engineered insertion or knock-in of the PV gene; and
ii. at a loci on a second member of the homologous pair of chromosomes, an engineered insertion or knock-in of the OV gene.

The methods and compositions described herein are particularly applicable to polyploidal plants. In some embodiments of any of the aspects, the male-fertile maintainer plant is hexaploid and the male-sterile plant comprises an engineered knock-out modification at each of the six alleles of the male-fertility gene (e.g., the Mf gene). In some embodiments of any of the aspects, the male-fertile maintainer plant is tetraploid and the male-sterile plant comprises an engineered knock-out modification at each of the four alleles of the male-fertility gene (e.g., the Mf gene). In some embodiments of any of the aspects, the male-sterile plant comprises an engineered knock-out modification at each allele of the Mf gene.

In some embodiments of any of the aspects, a male-sterile line may comprise knock-out and/or non-functional alleles of two or more Mf genes, e.g., due to redundancy and/or leaky phenotypes. In such embodiments, the maintainer line will comprise the same arrangement of Mf alleles described herein, but for both Mf genes, e.g. the pollen and ovule constructs will become 4-gene constructs instead of 3-gene constructs or comprises an engineered knock-out modification at each allele of each Mf gene in every genome.

As described elsewhere herein, the instant methods and compositions do not require the introduction of transgenic or exogenous sequences. Accordingly, in some embodiments of any of the aspects, the maintainer plant does not comprise any genetic sequences which are exogenous to that plant species. In some embodiments of any of the aspects, the maintainer plant does not comprise any genetic sequences which are ectopic to that plant species. In some embodiments of any of the aspects, the maintainer plant, like its male-sterile pair, is not transgenic.

In some embodiments of any of the aspects, the maintainer plant is substantially isogenic with the male-sterile plant with the exception of the engineered modifications in the first genome. In some embodiments of any of the aspects, the maintainer plant is substantially isogenic with the male-sterile plant with the exception of the engineered modifications of the ovule construct. In some embodiments of any of the aspects, the maintainer plant is substantially isogenic with the male-sterile plant with the exception of the engineered modifications of the knock-in pollen construct in the first genome.

It is noted that the methods and compositions described herein provide surprising advantages over existing approaches to cytoplasmic male-sterility. A major problem with cytoplasmic male-sterility is that one needs to breed the final ‘male’ pollinator-line, used to produce the F1 seed, to comprise a ‘restorer’ gene(s) to overcome the male-sterility of the ‘female line’ so that the customer's commercial crop has full fertility. In the systems described herein, the male-sterility is recessive so any cultivar other than the male-sterile cultivar and its maintainer will act as a restorer. This means that production of hybrid seed can be conducted normally by crossing the male-sterile line and a different cultivar of choice without the use of a particular restorer line.

Alternatively, the technology described herein can be used to improve such cytoplasmic male-sterility approaches. With cytoplasmic male-sterility, not only is necessary to ‘breed in’ a restorer for the final pollinator but, this restorer production is complicated by the fact that there can be more than one restorer gene required to effect full fertility-restoration; then these segregate independently requiring larger populations and making the whole process more difficult and expensive. Using two such restorer genes on the same chromosome arm, in conjunction with the techniques to decrease genetic linkage provided herein, can improve the efficiency of such systems.

The engineered modifications described herein can be generated by any method known in the art, e.g., by homolgous recombination-mediated mutagenesis, random mutagenesis, or by using a site-specific guided nuclease. In some embodiments of any of the aspects, at least one copy of any of the engineered modifications is engineered by using a site-specific guided nuclease. In some embodiments of any of the aspects, the engineered modifications are engineered by using a site-specific guided nuclease.

Various site-specific guided nucleases are known in the art and can include, by way of non-limiting example, transcription activator-like effector nucleases (TALENs), oligonucleotides, meganucleases, and zinc-finger nucleases. Toolkits and services for zinc-finger nuclease mutagenesis are commercially available, for example EXZACT™ Precision Technology, marketed by Dow AgroSciences.

In some embodiments of any of the aspects, the site-specific guided nuclease is a CRISPR-associated (Cas) system such as CRISPR-Cas9 (e.g., Cas9, a Cas9-derived nickase, or a Cas9 homolog (e.g., Cpf1)). CRISPR is an acronym for clustered regularly interspaced short palindromic repeats. Briefly, in order for a Cas nuclease (or related nuclease) to recognize and cleave a target nucleic acid molecule, a CRISPR RNA (crRNA) and trans-activating crRNA (tracrRNA) must be present. crRNAs hybridize with tracrRNA to form a guide RNA (sgRNA) which then associates with the Cas nuclease. Alternatively, the sgRNA can be provided as a single contiguous sgRNA. Once the sgRNA is complexed with Cas, the complex can bind to a target nucleic acid molecule. The sgRNA binds specifically to a complementary target sequence via a target-specific sequence in the crRNA portion (e.g., the spacer sequence), while Cas itself binds to a protospacer adjacent motif (CRISPR/Cas protospacer-adjacent motif; PAM). The Cas nuclease then mediates cleavage of the target nucleic acid to create a double-stranded break within the sequence bound by the sgRNA. Deletions can be generated by, e.g., using the nuclease to cut a genome at two specific locations targeted with two sgRNAs each specific to one of the two locations concerned, thereby excising the sequence between the two double-strand breaks. CRISPR-Cas technology for editing of plant genomes is fully described in Belhaj et al. (2015). This is a practicable, convenient and flexible method of gene editing. It has been shown to work well in plants, see for example in Belhaj et al. (2015); Wang et al. (2014; Nature Biotechnology 32:947-951); and Shan et al. (2014). The latter paper gives full protocols to enable the system to be applied to modify plant genomes (including wheat) as desired.

As described herein, an engineered modification can be introduced by utilizing the CRISPR/Cas system. In some embodiments of any of the aspects, the site-specific guided nuclease is a form of CRISPR-Cas, e.g., CRISPR-Cas9. In some embodiments of any of the aspects, the engineered modifications are created using a site-specific guided nuclease and a multi-guide construct.

In some embodiments of any of the aspects, a plant or plant cell described herein can further comprise an exogenous or introduced endonuclease or a nucleic acid encoding such an endonuclease (e.g., Cas9, a Cas9-derived nickase, or a Cas9 homolog (e.g., Cpf1)). In some embodiments of any of the aspects, a plant or seed as described herein can further comprise a CRISPR RNA sequence designed to target an endonuclease to the gene, e.g. (a crRNA and trans-activating crRNA (tracrRNA) and/or a guide RNA (sgRNA)). In some embodiments of any of the aspects, the sgRNA is provided as a single continuous nucleic acid molecule. In some embodiments of any of the aspects, the sgRNA is provided as a set of hybridized molecules, e.g., a crRNA and tracrRNA. In some embodiments of any of the aspects, the sgRNA is provided as a DNA molecule encoding a sgRNA and/or a crRNA and tracrRNA. Design of sgRNAs, crRNAs, and tracrRNAs are known in the art and described elsewhere herein. Exemplary sgRNA sequences are provided elsewhere herein. In some embodiments of any of the aspects, a multi-guide construct is provided, e.g., multiple sgRNA are provided in a single construct and/or nucleic acid molecule such that multiple target sequences are cleaved in the presence of a Cas enzyme and the multi-guide construct.

As used herein, “target sequence” within the context of a site-specific guided nuclease refers to a sequence in the relevant genome which is to be used to specify where the nuclease will generate a break or nick in the genome at a desired location. In the case of Cas (and related) nucleases, the guide RNA is designed to specifically hybridize to the target sequence, or in the case of multi-guide constructs, multiple guide RNAs are provided, each of which specifically hybrizes to a target sequence. Target sequences can be identified using the publicly available program DREG (available on the world wide web at emboss.sourceforge.net/apps/cvs/emboss/apps/dreg.html) to find sequences that match either ANNNNNNNNNNNNNNNNNNNNGG or GNNNNNNNNNNNNNNNNNNNNGG in both directions of the genomic sequence. As an illustrative example, guides can be selected from the results based on the following criteria: that the target sequence is conserved in all homoeologues which are to be modified, that it has a restriction enzyme site near the site of the protospacer associated motif (PAM) but in the sequence of the guide RNA and finally, prioritizing guides near the start of the coding sequences of each gene. An additional consideration can be to select sequences with either AN20GG and GN20GG as this stabilizes the construct for transformation in the plant.

By way of non-limiting example, exemplary guide sequences for generating the deletions between two genes (e.g., two of an OV, PV, and/or Mfw gene) are described in Example 2 herein.

Guide sequence expression can be driven by individual and/or shared promoters. Exemplary promoters include OsU3, TaU3, TaU6 and OsU6 promoters. Guide constructs, expressing one or more sgRNA sequences, can be cloned into a vector suitable for expressing the sgRNAs in the plant, e.g., a binary vector containing a wheat-optimized Cas9 enzyme driven by the rice actin promoter can be used in wheat. Vectors can be introduced into the plant or plant cell by any means known in the art, e.g. by Agrobacterium. Alternatively, the sgRNAs can be expressed in vitro and introduced into cells by, e.g., microinjection.

Cas9 and sgRNA sequences can be expressed either stably or transiently in a cell in order to generate the engineered modifications described herein. In one aspect of any of the embodiments, described herein is a plant cell comprising 1) an exogenous Cas9 protein and/or an exogenous nucleic acid encoding a Cas9 protein: and 2) at least one sgRNA capable of specifically hybridizing with at least one target sequence of a gene described herein under cellular conditions or a nucleic acid encoding such an sgRNA. In some embodiments of any of the aspects, the 1) exogenous nucleic acid encoding a Cas9 protein: and 2) the nucleic acid encoding at least one sgRNA capable of specifically hybridizing with the target sequence(s) under cellular conditions are provided in a vector or vector(s). In some embodiments of any of the aspects, the vectors are transient expression vectors. In some embodiments of any of the aspects, the 1) exogenous nucleic acid encoding a Cas9 protein: and 2) the nucleic acid encoding at least one sgRNA are integrated into the genome. It is contemplated herein that similar approaches to vector delivery, transient expression, and/or stable integration can also be utilized in embodiments relating to, e.g., inhibitory RNAs, TALENs, and/or ZFNs.

The Cas enzyme and guide sequences can be provided in non-integrating vectors, e.g., to avoid incorporation of these sequences in the genome of the plant.

In one aspect of any of the embodiments, described herein is a nucleic acid encoding at least one sgRNA capable of specifically hybridizing with at least one gene sequence described herein, e.g., under cellular conditions. In one aspect of any of the embodiments, described herein is a nucleic acid encoding at least one sgRNA capable of targeting Cas9 or a related endonuclease to at least one gene described herein, e.g., under cellular conditions. In some embodiments of any of the aspects, the nucleic acid further encodes a Cas9 protein. In some embodiments of any of the aspects, the nucleic acid is provided in a vector. In some embodiments of any of the aspects, the vector is a transient expression vector.

Following contact with a site-specific nuclease, e.g., a Cas (or related) enzyme and at least one guide RNA, plants can be screened for deactivating modifications, e.g., utilizing a PCR based method where the PCR product is digested with an appropriate enzyme previously identified to cut the DNA at a site near the PAM. PCR products which are not cut therefore contain a modification induced by the CRISPR construct.

In alternative embodiments, an engineered modification can be introduced by utilizing TALENs or ZFN technology, which are known in the art. Methods of engineering nucleases to achieve a desired sequence specificity are known in the art and are described, e.g., in Kim (2014); Kim (2012); Belhaj et al. (2013); Urnov et al. (2010); Bogdanove et al. (2011); Jinek et al. (2012) Silva et al. (2011); Ran et al. (2013); Carlson et al. (2012); Guerts et al. (2009); Taksu et al. (2010); and Watanabe et al. (2012); each of which is incorporated by reference herein in its entirety.

In some embodiments of any of the aspects, the endogenous Mf, PV, and OV genes are located on the same arms of the same homologous pair of chromosomes in the wild-type genome.

In some embodiments of any of the aspects, modifications comprising the knock-in pollen or ovule constructs can be introduced using any of homolgous recombination-mediated mutagenesis, random mutagenesis, or site-specific guided nuclease methods described elsewhere herein, combined with providing one or more template nucleic acids comprising the pollen or ovule construct to be introduced. The template nucleic acids can comprise one or more regions of homology to the target loci in the first genome to direct their introduction at the target loci. Such technologies, and the design of such constructs are known in the art.

In some embodiments of any of the aspects, knock-in modifications comprise wild-type or functional alleles of the relevant gene(s). Exemplary wild-type and functional alleles of exemplary Mf, OV, and PV genes are provided herein, or can be a naturally-occurring Mf, OV, or PV allele in a fertile plant. In some embodiments of any of the aspects, one or more knock-in modifications can comprise gDNA constructs derived from wild-type or functional alleles of the relevant gene(s) (e.g., introns are present). In some embodiments of any of the aspects, one or more knock-in modifications can comprise cDNA constructs derived from wild-type or functional alleles of the relevant gene(s) (e.g., introns are not present). In some embodiments of any of the aspects, knock-in modifications can comprise endogenous promoters and/or terminators in the normal sense orientation. In some embodiments of any of the aspects, the sequence which is introduced by a knock-in modification of a gene itself does not comprise any sequence which is foreign or exogenous to the knocked-in gene in a wild-type genome of the same or a crossable species, although the knock-in sequence may comprise deletions of endogenous sequence relative to a wild-type gene sequence (e.g., deletion of introns). By way of example, the genomic region of PV1 is about 5 kb, when including 1.5 kb of a promoter sequences and about 500 bp for a terminator sequence. With targeting regions flanking this 5 kb sequence (e.g., Mfw2 targeting regions for the approach illustrated in Example 3), the total construct size is approximately 6.5 to 7 kb, which is of suitable size for knock-in constructs as described herein. For OV1, a similar construct results in a knock-in construct of approximately 9 to 10 kb, which is also within acceptable size limits for the delivery systems described in Example 3.

In some embodiments of any of the aspects, the plant is polyploidal, e.g., tetraploid or hexaploid. In some embodiments of any of the aspects, the plant is wheat, e.g., hexaploid wheat, tetraploid wheat, Triticum aestivum, or Triticum durum. In some embodiments of any of the aspects, the plant is triticale, oat, canola/oilseed rape or indian mustard. In some embodiments of any of the aspects, the plant is an elite breeding line.

As used herein, a gene or Mf (for “male fertility) gene is a gene which, when its expression is inhibited, decreases male-fertility and which functions pre-meiosis. Mf genes can be specific for male-fertility, rather than female-fertility. In some embodiments of any of the aspects, a Mf gene, when fully deactivated in a plant, is sufficient to render the plant male-sterile, e.g., the Mf gene is strictly necessary for male-fertility. In some embodiments of any of the aspects, the Mf gene is a gene which has been identified to produce a male-sterile phenotype when a plant was modified to comprise knock-out alleles for that gene. In some embodiments of any of the aspects, the Mf gene is pre-meiotic, e.g., it functions before meiosis. “Mfw” is used at times herein interchangeably with “Mf” and may refer to wheat Mf genes, e.g., as in the Figures where the wheat genome is used as an illustrative embodiment. Where “Mfw” is used, one of skill in the art will understand that those embodiments are equally applicable in other plant species using suitable Mf genes for that species.

Mf genes for various species have been described in the art, and exemplary, but non-limiting, Mf genes include those described in International Patent Application PCT/US2017/043009 (referred to therein as Mpew or Mfw genes), as well as the Ms genes (e.g., Ms1, Ms26, and Ms45) described in Wang et al. PNAS 2017; Singh et al. PloS One 12(5) e0177632 (2017); Timofejva et al. G3: Genes-Genomes-Genetc 3:231-249 (2013); and Wu et al. Plant Biotechnology Journal 14:1046-1054 (2015); each of which is incorporated by reference herein in its entirety. In some embodiments of any of the aspects, the Mf gene is a gene which displays the same type of activity, and/or shares at least 80%, at least 85%, at least 90%, at least 95%, or greater sequence identity with a Mf gene of any of the foregoing references. In some embodiments of any of the aspects, a Mf gene can be the gene from a species, cultivar, or variety which has the highest degree of homology and/or sequence identity of the genes in that species', cultivar's or variety's genome with a gene selected from one of the foregoing references. In some embodiments of any of the aspects, a Mf gene can be the gene from a species, cultivar, or variety which has the greatest degree of homology and/or sequence identity of the genes in that species', cultivar's or variety's genome with a gene selected from one of the foregoing references.

A non-limiting list of exemplary pre-meiosis Mf genes is provided in Table 3. In some embodiments of any of the aspects, the Mf gene is a gene selected from Table 3. In some embodiments of any of the aspects, the Mf gene is a gene which displays the same type of activity, and/or shares at least 80%, at least 85%, at least 90%, at least 95%, or greater sequence identity with a Mf gene of Table 3. In some embodiments of any of the aspects, a Mf gene can be the gene from a species, cultivar, or variety which has the highest degree of homology and/or sequence identity of the genes in that species', cultivar's or variety's genome with a gene selected from Table 3. In some embodiments of any of the aspects, a Mf gene can be the gene from a species, cultivar, or variety which has the greatest degree of homology and/or sequence identity of the genes in that species', cultivar's or variety's genome with a gene selected from Table 3.

In some embodiments of any of the aspects, the Mf gene is a gene selected from Table 3 or 5. In some embodiments of any of the aspects, the Mf gene is a gene which displays the same type of activity, and/or shares at least 80%, at least 85%, at least 90%, at least 95%, or greater sequence identity with a Mf gene of Table 3 or 5. In some embodiments of any of the aspects, a Mf gene can be the gene from a species, cultivar, or variety which has the highest degree of homology and/or sequence identity of the genes in that species', cultivar's or variety's genome with a gene selected from Table 3 or 5. In some embodiments of any of the aspects, a Mf gene can be the gene from a species, cultivar, or variety which has the greatest degree of homology and/or sequence identity of the genes in that species', cultivar's or variety's genome with a gene selected from Table 3 or 5.

TABLE 3
Exemplary pre-meiotic Mf genes

| | | | TGAC v1 homoeologues*- | |
|---|---|---|---|---|
| Assigned | | | the copies on the other sub | |
| Mfw | | | genomes of wheat and their | |
| name | Blast hit | TGAC v1 gene model* | associated gene models | Reference Sequences |
| Mfw2-A | callose | TRIAE_CS42_7AS_TGACv1_ | TRIAE_CS42_7BS_TGACv1_ | |
| | synthase 5 | 569258_AA1811650 | 593715_AA1953990; | |
| | | | TRIAE_CS42_7DS_TGACv1_ | |
| | | | 622598_AA2042310 | |
| Mfw2-B | callose | TRIAE_CS42_7BS_TGACv1_ | TRIAE_CS42_7AS_TGACv1_ | |
| | synthase 5 | 593715_AA1953990 | 569258_AA1811650; | |
| | | | TRIAE_CS42_7DS_TGACv1_ | |
| | | | 622598_AA2042310 | |
| ″ | callose | TRIAE_CS42_7BS_TGACv1_ | TRIAE_CS42_7AS_TGACv1_ | |
| | synthase 5 | 593715_AA1953990 | 569258_AA1811650; | |
| | | | TRIAE_CS42_7DS_TGACv1_ | |
| | | | 622598_AA2042310 | |
| ″ | callose | TRIAE_CS42_7BS_TGACv1_ | TRIAE_CS42_7AS_TGACv1_ | |
| | synthase 5 | 593715_AA1953990 | 569258_AA1811650; | |
| | | | TRIAE_CS42_7DS_TGACv1_ | |
| | | | 622598_AA2042310 | |
| ″ | callose | TRIAE_CS42_7BS_TGACv1_ | TRIAE_CS42_7AS_TGACv1_ | |
| | synthase 5 | 593715_AA1953990 | 569258_AA1811650; | |
| | | | TRIAE_CS42_7DS_TGACv1_ | |
| | | | 622598_AA2042310 | |
| Mfw2-D | callose | TRIAE_CS42_7DS_TGACv1_ | TRIAE_CS42_7BS_TGACv1_ | |
| | synthase 5 | 622598_AA2042310 | 593715_AA1953990; | |
| | | | TRIAE_CS42_7AS_TGACv1_ | |
| | | | 569258_AA1811650 | |
| Mfw3-A | Aborted | TRIAE_CS42_6AS_TGACv1_ | TRIAE_CS42_6BS_TGACv1_ | |
| | microspore 1 | 486918_AA1566480 | 514404_AA1659330; | |
| | like | | TRIAE_CS42_U_TGACv1_6 | |
| | | | 43846_AA2135420 | |
| Mfw3-B | Aborted | TRIAE_CS42_6BS_TGACv1_ | TRIAE_CS42_6AS_TGACv1_ | |
| | microspore 1 | 514404_AA1659330 | _486918_AA1566480; | |
| | like | | TRIAE_CS42_U_TGACv1_ | |
| | | | 643846_AA2135420 | |
| Mfw3-D | Aborted | TRIAE_CS42_U_TGACv1_ | TRIAE_CS42_6AS_TGACv1_ | |
| | microspore 1 | 643846_AA2135420 | 486918_AA1566480; | |
| | like | | TRIAE_CS42_6BS_TGACv1_ | |
| | | | 514404_AA1659330 | |
| Mfw9-B | member of | TRIAE_CS42_2DS_TGACv1_ | TRIAE_CS42_2AS_TGACv1_ | |
| | the sweet | 177708_AA0582810 | 113352_AA0354890; | |
| | family | | TRIAE_CS42_2BS_TGACv1_ | |
| | | | 149844_AA0497680 | |
| Mfw10-A | member of | TRIAE_CS42_7AS_TGACv1_ | TRIAE_CS42_7BS_TGACv1_ | |
| | the sweet | 570345_AA1834200 | 591914_AA1925470 | |
| | family | | | |
| Mfw11-B | Similar to | TRIAE_CS42_U_TGACv1_ | no strong hit | |
| | OsSweet7e | 640821_AA2075730 | | |
| Mfw12-D | Sweet4 | TRIAE_CS42_1DL_TGACv1_ | TRIAE_CS42_1AL_TGACv1_ | |
| | | 065128_AA0236610 | 002319_AA0040790; | |
| | | | TRIAE_CS42_1BL_TGACv1_ | |
| | | | 030610_AA0095680 | |
| Ms8 | | | | See Wu et al. Plant |
| | | | | Biotechnology Journal |
| | | | | 14: 1046-1054 (2015) |
| Ms32 | | | | See Wu et al. Plant |
| | | | | Biotechnology Journal |
| | | | | 14: 1046-1054 (2015) |
| Ocl14 | | | | See Wu et al. Plant |
| | | | | Biotechnology Journal |
| | | | | 14: 1046-1054 (2015) |
| Mac1 | | | | See Wu et al. Plant |
| | | | | Biotechnology Journal |
| | | | | 14: 1046-1054 (2015) |
| Ms22 | | | | See Wu et al. Plant |
| | | | | Biotechnology Journal |
| | | | | 14: 1046-1054 (2015) |
| Ms23 | | | | See Wu et al. Plant |
| | | | | Biotechnology Journal |
| | | | | 14: 1046-1054 (2015) |

TABLE 5
Exemplary male fertility genes

| | | | |
|---|---|---|---|
| Mfw5-A | bHLH91 | TRIAE_CS42_2AL_TGACv1_ | TRIAE_CS42_2BL_TGACv1_ |
| | | 094707_AA0301850 | 129925_AA0399500; |
| | | | TRIAE_CS42_2DL_TGACv1_ |
| | | | 158620_AA0523420 |
| Mfw5-B | bHLH91 | TRIAE_CS42_2BL_TGACv1_ | TRIAE_CS42_2AL_TGACv1_ |
| | | 129925_AA0399500 | 094707_AA0301850; |
| | | | TRIAE_CS42_2DL_TGACv1_ |
| | | | 158620_AA0523420 |
| Mfw5-D | bHLH91 | TRIAE_CS42_2DL_TGACv1_ | TRIAE_CS42_2AL_TGACv1_ |
| | | 158620_AA0523420 | 094707_AA0301850; |
| | | | TRIAE_CS42_2BL_TGACv1_ |
| | | | 129925_AA0399500 |
| Mfw6-A | GAMYB | TRIAE_CS42_6AS_TGACv1_ | TRIAE_CS42_6DS_TGACv1_ |
| | (AtMYB101) | 485682_AA1550030 | 543879_AA1744870 |
| ″ | GAMYB | TRIAE_CS42_6AS_TGACv1_ | TRIAE_CS42_6DS_TGACv1_ |
| | (AtMYB101) | 485682_AA1550030 | 543879_AA1744870 |
| Mfw6-D | GAMYB | TRIAE_CS42_6DS_TGACv1_ | TRIAE_CS42_6AS_TGACv1_ |
| | (AtMYB101) | 543879_AA1744870 | 485682_AA1550030 |
| ″ | GAMYB | TRIAE_CS42_6DS_TGACv1_ | TRIAE_CS42_6AS_TGACv1_ |
| | (AtMYB101) | 543879_AA1744870 | 485682_AA1550030 |
| Mfw7-B | Hothead | TRIAE_CS42_4BL_TGACv1_ | TRIAE_CS42_4DL_TGACv1_ |
| | | 320326_AA1035360 | 343496_AA1135340; |
| | | | TRIAE_CS42_5AL_TGACv1_ |
| | | | 375593_AA1224180 |
| ″ | ″ | TRIAE_CS42_4BL_TGACv1_ | TRIAE_CS42_4DL_TGACv1_ |
| | | 320326_AA1035360 | 343496_AA1135340; |
| | | | TRIAE_CS42_5AL_TGACv1_ |
| | | | 375593_AA1224180 |
| Mfw7-D | Hothead | TRIAE_CS42_4DL_TGACv1_ | TRIAE_CS42_4BL_TGACv1_ |
| | | 343496_AA1135340 | 320326_AA1035360; |
| | | | TRIAE_CS42_5AL_TGACv1_ |
| | | | 375593_AA1224180 |
| Mfw8-D | Hothead | TRIAE_CS42_6DL_TGACv1_ | TRIAE_CS42_6AL_TGACv1_ |
| | | 527115_AA1698830 | 470984_AA1500160; |
| | | | TRIAE_CS42_6BL_TGACv1 |
| | | | 500863_AA1610910 |
| Mfw13-D | Hothead | TRIAE_CS42_1DL_TGACv1_ | TRIAE_CS42_1AL_TGACv1_ |
| | | 063432_AA0227210 | 001690_AA0034080; |
| | | | TRIAE_CS42_1BL_TGACv1_ |
| | | | 032570_AA0131570 |

As used herein, a pollen-vital gene or PV gene is a gene which, when its expression is inhibited, decreases the rate and/or success of pollen development and which functions post-meiosis. In some embodiments of any of the aspects, a PV gene, when fully deactivated in a plant, is sufficient to eliminate development of mature pollen, e.g., the PV gene is strictly necessary for pollen development. PV genes for various species have been described in the art, and exemplary, but non-limiting PV genes include those described in Golovkin and Redd et al PNAS 100(18) 10558-10563 (2003), which is incorporated by reference herein in its entirety. In some embodiments of any of the aspects, the PV gene is a gene which has been identified to produce a pollen-death phenotype when a plant was modified to a knock-out for that gene.

In some embodiments of any of the aspects, the PV gene is PV1, or pollen-grain—vital gene 1. Genomic, coding, and polypeptide sequences for the three homoeologues of PV1 occurring in the Chinese Spring genome are provided herein as SEQ ID Nos. 1-9. An PV1 gene or sequence can be a naturally-occurring PV1 gene or sequence occurring in a plant, e.g., wheat. In some embodiments of any of the aspects, an PV1 gene is a gene which displays the same type of activity, and/or shares at least 80%, at least 85%, at least 90%, at least 95%, or greater sequence identity with an PV1 gene of a sequence provided herein. In some embodiments of any of the aspects, a PV1 gene can be the gene from a species, cultivar, or variety which has the highest degree of homology and/or sequence identity of the genes in that species', cultivar's or variety's genome with an PV1 sequence provided herein.

The PV gene selected for use in the compositions and methods described herein can, e.g., have homology to a gene demonstrated to be vital for post-meiosis events such as pollen-grain development, germination, or pollen tube extension in a plant. A non-limiting list of exemplary PV genes is provided in Table 1. In some embodiments of any of the aspects, the PV gene is a gene selected from Table 1. In some embodiments of any of the aspects, the PV gene is a gene which displays the same type of activity, and/or shares at least 80%, at least 85%, at least 90%, at least 95%, or greater sequence identity with a PV gene of Table 1. In some embodiments of any of the aspects, a PV gene can be the gene from a species, cultivar, or variety which has the highest degree of homology and/or sequence identity of the genes in that species', cultivar's or variety's genome with a gene selected from Table 1. In some embodiments of any of the aspects, a PV gene can be the gene from a species, cultivar, or variety which has the greatest degree of homology and/or sequence identity of the genes in that species', cultivar's or variety's genome with a gene selected from Table 1.

TABLE 1
Exemplary PV genes

| | | | TGAC v1 homoeologues*- | |
|---|---|---|---|---|
| Assigned | | | the copies on the other sub | |
| Mfw | | | genomes of wheat and their | |
| name | Blast hit | TGAC v1 gene model* | associated gene models | Reference Sequences |
| Mfw1-A | RPG1 | TRIAE_CS42_7AL_TGACv1_ | TRIAE_CS42_7BL_TGACv1_ | |
| | (RUPTURED | 556969_AA1774370 | 580455_AA1914070; | |
| | POLLEN | | TRIAE_CS42_7DL_TGACv1_ | |
| | GRAIN1) | | 603435_AA1983700 | |
| | like | | | |
| Mfw1-B | RPG1 | TRIAE_CS42_7BL_TGACv1_ | TRIAE_CS42_7AL_TGACv1_ | |
| | (RUPTURED | 580455_AA1914070 | 556969_AA1774370; | |
| | POLLEN | | TRIAE_CS42_7DL_TGACv1_ | |
| | GRAIN1) | | 603435_AA1983700 | |
| | like | | | |
| Mfw1-D | RPG1 | TRIAE_CS42_7DL_TGACv1_ | TRIAE_CS42_7AL_TGACv1_ | |
| | (RUPTURED | 603435_AA1983700 | 556969_AA1774370; | |
| | POLLEN | | TRIAE_CS42_7BL_TGACv1_ | |
| | GRAIN1) | | 580455_AA1914070 | |
| | like | | | |
| Mfw4-D | RPG1 | TRIAE_CS42_5BS_TGACv1_ | TRIAE_CS42_5AS_TGACv1_ | |
| | (RUPTURED | 423307_AA1373980; | 393366_AA1271880; | |
| | POLLEN | | TRIAE_CS42_5DS_TGACv1_ | |
| | GRAIN1) like | | 457788_AA1489840 | |
| Ms26 | | TRIAE_CS42_4AS_TGACv1_ | | SEQ ID Nos: 36-44 |
| | | 308399_AA1027760 | | |
| | | TRIAE_CS42_4BL_TGACv1_ | | |
| | | 321123_AA1055760 | | |
| | | TRIAE_CS42_4DL_TGACv1_ | | |
| | | 345634_AA1154040 | | |
| Ms45 | | TRIAE_CS42_4AS_TGACv1_ | | SEQ ID Nos: 45-53 |
| | | 307709_AA1022920 | | |
| | | TRIAE_CS42_4BL_TGACv1_ | | |
| | | 320775_AA1048430 | | |
| | | TRIAE_CS42_4DL_TGACv1_ | | |
| | | 343561_AA1136570 | | |
| RPG1 | | | | NC_003076.8 |
| Ms1 | | | | SEQ ID Nos: 27-35 |
| | | | | See also Tucker et al. |
| | | | | Nature Communications |
| | | | | 2017 8: 869; which is |
| | | | | incorporated by |
| | | | | reference herein in its |
| | | | | entirety |
| PV1 | | | | SEQ ID Nos. 1-9 |
| (NPG1) | | | | |
| Apv1 | | | | See, e.g., Wu et al. Plant |
| | | | | Biotechnology Journal |
| | | | | 14: 1046-1054 (2015); |
| | | | | which is incorporated by |
| | | | | reference herein in its |
| | | | | entirety |
| Ipe1 | | | | See, e.g., Wu et al. Plant |
| | | | | Biotechnology Journal |
| | | | | 14: 1046-1054 (2015); |
| | | | | which is incorporated by |
| | | | | reference herein in its |
| | | | | entirety |

As used herein, an ovule-vital gene or OV gene is a gene which, when its expression is inhibited, decreases the rate and/or success of ovule development. In some embodiments of any of the aspects, an OV gene, when fully deactivated in a plant, is sufficient to eliminate development of mature ovules, e.g., the OV gene is strictly necessary for ovule development. OV genes for various species have been described in the art. In some embodiments of any of the aspects, the OV gene is a gene which has been identified to produce an ovule-death phenotype when a plant was modified to a knock-out for that gene.

In some embodiments of any of the aspects, the OV gene is OV1, or ovule-vital gene 1. Genomic, coding, and polypeptide sequences for the three homoeologues of OV1 occurring in the Chinese Spring wheat genome are provided herein as SEQ ID Nos. 14-22. An OV1 gene or sequence can be a naturally-occurring OV1 gene or sequence occurring in a plant, e.g., wheat. In some embodiments of any of the aspects, an OV1 gene is a gene which displays the same type of activity, and/or shares at least 80%, at least 85%, at least 90%, at least 95%, or greater sequence identity with an OV1 gene of a sequence provided herein. In some embodiments of any of the aspects, a OV1 gene can be the gene from a species, cultivar, or variety which has the highest degree of homology and/or sequence identity of the genes in that species', cultivar's or variety's genome with an OV1 sequence provided herein.

The OV gene selected for use in the compositions and methods described herein can, e.g., have homology to a gene demonstrated to be vital for post-meiosis events such as cell division of the initial archesporial haploid cell, differentiation into an egg cell, a central cell, two synergid cells and three antipodal cells or synthesis and export of pollen-tube attractant compounds in a plant. A non-limiting list of exemplary OV genes is provided in Table 2. In some embodiments of any of the aspects, the OV gene is a gene selected from Table 2. In some embodiments of any of the aspects, the OV gene is a gene which displays the same type of activity, and/or shares at least 80%, at least 85%, at least 90%, at least 95%, or greater sequence identity with a OV gene of Table 2. In some embodiments of any of the aspects, an OV gene can be the gene from a species, cultivar, or variety which has the highest degree of homology and/or sequence identity of the genes in that species', cultivar's or variety's genome with a gene selected from Table 2 In some embodiments of any of the aspects, an OV gene can be the gene from a species, cultivar, or variety which has the greatest degree of homology and/or sequence identity of the genes in that species', cultivar's or variety's genome with a gene selected from Table 2.

TABLE 2
Exemplary OV genes

| Gene Name | Exemplary Reference Sequences |
|---|---|
| OV1 | SEQ ID Nos. 14-22 |
| MADS13 | Designated TraesCS5A02G117500, |
| | TraesCS5B02G115100, and |
| | TraesCS5D02G118200 in the |
| | Ensembl database, which provides |
| | gDNA, CDS, and transcript |
| | sequence data. See also, e.g, Dreni |
| | et al. The Plant Journal 52: 690-699 |
| | (2007) which is incorporated by |
| | reference herein in its entirety |
| RKD2 | See, e.g., Tedeschi et al. New |
| | Phytologist doi: 10.1111/nph.14293 |
| | (2016); which is incorporated by |
| | reference herein in its entirety |

In one embodiment of any of the aspects, the Mf, OV, and PV genes are the combination of Mf, OV, and PV genes provided in Table 4.

TABLE 4
Exemplary combination of Mf, OV, and PV genes.

| Gene | Exemplary Reference Sequence |
|---|---|
| Mfw2 or Mfw10 | |
| PV1 | SEQ ID Nos: 1-9 |
| OV1 | SEQ ID Nos: 14-22 |

In one aspect of any of the embodiments, provided herein is a method of producing a male-fertile maintainer plant as described herein, wherein the method comprises:
a. engineering knock-out modifications in each allele of Mf, OV, and PV in the second any subsequent genomes, resulting in a fertile plant;
b. engineering the modifications in the first chromosome of the first genome; and
c. engineering the modifications in the second chromosome of the first genome.
   The modifications can be engineered by any single methodology or technology known in the art (which are described elsewhere herein) or a combination of any of those methodologies or technologies. In some embodiments of any of the aspects, the method comprises engineering one or more modifications, e.g., by contacting a plant cell with a site-specific guided nuclease. In some embodiments of any of the aspects, the method comprises engineering one or more modifications, e.g., by contacting a plant cell with a site-specific guided nuclease and at least one multi-guide construct. In some embodiments of any of the aspects, step a of the foregoing method comprises a single step of contacting a plant cell with a site-specific guided nuclease (e.g., a Cas enzyme) and one or more multi-guide constructs that target each allele of Mf, OV, and PV in the second and subsequent genomes.

In one aspect of any of the embodiments, provided herein is a method of producing a male-fertile maintainer plant as described herein, wherein the method comprises:
a. engineering knock-out modifications in each allele of Mf, OV, and PV in the second any subsequent genomes;
b. engineering the modifications in the first genome.

The modifications can be engineered by any single methodology or technology known in the art (which are described elsewhere herein) or a combination of any of those methodologies or technologies. In some embodiments of any of the aspects, step a of the foregoing method comprises a single step of contacting a plant cell with a site-specific guided nuclease (e.g., a Cas enzyme) and one or more multi-guide constructs that target each allele of Mf, OV, and PV in the genomes. The multiple engineered modifications can be generated in a single cell or plant (sequentially or concurrently) or created in multiple separate cells or plants which are then crossed to provide a final plant comprising all of the desired modifications. For example, in some embodiments of any of the aspects, a method of making a maintainer plant described herein can comprise: a) engineering the modifications in the first chromosome of the first genome in a first plant; b) engineering the modifications in the second chromosome of the first genome in a second plant; c) crossing the resulting plants; and d) selecting the F2 progeny of step c) which comprise the engineered first and second chromosomes of the first genome. Steps a) and b) can be performed sequentially or concurrently in the first and second plants. Alternatively, the modifications in the first and second chromosomes of the first genome can be engineered in a single step, e.g., by contacting a plant cell with a Cas enzyme and one or more multi-guide constructs that direct each engineered modification.

Selection and screening of plants which comprise the engineered modification(s) and/or progeny which comprise a combination of modifications can be performed by any method known in the art, e.g., by phenotype screening or selection, genetic analysis (e.g. PCR or sequencing to detect the modifications), analysis of gene expression products, and the like. Such methods are known to one of skill in the art and can be used in any combination as desired. In some embodiments of any of the aspects, the engineered modifications do not comprise introduction of an exogenous marker gene (e.g., a selectable marker or screenable marker such as herbicide resistance or fluorescence or color-altering genes), and any selection or screening step does not rely upon the use of a selectable marker gene.

In some embodiments of any of the aspects, the method comprises first generating the knock-out modifications in the Mf, OV, and PV genes in the second and third genomes, e.g., sequentially or concurrently. In some embodiments of any of the aspects, the method comprises first generating the knock-out modifications in the Mf, OV, and PV genes, e.g., sequentially or concurrently. In some embodiments of any of the aspects, each knock-out modification utilizes a guided nuclease (e.g., Cas9) and one, two, three, or more targeted sequences per gene. In some embodiments of any of the aspects, each knock-out modification utilizes a targeted nuclease (e.g., Cas9) and three targeted sequences per gene. In some embodiments of any of the aspects, the step of generating knock-out modification in the Mf, OV, and PV genes in the second and third genomes comprises concurrent or simultaneous knock-out modifications generated by contacting a cell with a guided nuclease (e.g., Cas9) and three guide RNA sequences for each target, e.g., nine guide RNA sequences total. In some embodiments of any of the aspects, the step of generating knock-out modifications in the Mf, OV, and PV genes in three genomes comprises concurrent or simultaneous knock-out modifications generated by contacting a cell with a guided nuclease (e.g., Cas9) and three guide RNA sequences for each target. In some embodiments of any of the aspects, the knock-out modifications can also be made in the first genome (e.g., knockout of Mf, OV, and PV genes on one chromosome of the first genome each, as described above herein), permitting fertility. The engineered deletions of the first genome can then be generated. In some embodiments of any of the aspects, described herein is a method of producing a male-fertile maintainer plant, wherein the method comprises:
a. engineering knock-out modifications in each allele of Mf, OV, and PV in the second any subsequent genomes, and engineering knock-out modifications of one allele of each of Mf, OV, PV, in a first genome, resulting in a fertile plant;
b. engineering at least one deletion of endogenous intervening sequences between the Mf, PV; and/or OV loci in the first genome.
   In some embodiments of any of the aspects, described herein is a method of producing a male-fertile maintainer plant, wherein the method comprises:
a. engineering knock-out modifications in each allele of Mf, OV, and PV in the second any subsequent genomes, and engineering knock-out modifications of one allele of each of Mf, OV, PV, in a first genome, resulting in a fertile plant;
b. selecting plants and/or progeny with the modifications recited in step a;
c. engineering at least one deletion of endogenous intervening sequences between the Mf; PV; and/or OV loci in the first genome; and
d. selecting plants and/or progeny with the modifications recited in step c

In one aspect of any of the embodiments, provided herein is a method of producing a male-fertile maintainer plant as described herein, wherein the method comprises: i) engineering the pollen construct and/or ovule construct in a first plant; ii) transferring the pollen construct and/or ovule construct to a second, wild-type cultivar plant by:
a) crossing pollen comprising the pollen construct from the first plant onto the second plant;
b) selfing the F1 generation
c) in the F2 generation, selecting plants homozygous for the pollen construct and crossing the pollen from the selected homozygous F2 plants onto third, wild-type cultivar plant of the same cultivar as the second plant; and
d) repeating this process until the crossed plants are substantially isogenic with the wild-type cultivar with the exception of the pollen construct; and
e) crossing pollen from a fourth wildtype cultivar plant onto a plant comprising the ovule construct;
f) selfing the F1 generation
g) in the F2 generation, selecting plants homozygous for the ovule construct and crossing pollen from a fifth, wild-type cultivar plant of the same cultivar as the fourth plant onto the selected homozygous F2 plants; and
h) repeating this process until the crossed plants are substantially isogenic with the wild-type cultivar with the exception of the ovule construct; and
i) crossing the pollen from the plant obtained from step (d) onto the plant obtained from step (h); and
j) selfing the F1 generation, whereby the resulting progeny will have a heterozygous genotype and produce pollen with the pollen construct only and ovules with the ovule construct only.
   Steps a-d and e-h can be performed concurrently (e.g., in parallel) or sequentially.

The foregoing methods of generating a male-fertile maintainer line can be readily adapted to generating a maintainer line for any trait or set of traits, e.g., for generating a maintainer line for any combination of Mf, PV, or OV genes, or any combination of two or more genes for which a maintainer line is desired.

Further provided herein are methods of selecting a chromosome arm in a genome as the site of production of a co-segregating construct and/or methods of selecting a set of two or more genes for production of a co-segregating construct. As used herein, “co-segregating construct” refers to a construct in which intergenic genomic sequences are removed between alleles of two or more genes, such that the genetic linkage of those genes is increased. As described elsewhere herein, such co-segregating constructs can be used in some embodiments to produce maintainer lines for certain traits and exemplary co-segregating constructs can include the pollen and ovule constructs described above herein. The following methods are exemplars which relate to the selection of a set of a Mf, a PV, and an OV gene, but the described methods can be adapted to the selection of a combination of any two or more genes for use in a co-segregating construct.

In one aspect of any of the embodiments, provided herein is a method of selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the method comprising:
a. selecting one of a Mf gene, PV gene, or OV gene;
b. identifying one of each of the two genes not selected in step a which map to the same arm of the same chromosome as the gene selected in step a;
c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
d. selecting the chromosome arm of a cultivar genome as an appropriate site of production if target sequences of step (c) can be identified.
   In one aspect of any of the embodiments, provided herein is a method of selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the method comprising:
a. selecting one of a Mf gene, PV gene, or OV gene;
b. identifying one of each of the two genes not selected in step a which map to the same arm of the same chromosome as the gene selected in step a;
c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
d. selecting the chromosome arm of a cultivar genome as an appropriate site of production if target sequences of step (c) can be identified.

In some embodiments of any of the aspects, intergenic sequence is to be deleted from between only two of the three genes, e.g., when two of the genes are adjacent and/or in high enough genetic linkage that deletion of intergenic sequence is deemed unnecessary or undesired. The threshold for a genetic linkage which is high enough depends upon, e.g., the rate of recombination in the particular plant genome/chromosome being used and the amount of screening and backcrossing that a particular user will find acceptable, e.g., on the basis of amount of seeds produced by a plant, the ease and speed of the selected screening/selection methods, the time which it takes for the particular plant to complete a single reproductive cycle (e.g., from seed to seed) and the amount of resources required (e.g., the space required to grow an individual plant) and the consequences or perceived consequences of an escaped non-conforming genotype (eg an Mfw allele in pollen grain) due to crossing-over recombination if the linkage is not close enough. One of skill in the art can determine an acceptable amount of genetic linkage for any given set of such circumstances.

In some embodiments of any of the aspects, two target sequences are selected, between either the distal and central or central and proximal genes. In some embodiments of any of the aspects, four target sequences are selected, two between the distal and central genes and two between the proximal and central genes. In some embodiments of any of the aspects, deletions of endogenous intervening sequence are made between each pair of the three genes.

In some embodiments of any of the aspects, more than two target sequences can be selected between two genes, e.g., to increase the rate of deletion.

The target sequences should be located outside of the coding sequence of the Mf, PV, and OV genes. In some embodiments of any of the aspects, the target sequences are located outside of any regulatory sequences (i.e. distal of any regulatory sequences with respect of the gene's coding sequence) associated with the Mf, PV, and/or OV genes. Coding sequences and regulatory sequences for any given gene can be identified using software routinely used for such purposes. For example, the end or boundary of a coding sequence/open reading frame can be identified by one of skill in the art by, e.g., consulting an annotated copy of the relevant genome, comparing the relevant genome and a related annotated genome, or using various sequence analysis computer programs that can identify and/or predict genetic elements such as transcriptional start and stop sequences.

Additionally, exemplary target sequence locations are provided for multiple exemplary genes elsewhere herein.

In some embodiments of any of the aspects, the target sequence is located at least about 1 kb from the boundary of the Mf, PV, and OV gene's coding sequence, e.g., at least about 1 kb, at least about 2 kb, at least about 3 kb, at least about 4 kb, or further from the boundary of the Mf, PV, and OV gene's coding sequence. In some embodiments of any of the aspects, the target sequence is located at least 1 kb from the boundary of the Mf, PV, and OV gene's coding sequence, e.g., at least 1 kb, at least 2 kb, at least 3 kb, at least 4 kb, or further from the boundary of the Mf, PV, and OV gene's coding sequence.

In some embodiments of any of the aspects, the target sequence is located at least about 5 kb from the boundary of the Mf, PV, and OV gene's coding sequence, e.g., at least about 5 kb, at least about 6 kb, at least about 7 kb, at least about 8 kb, at least about 9 kb, at least about 10 kb or further from the boundary of the Mf, PV, and OV gene's coding sequence. In some embodiments of any of the aspects, the target sequence is located at least 5 kb from the boundary of the Mf, PV, and OV gene's coding sequence, e.g., at least 5 kb, at least 6 kb, at least 7 kb, at least 8 kb, at least 9 kb, at least 10 kb or further from the boundary of the Mf, PV, and OV gene's coding sequence. In some embodiments of any of the aspects, the target sequence is located at about 5 kb from the boundary of the Mf, PV, and OV gene's coding sequence, e.g., at about 5 kb, at about 6 kb, at about 7 kb, at about 8 kb, at about 9 kb, or at about 10 kb from the boundary of the Mf, PV, and OV gene's coding sequence. The target sequence can be in intergenic sequence or in the sequence of an intervening gene (e.g., intragenic sequence). In some embodiments of any of the aspects described herein, the target sequence can be identified within from the sequence which is about 500 bp to about 10 kb from the end of the open reading frame, e.g., about 1 kb to about 9 kb, about 2 kb to about 8 kb, about 3 kb to about 7 kb, or about 4 kb to about 6 kb from the open reading frame. In some embodiments of any of the aspects described herein, the target sequence can be identified from within the sequence which is 500 bp to 10 kb from the end of the open reading frame, e.g., 1 kb to 9 kb, 2 kb to 8 kb, 3 kb to 7 kb, or 4 kb to 6 kb from the open reading frame.

In some embodiments of any of the aspects, the method of selecting a set of genes for a co-segregating construct and/or a chromosome arm for production of a co-segregating construct can further comprise a step of engineering the deletion modification(s) of endogenous intervening sequences between the Mf; PV; and/or OV loci by any of the methods described herein. In some embodiments of any of the aspects, the method of selecting a set of genes for a co-segregating construct and/or a chromosome arm for production of a co-segregating construct can further comprise a step of engineering the deletion modification(s) of endogenous intervening sequences between the Mf; PV; and/or OV loci by contacting a cell comprising the chromosome with a site-specific guided nuclease and one or more guide molecules which hybridize to the identified target sequences. In some embodiments of any of the aspects, the method of selecting a set of genes for a co-segregating construct and/or a chromosome arm for production of a co-segregating construct can further comprise a step of engineering the deletion modification(s) of endogenous intervening sequences between the Mf; PV; and/or OV loci by contacting a cell comprising the chromosome with a site-specific guided nuclease and a multi-guide construct which hybridizes to the identified target sequences.

In one aspect of any of the embodiments, provided herein is a method of selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the method comprising:
a. selecting one of a Mf gene, PV gene, or OV gene;
b. identifying one of each of the two genes not selected in step a which map to the same arm of the same chromosome as the gene selected in step a;
c. identifying at least one target sequence for a site-specific guided nuclease guide from the sequence distal of regulatory elements regulating expression of the start or end of the open reading frame on the distal side of the proximal gene of any subset of two genes identified in step b) and at least one target sequence for a site-specific guided nuclease guide from the sequence proximal of regulatory elements proximal of the start or end of the open reading frame on the proximal side of the distal gene of any subset of two genes identified in step b) and
d. selecting the chromosome arm of a cultivar genome as an appropriate site of production if target sequences of step (c) can be identified.
   In one aspect of any of the embodiments, provided herein is a method of selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the method comprising:
a. selecting one of a Mf gene, PV gene, or OV gene;
b. identifying one of each of the two genes not selected in step a which map to the same arm of the same chromosome as the gene selected in step a;
c. identifying at least one target sequence for a site-specific guided nuclease guide from the sequence distal of regulatory elements regulating expression of the start or end of the open reading frame on the distal side of the proximal gene of any subset of two genes identified in step b) and at least one target sequence for a site-specific guided nuclease guide from the sequence proximal of regulatory elements proximal of the start or end of the open reading frame on the proximal side of the distal gene of any subset of two genes identified in step b) and
d. selecting the chromosome arm of a cultivar genome as an appropriate site of production if target sequences of step (c) can be identified.
   The sequences which are distal of regulatory elements distal of the start or end of the open reading frame, or the sequence which are proximal of regulatory elements proximal of the start or end of the open reading frame typically being 5 kb from the boundary of the open reading frame.

It is noted that in the foregoing methods, where instructions are provided for selecting target sequences, the orientation of the Mf, PV, and OV genes are not implied. Regulatory sequences can be located either 5′ or 3′ of the open reading frame, and “boundary” can refer to either the 5′ start of the open reading frame or the 3′ terminus of the open reading frame. The three genes can be in the same or varying 5′ to 3′ orientations.

In some instances, more detailed genomic information is available for a reference genome rather than the cultivar genome itself. For example, in wheat, certain model strains have been subjected to extensive sequencing, while any given elite breeding line may not have been analysed to the same degree. In such cases, the method of selecting a set of genes for a co-segregating construct and/or a chromosome arm for production of a co-segregating construct can comprise identifying one or more genes (e.g., a Mf, PV, and/or OV gene) in a reference genome (e.g., from a different strain of the same species as the cultivar genome) and then searching the cultivar genome to determine if the set of genes identified in the reference genome is applicable to the cultivar genome. For example, the cultivar genome might comprise a translocation and/or mutation of the sequence of the one or more genes identified in the reference genome, which would make those genes inappropriate for use in the cultivar. In some embodiments of any of the aspects, identifying two genes of the set comprises first identifying the genes in a reference genome and then searching the cultivar genome to identify any translocations or mutations that would affect the two genes. When such translocations or mutations are identified, the genes identified in the reference genome are rejected for use in making a co-segregating construct in that particular cultivar genome.

In addition to the foregoing methods of selecting a set of genes for a co-segregating construct and/or a chromosome arm for production of a co-segregating construct, provided herein is a system for selecting a chromosome arm of a cultivar genome as the site of production of a co-segregating construct. The following systems are exemplars which relate to the selection of a set of a Mf, a PV, and an OV gene, but the described systems can be adapted to the selection of a combination of any two or more genes for use in a co-segregating construct. In one aspect of any of the embodiments, described herein is a system for selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the system comprising:
i. a memory having processor-readable instructions stored therein; and
ii. a processor configured to access the memory and execute the processor-readable instructions, which, when executed by the processor configures the processor to perform a method, the method comprising:
   A. receiving initial data relating to a co-segregating construct, the initial data including a) one of a Mf gene, a PV gene, and an OV gene; and b) a reference genome;
   B. processing, using the processor, the initial data to identify one each of the two genes not provided in step A which map to the same arm of the same chromosome as the gene provided in step A;
   C. processing, using the processor, the data obtained from step B) to identify, for each set of a Mf gene, a PV gene, and an OV gene, at least one target sequences for a site-specific guided nuclease guide, with one target sequence identified from each of:
      the sequence distal of regulatory elements regulating expression of the start or end of the open reading frame on the distal side of the proximal gene of any subset of two genes identified in step A; and
      the sequence proximal of regulatory elements proximal of the start or end of the open reading frame on the proximal side of the distal gene of any subset of two genes identified in step A;
   D. creating a library of sets of Mf, PV, and OV genes and associated target sequences;
   E. selecting, using the processor, a set of Mf, PV, and OV genes and associated target sequences with the minimal distance from the target sequences to the border of the open reading frame of the nearest gene from the library of sets.
      In one aspect of any of the embodiments, described herein is a system for selecting a chromosome arm of a cultivar genome as the site of production of a co-segregating construct wherein the co-segregating construct comprises
e. an endogenous, wild-type functional allele of a male-fertility gene (Mf gene);
f. an endogenous, wild-type functional allele of a pollen-grain-vital gene (PV gene);
g. an endogenous, wild-type functional allele of an ovule-vital gene (OV gene); and
h. at least one engineered modification comprising a deletion of endogenous intervening sequences between the Mf; PV; and/or OV loci;
   the system comprising:
iii. a memory having processor-readable instructions stored therein; and
iv. a processor configured to access the memory and execute the processor-readable instructions, which, when executed by the processor configures the processor to perform a method, the method comprising:
   A. receiving initial data relating to a co-segregating construct, the initial data including a) one of a Mf gene, a PV gene, and an OV gene; and b) a reference genome;
   B. processing, using the processor, the initial data to identify one each of the two genes not provided in step A which map to the same arm of the same chromosome as the gene provided in step A;
   C. processing, using the processor, the data obtained from step B) to identify, for each set of a Mf gene, a PV gene, and an OV gene, at least one target sequences for a site-specific guided nuclease guide, with one target sequence identified from each of:
      i. the sequence approximately 5 kb from the end of the open reading frame on the proximal side of the most distal gene; and
      ii. the sequence approximately 5 kb from the end of the open reading frame on the distal side of the central gene;
      and/or
      iii. the sequence approximately 5 kb from the end of the open reading frame on the proximal side of the central gene; and
      iv. the sequence approximately 5 kb from the end of the open reading frame on the distal side of the most proximal gene;
   D. creating a library of sets of Mf, PV, and OV genes and associated target sequences;
   E. selecting, using the processor, a set of Mf, PV, and OV genes and associated target sequences with the minimal distance from the target sequences to the border of the open reading frame of the nearest gene from the library of sets.
      In one aspect of any of the embodiments, described herein is a system for selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the system comprising:
i. a memory having processor-readable instructions stored therein; and
ii. a processor configured to access the memory and execute the processor-readable instructions, which, when executed by the processor configures the processor to perform a method, the method comprising:
   A. receiving initial data relating to a co-segregating construct, the initial data including a) one of a Mf gene, a PV gene, and an OV gene; and b) a reference genome;
   B. processing, using the processor, the initial data to identify one each of the two genes not provided in step A which map to the same arm of the same chromosome as the gene provided in step A;
   C. processing, using the processor, the data obtained from step B) to identify, for each set of a Mf gene, a PV gene, and an OV gene, at least two target sequences for a site-specific guided nuclease guide, with one target sequence identified from each of:
      i. the sequence at least about 5 kb from the end of the open reading frame on the proximal side of the most distal gene; and
      ii. the sequence at least about 5 kb from the end of the open reading frame on the distal side of the central gene;
      and/or
      iii. the sequence at least about 5 kb from the end of the open reading frame on the proximal side of the central gene; and
      iv. the sequence at least about 5 kb from the end of the open reading frame on the distal side of the most proximal gene;
   D. creating a library of sets of Mf, PV, and OV genes and associated target sequences;
   E. selecting, using the processor, a set of Mf, PV, and OV genes and associated target sequences with the minimal distance from the target sequences to the border of the open reading frame of the nearest gene from the library of sets.
      In one aspect of any of the embodiments, described herein is a system for selecting a chromosome arm of a cultivar genome as the site of production of a co-segregating construct wherein the co-segregating construct comprises
a. an endogenous, wild-type functional allele of a male-fertility gene (Mf gene);
b. an endogenous, wild-type functional allele of a pollen-grain-vital gene (PV gene);
c. an endogenous, wild-type functional allele of an ovule-vital gene (OV gene); and
d. at least one engineered modification comprising a deletion of endogenous intervening sequences between the Mf; PV; and/or OV loci;
   the system comprising:
i. a memory having processor-readable instructions stored therein; and
ii. a processor configured to access the memory and execute the processor-readable instructions, which, when executed by the processor configures the processor to perform a method, the method comprising:
   A. receiving initial data relating to a co-segregating construct, the initial data including a) one of a Mf gene, a PV gene, and an OV gene; and b) a reference genome;
   B. processing, using the processor, the initial data to identify one each of the two genes not provided in step A which map to the same arm of the same chromosome as the gene provided in step A;
   C. processing, using the processor, the data obtained from step B) to identify, for each set of a Mf gene, a PV gene, and an OV gene, at least two target sequences for a site-specific guided nuclease guide, with one target sequence identified from each of:
      i. the sequence at least 5 kb from the end of the open reading frame on the proximal side of the most distal gene; and
      ii. the sequence at least 5 kb from the end of the open reading frame on the distal side of the central gene;
      and/or
      iii. the sequence at least 5 kb from the end of the open reading frame on the proximal side of the central gene; and
      iv. the sequence at least 5 kb from the end of the open reading frame on the distal side of the most proximal gene;
   D. creating a library of sets of Mf, PV, and OV genes and associated target sequences;
   E. selecting, using the processor, a set of Mf, PV, and OV genes and associated target sequences with the minimal distance from the target sequences to the border of the open reading frame of the nearest gene from the library of sets.

The systems described herein can be provided, e.g., in a network environment in which various systems may select one or more sets, according to an embodiment of the present disclosure. In some embodiments of any of the aspects, the environment may include a plurality of user or client devices that are communicatively coupled to each other as well as one or more server systems via an electronic network. Electronic networks can include one or a combination of wired and/or wireless electronic networks. Networks can also include a local area network, a medium area network, or a wide area network, such as the Internet.

In some embodiments of any of the aspects, each of the user or client devices may be any type of computing device configured to send and receive different types of content and data to and from various computing devices via network. Examples of such a computing device include, but are not limited to, mobile health devices, a desktop computer or workstation, a laptop computer, a mobile handset, a personal digital assistant (PDA), a cellular telephone, a network appliance, a camera, a smart phone, an enhanced general packet radio service (EGPRS) mobile phone, a media player, a navigation device, a game console, a set-top box, a biometric sensing device with communication capabilities, or any combination of these or other types of computing devices having at least one processor, a local memory, a display (e.g., a monitor or touchscreen display), one or more user input devices, and a network communication interface. The user input device(s) may include any type or combination of input/output devices, such as a keyboard, touchpad, mouse, touchscreen, camera, and/or microphone.

In one embodiment, each of the user or client devices can be configured to execute a web browser, mobile browser, or additional software applications that allows for input of the specified data. Server systems in turn can be configured to receive the specified data. The systems can include a singular server system, a plurality of server systems working in combination, a single server device, or a single system. In some embodiments, the server system can include one or more databases. In some embodiments of any of the aspects, databases may be any type of data store or recording medium that can be used to store any type of data. For example, databases can store data received by or processed by server system including reference genome information, cultivar genome information, and one or more Mf, PV, or OV genes.

Additionally, server systems can include a processor. In some embodiments of any of the aspects, a processor can be configured to execute a process for selecting genes, sets of genes, and/or target sequences. In some embodiments of any of the aspects, a processor can be configured to receive instructions and data from various sources including user or client devices and store the received data within databases. Processors or any additional processors within server system also can be configured to provide content to client or user devices for display. For example, processors can transmit displayable content including messages or graphic user interfaces relating to genetic maps, target sequence locations, and gene locations.

In some embodiments of any of the aspects, the method entails creating a library of sets of Mf, PV, and OV genes and associated target sequences.

In some embodiments of any of the aspects, the method can entail receiving the receiving initial data relating to a co-segregating construct, the initial data including at least one gene and a reference genome. The received data may include receiving data related to a reference genome, cultivar genome, annotation or expression information relating to one or more genomes, and/or genes.

The processor can then, using the criteria described herein, identify sets of Mf, PV, and OV genes for each initially identified gene. The processor can then, using the criteria described herein, select target sequences for each set of genes. The set of genes and target sequences can then be entered into the library of sets. Sets can be ranked by e.g., distance between genes in the set, whether the target sequences exist in other copies of the genome, quality of the relevant sequence information in the cultivar genome, distance of the target sequences to the open reading frames, or other user-generated criteria. The sets in the library can then be utilized in the library to select the highest-ranking sets, e.g., by one or more of the foregoing categories. In additional embodiments, a plurality of sets are to be selected. In instances when more than one set exists for a given context, potential conflicts may be resolved by following certain rules of selection. For example, rules of selection may provide limitations for picking sets. The rules may include limitations regarding allowable and non-allowable sets or elements of sets, e.g., according to the foregoing criteria, or a ranked preference for any of the criteria. The rules also may prioritize a list of eligible sets or rules that may be applied. In embodiments, a threshold number of highly prioritized sets can be selected. The rules of selection also can be based on randomized logic. The system can include generating a notification when a set(s) is selected.

The system can be implemented using hardware, software modules, firmware, tangible computer readable media having instructions stored thereon, or a combination thereof and can be implemented in one or more computer systems or other processing systems. If programmable logic is used, such logic can be executed on a commercially available processing platform or a special purpose device. One of ordinary skill in the art may appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device. For instance, at least one processor device and a memory can be used to implement the above-described embodiments. A processor device may be a single processor, a plurality of processors, or combinations thereof. Processor devices may have one or more processor “cores.”

Various embodiments of the present disclosure, as described above can be implemented using computer system. After reading this description, it will become apparent to a person skilled in the relevant art how to implement embodiments of the present disclosure using other computer systems and/or computer architectures. Although operations can be described as a sequential process, some of the operations may in fact be performed in parallel, concurrently, and/or in a distributed environment, and with program code stored locally or remotely for access by single or multi-processor machines. In addition, in some embodiments the order of operations can be rearranged without departing from the spirit of the disclosed subject matter.

A computer system can include a central processing unit (CPU). A CPU can be any type of processor device including, for example, any type of special purpose or a general-purpose microprocessor device. As will be appreciated by persons skilled in the relevant art, a CPU can also be a single processor in a multi-core/multiprocessor system, such system operating alone, or in a cluster of computing devices operating in a cluster or server farm. A CPU can be connected to a data communication infrastructure, for example, a bus, message queue, network, or multi-core message-passing scheme.

A Computer system can also include a main memory, for example, random access memory (RAM), and also can include a secondary memory. Secondary memory, e.g., a read-only memory (ROM), can be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc. which is read by and written to by the removable storage drive. As will be appreciated by persons skilled in the relevant art, such a removable storage unit generally includes a computer usable storage medium having stored therein computer software and/or data.

In alternative implementations, secondary memory can include other similar means for allowing computer programs or other instructions to be loaded into computer system. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to computer system.

A computer system can also include a communications interface (“COM”). A communications interface allows software and data to be transferred between computer system and external devices. Communications interface can include a modem, a network interface (such as an Ethernet card), a communications port, a PCMCIA slot and card, or the like. Software and data transferred via communications interface may be in the form of signals, which may be electronic, electromagnetic, optical, or other signals capable of being received by communications interface. These signals can be provided to communications interface via a communications path of computer system, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. A computer system also may include input and output ports to connect with input and output devices such as keyboards, mice, touchscreens, monitors, displays, etc. Of course, the various server functions can be implemented in a distributed fashion on a number of similar platforms, to distribute the processing load. Alternatively, the servers may be implemented by appropriate programming of one computer hardware platform.

Program aspects of the technology can be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software can at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also can be considered as media bearing the software. As used herein, unless restricted to non-transitory, tangible “storage” media, terms such as computer or machine “readable medium” refer to any medium that participates in providing instructions to a processor for execution.

In one aspect of any of the embodiments, described herein is a plant or plant cell comprising a deactivating modification of at least one OV gene and/or at least one PV gene. In some embodiments of any of the aspects, the plant or plant cell can further comprise a deactivating modification of at least one Mf gene.

In some embodiments of any of the aspects, the plant comprising a deactivating modification of at least one OV gene and/or at least one PV gene permits seed segregation of its progeny. In some embodiments of any of the aspects, the plant comprising a deactivating modification of at least one OV gene and/or at least one PV gene comprises deactivating modifications of each of the copies of the at least one PV or OV gene. In some embodiments of any of the aspects, the deactivating modification is identical across each genome of the plant. In some embodiments of any of the aspects, each genome of the plant comprises a different deactivating modification.

In some embodiments of any of the aspects, the at least one PV and/or OV gene is selected from the genes of Tables 1 and/or 2. In some embodiments of any of the aspects, the at least one PV and/or OV gene has at least 60%, at least 80%, at least 85%, at least 90%, at least 95% or greater sequence identity with a gene of Tables 1 and/or 2. In some embodiments of any of the aspects, the at least one PV and/or OV gene has the same activity and at least 60%, at least 80%, at least 85%, at least 90%, at least 95% or greater sequence identity with a gene of Tables 1 and/or 2.

Individual modifications may be referred to herein as “deactivating modifications.” The phrase “deactivating modification” refers to a modification of an individual nucleic acid sequence and/or copy of a gene, which may or may not, on its own, result in deactivation of the desired gene. For example, deactivating modifications at all six copies of a given gene may be necessary to deactivate the gene. Furthermore, it is contemplated herein that the deactivating modification found at any given copy of a gene may or may not be identical to the deactivating modification found at the remaining copies of that gene. In some embodiments of any of the aspects, a knock-out or nonfunctional allele of a gene can comprise a deactivating modification at that allele.

In the context of a type of modification that is made at a location in the genome other than at the gene to be deactivated, a single modification may be sufficient to deactivate the gene (e.g, the introduction of an inhibitory nucleic acid). However, multiple copies of such modifications, e.g., at additional alleles and/or loci, may be desirable to prevent “leaky”, imperfect or unreliable phenotype or prevent loss of the desired phenotypes in subsequent generations.

In the context of a type of modification that is made at the gene to be deactivated, e.g, an indel at the coding sequence of the gene, it can be necessary to introduce deactivating modifications at additional copies of the gene (e.g., at all six copies of a given homoeologous gene set in wheat) in order to effect deactivation of the gene. Accordingly, a modification at the gene to be deactivated is considered a deactivating modification if it deactivates the copy of the gene in which it occurs, regardless of its effect on other copies of the gene.

As used herein, a “deactivated” gene is one that, due to engineering and/or modification of the genome (both chromosomal and/or extrachromosomal) of the cell in which the gene is found, is expressed at less than 35% of the wild-type level of functional polypeptide. In some embodiments of any of the aspects, a deactivated gene is expressed at less than 30% of the wild-type level of functional polypeptide. In some embodiments of any of the aspects, a deactivated gene is expressed at less than 25% of the wild-type level of functional polypeptide. In some embodiments of any of the aspects, a deactivated gene is expressed at less than 20% of the wild-type level of functional polypeptide. In some embodiments of any of the aspects, a deactivated gene is expressed at less than 15% of the wild-type level of functional polypeptide.

The wild-type level of functional polypeptide can be the level of functional polypeptide found in the same type of cell not comprising the modification. In some embodiments of any of the aspects, the level of functional polypeptide can be the level of full-length polypeptide with a wild-type sequence.

In some embodiments of any of the aspects, deactivation of a gene can comprise engineering, modifying, and/or altering the genome of the cell in which the gene is found such that the cell expresses no more than 35% of the wild-type level of the polypeptide, inclusive of both full-length and partial sequences of the gene. In some embodiments of any of the aspects, a deactivated gene is expressed at less than 30% of the wild-type level of polypeptide, inclusive of both full-length and partial sequences of the gene. In some embodiments of any of the aspects, a deactivated gene is expressed at less than 25% of the wild-type level of polypeptide, inclusive of both full-length and partial sequences of the gene. In some embodiments of any of the aspects, a deactivated gene is expressed at less than 20% of the wild-type level of polypeptide, inclusive of both full-length and partial sequences of the gene. In some embodiments of any of the aspects, a deactivated gene is expressed at less than 15% of the wild-type level of polypeptide, inclusive of both full-length and partial sequences of the gene.

In some embodiments of any of the aspects, deactivation of a gene can comprise engineering, modifying, and/or altering the genome of the cell in which the gene is found such that the cell expresses polypeptides comprising no more than 35% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 30% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 25% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 20% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 15% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 10% of the wild-type sequence of the polypeptide.

Ways of deactivating a gene can include modifying the genome so as to express RNA that inhibits expression of the targeted gene; or by gene-editing to prevent the gene carrying out its function. In some embodiments of any of the aspects described herein where a “knock-out allele” or “non-functional allele” is described, the deactivating modification is a modification at that allele and does not comprise the use of RNA interference or an inhibitory nucleic acid. The whole wheat genome has previously been sequenced and published. Sequences are given in Chapman et al (2014) and Clavijo et al, (2016) and were downloadable from, e.g., TGAC, The Genome Analysis Centre, Norwich in January 2016 and subsequently published in October 2016 as part of Clavijo et al., 2016. (available on the world wide web at ftp.ensemblgenomes.org/pub/plants/pre/fasta/triticum_aestivum/dna/). In the case of wheat, selecting sequences of targeted genes for use in the present invention, suitable coding sequences can be selected from Clavijo et al, (2016), Chapman et al (2014) or TGAC (or any other academic publication). Inhibitory RNA molecules or interfering mRNA (RNAi) that target a given gene can be designed by one of skill in the art from such coding sequence information.

In some embodiments of any of the aspects, a deactivating modification can be a modification that introduces an inhibitory nucleic acid into the cell, e.g, an RNAi, siRNA, shRNA, endogenous microRNA and/or artificial microRNA. The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target. An inhibitory nucleic acid mediates the targeted cleavage of a target RNA transcript, e.g., via an RNA-induced silencing complex (RISC) pathway, thereby inhibiting the expression and/or activity of the target, e.g., deactivating the target gene.

As described elsewhere herein, the plants can be polyploidal, e.g., wheat has a hexaploid genome. Accordingly, in some embodiments of any of the aspects, more than one copy of an inhibitory nucleic acid can be necessary in order to inhibit target gene(s) expression sufficiently to cause a phenotype. In some embodiments of any of the aspects, a deactivating modification can comprise 1 or more copies of nucleic acid encoding an inhibitory nucleic acid. In some embodiments of any of the aspects, a deactivating modification can comprise 2 or more copies of nucleic acid encoding an inhibitory nucleic acid. In some embodiments of any of the aspects, a deactivating modification can comprise 3 or more copies of nucleic acid encoding an inhibitory nucleic acid. Ibn some embodiments of any of the aspects, a deactivating modification can comprise 4 or more copies of nucleic acid encoding an inhibitory nucleic acid. In some embodiments of any of the aspects, a deactivating modification can comprise 5 or more copies of nucleic acid encoding an inhibitory nucleic acid. Multiple copies of a nucleic acid encoding an inhibitory nucleic acid can be integrated into the genome at the same loci (e.g., in series), or different loci.

Alternatively, genes may be deactivated by editing or deleting their associated promoter sequences or inserting a premature stop codon so that it no longer fulfils its function (‘gene knockout’). A variety of general methods is known for gene editing. Such editing may involve additions to or deletions from the gene coding sequence or from control (regulatory) sequences upstream or downstream of the coding sequence, but in any case is such as to inhibit production of functional RNA transcript. For example, a gene might be knocked out by inserting one or more additional base pairs of DNA resulting in coding for one or more unsuitable amino-acids, or by creating a premature stop codon so as to substantially shorten the resulting RNA transcript. In some embodiments of any of the aspects, such “gene editing” modifications comprise only deletion of DNA base sequence and not insertion of exogenous sequence. Such editing by deletion, because it contains no additional or heterogenous DNA, is often regarded as environmentally safer and so may require less extensive, and hence less expensive and time-consuming, regulation. Accordingly, in some embodiments of any of the aspects, a deactivating modification can be a modification that interrupts and/or alters the wild-type coding sequence of the gene, e.g., by deletions which generate a stop codon, transposon, deletion, or frameshift in the coding sequence of the gene. Methods of performing such modifications are described elsewhere herein.

In some embodiments of any of the aspects, engineered modifications, including deactivating modifications, can be introduced by means of a mutagen, e.g., ethyl methane sulphonate (EMS), radiation, UV light, aflatoxin B 1, nitrosoguanidine (NG), formaldehyde, acetaldehyde, diepoxyoctane (DEO), depoxybutane (DEB), diethyl sulphate (DES), methylnitrontrosoguanidine (NTG), N-ethyl-N-nitrosourea (ENU), and trimethylpsoralen (TMP). In some embodiments of any of the aspects, engineered modifications can be introduced, selected, and/or identified by means of TILLING (Targeted Induced Local Lesions IN Genomes) which uses mutagens to generate mutations. TILLING is described in detail, e.g., in Kurowska et al. J Appl Genet 2011 52:371-390 and McCallum et al. Plant Physiol 2000 123:439-442, which are incorporated by reference herein in their entireties.

In some embodiments of any of the aspects, engineered modifications can be introduced by non-transgenic mutagenesis, e.g., by a method which causes mutations of the nucleic acid sequences of the plant genome without introducing foreign and/or exogenous nucleic acid molecules into the plant cell. In some embodiments of any of the aspects, non-transgenic mutagenesis can comprise insertions and/or deletions due to mutagenic activity, e.g., indels arising from damage and/or repair processes in the cell. Non-transgenic mutagenesis can utilize, e.g., chemical mutagens (e.g., mutagens not comprising a nucleic acid sequence) and/or radiation sources (e.g., UV light). Non-transgenic mutagenesis excludes the use of, e.g., transposon insertions and/or RNAi. In some embodiments of any of the aspects, non-transgenic mutagenesis does not comprise the use of a site-specific nuclease, e.g., CRISPR-Cas. In some embodiments of any of the aspects, non-transgenic mutagenesis can be used in, e.g., TILLING approaches to generate and/or identify engineered modifications.

In some embodiments of any of the aspects, the engineered modification is not a naturally occurring modification, mutation, and/or allele.

In some embodiments of any of the aspects, the deactivating modification is excision of at least part of a coding or regulatory sequence; or the deactivated gene is deactivated by excision of at least part of a coding or regulatory sequence. In some embodiments of any of the aspects, the deactivating modification is insertion of RNAi-encoding sequences; or the deactivated gene is deactivated by inhibition by expression of RNAi. In some embodiments of any of the aspects, the deactivating modification is non-transgenic mutagenesis; or the deactivated gene is deactivated by non-transgenic mutagenesis.

In some embodiments of any of the aspects, genes can be deactivated by utilizing a CRISPR/Cas system to introduce deactivating mutations at these loci. For example, PV1 and OV1 can be targeted with four guide RNAs for each of the three sets of homoeologues and exemplary sets of such guide sequences are provided herein, e.g., guides having the sequences of SEQ ID Nos:10-13 can be used to target PV1 and guides having the sequences of SEQ ID Nos: 23-26 can be used to target OV1.

Exemplary guide sequences for targeting Mfw, PV, and OV alleles are described herein. Exemplary guide sequences for targeting Mfw alleles (either for knock-outs or simultaneous knockout/knock-ins) can also be found in International Patent Application PCT/US2017/043009, e.g., as SEQ ID NOs; 22-29 and 131-154 therein. The contents of International Patent Application PCT/US2017/043009 are incorporated by reference herein in their entirety.

In some embodiments of any of the aspects, the deactivating modification is a site-directed mutagenic event resulting from the activity of a site-specific nuclease; or the at least one gene is deactivated by site-directed mutagenesis resulting from the activity of a site-specific nuclease. In some embodiments of any of the aspects, the site-specific nuclease is CRISPR-Cas.

In order for a gene to be deactivated, it is necessary to reduce the expression from multiple alleles or copies, e.g., wheat is a hexaploid genome and it may be necessary to reduce expression from all six copies of a given gene. Accordingly, in some embodiments of any of the aspects, a deactivating modification is present at all six copies of a given deactivated gene. The individual deactivating modifications can be identical or they can vary.

In some embodiments of any of the aspects, the deactivation of a first gene can further comprise deactivation of one or more further related genes which display functional redundancy with the first gene. In some embodiments of any of the aspects, a plant or cell in which a given gene is deactivated can comprise deactivating modification(s) that deactivate all members of that gene's family. In some embodiments of any of the aspects, a plant or cell in which a given gene is deactivated can comprise deactivating modification(s) that deactivate all genes with at least 30% sequence identity at the amino acid level to the gene. In some embodiments of any of the aspects, a plant or cell in which a given gene is deactivated can comprise deactivating modification(s) that deactivate all genes with at least 40% sequence identity at the amino acid level to the gene. In some embodiments of any of the aspects, a plant or cell in which a given gene is deactivated can comprise deactivating modification(s) that deactivate all genes with at least 50% sequence identity at the amino acid level to the gene. In some embodiments of any of the aspects, a plant or cell in which a given gene is deactivated can comprise deactivating modification(s) that deactivate all genes with at least 60% sequence identity at the amino acid level to the gene. In some embodiments of any of the aspects, a plant or cell in which a given gene is deactivated can comprise deactivating modification(s) that deactivate all genes with at least 70% sequence identity at the amino acid level to the gene. In some embodiments of any of the aspects, a plant or cell in which a given gene is deactivated can comprise deactivating modification(s) that deactivate all genes with at least 80% sequence identity at the amino acid level to the gene. In some embodiments of any of the aspects, a plant or cell in which a given gene is deactivated can comprise deactivating modification(s) that deactivate all genes with at least 90% sequence identity at the amino acid level to the gene.

In some embodiments of any of the aspects, a plant or cell in which a given gene is deactivated can comprise deactivating modification(s) that deactivate all genes with at least 30% sequence identity at the nucleotide level to the gene. In some embodiments of any of the aspects, a plant or cell in which a given gene is deactivated can comprise deactivating modification(s) that deactivate all genes with at least 40% sequence identity at the nucleotide level to the gene. In some embodiments of any of the aspects, a plant or cell in which a given gene is deactivated can comprise deactivating modification(s) that deactivate all genes with at least 50% sequence identity at the nucleotide level to the gene. In some embodiments of any of the aspects, a plant or cell in which a given gene is deactivated can comprise deactivating modification(s) that deactivate all genes with at least 60% sequence identity at the nucleotide level to the gene. In some embodiments of any of the aspects, a plant or cell in which a given gene is deactivated can comprise deactivating modification(s) that deactivate all genes with at least 70% sequence identity at the nucleotide level to the gene. In some embodiments of any of the aspects, a plant or cell in which a given gene is deactivated can comprise deactivating modification(s) that deactivate all genes with at least 80% sequence identity at the nucleotide level to the gene. In some embodiments of any of the aspects, a plant or cell in which a given gene is deactivated can comprise deactivating modification(s) that deactivate all genes with at least 90% sequence identity at the nucleotide level to the gene.

It is contemplated herein that such further related gene(s) can be deactivated by the same type of modification (e.g., the first gene is deactivated by modifying the gene with CRISPR/Cas and the further related gene(s) are deactivated by modifying the further related genes(s) with CRISPR/Cas); with the same modification step (e.g., the first gene is deactivated by modifying the gene with CRISPR/Cas and the further related gene(s) are simultaneously deactivated by modifying the further related genes(s) with the same CRISPR/Cas array, wherein the array targets sequences shared between the first and further genes); or by separate types of modifications (e.g., the first gene is deactivated by modifying the gene with CRISPR/Cas and the further related gene(s) are deactivated by introducing an RNAi construct that targets the further related genes).

In embodiments where multiple genes are to be deactivated, e.g., multiple members of a gene family, deactivating modifications can be targeted to shared sequences to minimize the number of modifications and/or individual reagents. Alternatively, deactivating modifications can be targeted to areas that are unique to each gene and a multiplexed approach can be taken. By way of non-limiting example, a gene family can be deactivated utilizing a single CRISPR sgRNA (or equivalent) if the sgRNA is targeted to a sequence found in all members of the gene family; or the gene family can be deactivated utilizing multiple CRISPR sgRNAs (or equivalents) if the sgRNAs are each targeted to sequences not found in each member of the gene family.

In one aspect of any of the embodiments, described herein is a population of hybrid wheat plants comprising at least one copy of a deactivated gene described herein and at least one wild-type copy of the same gene. In one aspect of any of the embodiments, described herein is a population of hybrid wheat plants comprising at least one copy of a deactivated gene as described herein, where the gene locus comprises a deactivating modification and at least one wild-type copy of the same gene.

In some embodiments of any of the aspects, the engineered modifications described herein can be made directly in an elite breeding line. In some embodiments of any of the aspects, the engineered modifications described herein can be made in a first line or cultivar and then transferred to elite standard lines by normal backcrossing.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker, an “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

A “modification” in a nucleic acid sequence refers to any detectable change in the genetic material, e.g., a change or alteration relative to a reference sequence, e.g, the wild-type sequence. Modifications can be insertions, deletions, replacements, indels, SNPs, mutations, substitutions, or the like. A modification is usually a change of one or more deoxyribonucleotides, the modification being obtained by, for example, adding, deleting, inverting, or substituting nucleotides.

The term “wild type” refers to the naturally-occurring polynucleotide sequence encoding a protein, or a portion thereof, or protein sequence, or portion thereof, respectively, as it normally exists in vivo. It may also refer to the original plant genotype which was used for any transformation, gene-editing or gene-repression experiments herein, e.g., the genotype as it existed prior to any of the engineering steps described herein.

As used herein, “functional” refers to a portion and/or variant of a polypeptide or gene that retains at least a detectable level of the activity of the native polypeptide or gene from which it is derived. Methods of detecting, e.g. activity and/or functionality are known in the art for various types of polypeptides.

As used herein, “knock-out” refers to partial or complete reduction of the expression of a protein encoded by an endogenous DNA sequence in a cell such that the protein can no longer accomplish its function. In some embodiments, the “knock-out” can be produced by targeted deletion of the whole or part of a gene encoding a protein in an cell. In some embodiments, the deletion may prevent or reduce the expression of the functional protein in a cell in which it is normally expressed. A knock-out animal can be a transgenic animal, or can be created without transgenic methods, e.g. without the introduction of exogenous DNA to the genome.

As used herein, a “transgenic” organism or cell is one in which exogenous DNA from another source (natural, from another non-crossable species, or synthetic) has been introduced. In some cases, the transgenic approach aims at specific modifications of the genome, e.g., by introducing whole transcriptional units into the genome, or by up- or down-regulating pre-existing cellular genes. The targeted character of certain of these procedures sets transgenic technologies apart from experimental methods in which random mutations are conferred to the germline, such as administration of chemical mutagens or treatment with ionizing solution or gamma- or x-ray bombardment.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “ectopic” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

In some embodiments, a nucleic acid encoding a DNA or an RNA molecule or a polypeptide as described herein can be introduced into a cell by, e.g., biolistic delivery.

In some embodiments, a nucleic acid encoding an RNA or polypeptide as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. Exemplary vectors are known in the art and can include, by way of non-limiting example, pBR322 and related plasmids, pACYC and related plasmids, transcription vectors, expression vectors, phagemids, yeast expression vectors, plant expression vectors, pDONR201 (Invitrogen), pBI121, pBIN20, pEarleyGate100 (ABRC), pEarleyGate102 (ABRC), pCAMBIA, pUC-derived vectors, pSK-derived vectors, pGEM-derived vectors, pSP-derived vectors, pBS-derived vectors, the binary Ti plasmid (see, e.g., U.S. Pat. No. 4,940,838; which is incorporated by reference herein in its entirety), T-DNA, transposons, and artificial chromosomes.

As used herein, the term “expression vector” refers to a vector that directs expression of an RNA or polypeptide from sequences operably linked to transcriptional regulatory sequences on the vector. The term “operably linked” as used herein refers to a functional linkage between a regulatory element and a second sequence, wherein the regulatory element influences the expression and/or processing of the second sequence. Generally, “operably linked” means that the nucleic acid sequences being linked are contiguous or near contiguous and, where necessary to join two protein coding regions, contiguous and in the same reading frame. The regulatory sequence, e.g., a promoter, can be a constitutive, tissue-specific, and/or inducible promoter. The sequences expressed will often, but not necessarily, be heterologous to the cell. An expression vector may comprise additional elements, for example, the expression vector may have two replication systems, thus allowing it to be maintained in two organisms, for example in plant cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” refers to a sufficient degree of complementarity or precise pairing such that stable and specific binding occurs between the two nucleic acid sequences under the relevantly stringent conditions, e.g., in this case, in a plant cell. As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A male-fertile maintainer plant for a male-sterile polyploid plant, the maintainer plant comprising: in the first chromosome of a homologous pair in a first genome:
   a. an endogenous, wild-type functional allele of a male-fertility gene (Mf gene);
   b. an engineered knock-out modification at the allele of a pollen-grain-vital gene (PV gene);
   c. an endogenous, wild-type functional allele of an ovule-vital gene (OV gene); and
   d. at least one engineered modification comprising a deletion of endogenous intervening sequence between the Mf; PV; and/or OV loci;
   in the second chromosome of the same homologous pair in the first genome:
   e. an engineered knock-out modification at the allele of the Mf gene;
   f. an endogenous, wild-type functional allele of the PV gene; and
   g. an engineered knock-out modification at the allele of the OV gene;
   h. at least one engineered modification comprising a deletion of endogenous intervening sequence between the Mf; PV; and/or OV loci;
   in a second and any subsequent genomes:
   i. an engineered knock-out modification at each allele of the Mf gene;
   j. an engineered knock-out modification at each allele of the PV gene;
   k. an engineered knock-out modification at each allele of the OV gene;
whereby the pollen grains produced by the male-fertile maintainer plant comprise the second chromosome of the first genome and do not comprise the first chromosome of the first genome (hereinafter referred to as the pollen construct); and
the ovules produced by the male-fertile maintainer plant comprise the first chromosome of the first genome and do not comprise the second chromosome of the first genome (hereinafter referred to as the ovule construct).
2. The male-fertile maintainer plant of paragraph 1, wherein the first and second chromosomes of the first genome comprise at least one engineered modification comprising a deletion of endogenous intervening sequence between any two of the Mf; PV; and OV loci.
3. The male-fertile maintainer plant of any of paragraphs 1-2, wherein the plant is hexaploid and the male-sterile plant comprises an engineered knock-out modification at each of the six alleles of the male-fertility gene.
4. The male-fertile maintainer plant of any of paragraphs 1-2, wherein the plant is tetraploid and the male-sterile plant comprises an engineered knock-out modification at each of the four alleles of the male-fertility gene.
5. The male-fertile maintainer plant of any of paragraphs 1-4, wherein the maintainer plant is substantially isogenic with the male-sterile plant with the exception of the engineered modifications in paragraphs 1 or 2.
6. The male-fertile maintainer plant of any of paragraphs 1-5, wherein the male sterile plant comprises engineered knock-out modifications at each allele of the Mf gene.
7. The male-fertile maintainer plant of any of paragraphs 1-6, wherein at least one copy of any of the engineered modifications is engineered by using a site-specific guided nuclease.
8. The male-fertile maintainer plant of paragraph 7, wherein the site-specific guided nuclease is a form of CRISPR-Cas (such as CRISPR-Cas9).
9. The male-fertile maintainer plant of any of paragraphs 7-8, wherein a multi-guide construct is used.
10. The male-fertile maintainer plant of any of paragraphs 1-9, wherein the endogenous Mf, PV, and OV genes are located on the same arms of the same homologous pair of chromosomes.
11. The method of any of paragraphs 1-10, wherein the plant is wheat.
12. The method of any of paragraphs 1-11, wherein the plant is hexaploid wheat, tetraploid wheat, Triticum aestivum, or Triticum durum.
13. The method of any of paragraphs 1-10, wherein the plant is triticale, oat, canola/oilseed rape or indian mustard.
14. The method of any of paragraphs 1-13, wherein the PV gene has homology to a gene demonstrated to be vital for post-meiosis events such as pollen-grain development, germination, or pollen tube extension in a plant.
15. The method of any of paragraphs 1-14, wherein the PV gene is selected from the genes of Table 1.
16. The method of any of paragraphs 1-14, wherein the PV gene displays the same type of activity and shares at least 80%, at least 85%, at least 90%, at least 95%, or greater sequence identity with a PV gene of Table 1.
17. The method of any of paragraphs 1-16, wherein the OV gene has homology to a gene demonstrated to be vital for post-meiosis events such as cell division of the initial archesporial haploid cell, differentiation into an egg cell, a central cell, two synergid cells and three antipodal cells or synthesis and export of pollen-tube attractant compounds in a plant.
18. The method of any of paragraphs 1-17, wherein the OV gene is selected from the genes of Table 2.
19. The method of any of paragraphs 1-17, wherein the OV gene displays the same type of activity and shares at least 80%, at least 85%, at least 90%, at least 95%, or greater sequence identity with a OV gene of Table 2.
20. The male-fertile maintainer plant of any of paragraphs 1-19, wherein the plant does not comprise any genetic sequences which are exogenous to that plant species.
21. A method of producing a male-fertile maintainer plant of any of paragraphs 1-20, wherein the method comprises:
   a. engineering the knock-out modifications in each allele of Mf, OV, and PV in the second and any subsequent genomes, resulting in a fertile plant;
   b. engineering the modifications in the first chromosome of the first genome; and
   c. engineering the modifications in the second chromosome of the first genome.
22. The method of paragraph 21, wherein the knock-out modifications are engineered by contacting a plant cell with a site-specific guided nuclease.
   The method of paragraph 22, wherein the site-specific guided nuclease is a form of CRISPR-Cas (such as CRISPR-Cas9) and multi-guide constructs are used.
23. The method of any of paragraphs 22-23, wherein step a comprises a single step of contacting a plant cell with a Cas enzyme and one or more multi-guide constructs that target each allele of Mf, OV, and PV in the second and subsequent genomes.
24. The method of any of paragraphs 21-24, wherein:
   the modifications in the first chromosome of the first genome are engineered in a first plant;
   the modifications in the second chromosome of the first genome are engineered in a second plant;
   the resulting plants are crossed; and
   the F2 progeny which comprise the engineered first and second chromosomes of the first genome are selected.
25. The method of any of paragraphs 21-25, wherein step b and/or c comprises a single step of contacting a plant cell with a Cas enzyme and one or more multi-guide constructs that direct each engineered modification.
26. A method of producing a male-fertile maintainer plant of any of paragraphs 1-20, wherein the method comprises:
   engineering the pollen construct and/or ovule construct in a first plant;
   transferring the pollen construct and/or ovule construct to a second, wild-type cultivar plant by:
      a) crossing pollen comprising the pollen construct from the first plant onto the second plant;
      b) selfing the F1 generation
      c) in the F2 generation, selecting plants homozygous for the pollen construct and crossing the pollen from the selected homozygous F2 plants onto third, wild-type cultivar plant of the same cultivar as the second plant; and
      d) repeating this process until the crossed plants are substantially isogenic with the wild-type cultivar with the exception of the pollen construct; and
      e) crossing pollen from a fourth wildtype cultivar plant onto a plant comprising the ovule construct;
      f) selfing the F1 generation
      g) in the F2 generation, selecting plants homozygous for the ovule construct and crossing pollen from a fifth, wild-type cultivar plant of the same cultivar as the fourth plant onto the selected homozygous F2 plants; and
      h) repeating this process until the crossed plants are substantially isogenic with the wild-type cultivar with the exception of the ovule construct; and
      i) crossing the pollen from the plant obtained from step (d) onto the plant obtained from step (h); and
      j) selfing the F1 generation, whereby the resulting progeny will have a heterozygous genotype and produce pollen with the pollen construct only and ovules with the ovule construct only.
27. The method of paragraph 27, wherein steps a-d and e-h are performed concurrently.
28. A method of selecting a chromosome arm of a cultivar genome as the site of production of a co-segregating construct;
   wherein the co-segregating construct comprises
      a. an endogenous, wild-type functional allele of a male-fertility gene (Mf gene);
      b. an endogenous, wild-type functional allele of a pollen-grain-vital gene (PV gene);
      c. an endogenous, wild-type functional allele of an ovule-vital gene (OV gene); and
      d. at least one engineered modification comprising a deletion of endogenous intervening sequence between the Mf; PV; and/or OV loci;
   the method comprising:
   a. selecting one of a Mf gene, PV gene, or OV gene;
   b. identifying one of each of the two genes not selected in step a which map to the same arm of the same chromosome as the gene selected in step a;
   c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
   d. selecting the chromosome arm of a cultivar genome as an appropriate site of production if target sequences of step (c) can be identified.
29. The method of paragraph 29, wherein identifying the two genes not selected in step a comprises first identifying the genes in a reference genome and then searching the cultivar genome to identify any translocations or mutations that would affect the two genes.
30. The method of any of paragraphs 29-30, wherein the Mf gene is a gene which has been identified to produce a male-sterile phenotype when modified to a knock-out allele.
31. A system for selecting a chromosome arm of a cultivar genome as the site of production of a co-segregating construct;
   wherein the co-segregating construct comprises
      a. an endogenous, wild-type functional allele of a male-fertility gene (Mf gene);
      b. an endogenous, wild-type functional allele of a pollen-grain-vital gene (PV gene);
      c. an endogenous, wild-type functional allele of an ovule-vital gene (OV gene); and
      d. at least one engineered modification comprising a deletion of endogenous intervening sequence between the Mf; PV; and/or OV loci;
   the system comprising:
   i. a memory having processor-readable instructions stored therein; and
   ii. a processor configured to access the memory and execute the processor-readable instructions, which, when executed by the processor configures the processor to perform a method, the method comprising:
      A. receiving initial data relating to a co-segregating construct, the initial data including one of a Mf gene, PV gene, or OV gene and a reference genome;
      B. processing, using the processor, the initial data to identify one each of the two genes not provided in step A which map to the same arm of the same chromosome as the gene provided in step A;
      C. processing, using the processor, the data obtained from step B) to identify, for each set of a Mf gene, a PV gene, and an OV gene, at least one target sequences for a site-specific guided nuclease guide, with one target sequence identified from each of:
         the sequence distal of regulatory elements regulating expression of the start or end of the open reading frame on the distal side of the proximal gene of any subset of two genes identified in step A; and
         the sequence proximal of regulatory elements proximal of the start or end of the open reading frame on the proximal side of the distal gene of any subset of two genes identified in step A;
      D. creating a library of sets of Mf, PV, and OV genes and associated target sequences;
      E. selecting, using the processor, a set of Mf, PV, and OV genes and associated target sequences with the minimal distance from the target sequences to the border of the open reading frame of the nearest gene from the library of sets.
32. A method of producing a co-segregating construct in a chromosome arm of a cultivar genome; wherein the co-segregating construct comprises
   a. an endogenous, wild-type functional allele of a male-fertility gene (Mf gene);
   b. an endogenous, wild-type functional allele of a pollen-grain-vital gene (PV gene);
   c. an endogenous, wild-type functional allele of an ovule-vital gene (OV gene); and
   d. at least one engineered modification comprising a deletion of endogenous intervening sequence between any two of the Mf; PV; and/or OV loci;
   the method comprising:
   a. selecting one of a Mf gene, PV gene, or OV gene;
   b. identifying one each of the two genes not selected in step a which map to the same arm of the same chromosome as the gene selected in step a;
   c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
   d. selecting the chromosome arm of a cultivar genome as an appropriate site of production if target sequences of step (c) can be identified; and
   e. engineering the at least one modification comprising a deletion of endogenous intervening sequence between the Mf; PV; and/or OV loci by contacting a cell comprising the chromosome with a site-specific guided nuclease and the guides which hybridize to the target sequences identified in step (c).
33. A plant or plant cell comprising a deactivating modification of at least one OV gene.
34. The plant or plant cell of paragraph 34, further comprising a deactivating modification of at least one PV or Mf gene.
35. A plant or plant cell comprising a deactivating modification of at least one PV gene.
36. The plant or plant cell of paragraph 36, further comprising a deactivating modification of at least one OV or Mf gene.
37. The plant or plant cell of any of paragraphs 34-37, wherein the plant permits seed segregation of its progeny.
38. The plant or plant cell of any of paragraphs 34-38, comprising deactivating modifications of each of the copy of the gene(s).
39. The plant or plant cell of any of paragraphs 34-39, wherein the deactivating modification is identical across each genome of the plant.
40. The plant or plant cell of any of paragraphs 34-39, wherein each genome of the plant comprises a different deactivating modification.
41. The plant or plant cell of any of paragraphs 34-41, wherein the gene(s) is selected from the genes of Tables 1-3.
42. The plant or plant cell of any of paragraphs 34-42, wherein the gene(s) has at least 60%, at least 90%, or at least 95% identity with any of the genes of Tables 1-3.
43. The plant or plant cell of any of paragraphs 34-43, wherein the gene(s) has the same activity and at least 60%, at least 90%, or at least 95% identity with any of the genes of Tables 1-3.
44. The plant or plant cell of any of paragraphs 34-44, wherein the deactivating modification is a site-directed mutagenic event resulting from the activity of a site-specific nuclease; or
   the at least one gene is deactivated by site-directed mutagenesis resulting from the activity of a site-specific nuclease.
45. The plant or plant cell of paragraph 45, wherein the site-specific nuclease is CRISPR-Cas.
46. The plant or plant cell of any of paragraphs 34-46, wherein the deactivating modification is excision of at least part of a coding or regulatory sequence; or the at least one gene is deactivated by excision of at least part of a coding or regulatory sequence.
47. The plant or plant cell of any of paragraphs 34-47, wherein the deactivating modification is insertion of RNAi-encoding sequences; or the at least one gene is deactivated by inhibition by expression of RNAi.
48. The plant or plant cell of any of paragraphs 34-47, wherein the deactivating modification is non-transgenic mutagenesis; or the at least gene is deactivated by non-transgenic mutagenesis.
49. A plant or plant cell comprising a modification comprising the deletion of endogenous sequence between a first and second gene, whereby the co-segregation of the first and second genes is increased.
50. The plant or plant cell of paragraph 50, further comprising the deletion of a second endogenous sequence between the second gene and a third gene, whereby the co-segregation of the first, second, and third genes is increased.
51. The plant or plant cell of any of paragraphs 50-51, wherein the first, second, or third gene is a Mf, OV, or PV gene.
52. The plant or plant cell of any of paragraphs 50-52, wherein the at least one deletion is present on a first chromosome or genome, and the plant further comprises a deactivating modification of copies of the first, second, and/or third genes on another chromosome or genome.

Some embodiments of the technology described herein can be defined according to any of the following numbered paragraphs:
1. A polyploidal maintainer plant comprising:
   a first genome comprising an endogenous wild-type functional allele of a Mf gene;
   at least one further genome comprising only recessive or mutated alleles of the Mf gene,
   wherein the plant does not comprise exogenous sequences.
2. A male-fertile maintainer plant for a male-sterile polyploid plant, the maintainer plant comprising:
   in the first chromosome of a homologous pair in a first genome:
   a. an engineered knock-out modification at the allele of a pollen-grain-vital gene (PV gene);
   b. an endogenous, wild-type functional allele of an ovule-vital gene (OV gene); and
   c. at least one engineered modification comprising a deletion of endogenous intervening sequence between the PV; and OV loci;
   in the second chromosome of the same homologous pair in the first genome:
   d. an endogenous, wild-type functional allele of the PV gene; and
   e. an engineered knock-out modification at the allele of the OV gene;
   f. at least one engineered modification comprising a deletion of endogenous intervening sequence between the PV; and OV loci;
   in a second and any subsequent genomes:
   g. an engineered knock-out modification at each allele of the PV gene;
   h. an engineered knock-out modification at each allele of the OV gene;
whereby the pollen grains produced by the male-fertile maintainer plant comprise the second chromosome of the first genome and do not comprise the first chromosome of the first genome (hereinafter referred to as the pollen construct); and
the ovules produced by the male-fertile maintainer plant comprise the first chromosome of the first genome and do not comprise the second chromosome of the first genome (hereinafter referred to as the minimal ovule construct).
3. A male-fertile maintainer plant for a male-sterile polyploid plant, the maintainer plant comprising:
   in the first chromosome of a homologous pair in a first genome:
   a. an endogenous, wild-type functional allele of a male-fertility gene (Mf gene);
   b. an engineered knock-out modification at the allele of a pollen-grain-vital gene (PV gene);
   c. an endogenous, wild-type functional allele of an ovule-vital gene (OV gene); and
   d. at least one engineered modification comprising a deletion of endogenous intervening sequence between the Mf; PV; and/or OV loci;
   in the second chromosome of the same homologous pair in the first genome:
   e. an engineered knock-out modification at the allele of the Mf gene;
   f. an endogenous, wild-type functional allele of the PV gene; and
   g. an engineered knock-out modification at the allele of the OV gene;
   h. at least one engineered modification comprising a deletion of endogenous intervening sequence between the Mf; PV; and/or OV loci;
   in a second and any subsequent genomes:
   i. an engineered knock-out modification at each allele of the Mf gene;
   j. an engineered knock-out modification at each allele of the PV gene;
   k. an engineered knock-out modification at each allele of the OV gene;
whereby the pollen grains produced by the male-fertile maintainer plant comprise the second chromosome of the first genome and do not comprise the first chromosome of the first genome (hereinafter referred to as the pollen construct); and
the ovules produced by the male-fertile maintainer plant comprise the first chromosome of the first genome and do not comprise the second chromosome of the first genome (hereinafter referred to as the ovule construct).
4. The male-fertile maintainer plant of paragraph 2 or 3, wherein the first and second chromosomes of the first genome comprise at least one engineered modification comprising a deletion of endogenous intervening sequence between any two of the Mf; PV; and OV loci.
5. The male-fertile maintainer plant of any of paragraphs 1-4, wherein the plant is hexaploid and the male-sterile plant comprises an engineered knock-out modification at each of the six alleles of the male-fertility gene.
6. The male-fertile maintainer plant of any of paragraphs 1-4, wherein the plant is tetraploid and the male-sterile plant comprises an engineered knock-out modification at each of the four alleles of the male-fertility gene.
7. The male-fertile maintainer plant of any of paragraphs 1-6, wherein the maintainer plant is substantially isogenic with the male-sterile plant with the exception of the engineered modifications.
8. The male-fertile maintainer plant of any of paragraphs 1-7, wherein the male sterile plant comprises engineered knock-out modifications at each allele of the Mf gene.
9. The male-fertile maintainer plant of any of paragraphs 1-8, wherein at least one copy of any of the engineered modifications is engineered by using a site-specific guided nuclease.
10. The male-fertile maintainer plant of paragraph 9, wherein the site-specific guided nuclease is a form of CRISPR-Cas (such as CRISPR-Cas9).
11. The male-fertile maintainer plant of any of paragraphs 9-10, wherein a multi-guide construct is used.
12. The male-fertile maintainer plant of any of paragraphs 1-11, wherein the endogenous Mf, PV, and OV genes are located on the same arms of the same homologous pair of chromosomes.
13. The method of any of paragraphs 1-12, wherein the plant is wheat.
14. The method of any of paragraphs 1-13, wherein the plant is hexaploid wheat, tetraploid wheat, Triticum aestivum, or Triticum durum.
15. The method of any of paragraphs 1-12, wherein the plant is triticale, oat, canola/oilseed rape or indian mustard.
16. The method of any of paragraphs 1-15, wherein the PV gene has homology to a gene demonstrated to be vital for post-meiosis events such as pollen-grain development, germination, or pollen tube extension in a plant.
17. The method of any of paragraphs 1-16, wherein the PV gene is selected from the genes of Table 1.
18. The method of any of paragraphs 1-17, wherein the PV gene displays the same type of activity and shares at least 80%, at least 85%, at least 90%, at least 95%, or greater sequence identity with a PV gene of Table 1.
19. The method of any of paragraphs 1-18, wherein the OV gene has homology to a gene demonstrated to be vital for post-meiosis events such as cell division of the initial archesporial haploid cell, differentiation into an egg cell, a central cell, two synergid cells and three antipodal cells or synthesis and export of pollen-tube attractant compounds in a plant.
20. The method of any of paragraphs 1-19, wherein the OV gene is selected from the genes of Table 2.
21. The method of any of paragraphs 1-20, wherein the OV gene displays the same type of activity and shares at least 80%, at least 85%, at least 90%, at least 95%, or greater sequence identity with a OV gene of Table 2.
22. The male-fertile maintainer plant of any of paragraphs 1-21, wherein the plant does not comprise any genetic sequences which are exogenous to that plant species.
23. A method of producing a male-fertile maintainer plant of any of paragraphs 1-22, wherein the method comprises:
   a. Engineering the knock-out modifications in each allele of Mf, OV, and/or PV in the second and any subsequent genomes, resulting in a fertile plant;
   b. engineering the modifications in the first chromosome of the first genome; and
   c. engineering the modifications in the second chromosome of the first genome.
24. The method of paragraph 23, wherein the knock-out modifications are engineered by contacting a plant cell with a site-specific guided nuclease.
25. The method of paragraph 24, wherein the site-specific guided nuclease is a form of CRISPR-Cas (such as CRISPR-Cas9) and multi-guide constructs are used.
26. The method of any of paragraphs 24-25, wherein step a comprises a single step of contacting a plant cell with a Cas enzyme and one or more multi-guide constructs that target each allele of Mf, OV, and PV in the second and subsequent genomes.
27. The method of any of paragraphs 23-26, wherein:
   the modifications in the first chromosome of the first genome are engineered in a first plant;
   the modifications in the second chromosome of the first genome are engineered in a second plant;
   the resulting plants are crossed; and
   the F2 progeny which comprise the engineered first and second chromosomes of the first genome are selected.
28. The method of any of paragraphs 23-27, wherein step b and/or c comprises a single step of contacting a plant cell with a Cas enzyme and one or more multi-guide constructs that direct each engineered modification.
29. A method of producing a male-fertile maintainer plant of any of paragraphs 1-22, wherein the method comprises:
   engineering the pollen construct, minimal ovule construct, and/or ovule construct in a first plant;
   transferring the pollen construct, minimal ovule construct, and/or ovule construct to a second, wild-type cultivar plant by:
      a) crossing pollen comprising the pollen construct from the first plant onto the second plant;
      b) selfing the F1 generation
      c) in the F2 generation, selecting plants homozygous for the pollen construct and crossing the pollen from the selected homozygous F2 plants onto third, wild-type cultivar plant of the same cultivar as the second plant; and
      d) repeating this process until the crossed plants are substantially isogenic with the wild-type cultivar with the exception of the pollen construct; and
      e) crossing pollen from a fourth wildtype cultivar plant onto a plant comprising the minimal ovule construct or ovule construct;
      f) selfing the F1 generation
      g) in the F2 generation, selecting plants homozygous for the minimal ovule construct or ovule construct and crossing pollen from a fifth, wild-type cultivar plant of the same cultivar as the fourth plant onto the selected homozygous F2 plants; and
      h) repeating this process until the crossed plants are substantially isogenic with the wild-type cultivar with the exception of the minimal ovule construct or ovule construct; and
      i) crossing the pollen from the plant obtained from step (d) onto the plant obtained from step (h); and
      j) selfing the F1 generation, whereby the resulting progeny will have a heterozygous genotype and produce pollen with the pollen construct only and ovules with the minimal ovule construct or ovule construct only.
30. The method of paragraph 29, wherein steps a-d and e-h are performed concurrently.
31. A method of selecting a chromosome arm of a cultivar genome as the site of production of a co-segregating construct;
   wherein the co-segregating construct comprises
      a. optionally, an endogenous, wild-type functional allele of a male-fertility gene (Mf gene);
      b. an endogenous, wild-type functional allele of a pollen-grain-vital gene (PV gene);
      c. an endogenous, wild-type functional allele of an ovule-vital gene (OV gene); and
      d. at least one engineered modification comprising a deletion of endogenous intervening sequence between the Mf; PV; and/or OV loci;
   the method comprising:
   a. selecting one of a Mf gene, PV gene, or OV gene;
   b. identifying one of each of the two genes not selected in step a which map to the same arm of the same chromosome as the gene selected in step a;
   c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
   d. selecting the chromosome arm of a cultivar genome as an appropriate site of production if target sequences of step (c) can be identified.
32. The method of paragraph 31, wherein identifying the two genes not selected in step a comprises first identifying the genes in a reference genome and then searching the cultivar genome to identify any translocations or mutations that would affect the two genes.
33. The method of any of paragraphs 31-32, wherein the Mf gene is a gene which has been identified to produce a male-sterile phenotype when modified to a knock-out allele.
34. A system for selecting a chromosome arm of a cultivar genome as the site of production of a co-segregating construct;
   wherein the co-segregating construct comprises
      a. Optionally, an endogenous, wild-type functional allele of a male-fertility gene (Mf gene);
      b. an endogenous, wild-type functional allele of a pollen-grain-vital gene (PV gene);
      c. an endogenous, wild-type functional allele of an ovule-vital gene (OV gene); and
      d. at least one engineered modification comprising a deletion of endogenous intervening sequence between the Mf; PV; and/or OV loci;
   the system comprising:
   i. a memory having processor-readable instructions stored therein; and
   ii. a processor configured to access the memory and execute the processor-readable instructions, which, when executed by the processor configures the processor to perform a method, the method comprising:
      A. receiving initial data relating to a co-segregating construct, the initial data including one of a Mf gene, PV gene, or OV gene and a reference genome;
      B. processing, using the processor, the initial data to identify one each of the two genes not provided in step A which map to the same arm of the same chromosome as the gene provided in step A;
      C. processing, using the processor, the data obtained from step B) to identify, for each set of a Mf gene, a PV gene, and an OV gene, at least one target sequences for a site-specific guided nuclease guide, with one target sequence identified from each of:
         the sequence distal of regulatory elements regulating expression of the start or end of the open reading frame on the distal side of the proximal gene of any subset of two genes identified in step A; and
         the sequence proximal of regulatory elements proximal of the start or end of the open reading frame on the proximal side of the distal gene of any subset of two genes identified in step A;
      D. creating a library of sets of Mf, PV, and OV genes and associated target sequences;
      E. selecting, using the processor, a set of Mf, PV, and OV genes and associated target sequences with the minimal distance from the target sequences to the border of the open reading frame of the nearest gene from the library of sets.
35. A method of producing a co-segregating construct in a chromosome arm of a cultivar genome;
   wherein the co-segregating construct comprises
      a. optionally, an endogenous, wild-type functional allele of a male-fertility gene (Mf gene);
      b. an endogenous, wild-type functional allele of a pollen-grain-vital gene (PV gene);
      c. an endogenous, wild-type functional allele of an ovule-vital gene (OV gene); and
      d. at least one engineered modification comprising a deletion of endogenous intervening sequence between any two of the Mf; PV; and/or OV loci;
   the method comprising:
   a. selecting one of a Mf gene, PV gene, or OV gene;
   b. identifying one each of the two genes not selected in step a which map to the same arm of the same chromosome as the gene selected in step a;
   c. identifying at least two target sequences for a site-specific guided nuclease guide from the sequence between the distal and central genes of the set of genes identified in step b) and/or identifying at least two target sequences for a site-specific guided nuclease guide from the sequence between the central and distal genes of the set of genes identified in step b); and
   d. selecting the chromosome arm of a cultivar genome as an appropriate site of production if target sequences of step (c) can be identified; and
   e. engineering the at least one modification comprising a deletion of endogenous intervening sequence between the Mf; PV; and/or OV loci by contacting a cell comprising the chromosome with a site-specific guided nuclease and the guides which hybridize to the target sequences identified in step (c).
36. A plant or plant cell comprising a deactivating modification of at least one OV gene.
37. The plant or plant cell of paragraph 36, further comprising a deactivating modification of at least one PV or Mf gene.
38. A plant or plant cell comprising a deactivating modification of at least one PV gene.
39. The plant or plant cell of paragraph 38, further comprising a deactivating modification of at least one OV or Mf gene.
40. The plant or plant cell of any of paragraphs 36-39, wherein the plant permits seed segregation of its progeny.
41. The plant or plant cell of any of paragraphs 36-40, comprising deactivating modifications of each of the copy of the gene(s).
42. The plant or plant cell of any of paragraphs 36-41, wherein the deactivating modification is identical across each genome of the plant.
43. The plant or plant cell of any of paragraphs 36-42, wherein each genome of the plant comprises a different deactivating modification.
44. The plant or plant cell of any of paragraphs 36-43, wherein the gene(s) is selected from the genes of Tables 1-3.
45. The plant or plant cell of any of paragraphs 36-44, wherein the gene(s) has at least 60%, at least 90%, or at least 95% identity with any of the genes of Tables 1-3.
46. The plant or plant cell of any of paragraphs 36-45, wherein the gene(s) has the same activity and at least 60%, at least 90%, or at least 95% identity with any of the genes of Tables 1-3.
47. The plant or plant cell of any of paragraphs 36-46, wherein the deactivating modification is a site-directed mutagenic event resulting from the activity of a site-specific nuclease; or the at least one gene is deactivated by site-directed mutagenesis resulting from the activity of a site-specific nuclease.
48. The plant or plant cell of paragraph 47 wherein the site-specific nuclease is CRISPR-Cas.
49. The plant or plant cell of any of paragraphs 36-48, wherein the deactivating modification is excision of at least part of a coding or regulatory sequence; or the at least one gene is deactivated by excision of at least part of a coding or regulatory sequence.
50. The plant or plant cell of any of paragraphs 36-49, wherein the deactivating modification is insertion of RNAi-encoding sequences; or the at least one gene is deactivated by inhibition by expression of RNAi.
51. The plant or plant cell of any of paragraphs 36-50, wherein the deactivating modification is non-transgenic mutagenesis; or the at least gene is deactivated by non-transgenic mutagenesis.
52. A plant or plant cell comprising a modification comprising the deletion of endogenous sequence between a first and second gene, whereby the co-segregation of the first and second genes is increased.
53. The plant or plant cell of paragraph 52, further comprising the deletion of a second endogenous sequence between the second gene and a third gene, whereby the co-segregation of the first, second, and third genes is increased.
54. The plant or plant cell of any of paragraphs 52-53, wherein the first, second, or third gene is a Mf, OV, or PV gene.
55. The plant or plant cell of any of paragraphs 52-54, wherein the at least one deletion is present on a first chromosome or genome, and the plant further comprises a deactivating modification of copies of the first, second, and/or third genes on another chromosome or genome.
56. A male-fertile maintainer plant for a male-sterile polyploid plant comprising:
   a first and one or more further genomes, and
   modifications of a first and second gene, wherein the first and second genes are selected, in any order, from the group consisting of a PV gene and an OV gene,
   the modifications comprising:
      a. an engineered knock-out modification at each allele of a first gene in the further genomes;
      b. an engineered knock-out modification at each allele of a second gene in every genome; and
      c. engineered modifications in the first genome comprising:
         i. an engineered knock-out modification of at least one allele of the first gene;
         ii. at a loci on a first member of a homologous pair of chromosomes, an engineered insertion or knock-in of the second gene;
      wherein at least one functional copy of the first gene is present in the first genome.
57. The male-fertile maintainer plant of paragraph 56, wherein the engineered modifications in the first genome further comprise:
   a. an engineered knock-out modification of both alleles of the first gene in the first genome; and at a loci on a second member of the homologous pair of chromosomes which is homologous to the loci on the first member of the homologous pair of chromosomes, an engineered insertion or knock-in of the first gene; or
   b. wherein the loci on the first member of a homologous pair of chromosomes is the loci of the first gene and the wild-type functional allele of the first gene is not modified on the second member of the homologous pair of chromosomes.
58. A male-fertile maintainer plant for a male-sterile polyploid plant comprising:
   a first and one or more further genomes, and
   modifications of a first, second, and third gene, wherein the first, second, and third genes are selected, in any order, from the group consisting of a Mf gene, a PV gene, and an OV gene, the modifications comprising:
      a. an engineered knock-out modification at each allele of a first gene in the further genomes;
      b. an engineered knock-out modification at each allele of a second gene in every genome;
      c. an engineered knock-out modification at each allele of a third gene in every genome; and
      d. engineered modifications in the first genome comprising:
         i. an engineered knock-out modification of at least one allele of the first gene;
         ii. at a loci on a first member of a homologous pair of chromosomes, an engineered insertion or knock-in of the second gene; and
         iii. at a loci on a second member of the homologous pair of chromosomes, an engineered insertion or knock-in of the third gene;
      wherein at least one functional copy of the first gene is present in the first genome and in the first genome the one member of the homologous pair of chromosomes comprises a functional copy of the Mf and OV genes and the other member of the homologous pair of chromosomes comprises a functional copy of the PV gene.
59. The male-fertile maintainer plant of any of paragraphs 57-58, wherein the engineered insertion or knock-in of the second or third gene also comprises a knock-out modification of the first gene.
60. The male-fertile maintainer plant of any of paragraphs 56-59, wherein the loci on the first member of a homologous pair of chromosomes is the loci of the first gene.
61. The male-fertile maintainer plant of any of paragraphs 56-60 wherein the loci on the first member of a homologous pair of chromosomes is located within the intergenic space separating the loci of the first gene from the adjacent genes or within one of the adjacent genes.
62. The male-fertile maintainer plant of any of paragraphs 56-61, wherein one or more of the loci on the pair of homologous chromosomes are intergenic.
63. The male-fertile maintainer plant of any of paragraphs 56-61, wherein one or more of the loci on the pair of homologous chromosomes are intragenic.
64. The male-fertile maintainer plant of paragraph 58, wherein the first gene and third genes are, in either order, the Mf and OV genes, the engineered modifications of d. comprise:
   i. at the loci of the first gene on a first member of a homologous pair of chromosomes, an engineered insertion or knock-in of the second gene and an engineered knock-out of the first gene; and
   ii. at the loci of the first gene, within the intergenic space separating the loci of the first gene from the adjacent genes, or within one of the adjacent genes, on a second member of the homologous pair of chromosomes, an engineered insertion or knock-in of the third gene and either:
      1. no modification of the first gene itself; or
      2. a knockout modification of the endogenous loci of the first gene and a knock-in or insertion of the first gene.
65. The male-fertile maintainer plant of paragraph 58, wherein the first gene is the PV gene, the engineered modifications of d. comprise:
   i. at the loci of the first gene on a first member of a homologous pair of chromosomes, an engineered insertion or knock-in of the second and third genes and an engineered knock-out of the first gene; and
   ii. at the loci of the first gene, within the intergenic space separating the loci of the first gene from the adjacent genes, or within one of the adjacent genes, on a second member of the homologous pair of chromosomes either:
      1. no modification of the first gene itself; or
      2. a knockout modification of the endogenous loci of the first gene and a knock-in or insertion of the first gene.
66. The male-fertile maintainer plant of paragraph 58, wherein the plant comprises an engineered knock-out modification at each allele of the first gene in every genome and the engineered modifications of d. comprise:
   i. at a loci on one member of a homologous pair of chromosomes, an engineered insertion or knock-in of the second gene; and
   ii. at a loci on the other member of the homologous pair of chromosomes, an engineered insertion or knock-in of the third gene.
67. A male-fertile maintainer plant for a male-sterile polyploid plant comprising a first and one or more further genomes, the maintainer plant comprising:
   a. an engineered knock-out modification at each allele of a PV gene in every genome;
   b. an engineered knock-out modification at each allele of an OV gene in every genome; and
   c. engineered modifications in the first genome comprising:
      i. at a loci on a first member of a homologous pair of chromosomes, an engineered insertion or knock-in of the PV gene; and
      ii. at a loci on a second member of the homologous pair of chromosomes, an engineered insertion or knock-in of the OV gene.
68. The male-fertile maintainer plant of paragraph 67, further comprising:
   an engineered knock-out modification at each allele of a Mf gene in every genome.
69. The male-fertile maintainer plant of paragraph 68, wherein the modification of c.ii. further comprises an engineered insertion or knock-in of the OV gene and Mf gene.
70. A male-fertile maintainer plant for a male-sterile polyploid plant comprising a first and one or more further genomes, the maintainer plant comprising:
   a. an engineered knock-out modification at each allele of a Mf gene in the further genomes;
   b. an engineered knock-out modification at each allele of a PV gene in every genome;
   c. an engineered knock-out modification at each allele of an OV gene in every genome; and
   d. engineered modifications in the first genome comprising:
      i. an engineered knock-out modification of at least one allele of the Mf gene;
      ii. at a loci on a first member of a homologous pair of chromosomes, an engineered insertion or knock-in of the PV gene; and
      iii. at a loci on a second member of the homologous pair of chromosomes, an engineered insertion or knock-in of the OV;
      wherein at least one functional copy of the Mf gene is present in the first genome.
71. The male-fertile maintainer plant of paragraph 70, wherein the engineered insertion or knock-in of the PV gene also comprises a knock-out modification of the Mf gene.
72. The male-fertile maintainer plant of any of paragraphs 69-71, wherein the loci on the first member of the pair of chromosomes is located within the intergenic space separating the Mf loci from the adjacent genes or within one of the adjacent genes.
73. The male-fertile maintainer plant of paragraph 70, wherein the engineered modifications of d. comprise:
   i. at the Mf loci on a first member of a homologous pair of chromosomes, an engineered insertion or knock-in of the PV gene and an engineered knock-out of the Mf gene; and
   ii. at the Mf loci, within the intergenic space separating the Mf loci from the adjacent genes, or within one of the adjacent genes, on a second member of the homologous pair of chromosomes, an engineered insertion or knock-in of the OV gene and either:
      1. no modification of the Mf gene itself; or
      2. a knockout modification of the endogenous Mf loci and a knock-in or insertion of the Mf gene.
74. The male-fertile maintainer plant of paragraph 70, wherein the plant comprises an engineered knock-out modification at each allele of the Mf gene in every genome and the engineered modifications of d. comprise:
   i. at a loci on a first member of a homologous pair of chromosomes, an engineered insertion or knock-in of the PV gene; and
   ii. at a loci on a second member of the homologous pair of chromosomes, an engineered insertion or knock-in of the OV gene.
75. The male-fertile maintainer plant of any of paragraphs 56-74, wherein the loci on the first and second members of the pair of chromosomes are homolgous, inter-genic regions and not coextensive with the endogenous Mf, PV, and/or OV alleles.
76. The male-fertile maintainer plant of any of paragraphs 56-74, wherein the engineered knock-in modifications are on a different chromosome than the engineered knock-out modifications of the Mf, PV, and/or OV alleles.
77. The male-fertile maintainer plant of any of paragraphs 56-75, wherein the engineered knock-in modifications are located in intergenic sequences.
78. The male-fertile maintainer plant of any of paragraphs 56-75, wherein the engineered knock-in modifications are located in intragenic sequences.
79. The male-fertile maintainer plant of any of paragraphs 56-78, wherein the Mf, PV, and/or OV alleles are on the same chromosome.
80. The male-fertile maintainer plant of any of paragraphs 56-79, wherein the endogenous Mf, PV, and OV alleles are located on the same arms of the same homologous pair of chromosomes.
81. The male-fertile maintainer plant of any of paragraphs 56-80, wherein the endogenous PV and OV alleles are located on the same arms of the same homologous pair of chromosomes.
82. The male-fertile maintainer plant of any of paragraphs 56-78, wherein two alleles of the Mf, PV, and OV alleles are on the same chromosome, and the third allele is on a different chromosome than the two alleles.
83. The male-fertile maintainer plant of any of paragraphs 56-78, wherein the Mf, PV, and/or OV alleles are each on a different chromosome.
84. The male-fertile maintainer plant of any of paragraphs 56-83, wherein the plant is hexaploid and the male-sterile plant comprises an engineered knock-out modification at each of the six alleles of the male-fertility gene.
85. The male-fertile maintainer plant of any of paragraphs 56-83, wherein the plant is tetraploid and the male-sterile plant comprises an engineered knock-out modification at each of the four alleles of the male-fertility gene.
86. The male-fertile maintainer plant of any of paragraphs 56-85, wherein the maintainer plant is substantially isogenic with the male-sterile plant with the exception of the engineered modifications.
87. The male-fertile maintainer plant of any of paragraphs 56-86, wherein the male sterile plant comprises engineered knock-out modifications at each allele of the Mf gene.
88. The male-fertile maintainer plant of any of paragraphs 56-87, wherein at least one copy of any of the engineered modifications is engineered by using a site-specific guided nuclease.
89. The male-fertile maintainer plant of paragraph 88, wherein the site-specific guided nuclease is a form of CRISPR-Cas (such as CRISPR-Cas9).
90. The male-fertile maintainer plant of any of paragraphs 88-89, wherein a multi-guide construct is used.
91. The male-fertile maintainer plant of any of paragraphs 56-90, wherein the plant is wheat.
92. The male-fertile maintainer plant of any of paragraphs 56-92, wherein the plant is hexaploid wheat, tetraploid wheat, Triticum aestivum, or Triticum durum.
93. The male-fertile maintainer plant of any of paragraphs 56-90, wherein the plant is triticale, oat, canola/oilseed rape or indian mustard.
94. The male-fertile maintainer plant of any of paragraphs 56-93, wherein the PV gene has homology to a gene demonstrated to be vital for post-meiosis events such as pollen-grain development, germination, or pollen tube extension in a plant.
95. The male-fertile maintainer plant of any of paragraphs 56-94, wherein the PV gene is selected from the genes of Table 1.
96. The male-fertile maintainer plant of any of paragraphs 56-95, wherein the PV gene displays the same type of activity and shares at least 80%, at least 85%, at least 90%, at least 95%, or greater sequence identity with a PV gene of Table 1.
97. The male-fertile maintainer plant of any of paragraphs 56-96, wherein the OV gene has homology to a gene demonstrated to be vital for post-meiosis events such as cell division of the initial archesporial haploid cell, differentiation into an egg cell, a central cell, two synergid cells and three antipodal cells or synthesis and export of pollen-tube attractant compounds in a plant.
98. The male-fertile maintainer plant of any of paragraphs 56-97, wherein the OV gene is selected from the genes of Table 2.
99. The male-fertile maintainer plant of any of paragraphs 56-98, wherein the OV gene displays the same type of activity and shares at least 80%, at least 85%, at least 90%, at least 95%, or greater sequence identity with a OV gene of Table 2.
100. The male-fertile maintainer plant of any of paragraphs 56-99, wherein the plant does not comprise any genetic sequences which are exogenous to that plant species.
101. A method of producing a male-fertile maintainer plant of any of paragraphs 56-100, wherein the method comprises:
   a. engineering the knock-out modifications in each allele of Mf, OV, and/or PV in each genome;
   b. engineering the remaining modifications in the first genome.
102. The method of paragraph 101, wherein the knock-out modifications are engineered by contacting a plant cell with a site-specific guided nuclease.
   The method of paragraph 102, wherein the site-specific guided nuclease is a form of CRISPR-Cas (such as CRISPR-Cas9) and multi-guide constructs are used.
103. The method of any of paragraphs 101-102, wherein step a comprises a single step of contacting a plant cell with a Cas enzyme and one or more multi-guide constructs that target each allele of Mf, OV, and/or PV in the genomes.
104. The method of any of paragraphs 101-103, wherein step b comprises a single step of contacting a plant cell with a Cas enzyme and one or more multi-guide constructs that direct each engineered modification.

### EXAMPLES

### Example 1: Engineering Knock-Out Modifications

To produce plants with targeted mutations in PV1 and OV1 a CRISPR Cas9 system was utilized to introduce mutations in wheat plants. PV1 and OV1 were targeted with four guide RNAs for each set of homoeologues. To identify the target sequences in these genes the publicly available program DREG (available on the world wide web at emboss.sourceforge.net/apps/cvs/emboss/apps/dreg.html) was used to find sequences that match either ANNNNNNNNNNNNNNNNNNNNGG or GNNNNNNNNNNNNNNNNNNNNGG in either direction of the Fielder variety genomic sequence.

Four guides (e.g., sgRNAs) were then selected based on the following three criteria: that the target sequence was conserved in all three homoeologues, that it was (at least partially) in an exon of PV1 or OV1, and that homoeologue specific regions were readily identifiable for PCR identification of mutations. It was also attempted to use either AN20GG or GN20GG as this would stabilize the construct for transformation in the plant and allow for greater number of potential guides which could be used.

The guide sequences selected are shown in SEQ ID Nos 10-13 and 23-26. For targeting both PV1 and OV1, the four appropriate guides for each target wheat gene were expressed with promoters in the order: TaU6, TaU3, TaU6 and OsU6 promoters. The two promoters/guides constructs were synthesized and subsequently cloned into an intermediate vector containing L1 L5r flanking sites for multisite gateway recombination into the final binary vector containing a wheat-optimized Cas9 enzyme driven by the maize ubiquitin promoter flanked by L5 and L2 sites. This final vector was introduced into Agrobacterium for transformation into wheat.

Wheat transformation of Fielder spring wheat germplasm with the construct(s) was carried out using immature wheat embryos, following Ishida et al. (2015). Transformation can also be performed in accordance with Perochon, A. et al. (2015). Plant physiology, 169(4), 2895-2906. Transformed plants are then grown to seed and for mutations using a PCR based method where the PCR product was amplified for each homoeologue and sequenced to identify mutations. Each of the references referred to in this Example are incorporated in their entireties by reference herein.

### Example 2: Exemplary Intergenic Deletions

The genes PV1, Mfw2 and OV1 are all on the short arms of chromosomes 7A, 7B, and 7D except for PV1-B which is part of the translocation from chromosome 7B to chromosome 4A. They are in the order PV1 (distal end with respect to the centromere), Mfw2 and OV1 (proximal end); there are ˜1275 genes between PV1 and Mfw2, only 4 genes between Mfw2 and OV1. There will, therefore be significant crossing over and recombination between PV1 and Mfw2 but minimal between Mfw2 and OV1. So, in the case of these particular three genes it is feasible, for the invention to be effective, to produce a large deletion between PV1 and Mfw2 only. Accordingly, in the embodiments described in this example below, intergenic deletion(s) are made only between PV1 and Mfw2 but not between OV1 and Mfw2. In alternative embodiments, it is contemplated that intergenic deletion(s) are made between OV1 and Mfw2 and such deletion(s) can be generated using the approach described in this example.

To produce plants with the desired deletion(s) in the DNA between a PV1 and Mfw2 gene a CRISPR Cas9 system was used to introduce the deletions in wheat plants. The genes immediately following PV1 and preceding Mfw2 were targeted with six guide RNAs targeting the A and D homoeologues. To identify the target sequences in these genes the publicly available program DREG (available on the world wide web at emboss.sourceforge.net/apps/cvs/emboss/apps/dreg.html) was used to find sequences that match either ANNNNNNNNNNNNNNNNNNNNGG or GNNNNNNNNNNNNNNNNNNNNGG in either direction of the Chinese Spring genomic sequence.

Six guides were selected based on the following three criteria: that the target sequence was conserved in both homoeologues, the guides are close together to detect the deletions by PCR, and that homoeologue specific regions for PCR identification of mutations were readily identifiable. The design also included, in each targeting gene, one guide driven by TaU3, one by TaU6 and one by OsU6 to limit recombination in both Agrobacterium and plants. The guide sequences selected are shown in SEQ ID Nos 58-63 and 67-71.

For targeting the sequence following (from the distal end of the chromosome) PV1 and preceding Mfw2 the six appropriate guides for each target wheat gene were driven with promoters in the order: TaU3, TaU6 and OsU6. These promoters/guides' constructs were synthesized by Genewiz™ and subsequently cloned into an intermediate vector containing L1 L5r flanking sites for multisite gateway recombination into the final binary vector containing a wheat-optimized Cas9 enzyme driven by the maize ubiquitin promoter flanked by L5 and L2 sites. This final vector was introduced into Agrobacterium for transformation into wheat as documented in Example 1.

Plants were then screened for mutations using a PCR based methods where PCR products were designed to amplify flanking sequences of the targeted genomic regions as well as genes which reside in the targeted deleted area (established from Clavijo et al, 2017) to detect the deletions for each homoeologue and PCR products were sequenced to verify the deletions. Using such data, selections were made for deletions in either the A or D genome; this was repeated in subsequent generation(s) until the deletions were only in one genome.

Sequences

SEQ ID NO: 1 PV1-A CDS
ATGGCGGAGCCGGAGGACGGCGGCGAGGTCGCCCCTCCTGAGGCGGCGGCGGCGGCGACG
AGCGCGGCCGCCCATTCGTCTCCCCCTGCTAAGGAGGAGCCGGCGGCAGCGGCAGAGGCAA
AGCCGGCCAGCTCCGGCGAGGCGGTCTCCCTCAACTACGAGGAAGCGAGAGCTCTCTTAGG
AAGGCTGGAATTTCAGAAAGGCAATGTAGAAGATGCACTTTGTGTGTTTGATGGAATAGACC
TTCAAGCTGCCATTGAGCGCTTCCAGCCATCATCCTCGAAGAAAACAACAGAAGCTACTCTT
GTTCTCGAAGCCATTTACTTGAAAGCATTGTCCCTTCAGAAGCTAGGAAAATCAATAGAGGC
CGCTAAACAATGCAAAAGCGTCATCGATTCTGTTGAAAGTATGTTCAAGAATGGCACTCCTG
ACATCGAACAGAAGCTACAAGAAACTATCAATAAATCTGTGGAACTTCTCCCAGAGGCCTG
GAAAAAAGCTGGCTCTCTTCAGGAAACATTTGCTTCGTACAGACGCGCTCTTCTCAGCCCGT
GGAACCTCGACGAGGAATGCATTGCAAGGATTCAAAAGAGATTTGCTGCTTTCTTGTTGTAT
GGTTGTGTGGAGTGGAGTCCGCCCAGCTCTGGTTCACCAGCTGAAGGCACTTTTGTTCCCAA
GACAAATATTGAGGAAGCCATTCTACTCCTCACAACAGTATTGAAGAAGTTTTATCAGGGAA
AGACCCACTGGGATCCCTCGGTGATGGAACACTTGACCTACGCATTGTCGATTTGCAGCCGG
CCTTCTCTTATTGCAGATCATCTGGAGGAGGTTCTACCTGGGATATATCCTCGGACGGAGAG
ATGGAACACACTAGCATTTTGCTACTATGGTGTTGCTCAGAAAGAAGTCGCTCTAAATTTCC
TGAGGAAGTCCTTGAATAAGCATGAGAACCCAAAAGATACAATGGCATTGCTGTTAGCCGC
CAAGATATGTAGCGAGGACTGCCGTCTTGCCTCCGAGGGTGTCGAGTACGCAAGAAGAGCG
ATTGCAAACACGGAATCATTAGATGTTCATCTGAAGAGCACTGGCCTCCATTTCTTGGGGAG
TTGCCTGAGTAAGAAGGCCAAGATTGTTTCATCCGATCACCAAAGAGCTATGTTGCACGCAG
AAACTATGAAGTCCCTTACGGAGTCGATGTCTCTTGACCGCTACAACCCAAACCTAATATTC
GACATGGGAGTTCAATACGCTGAGCAGCGGAACATGAACGCCGCGCTGAGATGTGCCAAAG
AGTTTGTCGACGCGACCGGTGGAGCGGTCTCGAAAGGTTGGAGGTTTCTAGCCCTAGTCCTC
TCCGCACAGCAAAGATACTCCGAAGCAGAAGTGGCGACCAATGCCGCGTTAGACGAGACCG
CAAAGTGGGATCAAGGGTCACTGCTCAGGATAAAGGCTAAGCTGAAGGTCGCTCAATCGTC
GCCCATGGAGGCGGTGGAGGCATACCGGGTCCTCCTTGCTCTTGTTCAGGCCCAGAAGAATT
CGCCTAAAAAAGTGGAGGGAGAGGCTGGTGGAGTAACCGAGTTCGAAATCTGGCAAGGTCT
TGCAAATCTGTACTCCGGCCTCTCACACACCAGGGACGCCGAGGTATGTTTGCAGAAAGCCA
CAGCCCTGAAATCGTACTCCGCCGCGACACTCGAAGCCGAAGGTTACATGCACGAGGTGCG
CAAGGAGAGCAAGGAGGCGATGGCGGCCTACGTGAACGCCTCGGCGACGGAGCTGGATCAC
GTGTCGTCCAAGGTGGCCATCGGGGCTCTGCTCTCCAAGCAGGGGGGCAAGTACCTCCCGGC
GGCGAGGGCCTTCCTCTCGGACGCCCTGAGGGTCGAGCCGACGAACCGGATGGCGTGGCTC
AACCTGGGGAAGGTGCACAAGCTCGACGGGAGGATTTCCGACGCCGCCGACTGCTTCCAGG
CGGCGGTGATGCTCGAGGAGTCGGATCCCGTGGAGAGTTTTAGGACGCTCTCATGA
SEQ ID NO: 2 PV1-A polypeptide sequence
MAEPEDGGEVAPPEAAAAATSAAAHSSPPAKEEPAAAAEAKPASSGEAVSLNYEEARALLGRLE
FQKGNVEDALCVFDGIDLQAAIERFQPSSSKKTTEATLVLEAIYLKALSLQKLGKSIEAAKQCKSV
IDSVESMFKNGTPDIEQKLQETINKSVELLPEAWKKAGSLQETFASYRRALLSPWNLDEECIARIQ
KRFAAFLLYGCVEWSPPSSGSPAEGTFVPKTNIEEAILLLTTVLKKFYQGKTHWDPSVMEHLTYA
LSICSRPSLIADHLEEVLPGIYPRTERWNTLAFCYYGVAQKEVALNFLRKSLNKHENPKDTMALL
LAAKICSEDCRLASEGVEYARRAIANTESLDVHLKSTGLHFLGSCLSKKAKIVSSDHQRAMLHAE
TMKSLTESMSLDRYNPNLIFDMGVQYAEQRNMNAALRCAKEFVDATGGAVSKGWRFLALVLS
AQQRYSEAEVATNAALDETAKWDQGSLLRIKAKLKVAQSSPMEAVEAYRVLLALVQAQKNSP
KKVEGEAGGVTEFEIWQGLANLYSGLSHTRDAEVCLQKATALKSYSAATLEAEGYMHEVRKES
KEAMAAYVNASATELDHVSSKVAIGALLSKQGGKYLPAARAFLSDALRVEPTNRMAWLNLGK
VHKLDGRISDAADCFQAAVMLEESDPVESFRTLS
SEQ ID NO: 3 PV1-A genomic sequence Start codon at bases 3,142-3,144.
Stop codon at bases 9,522-9,524
CTCGAAGTGCGTTAACCAAAACAAATCCACCAAAGACGGCTCTGGACTGATATGGTGTTAA
ATAGCAAACTGAGTTTCAGAGGATGAATAGGAGAGGTCAGTTAGACAGAAATTGTGCACAA
ATCAACCAAAGACAGCTGTAGGCAAAAGTTCTGTTGAATGGCAAACAGGGTTTCAGAAAAG
GAACAGGATAGGTCAGTTAGTTGTGTACTAAGAACTCTCATCTACACTGCAGTTCACGAAAA
AGGAAGAACCACTCGGTGCACGACATACCCGAGCATCATCCTCCTCCTTTGAGACTTCTTTG
ACAACCACCTCCACTTCGCGTTTGTAAAGCTGATCAAACAAATGAGAGACTTGTAAGCCAGC
AAAGCAGTAATAGTTTACAATGTAAATATTCTTACGGTAACAGAACTTTACAAGAAGCAAAT
ACTTCAGTGGAGATGAACTAGAATGAACCAAAATAACTTCAGCACCAACTTGCTCACTGAAC
ACAAGTAGCATAGAGTTGTATATAAGCCTATTCTACCAAAGAGCTACTAAGATGCAACAAGT
ATTGGAGAGCTCGTAAAATTCATTCAATACGCAGATGAAGAACTGATAAACGAACTCTGGA
AAGCAGAGCCTCAAAAGCCAGCAGAGTAAGCTAGTAGTTAGTAAGCAAATGCTTGTGAGCC
GCGACGGAGCATTCCAAACTGCACGGCCATCGCGGCATGTTTATTTCTATCGGGGAAAAGAA
GGGGGAAGCTAACCTTGCTCTGCTCGCGCATGAGTATGGCGAACTTGTCGATGTTGGGGACG
GCGAGCATCTTGGGCATGAGGTAGTTGCGGAAGTGCCCCGGCGCCACCTTCACCGTCTCCCC
CGCCTTCCCCAGCTTGTCGATCGTCTGAGGTGAACAAGCGATGGGTGATGTCAAAGGTTAGT
TCCACTTCCCCGCACAATCTAAAATCTCTAGGGACATTGTTGAAATGAAAGGCCAAAACTGA
AGCTTTATCGGTCAAAAATACTACTGCTAGCTTAAAAAGTTTCAGAAATGCTGGAGATTTAT
CGGTCAAACTGTCGCTGAGGCGGCACCGGCCTCACCGGATCGAAAGCACCCCGCTCGAACT
GACCGGAAACGCAAGCGGCTAGCGAGATCGCGGGATGCATCCTGCAGAGGTGGAGGACCG
AGCGGAGCGTCGCGGGGGGAGGGGGGCAGGGGGGGTGGCTTACCGTGGTGAGGATGACCT
CGAGCTTGCGGTAGCGGAGGCCGTGGCCGGAGAAGAGGACGGGGTTGGTGGCGGCGGCGCC
GAGGCCGTGGCGGCGGAGGAGGGCGGCGCGGGCGGCGGCCATGGTGGAGTAGGGTTCAGG
GGAAGGAGGCGACGGGGGCCGGCGGCTGCCACCAACGGGTGCGCGAGTGAGAGTATTGGT
GGCTCGGCTTCCCGCCGGACCGGGCCGGTGCCAGGCCAGGCCCGCTAAGGGATCTCCATTTT
TTCCTTTGATTTTATTTTTAAAATCCTTCTGCTGCCCAAAAGAATTTGCATTTTGCACTTTCTT
GAGCCCTCTTTGATTTTATTTTTTAAATACTTCCGCTGCCATGAAAACTTTGCAGTTTCCACTT
TTTTGGATGAGGAAGTCGACCAGAGCGGAAATCTGGAAAAGAGCCAGGGTTCTTCTGCTGG
ATGCCAACACCCTCTGCAATCCAATAAAATCAAATCAAACATTCAAAATCTCATCAGAATAT
CAACTTTATGTTTTTTTCTTAAGGCACATAAATGCATTTTTTTGTAACATAAAAGGTTATGTG
AGTTTTTAGTCCAATTTGTTTCGTAGTTGGCAGGTTGAAACTCTAGGACTCGGATATGTGCTA
TACTCAAGCACCACATGTTACATTTTATTTTGCGCTGAAAATCAAGACATGCATCATTAACTT
TCATATTTCATGAAAGTTACAATAGTTAGACCCTCTCCATTTCAATTTCCAAAGATGTAGGAT
GCAACAATTCCTTTTACCACCAAGACATATTAATATTGTGTGGTTTCCGTGATATGAACTCCC
CTATCCCTTGGTGGCTATGGTAAATCTCCCCTCCAGGCTTCATCAATGAGACCGTGGATTCGC
CTCCCCTCTACCTGCCGCTCCGACGACTGGTGGCGGGGTTAGGGATCCCGGTGCTTTCGGTCT
GGTTAATAGTTTAGGTTAGGTTTTTTTAGTCTTCTTAGGTGTGGCGCTCAGATGGATGGCAGC
GCTTTTTCTCGAGTTTGTGTTTCGGTCTCCGATTCTCCTCAAGTTCGTTCATCTGAACGTAATT
GAAGGACCTCCGACGTAGATTTCTGTCGTCTCCTTGCTACGATGAGTTTAGTGTTTCTCGTCG
TGTGACGAGATTTGTTGTCAGGTGCTTCAGATCTATTGAAGGGTTCAACGGTGACGACTACG
ACTCTAGGGCACTAGTCCTTACGGGCACATGCATGAAGACTTCCCGACTGTCATCGTATGGT
CAAGCCGGCTACAGTAGGGGAACAACGGTAGTGGTCATTCGATGGTGAAGAGGCGTTCTTT
GTGGGCAAGCCAAATGATTCTGATCTATTATCAATGTTCACCAGAAAAAACAAAGCACCTTG
TTGTTTCAATTTTGCGAAAAATGATTCAAATCTATTATCAATGTTCACCAGCAAAAAAGAAA
AATAAAGCACCTTTTGCTTTGCTCTTGAGCAAAAATTCTTTTGAGTGGAAAAAATACACCCT
GTTGTTTCTCTTTGCAGCCAAGGACAAAGCATAGGTTACGATCACATCTTGGTCAACATATG
TGGCCGTTCAAGATCATCCATGCATGCATTTGTATTGGTGGAGCTAGACAATCTATTTTTAGC
TTGTCTTAGAAAAAAATCTATCATTAGCTCGAATTTTCTGGAAAAAATTGTAAGGACTCCCTT
AAATTTCTATCGGTATTCCTGATGAACTTTCCACGTGGCAACAAGCAGTAGAGAGAGTAGTC
GCAAAAGAGTAAAAATAGAAGACAGAAAAATTAGTGGAAAAGGGTACGCATGCGAACCGT
GGAAGAAGTTGCCGCCTCCGCTCCTCTCCATCGACGACGAAACCGAGCACCTCCCAGCTCGA
CGAGATCGGGTGCCCGTCGGTGCGATCCCTCTGCTGCAGCGGCATGGCGGAGCCGGAGGA
CGGCGGCGAGGTCGCCCCTCCTGAGGCGGCGGCGGCGGCGACGAGCGCGGCCGCCCATT
CGTCTCCCCCTGCTAAGGAGGAGCCGGCGGCAGCGGCAGAGGCAAAGCCGGCCAGCTCC
GGCGAGGCGGTCTCCCTCAACTACGAGGTCCGAAATATCTGAAACTCTTTTATGAATGTTT
GTTTGATACGTAGTACGGTGCTTGTCCTATATAATGCTGCTATGATGTGAACTTGGTTTGCAA
GAAATTGCCATGTTTGAAGTGTTTGGTCAGTGCCGCCAATGTTATGTCAAATTTCGTATTGCC
GGCGATGATGGTGTCAATTCAATTAAGCGATGACTTTGATTGTTCTCACATAAACCGAAAAT
GTAAAGATGCCAACGTTGGTCGTGCGTTTTTTTCAAAAAATATTGTTTGAGAGGCTTTGTGTG
GGAAATGTGTTCCTTTCTTGGGGATGTCAAATGCTGAATTGTGATTCCATTTCAGTTCTGGTT
CTATTTCATTGATTGGTTTATCCAATTGCGAATTATTCGGCAAGTTTATAAGACATGCACCTT
TTTTTGTTCTTTATATATTTGGGTGAGTGAATTATAACACGATGGTGTCAATCAAAATGCTTT
TTATTGGGTGAGTGAATTGTGAATAATCTTAATGCCAGTATAGGTAGCAAGATTTTACTGAA
TGATGTGTAATCATACGGAGAAAGGGACATTTTCTTTGTCCAGATTATGAAGAACTGATCAT
ATTTCTATTCCCATGAACCATGCTATTGATCTCCATTGCAATTATTAATTTCCAAAAATGAAG
TTCAAACTTAGCTTAATACATGGAGAATTCCAACCGTCATGCTTTCTCGGGTTTATTACACCA
AGTTATTTTTTTGCGGGTTTATTACACCAAGTTCGTTTATACATCTATCGGTAACAGGAAGCG
AGAGCTCTCTTAGGAAGGCTGGAATTTCAGAAAGGCAATGTAGAAGATGCACTTTGTGTGTT
TGATGGAATAGACCTTCAAGCTGCCATTGAGCGCTTCCAGCCATCATCCTCGAAGAAAACAA
CAGAAGCTACTCTTGTTCTCGAAGCCATTTACTTGAAAGCATTGTCCCTTCAGAAGCTAGGA
AAATCAATAGGTAACAAAATTGCTTTATACCGTTGTTTAAGTTTAAAACAAATTGCTTTAATT
GTGTTTTACAAAAATAAATTATCATTTGGAAGTTGTTCTTTTTTTTAGCTTATTCTTTGACTTG
TAACAAATTACTGAAATACCTGTTGAACATGCAGAGGCCGCTAAACAATGCAAAAGCGTCA
TCGATTCTGTTGAAAGTATGTTCAAGAATGGCACTCCTGACATCGAACAGAAGCTACAAGAA
ACTATCAATAAATCTGTGGAACTTCTCCCAGAGGCCTGGAAAAAAGCTGGCTCTCTTCAGGA
AACATTTGCTTCGTACAGACGCGCTCTTCTCAGCCCGTGGAACCTCGACGAGGAATGCATTG
CAAGGATTCAAAAGAGATTTGCTGCTTTCTTGTTGTATGGTTGTGTGGAGTGGAGTCCGCCC
AGCTCTGGTTCACCAGCTGAAGGCACTTTTGTTCCCAAGACAAATATTGAGGAAGCCATTCT
ACTCCTCACAACAGTATTGAAGAAGTTTTATCAGGGAAAGACCCACTGGGATCCCTCGGTGA
TGGAACACTTGACCTACGCATTGTCGATTTGCAGCCGGCCTTCTCTTATTGCAGATCATCTGG
AGGAGGTTCTACCTGGGATATATCCTCGGACGGAGAGATGGAACACACTAGCATTTTGCTAC
TATGGTGTTGCTCAGAAAGAAGTCGCTCTAAATTTCCTGAGGAAGTCCTTGAATAAGCATGA
GAACCCAAAAGATACAATGGCATTGCTGTTAGCCGCCAAGATATGTAGCGAGGACTGCCGT
CTTGCCTCCGAGGGTGTCGAGTACGCAAGAAGAGCGATTGCAAACACGGAATCATTAGATG
TTCATCTGAAGAGCACTGGCCTCCATTTCTTGGGGAGTTGCCTGAGTAAGAAGGCCAAGATT
GTTTCATCCGATCACCAAAGAGCTATGTTGCACGCAGAAACTATGAAGTCCCTTACGGAGTC
GATGTCTCTTGACCGCTACAACCCAAACCTAATATTCGACATGGGAGTTCAATACGCTGAGC
AGCGGAACATGAACGCCGCGCTGAGATGTGCCAAAGAGTTTGTCGACGCGACCGGTGGAGC
GGTCTCGAAAGGTTGGAGGTTTCTAGCCCTAGTCCTCTCCGCACAGCAAAGATACTCCGAAG
CAGAAGTGGCGACCAATGCCGCGTTAGACGAGACCGCAAAGTGGGATCAAGGGTCACTGCT
CAGGATAAAGGCTAAGCTGAAGGTCGCTCAATCGTCGCCCATGGAGGCGGTGGAGGCATAC
CGGGTCCTCCTTGCTCTTGTTCAGGCCCAGAAGAATTCGCCTAAAAAAGTGGAGGTTTGTTTT
CTTAATCAAATGCAGCAAAAAAAAAGTACCATCCGTATACTATTTTTCTCTTGGCACTTTCTC
CATTAGTTCACATACCGATGCTTCAGGGAGAGGCTGGTGGAGTAACCGAGTTCGAAATCTGG
CAAGGTCTTGCAAATCTGTACTCCGGCCTCTCACACACCAGGGACGCCGAGGTATGTTTGCA
GAAAGCCACAGCCCTGAAATCGTACTCCGCCGCGACACTCGAAGCCGAAGGTGAGCCGAAA
GCTCAGGTCACCAAACCCTTACAAAATTTCACCCCGATCGATGTACGAGTCGATGCAATGCA
ATGCAGGTTACATGCACGAGGTGCGCAAGGAGAGCAAGGAGGCGATGGCGGCCTACGTGAA
CGCCTCGGCGACGGAGCTGGATCACGTGTCGTCCAAGGTGGCCATCGGGGCTCTGCTCTCCA
AGCAGGGGGGCAAGTACCTCCCGGCGGCGAGGGCCTTCCTCTCGGACGCCCTGAGGGTCGA
GCCGACGAACCGGATGGCGTGGCTCAACCTGGGGAAGGTGCACAAGCTCGACGGGAGGATT
TCCGACGCCGCCGACTGCTTCCAGGCGGCGGTGATGCTCGAGGAGTCGGATCCCGTGGAGA
GTTTTAGGACGCTCTCATGAGATTATCACAACATACAGGAACTCCTTACTTTTTCTACCCTCC
ACATTACTCCTCTACTCTCCTTGTTTCTCTCTCTTGTTGTAGTTCAATGCATGTAAAGTTAACC
GATGTGTATAGGCACAATTGTTTTGCATATTTATTTATTTTGCCGTGGGACCTGTATTTGCTC
ATGGAAAGTGTGATGCTTTCAGAAAATGCAAGTGTGATGGCAGCTGAACTGTTACTTGAATT
TCGCTTTTACTTGCACTGTTTTAATTTTGTATGAGAATGATGACGCCAAAGCCTTGAGTTAAC
AGCGCTATTTAATTTACACTTCACACTGCACACTCTCTATACTATTGATAGCCTGGGCTTATT
TTTTGTTGCCCTCTATACATTCTGCATAGCCATTTTTTTCTCTTTTTTTTGCGAGGGTAAAGAG
TTTTGATAGTAATTGTGGACTTATCAGGAAGCTGAACATATATAGCAAATGTATTGTAAAGA
TGGACCTGCCCTATGCTGTTCTTCATCTTGGCGGAGGACCGGCGGTGGGGAGTGGTTTCGGG
GAGGCCGGGGCGGCAAGGCGGCACGGGAGCGCTGCCGGCTGCGGGCGGCGGAGGCCGGCG
GTTTGGCCAGTGGTGGCTGGCGGCTTAAGGGGGTGAAGGTTGAAGAAGCACTGTAGGCCTTT
GATTTCACATCCAATGGCTCAGAAATCGACTGACCACAAATGAAAATTTTAGCTGACTGATT
TCTAGCCATTTCCGTCAACAACACCTGGGTTCTGATTAGTTTCTTCAGGAAAGCTAGAATCA
GCTACTGCCTTCAAGAAACAAAAATGGTCGACGGAGGGGGACAGGCCAGAACCATAGAACC
ATTCGTGTTATCACCCCTGATCACTGCAGTTGTGATGCTTCGGGCGGGAACAAGAATGGACG
GAGGAGGACAAGCTGGAGATGGAGGCCGAGCTACAAGCAGCCGGGCATCAAACAACTAAA
TTTCTCAACCTAAATGGCCCTGTGCCCCTGTCCTAGTGTCGAATTTGAATAGAATGATGCAAT
CAATTCTTCTGTACCGCTCAAAAGAGTGATAGGATACATAGTTGCATCGCATGCTGGGACAC
AGATCCTCTGGCTAACCCTGCCTTACCCTGCCTTTGGGTCGCTGACAAGTGGGCCCCACGCTT
GGTGGGACCCATGTGTCAGTGTCTCAATGGCAGGTTAGCCAAAGTCAGGGGATCCTCGTCCC
ACATGCTGATCACCAAAGGAGTACAATCAATATAAGTCGAACGTACTTGAGAACATACACA
GGCAAAATAAGACAATTCTTGTAAATTCATCAGTCGCAGGACATGGATTTTATGCATTCTAA
AGATATCAACATGAGCTTGTAGATGCGGGGGAATGAACAACCAGTTTCACACTATTAGATTT
ATTTTAGTTAAGCACTCAAGTCAGCACAAGCTAAACCATGCTATAAGCTGGGCATAAGAACA
ACCAAACTTGAGGGAAAAGGGCTAAAAAATGAAGGCTTCTGCGATAATTAAAATGACAAGC
CACCACGCTTGCTACAAAATAGTATGTGTACCAGAGGATTCTTGTTAGAGGCACGGATGCAT
ATTCACAATTCCATTTTACTCAAAAAATTGTTATAACCACTTTAAGGATTCTTTCATATCTAT
TCCACCAAGGCATGAACTGCTTAATATTGCTAAGTTGCAACTGAAACACAAGTTATAACATG
TCACAACTAAGCCACTAGAAAATAGAATCACAACGTGTCACAAAACTGAAAAGATTGTGAA
ATAAAAAGAAATGGGAAAAAAGTTGCAATCTCAAAAAGGAGAGATTGTGCAGTAAAAAAG
AGAAAAGAAACAACTTGCTATCGCCAGTTACCAGATCTTGCTAGATGTATCTACTACCCTTA
TAGAAACACCTCAACGCCTCTAAGAACACGTGCCTGTCCACGCGGCTCCTCCTCGCCCGCCT
GCCGCGTCTCCTTCGCCGCGCCTCACCCGCCCATGCTAGAAGAAATCAAACCCCCACTGCGG
CGCACGACCACGTGCCACTCGCCCTGCTCAACGCAGCCCTCCCAGCGTCCGTCCTCCTGGGC
CACCGCCGCAGCCGTTGCCATGTGTGGATCCTGGACATCCTCGTCGTTTCATGGAACTGCTTC
CAACACAGTCGCCGGCTGAGTCATTCACACGCCGAAGGGGGCCGTCATCCCCATGCTATGAA
CAATCATAAGTTCATTCCTTTTGTCTTCTGGCTAAAATCACTTTGAATCCACCTCTGTATACG
AGACTGTAATCTCCAGAGTCTCAAGATACAAGACCAAGCTTGTTATTTTTCCAAGTTGTTCTT
GCAAGGTCAAGATATAGTGGCAGTTTCTTTTTCGAGTGTGGTTTTTGTGCACCCACGGACATT
CCACCCACGGTGCACCCACGATAAAAAACTTAGCAAAACATTTAAAAAAATTCTGAAATTTT
GTGGATGTGATTATGACCAAATGTTTTAGGCGCTTGCAAAATTTGGTTGCAAAATGACACCC
ATAGAGCTTTGTACAAAAAACAAAGTTTGTGTTGAAAACATTTGAACAGTAAGGTAGGTGC
AGAGCATCATTTGTATTTCGTTTATATGGAGATCATTTCATATTTTTCAGTGACCAAACTTTG
CAAGCTCCTAAAACATTGGCTCATAATCACATCCACGAAGTTTCAGAATTTTTTTAGTTTGTT
TACATTTTTTTCTTCGAATTTACTGTTCACTCCATAGGTGCGCCGAAGGTGGATGCATCCACT
ACTTTTCTTTCCTTTCCTTTTTCTCTGTGTATTTTACATGTTCGTACGTTTGCACCCTGCTCTGA
CTGCTTTCTTGTTCCAAGGCTGGTGATTCTACTCCAGAGCTTGCTACGGCCATCCAGGCCCAG
GGCGACCATCACTCGCGGTGGCGAGAAGCACTTGGTCGAAGTTGTGAAGGTTATAGATGCG
TACAAGGTATACGGCAAGCTCCGTGTTGAGAGGATGAACCGGCACCAATTGGGAGCTTGGA
TGAAGAAGGCTACCCGTGTGGAGAAAGTGGAGAAGAAGTGATGAGATGTTTATGACAGCTA
ATTGATGTTGTTATCTAAGTTTCTGAATGTGTGTTTTGGTCTGCTCGGATACCTTGTTTGATAT
CAAATAGCCCTTTCTTCCCACTGTTCAAATCAGCTCTTCATTGATATGCAAATGTTCAAACAA
TGTAGTTCAAATAGTTAAGTTGTTATGCCAGGAA
SEQ ID NO: 4 PV1-B CDS
ATGGCGGAGCCGGAGGACGGCGGCCAGGTCGCCCCTCCTGAGGCGGCGGTGGCGGCGACGA
GCGCGGCCGCCCATTCGTCTCCCCCTGCTAAGGAGGAGCCGGCGGCAGCGGCAGAGGCAAA
GCCGGCCAGCTCCGGCGAGGCGGTCTCCCTCAACTATGAGGAAGCGAGAGCTCTCTTGGGA
AGGCTGGAATTTCAGAAAGGCAATGTAGAAGATGCACTTTGTGTGTTTGATGGAATAGACCT
TCAAGCTGCCATTGAGCGCTTCCAGCCATCATCCTCGAAGAAAACAACAGAAGCTACTCTTG
TTCTTGAAGCCATTTACTTGAAAGCATTGTCCCTTCAGAAGCTAGGAAAATCAATGGAGGCC
GCTAAACAATGCAAAAGCGTCATCGATTCTGTTGAAAGTATGTTCAAGAATGGCACTCCTGA
CATCGAACAGAAGCTACAAGAAACTATCAATAAATCTGTGGAACTTCTCCCAGAGGCCTGG
AAAAAAGCCGGTTCTCTTCAGGAAACATTTGCTTCGTACAGACGCGCTCTTCTCAGCCCGTG
GAACCTCGATGAGGAATGCATCGCAAGGATTCAAAAGAGATTTGCTGCTTTCTTGTTGTATG
GTTGTGTGGAGTGGAGTCCGCCCAGCTCTGGTTCACCAGCTGAAGGCACTTTTGTTCCCAAG
ACCAATATTGAGGAAGCTATTCTACTCCTCACAGTAGTATTGAAGAACTTTTATCAGGGAAA
GACCCACTGGGATCCCTCGGTGATGGAACACTTGACCTACGCATTGTCGATTTGCAGCCAGC
CTTCTCTTATTGCAAATCATCTGGAGGAGGTTCTACCCGGGATATATCCTCGGACGGAGAGA
TGGAGCACACTAGCATTTTGCTACTATGGTGTTGGTCAGAAAGAAGTCGCTCTGAATTTCTT
GAGGAAGTCCTTGAATAAGCATGAGAACCCAAAAGATACAATGGCATTGCTGTTAGCCGCC
AAGATATGCAGCGAGGACTGCCGTCTTGCTTCCGAGGGTGTCGAGTATGCACGAAGAGCGA
TTGCAAACACGGAATCGTTAGATGTTCAACTGAAGAGCACCGGCCTCCATTTCTTGGGGAGT
TGCCTGAGTAAGAAGGCTAAGGTTGTTTCATCCGATCATCAAAGAGCTATGTTGCACGCAGA
AACTATGAAGTCGCTTACGGAGTCGATGTCTCTTGACCGCTACAACCCAAACCTAATATTCG
ACATGGGAGTTCAATACGCTGAGCAGCGGAACATGAATGCCGCGCTGAGATGTGCCAAAGA
GTTTGTCGACGCAACCGGTGGAGCGGTCTCGAAAGGTTGGAGGTTTCTAGCACTAGTCCTCT
CCGCACAGCAAAGATACTCCGAAGCAGAAGTGGCGACCAATGCCGCGTTAGACGAGACCGC
AAAGTGGGATCAAGGGTCACTGCTCAGGATAAAGGCTAAGCTGAAGGTCGCTCAATCATCG
CCCATGGAAGCGGTGGAGGCATACCGGGTCCTTCTTGCTCTTGTTCATGCCCAGAAGAATTC
GCCTAAAAAAGTGGAGGGAGAGGCTGGTGGAGTAACCGAGTTCGAAATCTGGCAAGGTCTT
GCAAATCTGTACTCCAGCCTCTCACACTGCAAGGACGCCGAGGTATGTTTGCAGAAAGCCAG
GGCCCTGAAATCATACTCCGCCGCGACACTCGAAGCCGAAGGTTACATGCACGAGGTGCGC
AACGAGAGCAAGGAGGCGATGGCGGCCTACGTGAACGCCTCGGCGACGGAGCTGGAGCAT
GTGTCGTCCAAGGTGGCCATAGGGGCGCTGCTCTCCAAGCAGGGGGGCAAGTACCTCCCGG
CGGCGAGGGCCTTCCTCTCAGACGCCCTGAGAGTCGAGCCGACGAACCGGATGGCGTGGCT
CAACCTGGGGAAGGTGCACAAGCTCGACGGGAGGATTTCCGACGCCGCCGACTGCTTCCAG
GCAGCGGTGATGCTCGAGGAGTCAGATCCCGTGGAGAGTTTTAAGACGCTCTCATGA
SEQ ID NO: 5 PV1-B polypeptide sequence
MAEPEDGGQVAPPEAAVAATSAAAHSSPPAKEEPAAAAEAKPASSGEAVSLNYEEARALLGRLE
FQKGNVEDALCVFDGIDLQAAIERFQPSSSKKTTEATLVLEAIYLKALSLQKLGKSMEAAKQCKS
VIDSVESMFKNGTPDIEQKLQETINKSVELLPEAWKKAGSLQETFASYRRALLSPWNLDEECIARI
QKRFAAFLLYGCVEWSPPSSGSPAEGTFVPKTNIEEAILLLTVVLKNFYQGKTHWDPSVMEHLTY
ALSICSQPSLIANHLEEVLPGIYPRTERWSTLAFCYYGVGQKEVALNFLRKSLNKHENPKDTMAL
LLAAKICSEDCRLASEGVEYARRAIANTESLDVQLKSTGLHFLGSCLSKKAKVVSSDHQRAMLH
AETMKSLTESMSLDRYNPNLIFDMGVQYAEQRNMNAALRCAKEFVDATGGAVSKGWRFLALV
LSAQQRYSEAEVATNAALDETAKWDQGSLLRIKAKLKVAQSSPMEAVEAYRVLLALVHAQKNS
PKKVEGEAGGVTEFEIWQGLANLYSSLSHCKDAEVCLQKARALKSYSAATLEAEGYMHEVRNE
SKEAMAAYVNASATELEHVSSKVAIGALLSKQGGKYLPAARAFLSDALRVEPTNRMAWLNLGK
VHKLDGRISDAADCFQAAVMLEESDPVESFKTLS
SEQ ID NO: 6 PV1-B genomic sequence. Start codon at bases 3,000-3,002.
Stop codon at bases 6,086-6,088.
TCGCTAAAACACCTGCCCCACGGTGGGCGCCAACTGTCGTGGTTCTAAGTCTGACAGTAGAG
TGGGGGGGTAGGTATGGAGAGGCAAGGTCCTAGCTATGGAGAGGTTGTAAACACAAGAGAT
GTACGAGTTCAGGCCCTTCTCGGAGGAAGTAAAAGCCCTACGTCTCGGAGCCCGGAGGCGG
TCGAGTGGATTATGTTTATATGAGTTACAGGGTGCCGAACCCTTCTGCCTGTGGAGGGGGGT
GGCTTATATAGGGTGCGCCAGGACCCCAGCCAGCCCACGTAATGAAGGGTTTAAGGGTACA
TTAAGTCCGAGGCGTTACTGGTAACGCCCCACATAAAGTGTCTTAACTATCATAAAGTCTAC
TTAATTACAGACCGTTGCAGTGCAGAGTGCCTCTTGACCTTCTGGTGGTCGAGTGAGACTTC
GTGGTCGAGTCCTTCAATTCAGTCGAGTGAGTTCCTCGTAGGTCGACTGGAAGGTGATCTCT
TCTAAGGGTGTCCTTGGGCAGGGTACTTAGATCAGGTCTGTGACCCTACCCTAGGTACATGA
CTCCATCAGGGCCGGAGTGCCGGAGGAGTGCGACGAGGATCGGGAGGAAGAAGAGGAGGA
GGAGGAGCCGAACCTCCTTGGCACCCATGGCCCGACGCGTCAGTGCTGCGCCGGGGGGTAC
GCCAATGGCGGGTCGTTGCCGCCCTCAAGGTACGTGAGCACGCCCTCGAGTGTGCGGCCGG
GAACGCCCCACCAGAGGTGACGCCCCTCGTTGTTCTGTCGGCCGCCGAACACCGGCGCACCG
TTGGTGGACGCCAATCGCTGCTGCTGGCGGCGCTCGAAATACGCCGCCCAGGCCGCGTGGTT
GTCGGCGGCGTACTGGGGGAGGGAGAGTTGGGCATCGGTGAGGGACGCGCGCACGATCTCG
ACCTCCTCGGCGAAGTACTTCGGCTTCGCCACGGCGTCGGGCAACGGGGGAATGGGTACTCC
CCCGGCGCTGAGCCTCCACCCCGATGGCCCGGCGCGCATGTCCGGCGGCGCCGGGATGTTCG
CCTGGAACAGGAGCCAGGACTCCTGTTCACGGAGCGAACGGCGGCCGAAGCCGTTGGCCGC
CGCCTCGTCTCCGGGGAAGCGTTCTGCCATGGCAACGGCGGGGTGGGGCGGGCTCGGGAGA
GGTAGAGGGAGGGGCCGGAGGGCGGCGCTCGGGAGAGGCAGGGAGAGGGAGGGGTTGGAC
GGCGGCGAGGGGGGGACTGGTCTGGGCACAGGCGAGTGGAGGCCGCTGGCTTTTATAGCCG
GGCCGCGCCCGTGTGTACGCGTGCGCGGGAAGGGAGGCGTCGGCGCGCCGCCCCGTGAAGC
GCCGCTCGTGAGGAATCAATGGCAAGGCTGACCGGCGGCAGCCTTGCCATTGATTCTCCGCG
GAAAACCGAGGCCGTTGGGGGAAGACGAGGCGCCGAGTCGCTGACGCGGCTGGCCCGCGTC
TTTTTCACGCCAAAACAGCTCGCCCCGGCACCCCCGGGCGCCCCCCAGCGCGCCGGGTTCGG
GCTAGGTCCGCCGGCGCTGTTTTCGGCCCAAGCCGGCGAAAATCGGGCTCCTGGGTGCGCGA
CTGGGCCGTTTTTCGGCGCCGGCGCGAAAAAAACGCCTGGGGAGGCCTTCCTGGGGCGCGG
CTGGAGATGCCCTAAACTTGCGCACCGCACCTGGGCCAACGCACCCCCTTTAGTACCGGGTC
GTAGCTCTAATCGGTACTAAAGGTGGGGTCTTTTGGTTCTCCGATGATCGTTTATTCTACAAT
TGCCCGATTTTAACTAGATTTGCTGCTAGTCCGAAGATCTACTTCCGTTCATTTCCATATGTG
CATGTGTTGCATGGATATGAGAAGCCGTTGAGATACACGGGTATGGACGCAACAAAATGAG
GCGTGCCCGGTCACTGCCCGCGGACGCGACCGGATACGTCCGCGGACGTTTGAGGGGCCAT
ATTTGTCATATGCGGCTGTAGATGCTCTAACGTGGCAGTAACGACCGTGAGCAGTTGGCACG
TGACGGCCGGCCTTAATCAACATGTTTCTCCATGCCATGGGCATCTGTCATCTGCGCCATTGG
TAGTGCGAGGAGATGGGACGCGGGTGACCCTGAGGAGGGAGGTAAAACCTCCTCCTGCGCA
AGCAGTTGATGGATGGAGCGCCCTTCAACCCAATGCTCCATAATCCCCAAATATGGAGGCTC
GTGGGCTTGATATGCAACGCCTTCATAAATGATAACTATCAAAGCCGTATGGCTGGCGTGTC
TGATATAGTGATTTTTGGTCCAAAAGGCGTTACTACGACTTTGTTAAAGTTGCTCTAATTGCA
TGCATGACCATCCGGTCATCTTATCTGTGCCACACAATGAAATCGCTCGGCATGCAATTTCTG
AAGGCTCCTGAGCAATTTCTACTTGTAGGCACCACACGAACGTTGTGCACTTTTTTTGGGATC
ACATCAACTGGCCTTCACTAAATACTACTCAGAACAAGCCACTACACGTTTTGTCTTGCACT
GTATATGTTTTCTCCAACGTCAGACTATTTTGAGAGAGAAAAAACACCTTGTTGTTTCTCTTT
TGCAGCCAAGGGCAAATCAAAAGTAATGGGATCGATCACATCTTGGTCAACATAAGTGGCC
GTTCAAGAGCAATGTATTGGCGGAGCTAGACAACCTATTYTTACCTTTCTCTAAAAAATAAT
CTATCATTAACTCAAATTTTCCGGAAAATGGCAGGACTCCCTTAAATTTCTCTCGGTATTCCT
GGCGAACTTTACACGTGGCAACAAGCAGTAGAGAGATAGGTAGAGAGAGTAGTCGCAAAA
GACTAAAAATAGAAGACAGAAAAATTAGTGGAAAAAAAGGTAAACATGTGAACCGTGGAA
GAAGTTGCCGCCTCTGTTTCTCTCCATCGACGACGAAACCGAGCACCTCCAAGCTCGACGAG
ATCGGGTGCCCGTCGGTGCGATCCCTCTGCTGCATTGGCATGGCGGAGCCGGAGGACGGC
GGCCAGGTCGCCCCTCCTGAGGCGGCGGTGGCGGCGACGAGCGCGGCCGCCCATTCGTCT
CCCCCTGCTAAGGAGGAGCCGGCGGCAGCGGCAGAGGCAAAGCCGGCCAGCTCCGGCG
AGGCGGTCTCCCTCAACTATGAGGTCCGAAATATCTGAAATTCTTTTATGAATGTTTGTTTG
ATAGTACGGTGCTTGTCCTATATAATGCTGCCATGCTGTGAATTTGGTTGACAAGAAATTGC
CATGTTTGAAGTGTTTGGTCAGTGCCACCAATGTTATGTCAAATCTCGTATTGCCGACGATGA
TGATGCCAATTCAGTTTAGCCATGACTTTGATTGTTCTCACATGAACCGAAATGTAAAGATG
CCAACGTTGGTCGTGCGTTTTCCTTGAAAAATATTGTTTGAGAGGCTTTGTGTGGGAAATTTG
TTCCTTTCTTGGGGATGTCAAATGCCGAAGTGTGATTTCATTTCAGTTCTGGTTCTATTTCATT
GATTGGTTTATCCAATTGTGAATTATTCGGCAAGCTTGTAGACATGGACCTTTTTTGTTCTTT
AAATATTTGGGTGAGTGAATTGTGATTTGTGAATAATCTTAATGCCAGTATAGGTAGCAAGA
TTTTACTGAATAATGTGTAATCATATGGAGAAAGGGACATTTTCTTTGTCCAGATTATGAAG
AACTGACCATATTTCTATTCCCACGAACCGTGCTATTGTATCTCCATTGCAATTATTAATTTC
CAAAAATGAAATTCAAACTTAGCTTAATACATGGAGAATTCCGACCGTCATGCTTTCTCCGG
TTTATTACACCAAGTTCTTTTGTTTTTGCGGGTTTATTACACCAAGTTCGTTTATACATCTATC
AATAACAGGAAGCGAGAGCTCTCTTGGGAAGGCTGGAATTTCAGAAAGGCAATGTAGAAGA
TGCACTTTGTGTGTTTGATGGAATAGACCTTCAAGCTGCCATTGAGCGCTTCCAGCCATCATC
CTCGAAGAAAACAACAGAAGCTACTCTTGTTCTTGAAGCCATTTACTTGAAAGCATTGTCCC
TTCAGAAGCTAGGAAAATCAATGGGTAACAAAATTGCTTTATACCGTTGTTTAAATTTAAGA
CAAATTTCTTTAATTGTGTTTTACAAAAATAAATCATCATTTGGAAGTTGTTCTGTTTTTAGC
ATATGTTTGACTTGTAACAAATTATTGAAATACCTGTTGAACATGCAGAGGCCGCTAAACAA
TGCAAAAGCGTCATCGATTCTGTTGAAAGTATGTTCAAGAATGGCACTCCTGACATCGAACA
GAAGCTACAAGAAACTATCAATAAATCTGTGGAACTTCTCCCAGAGGCCTGGAAAAAAGCC
GGTTCTCTTCAGGAAACATTTGCTTCGTACAGACGCGCTCTTCTCAGCCCGTGGAACCTCGAT
GAGGAATGCATCGCAAGGATTCAAAAGAGATTTGCTGCTTTCTTGTTGTATGGTTGTGTGGA
GTGGAGTCCGCCCAGCTCTGGTTCACCAGCTGAAGGCACTTTTGTTCCCAAGACCAATATTG
AGGAAGCTATTCTACTCCTCACAGTAGTATTGAAGAACTTTTATCAGGGAAAGACCCACTGG
GATCCCTCGGTGATGGAACACTTGACCTACGCATTGTCGATTTGCAGCCAGCCTTCTCTTATT
GCAAATCATCTGGAGGAGGTTCTACCCGGGATATATCCTCGGACGGAGAGATGGAGCACAC
TAGCATTTTGCTACTATGGTGTTGGTCAGAAAGAAGTCGCTCTGAATTTCTTGAGGAAGTCCT
TGAATAAGCATGAGAACCCAAAAGATACAATGGCATTGCTGTTAGCCGCCAAGATATGCAG
CGAGGACTGCCGTCTTGCTTCCGAGGGTGTCGAGTATGCACGAAGAGCGATTGCAAACACG
GAATCGTTAGATGTTCAACTGAAGAGCACCGGCCTCCATTTCTTGGGGAGTTGCCTGAGTAA
GAAGGCTAAGGTTGTTTCATCCGATCATCAAAGAGCTATGTTGCACGCAGAAACTATGAAGT
CGCTTACGGAGTCGATGTCTCTTGACCGCTACAACCCAAACCTAATATTCGACATGGGAGTT
CAATACGCTGAGCAGCGGAACATGAATGCCGCGCTGAGATGTGCCAAAGAGTTTGTCGACG
CAACCGGTGGAGCGGTCTCGAAAGGTTGGAGGTTTCTAGCACTAGTCCTCTCCGCACAGCAA
AGATACTCCGAAGCAGAAGTGGCGACCAATGCCGCGTTAGACGAGACCGCAAAGTGGGATC
AAGGGTCACTGCTCAGGATAAAGGCTAAGCTGAAGGTCGCTCAATCATCGCCCATGGAAGC
GGTGGAGGCATACCGGGTCCTTCTTGCTCTTGTTCATGCCCAGAAGAATTCGCCTAAAAAAG
TGGAGGTTTGTTTTCTTAATCAAATGCAGCAAAAAAAAAAGAGAGAGTACCATTCGTGTACT
ATTTTTCTCTTGGCACATTCTCCATTAGTTCACGTACTGATGCTTCAGGGAGAGGCTGGTGGA
GTAACCGAGTTCGAAATCTGGCAAGGTCTTGCAAATCTGTACTCCAGCCTCTCACACTGCAA
GGACGCCGAGGTATGTTTGCAGAAAGCCAGGGCCCTGAAATCATACTCCGCCGCGACACTC
GAAGCCGAAGGTGAGCCAAAGGTTCAGGTCACCAAAGTCTTACAAAATTTCACCCGATCGA
TGCACGATTCGATGCAATGCAGGTTACATGCACGAGGTGCGCAACGAGAGCAAGGAGGCGA
TGGCGGCCTACGTGAACGCCTCGGCGACGGAGCTGGAGCATGTGTCGTCCAAGGTGGCCAT
AGGGGCGCTGCTCTCCAAGCAGGGGGGCAAGTACCTCCCGGCGGCGAGGGCCTTCCTCTCA
GACGCCCTGAGAGTCGAGCCGACGAACCGGATGGCGTGGCTCAACCTGGGGAAGGTGCACA
AGCTCGACGGGAGGATTTCCGACGCCGCCGACTGCTTCCAGGCAGCGGTGATGCTCGAGGA
GTCAGATCCCGTGGAGAGTTTTAAGACGCTCTCATGAGATTATCACAACATACATGAACTCC
TTACTTTTTTTACCCTCTACATTACTCCTCTACTCTCATCGTTTAGCTTCCCTGTTGTAGTTCA
ATGCATGTAAAGTTAATCGATGTGTATAGGCGCAATTTTTTTTACGTATTTATTTATTTTGCC
GTTGGACCCTCTATACATACTATTGATTGCCTGGGCTTATTTCTTGGTGCCCTCTATATATTCT
GCATAGCCATTTCTTAGGGAGATCGTAATTGTCGACTTATCAAGAAGTTGAGCCTATATAGC
AAAATGTATTGTATAGCTGGACCAGCCCTATGGTGTTCCTCATCTTGGCATAATGGGGACAC
CACTACACTAGCCTCTTCACTGCTTCACAAGGTGTCAACTGTCAAACCAGCAAAACAAAGAG
CAAACAAACCAATTACTTGCATATTAAAACACATCTCCAGTTTCAGGTCCATGTGTTCTTATT
CATCAATTCACGCCCGAGGGACTACTTTGGATGGGATCTCGACCACATACCTCCTGTCCAAG
GCTATATATGATTTTAGAAACCATAATGTTGAGTGAACCTGAGAGGTTTTTGGATCGCCAGT
TGGACAACAACCAAACTGTGGTTGAGTTGGTTAGTAGGCCACACTACCGGAGTTCAAGTCCT
ATCAGGCACAACATATTTTTACGTTCCACAAGAGAAAACTGCCTCCAGGAAACCTACCCCAG
CCCATGTTCAAGCCATCACAGAAAACAAGAACAATTTGATGCTGCAGCTAACAAGAACAAT
TAACTCACTCGTTGGCATTTCCACTAAACTGTTCCAAAGAAAATATGATGCCTAAAAAGGAA
TGTCATCTCCGTATTCGTACACACGCTTCGAATGCATGTGCTACTCAGCGATATGCGGATCCA
GCGCCATAACCTTGTCGAATAGGGATCTAACATACCCATATGACCGCATCTGCAGAATGCAT
AAATAAATATATCTTTACATGAGATCCATTCAACGGACACTCCTGCCGTGCATCCAACTGCA
AGATTGCATCCGGAATTCATAAAACAAATACTGTACTATCATCCAGGAGAATGGAGTATATA
TATATAACACCAGGCTGAGGAAGGAGGACACAAATTCAACCGAATACGGACGTACATGGCG
GGAGAAACAACTACTATGAAAGATCTTCCCGCTTATTATTAATTATATTATTTGACTGAGGA
AATAACACAACACAAGCAAGCAAGCAAGGAAATTAAGCGCGGGAGGAATAGGTAGTACAT
GCAATCACGCGGACGGACGGACGGCGTCCTCGCACTCGTCAATCTCGGCGAGGCTGCCAGA
CTCAACCAGACCCACACGGAACAACTTGGAGTAGGAGATGTCCCCGGAGACGGTGACGTCG
ACGGAGTTGCAGACCCAGTCCTGGCGTTCCCGGGTGAGGACGAGGAGGCACGGCCGGATGC
ATCTCGGGTCCGTGAAGCTGAATTCGTCGGTGCTGCCACGGTTGAACCTGGCACCGTCGCGG
TCGTCTTCCCAGTGTGTCACGACGGGGCTGCCGTCCTGCAGGTTGTCGCCGTACAACCGGAA
CTCCACGAATGCCTCCGTGCCGGCCGGAGGCCAGAGCCCCGTCTTCACCTTCACCCGGTACT
CACAGTCCCCCTTGGTGGTGCCGCTGCCGGCGAGGAAGGCGGCCACCAGAATGACAAGGGC
GAGCTTGGGCATGCCCATGGCCACCTGCGTTAGTTTAGTAGCTACGATAGATAGATAGATAG
ATAGATATATGACGATGACGGTTGATGGATGGATGGATCAGCTTCCCACCGGCATTTATATA
GGGTGTTTATTTGCCCAGCTCCAGCTGCATTTATATAGGGTGTTTATTTGCCCAGCTCCAGCT
GCTGCCCCTAACCCATATTAATAAGCTAGCTTATTATCCCTGATTCGCATACAGCCGTGATCG
ATACCAGACATCACATGATGAGATCAGATCAGGTCAGATCGATCAGATGGATGATAAGCTTT
ATCAATTCCCGGCCGGACACGCAAGTTGGTCTCCCGAGACCGACCGGCAAATCAAGCGCCC
GATCGCATCACATGCACAACATCAATCTTCCCTTTCTGGGCTTACCAATAACATTAACTAACT
ATATACATTTCCATGCAGTGCAGAGCTTCATTTACCACTAATAATGGAATGGAATACAAGTA
TTGGATGGGATCGGGTCTGGATTAATTGTATATATTTTCTCTCTGAAAAAACGATCGATCTGA
CAGAGTTGCGCGCCGGAGCTGCAGCAACACGACGGTGGGAGTAGATTGAGAACTCGGGATA
CGTTTTCTGGTATTTTTTTCACGAAATTTCACAGGGGAGTAGATTGAGAACCCAAGTTCAATA
TCCCAAAATTTCAGTTTATTTTTTAAAAAAATTACTATTTTTTTATATTTAATATTCGTATAGG
GGGGTGGAGCACCCAGAAACTCTTGTGTATTTGTCCCTTGCTCTTCTTTATGCAAATTTTACA
AATGTAGTCAGTATAATAGGCTTTTTAATACTTATGTTCCTCTTACCGCATTTTATTTTACCTC
GCAAGGCAAAACTGACCAAGCTAAGCCAATCTGCTCTCTATCACAATCATTTATTTAGGACA
TGGAGCTAAGGGCATCTCCAAGGTGGACCCACAAGCCTCCCACAATCATCCCGACTGTGCTG
TCCGGACCGCCGAAGCCATCCAACGCGGTCTCGTATCGGTCCGCGGGGCGGCCCGGACGCG
ATTTCTCCAGCAAAACGGAGACAAAAGTGGGGGAGCTTTGCAGGAGTCCGAAACACGAAAC
GTAGAAGTCCAACACCCTAGGCCCACCCAAAACCCTTCCCGGACCCCGCGACTCCTTCCTTC
TTTCTCTGTTGCCGTTGCCGCCACTCCACCACCCCGGCCGCCACGCCACACCCCTGCCGAAA
ATCTGCGTCTCCATCACCTCCGGCGCTCCAGCAGGGCTCCCCGCCGCTTCTCCTCCGTCTCCG
TTCAACCCCCTGTCCTCCAACCGCCCCGCCATATACGATCCTCTCCTGTGCCTGGCAACACTT
CAATGAATCGCCGGAGCTCAGAACTGTGTCCCTTCTTTTTAGCAATGGATTCCAATTTGGAGT
ACATATAGGAGCATCTTTATAATCACCTAGTTACGTTGTAGGCCGTTTGTGCACCATGATGC
GGTGACGTGCTGCCCTGTGCCTCCTTCCCTCCTCGGCACCACGTCGTCGCCAGTCCACCA
SEQ ID NO: 7 PV1-D CDS
ATGGCGGAGCCGGAGGACGGCGGCCAGGTCGCCCCTCCTGAGGCGGCGGCGGCGGCGACGA
GCGCGGCCGCCCATTCGTCTCCCCCTGCTAAGGAGGAGCCGGCGGCAGCGGCAGAGGCAAA
GCCGGCCAGCTCCGGCGAGGCGGTCTCCCTCAACTACGAGGAAGCGAGAGCTCTCTTGGGA
AGGCTGGAATTTCAGAAAGGCAATGTAGAAGATGCACTTTGTGTGTTTGATGGAATAGACCT
TCAAGCTGCCATTGAGCGCTTCCAGCCATCATCCTCGAAGAAAACAACAGAAGCTACTCTTG
TTCTTGAAGCCATTTACTTGAAAGCATTGTCCCTTCAGAAGCTAGGAAAATCAATAGAGGCC
GCTAAACAATGCAAAAGCGTCATCGATTCTGTTGAAAGTATGTTCAAGAATGGCACTCCTGA
CATCGAACAAAAGCTACAAGAAACTATCAATAAATCTGTGGAACTTCTCCCAGAGGCCTGG
AAAAAAGCTGGTTCTCTTCAGGAAACATTTGCTTCATACAGACGCGCTCTTCTCAGCCCATG
GAACCTCGACGAGGAATGCATCGCAAGGATTCAAAAGAGATTTGCTGCTTTCTTGTTGTATG
GTTGTGTGGAGTGGAGTCCGCCCAGCTCTGGTTCACCAGCTGAAGGCACTTTTGTTCCCAAG
ACCAATATTGAGGAAGCTATTCTACTCCTCACAACAGTATTGAAGAAGTTTTATCAGGGAAA
GACCCACTGGGATCCCTCGGTGATGGAACACTTGACCTACGCATTGTCGATTTGCAGCCGGC
CTTCTCTTATTGCAGATCATCTGGAGGAGGTACTACCTGGGATATATCCTCGGACGGAGAGA
TGGAACACACTAGCATTTTGCTACTATGGCGTTGGTCAGAAAGAAGTCTCTCTGAATTTCTTG
AGGAAGTCCTTGAATAAGCATGAGAACCCAAAAGATACAACGGCATTGTTGTTAGCTGCCA
AGATATGTAGCGAGGACTGCCGTCTTGCTTCCGAGGGTGTCGAGTATGCAAGAAGAGCGATT
GCAAACACGGAATCATTAGATGTTCATCTGAAGAGCACCGGCCTCCATTTCTTGGGGAGTTG
CCTGAGTAAGAAGGCCAAGATTGTTTCATCCGATCACCAAAGAGCTATGTTGCACGCAGAA
ACTATGAAGTCGCTTACGGAGTCGATGTCTCTTGACCGCTACAACCCAAACCTAATATTCGA
CATGGGAGTTCAATACGCTGAGCAGCGGAACATGAACGCCGCGCTGAGATGTGCCAAAGAG
TTCATCGACGCAACCGGTGGAGCGGTCTCGAAAGGTTGGAGGTTTCTAGCACTAGTCCTCTC
CGCACAACAAAGATACTCCGAAGCAGAAGTGGCGACCAATGCCGCGTTAGACGAGACCGCA
AAGTGGGATCAAGGGTCACTGCTCAGGATAAAGGCTAAGTTGAAGGTCGCTCAATCATCGC
CCATGGAGGCGGTGGAGGCATACCGGGTCCTTCTTGCTCTTGTTCAGGCCCAGAAGAATTCG
CCTAAAAAAGTGGAGGGAGAGGCTGGTGGAGTAACCGAGTTCGAAATCTGGCAAGGTCTTG
CAAATCTGTACTCCAACCTCTCACACTGCAGGGACGCCGAGGTATGTTTGCAGAAAGCCAGA
GCCCTGAAATCGTACTCCGCCGCGACACTCGAAGCCGAAGGTTACATGCACGAGGTGCGCA
ACGAGAGCAAGGAGGCGATGGCGGCCTACGTGAACGCCTCAGCGACAGAGTTGGAGCACGT
GTCGTCCAAGGTGGCCATCGGGGCGCTGCTCTCCAAGCAGGGGGGCAAGTACCTCCCGGCG
GCGAGGGCCTTCCTCTCGGACGCCCTGAGGGTCGAGCCGACGAACCGGATGGCGTGGCTCA
ACCTGGGGAAGGTGCACAAGCTCGACGGGAGGATCGCCGATGCCGCCGACTGCTTCCAGGC
GGCGGTGATGCTCGAGGAGTCGGATCCCGTGGAGAGTTTTAGGACGCTCTCATGA
SEQ ID NO: 8 PV1-D polypeptide sequence
MAEPEDGGQVAPPEAAAAATSAAAHSSPPAKEEPAAAAEAKPASSGEAVSLNYEEARALLGRLE
FQKGNVEDALCVFDGIDLQAAIERFQPSSSKKTTEATLVLEAIYLKALSLQKLGKSIEAAKQCKSV
IDSVESMFKNGTPDIEQKLQETINKSVELLPEAWKKAGSLQETFASYRRALLSPWNLDEECIARIQ
KRFAAFLLYGCVEWSPPSSGSPAEGTFVPKTNIEEAILLLTTVLKKFYQGKTHWDPSVMEHLTYA
LSICSRPSLIADHLEEVLPGIYPRTERWNTLAFCYYGVGQKEVSLNFLRKSLNKHENPKDTTALLL
AAKICSEDCRLASEGVEYARRAIANTESLDVHLKSTGLHFLGSCLSKKAKIVSSDHQRAMLHAET
MKSLTESMSLDRYNPNLIFDMGVQYAEQRNMNAALRCAKEFIDATGGAVSKGWRFLALVLSAQ
QRYSEAEVATNAALDETAKWDQGSLLRIKAKLKVAQSSPMEAVEAYRVLLALVQAQKNSPKK
VEGEAGGVTEFEIWQGLANLYSNLSHCRDAEVCLQKARALKSYSAATLEAEGYMHEVRNESKE
AMAAYVNASATELEHVSSKVAIGALLSKQGGKYLPAARAFLSDALRVEPTNRMAWLNLGKVH
KLDGRIADAADCFQAAVMLEESDPVESFRTLS
SEQ ID NO: 9 PV1-D genomic sequence. Start codon at bases 3,201-3,203.
Stop codong at bases 7,078-7,080.
ACACTACATTCTAAACATAATATCTAGAAGCCGAGAGGTAGAAGAAGACTTTTTCAAGGCA
AAATATTCAATATTTTCAACACCAGATTTAGAATGGGCTTGAAGTGCGTTAACAACAGATCC
TCCAAAGACAGATCTGGGCAGAAATTGTGTTAAATGGCAAACAGGGTTTCAGAGAAGGAAC
AGGACAGGTCAGTTAGTTGTGTGCTAAGAACTCATCGACACTTCAGTTCATGAAAAAGGAA
GAACTAATCAGTGCACGACATACCCGAGCATCATCCTCCTCCTTTGAGACTTCTTTGACAAC
CACCTCCTCTTCACGCTTGTAAAGCTGATCAAACAAATGAGAGACTTGTAAGCCTAAAAAGT
AACAGTTTACACTGCAAATATTCTTACAGTGACTGAACTCTACAAGAAGCGTACTTCAGTGG
AGATGAACTAGAATGAACCACGATGACTTCAGTACAACTTCCTCACTGAACACTAGCATAGA
GTTGCATATAAGGCTATTCTACCAAAGAGCTAAGGTGCAACAACTATTGGAGAACTCGTACA
AATCATACAATACACAGAGGCAGAACTGATATACGAAACTCCGGAAAGCATAGCCTCAAAA
GCCAACAAGAGTAAGCTAGTAAGTAATGCTTGTGAGCTGCAACCGAGCATTCCAAAACTGC
ACGGCCATCGTAGCATGTTTATTTCTATCGGGGAAAAGGAGGAAGCTAACCTTGCTCTGCTC
GCGCATGAGTATGGCGAACTTGTCGATGTTGGGGACGGCGAGCATCTTGGGCATGAGGTAG
TTGCGGAAGTGCCCCGGCGCCACCTTCACCGTCTCCCCCGCCTTCCCCAGCTTGTCGATCGTC
TGAAGTGAACAAGCGATGGACGATGGCAAAGGTTAATAATTCCACTTCCCGGCACATTGAA
AATCTCTAGGGATATTGTTGAAATGAACAGCCAAAACCGAAGCTTTACCGGTCAAGAATACT
ACTGCTAGCTTAAAAAGTTTCAGAAATGCTGAAGATTTATCGGTCAAACTGTCGCTGAGGCG
GCACCGGCCTCACCGGATCGGAAACATCCCGCTCGAACTGACCGGAAACGCAAGCGGGATG
CATCCTGCAGAGGTGGAGGACCGAGCGGAGGGTCGCGGGTTGAGATTTGGAGGAGAAGGG
GAGGGAGGGGGCAGGGGGGCTGGCTTACCGTGGTGAGGATGACCTCGAGCTTGCGGTAGCG
GAGGCCGTGGCCGGAGAAGAGGACGGGGTTGGCGGCGGCGGCGCCGAGGCCGGGACGGCG
GAGGAGGGCGGCGCGGGCGGCGGCCATGGTGGAGTAGGGTTCAGGGGAAGGGGGCCGGCG
GCTGCCACAAAACGGGTGCGCGAGGGAGAGTATTGGTGGCTTCCCGCCGGACCGGGCCGGT
GCCAGGCCAGGCCCGCTAAGGGATCTCCATTTTTTCCCTTTGAATTTATTTTTAAAACACTTC
TGCTGCCCAAAAGAATTTGCATTTGCATTTTCTTGAGTCCCTTTGATAGACTAAAAAAAATCT
CGAGTCCCTTTGATTTATTTTTCAAAATTCTTCTGCTGCCATGAAAACTTTGCAATTTGCACTT
TCCTGAGCGAGGTAGTAGACCAGGAAAGAAATCCGGAAAAGAGTAGGGATTCTTCTGCCGG
ATGCCAGCACCCTCCGCAATCCAATAAAAATCAAATCAGACATTCAAAATCTCATCAAAATA
TCAACTTTAGGCCTTTTTTCTGAAGGCACATAAATGCTATTTTTCGTAACATAAAGGTTATGT
GAGTTTTTAGTCCAATTTGTTTCATAGTTGGCAAGTCAAAACTCTTGGACTTGGTTATCTGAT
ATATTCAAGCACCACATGTTACATGTTATTTTGCGCTGAAAATCAAGATATGTATCATTAATT
TTCCTATTTCAGGAAAGTTACAATAGTTAGACCCTCTCCATTTCAATTTCCAAAGATGTAGGA
TGCAACAATTTTTCTTACCACCAAGATATATTAATATTGTGTGGTTTTCCGTGATATGAACTC
CCCTATCCCTTGGCAGCTATGGTAAAATCTCCCCTCCAGGCTTCATCGACGAGACCGTGGAT
TCGCCTCCCCCTTACCTGCCGATCTGACGACCGGTGGCGGGGTTAGGCATCCCGGTGCTTCC
GCTGCGGTTAATAGTTTAGGTTAGTTTTCTTTTAGTCCTCTTAGGTGTGGCGCTCATATGGAT
GGCAGCGCTTTTTCTTCGAGTTTGTCTTTTGGGCTCCGATGCTCCTCGAGTTCGTCCATTAGA
ACGTAATTGACGGAGCTCCAACGTAGATTCCTACCGTCTCCTTGGGGCAGTGAGTTTAGTGT
TTCTCGTCGTGTGATGAGATTTGATGTCAGGTGCTTCAGATCTATTGAAGGGTTCAACAATG
ACGACTGCGGCTCTAGGGCGCTGGTCCTTACAGGCGGCTCTAGGGCGTTGGTCCTTACGGGC
ACATGCACGAAGCCTTCCCGACTGTCATCGATAATGTCAAGCCGGCTACAGTAGGGGAGCG
GTGACAGCGACGTGTCGGCAGCTCGTTCTGACGGCGGAATTGGTCGTTCGGTGGTGAAGAG
GCGTTCTTCGTGGGCAAGCCAAATGATTCAGATCTATTATCAATGTTCACCAGAAAAATTAC
AGCACCTTGTTGTTTCAATTTTGCGAAAATGATTGAAATGTATTATCAATGTTCACCAGTAAA
AAAACAAAGCACCTTAAAAAATTTCAGAGGAAAAAAAACACCCTGTTGGACAAAGAATAGG
TTACGATCACATCTTGGTCAACATATGTGGCCGTTCAAGATCAATGTGTTGACGGCGCCCAC
GATCCATGCATGCATTTGTATCGGTGGAGCTAGACAATCTATTTTTAGCTTTTCTCTTAGAAA
AAAAAAACTATCATTAGCTCGAATTTTCTGGAAAAAATTGTAAGGACTCCCTTAAATTTCTA
TCGGTATTCCTGATGAACTTTACACGTGGCAACAAGCAGTATGGAGATAGCTAGAGAGAGT
AGTCGCAAAAGACTAAAAATAGAAGGCAGAAAAATTAGTGGAAAAAGGTACGCATGCGAA
CCGTGGAAGAAGTTGCCGCCTCTGTTTCTCTCCATCGACGACGAAACCGAGCACCTCCCAGC
TCGACGAAATCGGGTGCCCGTCGGTGCGATCCCTCTGCTGCATTGGCATGGCGGAGCCGGA
GGACGGCGGCCAGGTCGCCCCTCCTGAGGCGGCGGCGGCGGCGACGAGCGCGGCCGCCC
ATTCGTCTCCCCCTGCTAAGGAGGAGCCGGCGGCAGCGGCAGAGGCAAAGCCGGCCAGC
TCCGGCGAGGCGGTCTCCCTCAACTACGAGGTCCGAAATATCTGAAACTCTTTTATGAATG
TTTGTATGATAGTAGGGCGCTTGTCCTATAGTATAATGCTGCTATGCTGTGAACTTGGTTGAC
AAGAAATTGCCATGTTTGAAGTGTTTGGTCAGTGCCACCAATGTTATGTCAAATTTCGTATTG
CCGGCGATGATGATGTCAATTCAATTAAGCCATGACTTTGATTGTTCTCACATGAACCGAAA
ATGTAAAGATGCCAACGTTGGTCGTGCGTTTTTCTCGAAAAATATTGTTTGAGAGGCTTTGTG
TGGAAATTTGTTCCTTTCTTGGGGATGTCAAATGCCGAAGTGTGATTTCATGTCTGTTCCGGT
TCTATTTCATTGATTGGTTTATCCAATTGTGAATTATTCGGCAAGCTTATAAGACATGTACCT
TTTATGTTCTTTAAATATTTGGGTGAGTGAATTATAACACGATGGTGTCAATCAAAATGCTTT
TTATTGGGTGAGTGAATTACGAATAATCTTAAGAGTGAATTCCGGTTTTTACCCCTAATTTAG
CATTTTTACACTAGTTACCCCCATTGAACAATTTTTCATCCAGATTACCCCACTTAGTGACAA
TCTTGACTGTTTTTACCCCTTTTAATTTTTATAAGAGCCTTCTTGCAAAGTTCGTGTTTTACTG
AGAGTCCAGACTAAGTGCCCAAGCATATATTTATATTAAAACAAAAAATCCGATCCTATTAT
GTTAATAACTGGCAGTACTAATTTTAAATGGACACACATCATTGGAGCCCAACTGAAAAACA
CATGTTTGACAATAGCACTACAGATCTGTAAAATAGAATGTGATGTTTTTATTTGATGATTTC
CCAAAGCCTAAAATAAATGCTTCATCTGATGTTTTGCATCAAGGAAGAAAACTAAAATATCA
TCACACTAAACCTACGAACCACAACCCACAATGCAAAATTGAATGTACTTTACCGCTCAAGA
TAATTGTTCGTCTTTCCTGCAATGTGGTGCACACATACACCTGACAGCCATGAGAAAAAAAA
AACGAGTGCGGCTAAACCAAGTGACCGGTTTGGGCATTGGAAAAAAAATGCCAAGAGGAAT
CTGATGGCAGGGAATTAGCTGACAGATCGCTCTCAAAAGAATTACTGGGGTAAAAACTGGC
AAACTTTTGCTAAATGGGGTAACCAGGATGAAAATATATTTAATGGGGTAGTTAGTGTAAAA
AATGTTAAAGTAGGGGGTAAAAACTGGAATTCACTCTAATCTTAATGCCAGTATAGGTAGCA
AAATTTTAACTGAATAATGTGTAATCATACGGAGAAAGGGGCATTTTCTTTGTGCAGATTAC
GAAGAACTGATCATATTTCTATTCCCATGAACCGTGCTATTGTATCTCCATTGCAATTATTAA
TTTCCAAAAAGGAAGTTCAAATTTAGCTTATACATGGAGAATTCCAACCGTCATGCTTTCTCC
GGTTTATTACACCAAGTTTTTTTTTTGTGGGTTTATGACACCAAATTCGTTTATACATCTATCA
ATAACAGGAAGCGAGAGCTCTCTTGGGAAGGCTGGAATTTCAGAAAGGCAATGTAGAAGAT
GCACTTTGTGTGTTTGATGGAATAGACCTTCAAGCTGCCATTGAGCGCTTCCAGCCATCATCC
TCGAAGAAAACAACAGAAGCTACTCTTGTTCTTGAAGCCATTTACTTGAAAGCATTGTCCCT
TCAGAAGCTAGGAAAATCAATAGGTAACAAAATTGCTTTATACCGTTGTTTAAGTTAAAAAA
AATGCTTTAATTGTGTTTTACAAAAATAAATTATCATTTGGAAGTTGTTCTGTTTGTAGCTTA
TGTTTGACTTGTGACAAATTATTGAAATACCTGTTGAACATGCAGAGGCCGCTAAACAATGC
AAAAGCGTCATCGATTCTGTTGAAAGTATGTTCAAGAATGGCACTCCTGACATCGAACAAAA
GCTACAAGAAACTATCAATAAATCTGTGGAACTTCTCCCAGAGGCCTGGAAAAAAGCTGGTT
CTCTTCAGGAAACATTTGCTTCATACAGACGCGCTCTTCTCAGCCCATGGAACCTCGACGAG
GAATGCATCGCAAGGATTCAAAAGAGATTTGCTGCTTTCTTGTTGTATGGTTGTGTGGAGTG
GAGTCCGCCCAGCTCTGGTTCACCAGCTGAAGGCACTTTTGTTCCCAAGACCAATATTGAGG
AAGCTATTCTACTCCTCACAACAGTATTGAAGAAGTTTTATCAGGGAAAGACCCACTGGGAT
CCCTCGGTGATGGAACACTTGACCTACGCATTGTCGATTTGCAGCCGGCCTTCTCTTATTGCA
GATCATCTGGAGGAGGTACTACCTGGGATATATCCTCGGACGGAGAGATGGAACACACTAG
CATTTTGCTACTATGGCGTTGGTCAGAAAGAAGTCTCTCTGAATTTCTTGAGGAAGTCCTTGA
ATAAGCATGAGAACCCAAAAGATACAACGGCATTGTTGTTAGCTGCCAAGATATGTAGCGA
GGACTGCCGTCTTGCTTCCGAGGGTGTCGAGTATGCAAGAAGAGCGATTGCAAACACGGAA
TCATTAGATGTTCATCTGAAGAGCACCGGCCTCCATTTCTTGGGGAGTTGCCTGAGTAAGAA
GGCCAAGATTGTTTCATCCGATCACCAAAGAGCTATGTTGCACGCAGAAACTATGAAGTCGC
TTACGGAGTCGATGTCTCTTGACCGCTACAACCCAAACCTAATATTCGACATGGGAGTTCAA
TACGCTGAGCAGCGGAACATGAACGCCGCGCTGAGATGTGCCAAAGAGTTCATCGACGCAA
CCGGTGGAGCGGTCTCGAAAGGTTGGAGGTTTCTAGCACTAGTCCTCTCCGCACAACAAAGA
TACTCCGAAGCAGAAGTGGCGACCAATGCCGCGTTAGACGAGACCGCAAAGTGGGATCAAG
GGTCACTGCTCAGGATAAAGGCTAAGTTGAAGGTCGCTCAATCATCGCCCATGGAGGCGGT
GGAGGCATACCGGGTCCTTCTTGCTCTTGTTCAGGCCCAGAAGAATTCGCCTAAAAAAGTGG
AGGTTAGTTTTCTTAATCAAATGCAGCAAAAAAAGTACGATCCGTATACTATTTTTCTCTTGG
CACTTTCTCCATTAGTTCACGTACTGATGCTTCAGGGAGAGGCTGGTGGAGTAACCGAGTTC
GAAATCTGGCAAGGTCTTGCAAATCTGTACTCCAACCTCTCACACTGCAGGGACGCCGAGGT
ATGTTTGCAGAAAGCCAGAGCCCTGAAATCGTACTCCGCCGCGACACTCGAAGCCGAAGGT
GAGCCGAAGGTTCATGTCACCAAACCCTCAAAAAGTTTCACCCAATTGATGTACGATTCGAT
GCAATGCAGGTTACATGCACGAGGTGCGCAACGAGAGCAAGGAGGCGATGGCGGCCTACGT
GAACGCCTCAGCGACAGAGTTGGAGCACGTGTCGTCCAAGGTGGCCATCGGGGCGCTGCTC
TCCAAGCAGGGGGGCAAGTACCTCCCGGCGGCGAGGGCCTTCCTCTCGGACGCCCTGAGGG
TCGAGCCGACGAACCGGATGGCGTGGCTCAACCTGGGGAAGGTGCACAAGCTCGACGGGAG
GATCGCCGATGCCGCCGACTGCTTCCAGGCGGCGGTGATGCTCGAGGAGTCGGATCCCGTGG
AGAGTTTTAGGACGCTCTCATGAGATTATCACAGCATACATGAACTCCTTACTTTTTTTTACC
CTCCACATTACTCCTCTACTCTCCTTGTTTATCTCTCCTGTTGTAGTTCAATGCATGTAAAGTC
TTTTTTTCGGGAAATCTTCCGATCTATTCATCTTCAATCATGGCAGTACAACGAATACCAAAA
ATAATAAAAATTACATCCAGATCCGTAGACCACCTAGCGATGACTACAAGCACTGAAGCGA
GCCGAAGGATCGCCGTCGTCATCGCCCCTCCATTGTCAGAGTCGGGCACAACTTGTTGTAGT
AGACAGTCGGGAAGTCGTCGTGCTAAGGCCTCATAGGACCAGCGCACCAGAACAGCAATCG
CAGCAGATGAAGAATAACATAGATCGGAAGGATCCAATCCGAAGACACACGAACGTAGAC
GAACACCAACGAGATCCGAGCAAATCCACCAAAGTTAGATCCGCCGGAGACACACCTCCAC
ACGCCCACCAACGATGCTAGACGCACCACTGGAACGGGGGCTAGGCGGGGAGACCTTTATT
CCTGTTGGGGAACGTAGCAGAAATTCAAAAAATTTCTACGCATCACCAAGATCAATCTATGG
AGTACTCTAGCAACGAGGGGAAGGGGAGTGGATCTACATACCATTGTAGATCGCGATGCGG
AAGCGTTGCAAGAACGTGGATGAGGGAGTCGTACTCGTAGTGATTCAGATCGCGGTTGATTC
CGATCTGAGCACCGAAGAACGGTGCCTCCGCGTTCAACACACGTACAGCCCGGTGACGTCTC
CCACGCCTTGATCCAGCAAGGAGAGAGGGAGAGGTTGGGGAAGACTCCATCCAGCAGCAGC
ACGATGGCGTGGTGGTGATGGAGGAGCGTGGCAATCCCGCAGGGCTTCGCCAAGCACCGCG
GGAGAGGAGGAGGAGGGAGAGGGGTAGGGCTGCGCCGAAAGAGAGACGTTCTCGTGTCTC
TTGGGCAGCCCAAACCTCAACTATATATAAGGGGGGAGGGGGCTGCGCCCCCTCTAGGGTTC
CCACCCCAAGAGGAGGCGGCTAGCCCTAGATCCCATCCAAGGGGGGCGGCCAAGGGGAGG
AGAGGGGGGGGGCGCCACTAGGGTGGGCCTCAAGGCCCATCTGGACCTAGGGTTTGCCCCC
TCCCACTCTCCCATGCGCTTGGGCCTTGGTGGGGGTGGGGGCGCACCAGCCCACCTGGGGCT
GGTCCCCTCCCACACTTGGCCCACGCAGCCTTCTGGGGCTGGTGGCCCCACTTGGTGGACCC
CCGGGACCTTCCCGGTGGTCCCGGTACATTACCGATATCACCCGAAACTTTTCCGGTGACCA
AAACAGGACTTCCCATATATAAATCTTTACCTCCGAACCATTCCGGAACTCTCGTGACGTCC
GGGATCTCATCCGGGACTCCGAACAATATTCGGTAACCACGTACATGCTTTCCCTATAACCC
TAGCGTCATCGAACCTTAAGCGTGTAGACCCTACGGGTTCGGGAACTATGTAGACATGACCG
AGACGTTCTCCGGTCAATAACCAACAGCGGGATCTGGATACCCATGTTGGCTCCCACATGTT
CCACGATGATCTCATCGGATGAACCACGATGTCGGGGATTCAATCAATCCCGTAT
PV1 guides (the fourth guide is in the reverse direction relative to
the coding sequence)
SEQ ID NO: 10 GCATGGCGGAGCCGGAGGACGG
SEQ ID NO: 11 GTCGCCCCTCCTGAGGCGGCGG
SEQ ID NO: 12 AAGGAGGAGCCGGCGGCAGCGG
SEQ ID NO: 13 GAGACCGCCTCGCCGGAGCCGG
SEQ ID NO: 14 OV1-A CDS
ATGGCTGGAGAGGTTGGCAAGTGGGGTAGTTCCTTCAAACGTTCTTGGGCTTTAATCCCACT
GGTCGCCCATGGAATCATCGTGGTCGTAGTGGGTCTGGCTTACTCTTTCATCTCGTCGCACAT
AAATGATGATGCCGTGAGCGCCATGGACGCGTCGCTGGCGCACGTCGCCGCCGGCGTGCAG
CCTCTAATGGAAGCCAACCGCTCCGCCGCCGTCGTCGCGCACTCTCTGCAGATCCCCAGCAA
TGAATCATCTTATTTCCGATACGTGGGACCATACATGGTCATGGCGTTGGCCATGCAGCCGC
AGCTGGCCGAGATATCATACACCAGCGTGGACGGCGCCGCGTTGACGTACTACCGCGGCGA
GAACGGCCAGCCGAGAGCCAAGTTCGGGAGCCAGAGCGGCCAATGGCACACCCAGGCCGTT
GATCCGGTGAACGGCCGTCCCACCGGCCGCCCTGACCCAGGGGCGAGTCCGGAGCACCTAC
CCAACGCGACGCAGGTCCTCGCCGACGCCAAGAGCGGCTCGCCCGCGGCCCTCGGGTCCGG
GTGGGTCAGCTCCAACGTCCAGATGGTGGTCTTCTCCGCGCCTGTCGGTGACACTGCGGGCG
TGGTCTTCGCAGCGGTCCCCGTCGACGTCCTGGCGATCGCCAGCCAGGGCGACGCCGCCGCC
GATCCCGTCGCGCGGACGTACTACGCGATCACCGACAAGCGCGACGGCGGCGCCCCGCCGG
TTTACAAGCCTTTGGACGGCGGGAAGCCCGGCCAGCACGACGCGAAGCTGATGAAGGCCTT
TCCCTCGGAGACCGAATGCACCGCGTCCGCCATTGGCGCGCCCGGCAAGCTCGTGCTCCGCG
CCGTCGGGGCGGACCAAGTCGCGTGCACGAGCTTCGACCTCTCCGGAGTGAACCTGGGAGTT
CGTCTTGTGGTCAGCGACTGGAGCGGGGCAGCCGAGGTCCGGCGAATGGGGGTGGCCATGG
TGAGCGTCGTGTGCGCGGTCGTGGCGATCGCGACGCTGGTGTGCATCCTTATGGCACGGGCG
CTGTGGCGGGCCGGGGCGCGGGAGGCGGCTCTAGAGGCTGACCTGGTGAGGCAGAAGGAG
GCGCTCCAGCAAGCGGAGCGCAAGAGCATGAACAAGAGCAATGCCTTCGCCCGCGCCAGCC
ACGACATCCGCTCCTCACTCGCTGCCGTCGTTGGACTCATCGACGTTTCCCGGGTAGAGGCC
GAGAGCAACGCCAATCTCACCTATAACCTCGACCAGATGAACATTGGCACAAACAAGCTCTT
GGATATACTTAACACGATACTGGACATGGGCAAGGTGGAGTCCGGGAAGATGCAGCTAGAG
GAGGTGGAGTTCAGGATGGCAGACGTCCTTGAGGAATCCATGGACCTGGCGAACGTCGTCG
GCATGTCAAGAGGCGTCGAAGTGATCTGGGACCCTTGTGATTTCTCCGTGCTCCGGTGCACC
GCCACCATGGGCGACTACAGGCGTATCAAACAAATCCTTGACAACCTACTCGGCAACGCCAT
CAAGTTCACACACGACGGCCACGTCATGCTTCGAGCATGGGCCAACCGTCCCATCATGAGGA
GCTCCATAATCAGCACCCCATCGAGGTTCACCCCCCGTTGCCGCACGGGTGGGATCTTTCGG
CGGCTGCTTGGAAGGAAGGAGAACCGTTCGGAACAAAATAGCCGAATGTCATTACAAAATG
ATCCCAATTCGGTCGAGTTCTACTTCGAGGTGGTTGACACTGGTGTGGGCATACCCCAGGAA
AAGAGGGAGTCTGTGTTTGAGAACTACGTTCAAGTGAAGGAAGGGCATGGTGGCACCGGGC
TCGGACTTGGAATTGTGCAATCCTTTGTTCGTCTGATGGGAGGAGAAATTAGCATCAAGGAC
AAGGAGCCAGGAGAAGCGGGGACGTGCTTCGGCTTCAACATCTTCCTCAAGGTCAGCGAGG
CATCGGAGGTGGAAGAGGACCTCGAGCAAGGGAGGATGCCGCCGTCGCTGTTCAGGGAGCC
CGCCTGCTTCAAGGGCGGGCACTGCGTCCTCCTCGCCCACGGCGACGAGACCCGCCGGATCC
TGTACACGTGGATGGAGAGCCTCGGGATGAAGGTCTGGCCCGTCACGCGCCCCGAGTTCCTC
GTCCCGACCCTCGAGAAGGCGCGCTCCGCCGCCGGCGCCTCGCCGTTGAGGTCGGCGTCGAC
GTCGTCGCTGCATGGCGTCGGCAGCGGCGACTCCAACATTACGACGGACCGGTGCTTCAGCT
CCAAGGAGATGGTCAGCCACCTGCGGAACAGCAGCGGCATGGCCGGCAGCCACGGCGGGCA
CCTCCACCTCTTCGGCCTGCTCGTCATCGTCGACGTCTCCGGCGGGAGGCTCGACGAGGTCG
CCCCCGAGGCGGCCAGCTTGGCGAGGATCAAGCAGCAGGCGCCGTGCAGGATCGTCTGCCT
GACGGACCTCAAGACCCCCTCCGAGGATCTGAGGAGGTTCAGTGAGGCGGCGAGCATCGAC
CTCAACCTGCGCAAGCCCATCCACGGCTCCCGGCTGCACAAGCTACTCCAGGTCATGAGAGA
CCTCCATGCCAACCCGTTTACGCAGCAGCAGCCGCAGCAGCTCGGTACAGCCATGAAAGAA
CTGCCGGCTGCTGATGAGACCTCTGCGGCTGAGGCGTCTGAGATCACGCCCGCGGCGGAGG
CGTCTTCTGAAATCACGCCCGCGGCGGAGGCGTCTGAAATCACGCCGGCAGCGCCGGCGCC
GGCGCCCCAGGGAGCGGCCAATGCTGGAGAGGGCAAGCCGCTGGAGGGGATGCGCATGCTG
CTGGTGGACGACACCACGCTGCTGCAGGTAGTCCAGAAGCAGATACTGACCAATTACGGGG
CAACCGTGGAGGTCGCCACGGATGGCGCCATGGCCGTGGCCATGTTTACAAAGGCTCTTGAG
AGCGCAAATGGCGTCTCAGAGAGCCATGTGGACACAGTGGCCATGCCCTACGACGTCATCTT
CATGGATTGCCAGATGCCAGTGATGAATGGGTATGATGCGACGAGGCGCATCCGGGAGGAA
GAAAGCCGCTACGGCATCCGCACCCCGATCATCGCGCTCACCGCTCATTCCGCGGAGGAGG
GGCTGCAGGAGTCCATGGAGGCAGGGATGGATCTTCACCTGACCAAGCCAATACCCAAGCC
GACAATCGCACAGATTGTTCTTGACCTCTGCAGCCAAGTTAATAACTGA
SEQ ID NO: 15 OV1-A polypeptide sequence
MAGEVGKWGSSFKRSWALIPLVAHGIIVVVVGLAYSFISSHINDDAVSAMDASLAHVAAGVQPL
MEANRSAAVVAHSLQIPSNESSYFRYVGPYMVMALAMQPQLAEISYTSVDGAALTYYRGENGQ
PRAKFGSQSGQWHTQAVDPVNGRPTGRPDPGASPEHLPNATQVLADAKSGSPAALGSGWVSSN
VQMVVFSAPVGDTAGVVFAAVPVDVLAIASQGDAAADPVARTYYAITDKRDGGAPPVYKPLD
GGKPGQHDAKLMKAFPSETECTASAIGAPGKLVLRAVGADQVACTSFDLSGVNLGVRLVVSDW
SGAAEVRRMGVAMVSVVCAVVAIATLVCILMARALWRAGAREAALEADLVRQKEALQQAER
KSMNKSNAFARASHDIRSSLAAVVGLIDVSRVEAESNANLTYNLDQMNIGTNKLLDILNTILDM
GKVESGKMQLEEVEFRMADVLEESMDLANVVGMSRGVEVIWDPCDFSVLRCTATMGDYRRIK
QILDNLLGNAIKFTHDGHVMLRAWANRPIMRSSIISTPSRFTPRCRTGGIFRRLLGRKENRSEQNS
RMSLQNDPNSVEFYFEVVDTGVGIPQEKRESVFENYVQVKEGHGGTGLGLGIVQSFVRLMGGEI
SIKDKEPGEAGTCFGFNIFLKVSEASEVEEDLEQGRMPPSLFREPACFKGGHCVLLAHGDETRRIL
YTWMESLGMKVWPVTRPEFLVPTLEKARSAAGASPLRSASTSSLHGVGSGDSNITTDRCFSSKE
MVSHLRNSSGMAGSHGGHLHLFGLLVIVDVSGGRLDEVAPEAASLARIKQQAPCRIVCLTDLKT
PSEDLRRFSEAASIDLNLRKPIHGSRLHKLLQVMRDLHANPFTQQQPQQLGTAMKELPAADETSA
AEASEITPAAEASSEITPAAEASEITPAAPAPAPQGAANAGEGKPLEGMRMLLVDDTTLLQVVQK
QILTNYGATVEVATDGAMAVAMFTKALESANGVSESHVDTVAMPYDVIFMDCQMPVMNGYD
ATRRIREEESRYGIRTPIIALTAHSAEEGLQESMEAGMDLHLTKPIPKPTIAQIVLDLCSQVNN
SEQ ID NO: 16 OV1-A genomic sequence. Start codon at bases 3,178-3,180.
Stop codon at 9,837-9,839.
TTGCTTTTAAGTTGTAAATGTCGTAGGCTTCCTTCTCACGTTATTTTTCTTTTCTTTTAGTCGG
AGGGTGTGTGTTGTGGTCTGCTGGGAAAAGCTTCCCTGCCCTAATTGGGTCCACTACTTCTTT
AACGTTTACCACTTCAATTAAACGAGTTCAATAACGAAACGCTTTTGTACAAATGTACCAGC
CTTTATGGTTTATTTATGTAATCAATCATGACGTATTCACCCAAGTACATTCTGATATTTATG
TTGAATGTGAACATTGTCTATTAATCATGGGGTAGTGTATATACTCACTAGGGTGCTCATGTG
CTTAAGTTGCATCCCCACAATTGTTTATATTTACTACAAAACAAAGATAACTGGATCAACGA
ACGAATAAATTGACGGGTGGTCCTTTCATGCTATCCACCAGATGGGGCAATTGCTTTTAAGT
TGTAGATTTCGTAGGCTTCCTTTTCATGTTATTTTTATTTTTGATTAGTCAGATGGTGTGTGTT
GTGATCTGCTGGGAAAAATCTCCCCCCTCCATTGGTGGCAATAAACATAAAAAGGGTCAGCT
CTCACGTCATACAAAAATAAAAGAAAACAAATTTGAATATAAATCAATATAATTTACAATA
ACACACAAGACCGTCCCATTACCATTGTAGGAAACGCCACACCCTTTCCCATTTTTGGAAAT
TGACCACCACCGGTGGCTCATCCTGTAAACCTTCCCTTCAACTTCTTGGTTGTTCTCATCTAC
TTCTAACTTAATTATTTAAGGGACATGCATAGAAGGTGACTACCAGTGATGAGCACGGCGTC
ATGGGAGCCTATGAACAACTTTCAACCATACATGACACACCTTTTATAAAGGGAAACCCATA
TTTCTAACTAAACTTCAACAATATTCATAAAAAATAGCATGTGATGCCACTTTCAACCAAAA
TTTAGTATCCAAAAATCTACAATTTTTTGAATTAATTGTTTAATTTTGTACAAAATTCAGATG
GCTTAAATAAACATTCATGCATTTCTAACTAAAAATTCTCACAAAAAATTCTTCCAACTTTCA
ACTCAAGGGAAACCGAAAGATGTGGCCAATCCTACCTTAGAGATGGCTTTATCAAGGCATG
ATCATGATAATGGGACAAGTATAGCCTCCTAAGGGTTGTTAAAGAACGTGCATGTTGAAATT
ACCATCATCATAAGATCCATGCCCCCCCCCCCACACACACACATACCTACATGATCCAATCA
CAAGAGATTTGGTGGGACATGGTATTATATTTTCTTGGGTGTGGTTTGAAAAGTTCAAGCCA
ACCTTGTCTATTATGTCTTAATTAAAAGTTGTGTCGTGCAATCGTTTAAATGCAGTTCCATTT
TTCTCCAAAAAGACAAATGACCAAAACAACACCTTCATTTACCATCGCTACCTTCTACCCAT
ATCCATTCCCTGCTCCCATCCGTCCTCGGTCACAGCTCCTACACCATGAGACAGAGGGAGGG
GATCCTCATCTCTCATTTGATGTGTAGGAAAAGTTCGTTGGTTAGTTTTTGGTGTCACGGACA
TCACAATAACAGTTACGACTCCAATACTGTCTTGACGGTGTTTGGTGAGGTGTTGCCAAGGA
GATTGTGAGTATTTTTTGTTCTCGTCGGCCTTTTTGGACGACGTTGTGGTTCTTGTTGCTATTT
TGGCGAGGCATCGTTGACTGGTGTCGTCTTCTTCAACAACTCTTTCCTTCGAGCCTTTGCGAG
CAATGTCAGTGGCCTAGTTTGGCAATATGAGTGTGGCTGCCTTCCTAGATCCCCCATGCCAA
ATGTTCTTGGCATGATTGCTAATACCTGTTATACACGGCCGTCTACCATTGCGACCATTCAAG
ATGTGGTCCTCTAGAGGTCTTCACTGACTAAGATTCTCGATGTTGTTCTCCGCCGGCGAGAGC
TAGGTGGGGCAACGACGACGAGTTTGGTTACGGTTTGGCTTGAATTTTGCAGCCGACGGTGT
TGGTTATAATTCATCATTTGTTTATGGTTATTTCTATGCTTGTGGGTTTAGTTACCTCGTTATT
TGAATGTATTCGCTTTTTTCTTTGTGATATAAGATAGAACTGATTAAAAAAATTGTGCAAGTA
ATAGTGAGGCAAATAAGCTACATATGTACATTGAAAACAATATGACATATCCACTAACTATA
ATACGCACAAAAGAATTGCGTATGAGTCTTGTATTTTTTTTTTCTTATTTTCAATGGAATATA
GGTGACATGTTGAACGGTCTCACACGAACGGCTCTACATATTGCCCACTCGGCACACAAGAG
GAACGCTCGAACTTGTCGTGTGTGTGCGGACAAAATAAATAGAACTTTTCAATTTCCGCGCT
TTGAGATTTTCAGCTGATAATTAACGCATTAGACAGAGCATCGAACGAGTCGATCAAGTTTG
AATAAGTAAGTCACAGGCTAAAAAAAGCAAGACACAGTTCATCTTTTTTTTAGGAAAGGACT
TGGTTCATGTTTATTTTATTTTTTGAGGAAAAGGGCCTGGTTCATCCTAGCTGTCCTGGTAAC
GGAGCTAGTTCAACATAAGTCGAGCCTGTGCTTCTATATTTAGCATCAAACGTAACGAAGAG
TTAACTTAACAAAACTAATGATAATGCTATCTTAAGACAGGAAGCTAATTGATCGGTGTTCA
CTTGCACAACAGGATGGCCTTATGGCGATCGTGCGGCTGCCAACACTGCCTCACGCCCACCC
AAACTATTCGAACGGGAGGAAAACATAATTCATTGTGCTCAGATTTGGCAATTGATTACAAG
ATCGCGTAGTATCCCTTTTTCTATCTCGTCGTGTGTCTACTACCGAGCCGTGCATTGATATTA
GATATCGAAGTTAAATGTTGATTTTTTTAGAATAAATACTTCTTTTTTATTGCGGTGCAGGGG
TAAATATCGATTTTTTTTCTGCGGTACAGGAGAAATACTGCCAAAAGTGGTATAATAAACCG
CGACGGCGGATTTAAAAAACCGGCGGCGCTCTGAGCCCACAATTCAGAGCGGAAAAAACCT
TCAATGTGTAAAGTGTATTCCTCCGAGCATCTATAAAAGGCCGTATACCTAGGAAACAAATA
CTCACGAACACCTAACAAAGATTTGCTTCTGTAGAATACTTTACAATACTTCCGCGATGGCT
GGAGAGGTTGGCAAGTGGGGTAGTTCCTTCAAACGTTCTTGGGCTTTAATCCCACTGGTATG
AGAACATTCATAGTACTTGGCTTCTTTTTGTGAAACTCATGGTTAATACTTGTTCTTCTATAT
GAACTTCATGGCTATTCTCTTAAAAAAAAACTTCATGGCTTTTCTCATTTCTTGGTTCTTGTCC
TTCCACCTACAACTATTGCTTACCTGAGCGGAAACTAGATCTGCGAGGTTATCATCTGCTTAA
GCTTTTTTTTTTTGAGGGATTCATCTTACTTAAGCTTAGTGCAACAAAGACATCCTTCCGCAT
ATGTGGGTATGTGATCTCCACAATAGATCTATGTAGTGGTACGGTATTCTTTTGGAAGAGGA
GGATTACCCCCCGGTCTCTTGGCATAGGTGTGAGTTCAAAAGTAATTTTAATGATTTTTGTCT
TACAATTAAATTCTGTTTCGTACGATATAGATATCTTCCTATGCCTGTAAGGCGCAGATTTGA
AAAGTAGATCTAACTGATTTTTATTGTTTATGTTTCATTTCTGTATATTACAGATATCATCCTG
CACGTGTCAGGCATGCGAGATCTCTTTCTTTAGATAACAATGAAAACTAGATCTTCATAATTT
TTCATCTTGTAATTGAAGATTTTGTTCCGTACAAGATGGATATCCTCCGACGTGTGTTTTAGG
CATGAATTCAAAAAGTAAATATAATTTTATTTTATCTTGTAATTAGGAATCCGCTCCGTACAA
CATTAGTGTCCTTTCACCTGTGTAAGGTGTGCTCTAAAAATACATCTATGTAGATTTTGATAT
ATAATTTGGCTTCTGCTCCGTACATCACAGATATCTCCCCACATGTGTGAGGCAGTGGCGAG
TCAAAGAACTCCCTTTTGTCGTATGATCCTATGTTGTTTTTCTGGTCACCGTGTGTTAGATCTA
CCCATGGAATTGTTTTTCTGGTCACCGTGTGTTAGATCTACCCATGGAATGTCACGACAAATG
TTGTCGTTAGATGAGCCCAAAGGCGATCTGTTAGCGGGATGTTCACGACAAATTATATCTTT
AGATTAGTGAAAACCCTCGAAATCTTGACTTATTACTTGTGCAACAAATGTTATCGTTAGAC
GAGTTGAAACACAATGCAACGCCGCTTGTCGAGGCCTCGTCAGCCTAAAAGACAGTTTAATT
TATCCGCAAAAAAAAGACACTTTAATTTAATATTTATGCATTTTCTATTTTACTTTTTACATGT
TGCAAAAAAAAATCTGACGTCACACTTTTATTGCACTTGCACGCATGCAGGTCGCCCATGGA
ATCATCGTGGTCGTAGTGGGTCTGGCTTACTCTTTCATCTCGTCGCACATAAATGATGATGCC
GTGAGCGCCATGGACGCGTCGCTGGCGCACGTCGCCGCCGGCGTGCAGCCTCTAATGGAAG
CCAACCGCTCCGCCGCCGTCGTCGCGCACTCTCTGCAGATCCCCAGCAATGAATCATCTTATT
TCCGATACGTAATTAACCAAGAACCTTTGGGTTAATTAAATTATGCATTTTTTCTATGTAAAA
CGTGATTAGTTTCATCACGTATATGCACTTCCTTTTTGAACCACAATTATTTCCTTACTTTAAA
TAACAAATCTTAATTACTAGCCGGGCCGACCCGGTAACTGGTTATTGTGTATGATTCTGTTCT
GATTTTCGTAGTAATGCGAGCATTGATATGAATATACGCATGCATATACAAGCAAATAATTT
TTGCGTGCATTTTTTTTTATGTAGGACACGTCCAAGATAACATAGCAACACGTACTACGTGC
AAATATGCATCTAACATTTACGTATATGTTTGACCTGACAGGTGGGACCATACATGGTCATG
GCGTTGGCCATGCAGCCGCAGCTGGCCGAGATATCATACACCAGCGTGGACGGCGCCGCG
TTGACGTACTACCGCGGCGAGAACGGCCAGCCGAGAGCCAAGTTCGGGAGCCAGAGCGGCC
AATGGCACACCCAGGCCGTTGATCCGGTGAACGGCCGTCCCACCGGCCGCCCTGACCCAGG
GGCGAGTCCGGAGCACCTACCCAACGCGACGCAGGTCCTCGCCGACGCCAAGAGCGGCTC
GCCCGCGGCCCTCGGGTCCGGGTGGGTCAGCTCCAACGTCCAGATGGTGGTCTTCTCCGCG
CCTGTCGGTGACACTGCGGGCGTGGTCTTCGCAGCGGTCCCCGTCGACGTCCTGGCGATCGC
CAGCCAGGGCGACGCCGCCGCCGATCCCGTCGCGCGGACGTACTACGCGATCACCGACAA
GCGCGACGGCGGCGCCCCGCCGGTTTACAAGCCTTTGGACGGCGGGAAGCCCGGCCAGCAC
GACGCGAAGCTGATGAAGGCCTTTCCCTCGGAGACCGAATGCACCGCGTCCGCCATTGGCGC
GCCCGGCAAGCTCGTGCTCCGCGCCGTCGGGGCGGACCAAGTCGCGTGCACGAGCTTCGAC
CTCTCCGGAGTGAACCTGGTAACGCGTCCATCTAGCATCGATCACAGGCCATCCATATATGC
ATACGTACACCAACGTGCACACAGCCTATCTAACGTAATTCCTGTGCATTATTTTTGTCAAGA
ACTAATCCCAGCATGTAATATTTCTTCCAAGTTTGCTGTTTATACATTAAAAAGCAACGGATA
ATGAAAAAAGGTTAGATGAGCTAAGGGGACTTTGGCAAAAAAAAAAACTAATAAAACGTTT
TTTTGTTTTGGCGACCTCACTAAACATCCTTCCGACTTGGAGTGAGGAAAAAAAACAGGGAT
CGCTCCTAGCTATTGACAGTACGTACACAATCTTGCTTCCTTCCTTTCGCATGTAAAAAAACT
GAAAACTTTCCAAATCAAGGGATCCCAAAATTAGGAAGAAAATCTTAATGGAGAGTACAAA
GTCTTCTTCTTCCTCTTCTTCCCCAAAGCAAGATTTCTCATTCTGTTCTTCCCCAAACGGAGCT
CTGGCACAAAACTGTGGTGAGCTCGATCGTCTCCTACGTACTTTTCTTGCATCTGCTAGTGTC
TTGCATGCATATCACCGGTTGTCGTTCATGGATATCTCCCATCAGTTCTTTTGCAATTTATTTA
CAGCGTATGAACGAGCGCTGGTTTGAAACTGATCTCCCATCTGCTAGTGCAATTAGGATATC
TCCCATCTGCTAGTGTACTTTTATTGAAACTGATCTTGCCATCCGTCGCCTATGAACGAGCGC
TGGTTTGATAATTCTCCGACCAGGCCGGGCGGCGCCTCGCGCGCCATAGAAACAGTCTTTTT
TTTCGATAAAGGCCATGGAAACAACCTGACGTACAGCCTCTTGTAAAAAACAATTATTTTCT
TCGTAGTATAGCACGCATATGCATGTTTGAGAATTTTTATCGGGACGGCTGACAAGTATCTC
CGGTTGTATTTCTTCTTGTTTTTCAGGGAGTTCGTCTTGTGGTCAGCGACTGGAGCGGGGCAG
CCGAGGTCCGGCGAATGGGGGTGGCCATGGTGAGCGTCGTGTGCGCGGTCGTGGCGATCGC
GACGCTGGTGTGCATCCTTATGGCACGGGCGCTGTGGCGGGCCGGGGCGCGGGAGGCGGCT
CTAGAGGCTGACCTGGTGAGGCAGAAGGAGGCGCTCCAGCAAGCGGAGCGCAAGAGCATG
AACAAGAGCAATGCCTTCGCCCGCGCCAGCCACGACATCCGCTCCTCACTCGCTGCCGTCGT
TGGACTCATCGACGTTTCCCGGGTAGAGGCCGAGAGCAACGCCAATCTCACCTATAACCTCG
ACCAGATGAACATTGGCACAAACAAGCTCTTGGGTCAGTCTGCATCCATGCCCTACGTACCA
TGCATGACAATACCATGAATAGCTTGCGCTACCTTTTAGTAGATCTATCCGTACTTGGCAATT
TAGCTAATGTCATCATAGCATTATAAAATTGCATGTCATAGAAGTAAAGTTTCTGTAAATAA
TTTAATTACAGTCTTAGGAGTAGGGTATGCAATATCCCAGCTGTTATACATTTAAGGTATCA
AATTTGCTCATAAAATTTAAAATATGCAAGAAATCAATCTCTGTTTGGTAAAGAAATAGCAT
TTTATTTGTACAAGAAATAAAGTTTAGGATAGTTCAAATCGAATTGTCAGATATCACTATGTT
AGCAGCCAGTAAACTTATGAAGTTTCAATATTTCTATCTATTTTGCTGCTGGGCAAATTTTGT
CACATTTACGCATCATGTTTTTTGTGCGTGTCTTTGAGTGCATAAAGCAAAAAAGTTTATTAT
TTCCGAAAAAATGAACTTTATACCATATTTGTGTTCTCAACTGAGCATATGTTGACTCATCAT
CCATGTTTATACATGTGTGTGTACATGCAGATATACTTAACACGATACTGGACATGGGCAAG
GTGGAGTCCGGGAAGATGCAGCTAGAGGAGGTGGAGTTCAGGATGGCAGACGTCCTTGAGG
AATCCATGGACCTGGCGAACGTCGTCGGCATGTCAAGAGGCGTCGAAGTGATCTGGGACCC
TTGTGATTTCTCCGTGCTCCGGTGCACCGCCACCATGGGCGACTACAGGCGTATCAAACAAA
TCCTTGACAACCTACTCGGCAACGCCATCAAGTTCACACACGACGGCCACGTCATGCTTCGA
GCATGGGCCAACCGTCCCATCATGAGGAGCTCCATAATCAGCACCCCATCGAGGTTCACCCC
CCGTTGCCGCACGGGTGGGATCTTTCGGCGGCTGCTTGGAAGGAAGGAGAACCGTTCGGAA
CAAAATAGCCGAATGTCATTACAAAATGATCCCAATTCGGTCGAGTTCTACTTCGAGGTGGT
TGACACTGGTGTGGGCATACCCCAGGAAAAGAGGGAGTCTGTGTTTGAGAACTACGTTCAA
GTGAAGGAAGGGCATGGTGGCACCGGGCTCGGACTTGGAATTGTGCAATCCTTTGTAAGTG
ATCTCATCTTTTTTCATCCATGTTAAAATCTTGTCAAGTGCATCAACGTTAACTAGCCGTAAC
TGTATTCTTCATGGGTAGGATGTGTGTGTGTTCGTGTTTGTTTGTTTGGAAAAGAAAATTATA
TTTTTCACTAACGTTTTCGTTTTTTCTTGTTTACTTATAGTTTTGTTTGCTGTTGTTGTTGATGT
AAACATAGGTTCGTCTGATGGGAGGAGAAATTAGCATCAAGGACAAGGAGCCAGGAGAAG
CGGGGACGTGCTTCGGCTTCAACATCTTCCTCAAGGTCAGCGAGGCATCGGAGGTGGAAGA
GGACCTCGAGCAAGGGAGGATGCCGCCGTCGCTGTTCAGGGAGCCCGCCTGCTTCAAGGGC
GGGCACTGCGTCCTCCTCGCCCACGGCGACGAGACCCGCCGGATCCTGTACACGTGGATGGA
GAGCCTCGGGATGAAGGTCTGGCCCGTCACGCGCCCCGAGTTCCTCGTCCCGACCCTCGAGA
AGGCGCGCTCCGCCGCCGGCGCCTCGCCGTTGAGGTCGGCGTCGACGTCGTCGCTGCATGGC
GTCGGCAGCGGCGACTCCAACATTACGACGGACCGGTGCTTCAGCTCCAAGGAGATGGTCA
GCCACCTGCGGAACAGCAGCGGCATGGCCGGCAGCCACGGCGGGCACCTCCACCTCTTCGG
CCTGCTCGTCATCGTCGACGTCTCCGGCGGGAGGCTCGACGAGGTCGCCCCCGAGGCGGCCA
GCTTGGCGAGGATCAAGCAGCAGGCGCCGTGCAGGATCGTCTGCCTGACGGACCTCAAGAC
CCCCTCCGAGGATCTGAGGAGGTTCAGTGAGGCGGCGAGCATCGACCTCAACCTGCGCAAG
CCCATCCACGGCTCCCGGCTGCACAAGCTACTCCAGGTCATGAGAGACCTCCATGCCAACCC
GTTTACGCAGCAGCAGCCGCAGCAGCTCGGTACAGCCATGAAAGAACTGCCGGCTGCTGAT
GAGACCTCTGCGGCTGAGGCGTCTGAGATCACGCCCGCGGCGGAGGCGTCTTCTGAAATCAC
GCCCGCGGCGGAGGCGTCTGAAATCACGCCGGCAGCGCCGGCGCCGGCGCCCCAGGGAGCG
GCCAATGCTGGAGAGGGCAAGCCGCTGGAGGGGATGCGCATGCTGCTGGTGGACGACACCA
CGCTGCTGCAGGTAGTCCAGAAGCAGATACTGACCAATTACGGGGCAACCGTGGAGGTCGC
CACGGATGGCGCCATGGCCGTGGCCATGTTTACAAAGGCTCTTGAGAGCGCAAATGGCGTCT
CAGAGAGCCATGTGGACACAGTGGCCATGCCCTACGACGTCATCTTCATGGATTGCCAGGTA
CATTTCTCCAGCAAACAACGTGCCAAGCACATCAGCCCCATCTCTCTTGTTCCTGAAGATGA
TTTAATCTGACGTTGCTGACAATTCGATCTTCTTTGTTTCAGATGCCAGTGATGAATGGGTAT
GATGCGACGAGGCGCATCCGGGAGGAAGAAAGCCGCTACGGCATCCGCACCCCGATCATCG
CGCTCACCGCTCATTCCGCGGAGGAGGGGCTGCAGGAGTCCATGGAGGCAGGGATGGATCT
TCACCTGACCAAGCCAATACCCAAGCCGACAATCGCACAGATTGTTCTTGACCTCTGCAGCC
AAGTTAATAACTGATCGCGGAGATTCTTCGTTCCCTGTTCCCTGTTCCCCGGTCACATGATCA
AATATCAAGATAGGTGTAGGTGGTTTTTCAGCCAGCGAATGCAGTTGTCATCCTAGTCACTG
AAAACCCACCTACATCTCGAGTTTTGATCATGCGACCAGGGGCATTATCGTAGTTTGTAGCA
TTTAGCAGCAGCAGCTGAACTTTGTTGTTGTATCAAGATCAGGTCAGGTTTATTTCCAGAATT
ACTCTTGGACAATGTATTGTCAATTTTGAATTTCCAGAAACAATTATGGTTAAGTTTTGAGTT
CCAGAGTTGGTGTTTTCAGAGTTCTTTTTTTTCCGGAGTTTGTGTTTGGGTCTGTCTAGCACAC
ATCTAGATGTGACATAGTTATGTCACATCTAACCTGATAAGCACTATGTTTGTGGTCTATTTT
TTTTGTCCCAGCTTTTTTTTTATTTCTTGTTGCTGTGTAGTTATTTTTAGAAGGTTAGATGTGA
CATCACTAAAAAACATCTAGATGTGAATTAGACAAACTGTTTGTGTTTTCAGAGCATGTGAT
TAGACGCCATATATTTGCTTCCATTGCCCATTCTCTGGAGAAAGAAAGTACAGATTCCTACA
AGCTATGAAATCCCTGGCTAGCTACCTTGTATATCTAGTAGTGTACACAAGCATAGCTGATA
AATACCCATAGGAATAACTGTACAGTCTCCTCTAGGTCTGCAGTGGACTTGCCTAAATACTA
GTACTATCTCTCATATGCACGCACCAAGTGGGAAAAGTTCACACCCGAGGTCATTTTCATTG
AATGGCACTTCGTCGTTCTCCTGCGTTGAAATCAGAAAAAGGGTTCAGAAGAAACACCATTG
AAAATCTAGGAACATAGGGTTTACTTAGCTTCGATCAGTGCAAACGATTTGAAAGGAAATAT
GCCTATCCGAAATAGTGAAATTTTGAGGGGGGAAGAGTAAAGTCAAGCATAACTGAGGTTC
TTACCACTTTTATTATAGAATTGAGAAGACCATCAAAGTTGCAGCTGCTGGAATTGAACCCA
CCCTGCAGCTTTGGAACTCCCAAATCATACATATCGGTCTGCTCAGGAACAATGGCACCACC
ATCATAGCTAAAACCATCATTGTTCAGTCTATGGTCTTGTCTCATTCCATCCATTCTATGTCT
GGAGTTGCTTGCAGCCCCAAACTGGAGGTATTTTGAATGTCTTTCGGTATCATGAGGAAGGG
GCAGAATTGTACTGCCAGAGGAACTAGCTCTACATTTGGCATTGCTGGCTACTATAAGGCTC
TCAGAAGGACAAACACTAACACCCATTCTTTCCGAAAATATCCCCTTTTGGTCCATCTCATGT
TCTCCAGCCACAAAGCTAGCGTCAAGTTTTGTCGCGCCAACTCGATCGAACGGGATGTGCAA
CGGTAACTTGTCAACAGAAAACCCATCACTGATTAGAAGAGCACACCGCTGAGATATACAA
GAATCCCGGAAATCGGCGGAAACTCCGACAGACCTTTCAAGCAGCCCTGAACTTGCAGTTG
GTATTCCTATTGATGGATGGGCTCCAACTTTGATGTGTGTAGTGCATTCCAAAAGATCTTCTG
GTGGCAATGAACTGCTTGTAACTCTTTGGAGTGTGCCAGACAGAGTGTTAGCCAGAGCACCC
CCGGAAAAGACAGAGCACAGGTCATTGGTTTCTTGATGGATCCACTTCTGCTGGAGCTGAGG
CTGCCCAAGCGACGATGAGGTCAAGCCTTGCGATAAATCTGCCTGTTGGTTGTCTTGTAAGC
TCACAATGTGGAACTTATCGGTATCGCCGGCGCAATGACTTACTGCGTCGTTGCTGCTAGAA
CACTGGTTTGCCTTGGGAGAAGCAAGGTCCTGAAGCCCAAATGCTGCTGCACCAGCACTACT
CAGCAGTCCATGTGGATTGAAAGATGGAAGAGCAGCAGAAGGGGCAAAGGGTTGATGATA
ACTATGAAGTCCTTCAAATGCTCCCATGTGCAAGAAGGGGTCTCTGCCTCCAAAAGCAGCAG
CAATGCTGGCTTGCTGTGATGCCACGGCACTTAGCCGTCTGAGGTATAGCCTGTACTTCTGC
ATTGGCATACAAAGTAACCTGATTAGACATGGAGGAACTTATGTAATCATCAGAATTCTGAA
ACATGAGATATACAGTCTTAAGATAACTGCTTCCTGGTTTGGCATGGAGATTCTTGCTAAAC
TGGTACATAAAAGCACTCCAGGATATAGAAAAGGCTGATGGATTTTCTTAACCTTTTAATGT
AGGCTTGTTAATAAATTTTCTATTTGTGGATTCATTGACATGCAAAGCTTTTGCATTTCTCTA
AAAAATATTTCAGTTTACATATGTAGCGCTGTAATCTGGACAGGGTGGTGTCAGTGTCTTCCT
TCTAGCAGTTTTACAGAAGTGAAACAATGTAGCCAAAATACTACAATAAAGTCAGCAGTAC
ATACCAAAACACCACATTAAACATGTACAGCAGTACATACCAAAATACAACATTAACTTGTA
CAGCAGTGCATGCCAAAATACTGTAGTAAACTTGTACAGCAGTATTACCTAATTACTCTGCA
TTAAACCTGTACAACAGTACATACTAATTTGAGCATTTTATACCTGTGTAGTTAGATATAATC
TACTAGCCTACATGGTTGGGAGAATAATCAGATATTTGTATTAGATATCTCCTTCAGGTTTTC
TGAAAGATTCAGTATAACTGAACTGAATGTTTCTTTGTTTTCAGACCAACTGAACATATTCAA
CTTGTCAAGAAAAAGGAAGAAATGTGAAGGTGAAACTATGTAGCCGCAAAATTAAACTACT
TAACTCATTTTGCACGAACACCAGGAACATGTTAACTTATTTGTTGAAAGAGTTCTGCTCTGC
AACACCATGATATCAAATTAAGCTCTGCTCTGCAACTAATAGGTGTGTCGATACCAAATACA
GTAAGGGGATAAGGACTCTGTTATAAGTTGGCCCTCTTAATTCGACACAGGAAAATAAGGCC
ATTTGCAGATAATTTTACCAAAAGTTCTGTAAAAGTTTTTCTTCCTGTGACCAACGAAAGAG
ATAACACAGGTAAAGGTGACGATGGAAGTAAATGCCATGTCGCTATACCTGCAGATGGCTC
GCAACATTTTCCCTGGTGAGCTTCTCCACGTTCATAAGCTCGAGTATTCTTTTGGGCACAGCC
TCTGCAAACAGCAGCAAGAGAAAGGACACGAGCATAAATGGGTATGCTGTAGCCTGCAAGA
ACATTTAACATAAAGCAAAAGGCAACGTGGGGAGGTGTTTTTTTTTTCTTTCTTACTGTCGAT
CCCGAGCTGGTTGACGGCGGCGACGAACTTCCGGTGCAGCTCCACTGACCACACCACCCTCG
GCCTCTTCGA
SEQ ID NO: 17 OV1-B CDS
ATGGTTGGAGAGGTTGGCAAGTGGGGTAGTTCCTTCAAACATTCGTGGGCTTTAATCCCACT
GGTTGCCCATGGAATCATCGTGATCGTAGTGGCTCTCGCTTACTCTTTCATCTCGTCGCACAT
AAATGATGATGCCACGAGCGCCATGGACGCGTCGCTGGCGCACGTCGCCGCCGGTGTGCAG
CCGCTCGTGGAAGCCAACCGCTCCGCCGCCGTCGTCGCACACTCTCTGTTCATCCCCAGCAA
CGAATCATCTTATTTCCGATACGTGGGACCGTATATGGTCATGGCGTTGGCCATGCAGCCGC
AGGTGGCCGAGATATCATACGCCAGCGTGGACGGCGCCGCGTTGACGTACTACCGCGGCGA
GAACGGCCAGCCGAGAGCCAAGTTCGTGAGCGAGAGCAGCGAATGGTACACCCAGGACGTT
GATCCTGTGAACGGCCGTCCCACCGGCCGCCCCGACCCGGCGGCTCAGCCGGAGCACCTACC
CAACGCGACGCAGGTCCTCGCCGACGCCAAGAGCGGCTCGCCCGCCGCCCTCGGGGCCGGG
TGGGTCAGCTCCAACGTCCAGATGGTGGTCTTCTCCGCGCCTGTCAGTGACACTGCCGGCGT
GGTCTCCGCCGCGGTCCCCGTCGACGTCCTCGCGATCGCCAACCAGGGCGATGCCGCCGCCG
ATCCCGTCGCGCGGACGTACTACGCGATCACCGACAAGCGTGACGGCGGCGCCCCGCCAGT
TTACAAGCCTTTGGACGCCGGGAAGCCCGGCCAGCACGACGCGAAGCTGATGAAGGCCTTT
TCCTCGGAGACCAAATGCACCGCGTCCGCCATTGGCGCGCCCAGCAGCAAGCTCGTGCTCCG
CACCGTCGGGGCGGACCAAGTCGCGTGCACGAGCTTCGACCTCTCCGGAGTAAACCTGGGT
GTTCGTCTTGTGGTCAGCGACTGGAGCGGGGCAGCCGAGGTCCGGCGGATGGGGGTGGCCA
TGGTGAGCGTCGTGTGCGTGGCCGTGGCGGTCGCGACGCTGGTGTCCATCCTTATGGCACGG
GCGCTGTGGCGGGCCGGGGCACGGGAGGCGGCTCTAGAGGCTGACCTCGTGAGGCAGAAGG
AGGCGCTCCAGCAAGCGGAGCGCAAGAGCATGAACAAGAGCAATGCCTTCGCCCGCGCCAG
CCACGACATCCGCTCCTCACTCGCTGTCGTCGTTGGACTCATCGACGTTTCCCGGATAGAGG
CCGAGAGCAACCCCAACCTCAGCTATAACCTCGACCAGATGAACATTGGCACCAACAAGCT
CTTCGATATACTTAACACGATACTGGACATGGGCAAGGTGGAGTCCGGGAAGATGCAGCTA
GAGGAGGTGGAGTTCAGGATGGCAGACGTCCTTGAGGAATCCATGGACCTGGCGAACGTCG
TCGGCATGTCAAGAGGCGTCGAAGTGATCTGGGACCCTTGTGACTTCTCCGTGTTGCGGTGC
ACCACCACCTTGGGCGACTGCAAGCGTATCAAACAGATCCTTGACAACCTACTTGGCAACGC
CATCAAGTTCACACACGAAGGCCACGTCATGCTTCGGGCATGGGCCAACCGCCCCATCATGA
GGAGCTCCGTGGTCAGCACCCCATCGAGGTTCACCCCCCGTCGCCCCGCGGGTGGGATCTTT
CGGCGGCTGCTTGGAAGGAGGGAGAACCGTTCTGAACAGAATAGCCGAATGTCCTTACAAA
ATGATCCGAATTCGGTTGAGTTTTACTTTGAGGTGGTTGACACTGGTGTGGGGATACCCCAG
GAAAAGAGGGAGTCCGTGTTTGAGAACTACGTTCAAGTGAAGGAAGGGCATGGTGGCACCG
GGCTCGGACTTGGAATTGTGCAATCCTTTGTTCGTTTGATGGGAGGAGAAATCAGCATCAAG
GACAAGGAGCCAGGAGAAGCGGGGACGTGCTTTGGCTTCAACATCTTCCTCAAGGTCAGCG
AGGCGTCAGAGGTGGAAGAGGACCTCGAGCAAGGGAGGACGCCGCCGTCGCTGTTCAGGGA
GCCCGCCTGCTTCAAGGGCGGGCACTGCGTCCTCCTCGCCCACGGCGACGAGACACGCCGG
ATCCTGTATACATGGATGGAGAGCCTCGGGATGAAGGTCTGGCCCGTCACGCGCGCCGAGTT
CCTCATCCCGACCCTCGAGAAGGCGCGCTCCGCCGCCGGCGCCTCGCCGTTGAGGTCGGCGT
CGACGTCGTCGCTGCATGGCGTTGGGAGCGCCGACTCCAACATTACGACGGACCGGTGCTTC
AGCTCCAAGGAGATGGTCAGCCACCTGCGGAACAGCAGCGGCATGGCCGGCAGCCACGGCG
GGCACCTCCACCCCTTCGGCCTGCTCGTCATCGTCGACGTCTCCGGCGGGAGGCTCGACGAG
GTTGCCCCCGAGGCGGCGAGCTTGGCAAGGATCAAGCAGCAGGCGCCGTGCAGGATCGTCT
GCCTGACGGACATCAAGACCCCCTCCGAGGATATGAGGAGGTTCAGTGAGGCGGCAAGCAT
CGACCTCAACCTGCGCAAGCCCATCCATGGCTCCCGGCTGCACCAACTCCTCCAGGTCATGA
GAGACCTCCAGGCCAACCCGTTTACACAGCAGCAACCACATCAGTCCGGCACGGCCATGAA
AGAACTGCCGGCTGCTGATGAGACCTCTGCGGCGGAGGCGTCTTCTGAAATCACGCCCGCGG
CGGAGGCGTCTTCTGAAATCACTCCCGCGGCGGAGGCGTCTTCTGAAATCACTCCCGCGGCG
GAGGCGTCTTCTGAAATCACTCCCGCGGCGGAGGCGTCTGAAATCATGCCGGCAGCACCGG
CGCCAACTCCCCAGGGACCGGCCAATGCTGGAGAAGGCAAGCCGCTGGAGGGGATGCGCAT
GCTGCTGGTCGACGACACCACGCTGCTGCAGGTAGTCCAGAAGCAGATACTGACCAATTAC
GGGGCAACCGTGGAGGTCGCCACGGACGGCTCCATGGCCGTGGCCATGTTTACAAAGGCTC
TTGAGAGCGCAAATGGCGTCTCAGAGAGCCATGTGGACACAGTGGCCATGCCCTACGATGT
CATCTTCATGGATTGCCAGATGCCAGTGATGAATGGCTACGATGCTACGAGGCGCATCCGCG
AGGAAGAAAGCCGCTACGGCATCCGCACCCCGATCATCGCGCTGACCGCGCATTCCGCGGA
GGAGGGGCTGCAGGAGTCCATGGAAGCAGGGATGGATCTTCACCTGACGAAGCCCATACCC
AAGCCGGCAATCGCACAGATTGTTCTAGACCTCTGCAACCAAGTTAATAACTGA
SEQ ID NO:18 OV1-B polypeptide sequence
MVGEVGKWGSSFKHSWALIPLVAHGIIVIVVALAYSFISSHINDDATSAMDASLAHVAAGVQPL
VEANRSAAVVAHSLFIPSNESSYFRYVGPYMVMALAMQPQVAEISYASVDGAALTYYRGENGQ
PRAKFVSESSEWYTQDVDPVNGRPTGRPDPAAQPEHLPNATQVLADAKSGSPAALGAGWVSSN
VQMVVFSAPVSDTAGVVSAAVPVDVLAIANQGDAAADPVARTYYAITDKRDGGAPPVYKPLD
AGKPGQHDAKLMKAFSSETKCTASAIGAPSSKLVLRTVGADQVACTSFDLSGVNLGVRLVVSD
WSGAAEVRRMGVAMVSVVCVAVAVATLVSILMARALWRAGAREAALEADLVRQKEALQQAE
RKSMNKSNAFARASHDIRSSLAVVVGLIDVSRIEAESNPNLSYNLDQMNIGTNKLFDILNTILDM
GKVESGKMQLEEVEFRMADVLEESMDLANVVGMSRGVEVIWDPCDFSVLRCTTTLGDCKRIKQ
ILDNLLGNAIKFTHEGHVMLRAWANRPIMRSSVVSTPSRFTPRRPAGGIFRRLLGRRENRSEQNSR
MSLQNDPNSVEFYFEVVDTGVGIPQEKRESVFENYVQVKEGHGGTGLGLGIVQSFVRLMGGEISI
KDKEPGEAGTCFGFNIFLKVSEASEVEEDLEQGRTPPSLFREPACFKGGHCVLLAHGDETRRILYT
WMESLGMKVWPVTRAEFLIPTLEKARSAAGASPLRSASTSSLHGVGSADSNITTDRCFSSKEMVS
HLRNSSGMAGSHGGHLHPFGLLVIVDVSGGRLDEVAPEAASLARIKQQAPCRIVCLTDIKTPSED
MRRFSEAASIDLNLRKPIHGSRLHQLLQVMRDLQANPFTQQQPHQSGTAMKELPAADETSAAEA
SSEITPAAEASSEITPAAEASSEITPAAEASSEITPAAEASEIMPAAPAPTPQGPANAGEGKPLEGMR
MLLVDDTTLLQVVQKQILTNYGATVEVATDGSMAVAMFTKALESANGVSESHVDTVAMPYDV
IFMDCQMPVMNGYDATRRIREEESRYGIRTPIIALTAHSAEEGLQESMEAGMDLHLTKPIPKPAIA
QIVLDLCNQVNN
SEQ ID NO: 19 OV1-B genomic sequence. Start codon at bases 3,055-3,057.
Stop codon at bases 9,664-9,666.
GTTATATACTCACTGGGGTGCTCATGTGCTTAAGTCGTGTCCCCACAACTGTTCATATTTACT
GCAAAACTAAGATAGCCGGATCAACAAACGAATAAATTGACGGGTGGTCCTTCCATGCTAT
CCACCATATGGCGTAATTGCTTTTAAGTTGTAGACTTCGTAGGCTTCCTTTTCACGTTATTTTT
TCATTTAGTCGGATGGTGTGTGTTGTGATCTGCTGGAAAAAAGACCCCCATTAGTGGAAATA
AACATAGAAATGGCAGGCTCTCATGTCGTACAAAAATAAAAAAACAATTTTGAATAAAAAT
CAATATAATTCACAATAACACACAAGACCATCCCATTACCATAGTAGGAAATGCCACATCCA
TTTCCCTTTTTTGGAAATTGACCACCAATGGTGGCTCATCTTGTAAACTTTCCCTTCAATTCTC
AATTGTTCTCATCTACGTACTTCTAACTTAATTATTTTAGGGACATGCATACAAGGTGACTAG
CAGTAATGAGCACAACGTCATGGGAGCCTGTGAATCACTTTCAACCATAAAGGGGGAACCA
TATTTCTAACTAAACTTCAAAAATATTCGTAAAAAACCAATGTGATGCCACTTTCAACCAAA
ATTTAGTAACCAAAAATCTACAAAAAAATTTGACTCGGTCATTTAATTTTGTATAAAGTTCA
GATGGCTTAAAAAACATTCATGCATTTCTAACTGAAATTTGTCACAAAAATTCTTACGGCTTT
CAACTGAAGAAAAGGAAACCGAAAGATGTGGCCAATCCTACCTTAGAGATGGCTTTAGCAA
GGCATGCTCATGGTAATGGGACAAGTATAGCCTCCCAAGGTTGGTAAAGAGTGCATGTTGA
AATTACCATCATCATAAGAACCATGGACGCGACCCCCCACCCCCCACCCCCCACCTACATGA
TCCCATATTACTAGAGAGGAGGGGACATCATCAAGGAGACATGGAAGATGTTGGTGTGTCG
ATGACCTTGTTGTGGGTGGCACAAATACGTGACATGATAGACACGAGAGAATTCGTAAGAT
GGTGAAAATCACTAGAGATTTGGTGGGACAAGGTATTAGATTTTCTTAGGTGTGGTTTGAAA
ATTTCAAACCAAGCTTGTCTATTATGTCTTAATTAAACATTGTGTCATGCAATCGTTTAAGCG
AAGTTCCATTTTTCTCCAAAAATACAACTGGCCAAAACAACGACTTCATTTACCATCGCTAC
CCTCTACCCATATCCAATCCTTGATCCCATCCATCCTCGGATCACAGCTCCTACACTGTGATG
GAGGGGAAGGGATCCTCATCTCTCATCTGATGTGTAGGGAATGTTATTTGGTTAGTTTTTGGT
GTCATGGTGACATCACAATGATGGTGACAACTCCAATACTGTCTCGGTGGTGTTTGGTCAGG
TGTTGCCAAGGACATTGTGATTATTTTTTGTTCTCGTTGACCTTTTTGGACGACATTGTGGTTC
TTTCTGCTATATTTTCGTGAGGCATCATTGACCGCGGTCATCTTCTTCGACAACCCTTTCCGTC
GAGCCTTTGCGAGCAATGTCAGTGGCCTAGTTTGGCAATATGAGTGTCGTTGCCTTTCTAGAT
CCCCCATGCTAATGTTCTTGGCATGATTGCTAATATTTGTTATACACGACCGACCGCCTACCA
TTGCGAACATTCAAGATGTGTTTCTGTAGAGGTCTTCACTGACTCAGATTCTCGATGTTGTTC
TCCGCTTGCGAGATCTAGGTGGGGCAACGACAACGAGTTTGGTTACGGTTTGGCTTGAATTT
TACGGCCTACAGTGTTGGATAGAATTCCATTTGTTTATGGTTAATTCTATGCTTGTGGGTTTA
GTTACCTCGTTATTTGAATGTATTCGCTATTTTCTTTGTGATATAAGATAGAACTAATTTTAA
AAAGAATTATGCTAGTCAATAGTGATGAGGCAATTAAGCTACATATGTACATCAAAAACAG
TACGACAGATCCACTAACTATAATATGCACAGAAGAATTGCGTATGAGTCTTGTATCTTCTTT
CATCATTTATTTTCCATGGAATATAGGTGACATGTTGTACGGTCTCACATGAACAGTCCTACA
TATTGCCCACTCAGCGCACAAGAGGAACGCTCGAACTTGTCGTGTGTGTGCGAACAAAATAA
ATATAACTTTTCAATTTCCGTGCTTCAAGATTTTCACATGATGATCGAGTTTGAATAAGTATG
ACACACTACAAAAAAATCAGCTGATGATCGAGTTTGCATAAGTAAGAAACAGGCTACAAAA
AGAAAGTAAGACACAATTCATCGTTTTTTTTTCAGGAAAGGACCTGGTTCATGTTCATTTTAT
TTTTTTGAGGGAAAGGACCTGGTTCATCAGTCTTAGCTGTCCTGGCAACGGAGCTAGTTCAA
CATAAGTCGAGCCTGTGCTTCTATATTTAGCATCAAACGTAACCAAGAGTTAACTTATAACA
AAACTAATGATCATGCTATCTAAGACACGAAGCTAATGGATCGGTGTACACTTGCACAACAG
GATGGCCTTATGACGATCGTGCGGCTGGCAACACTGCCCCCATGCAAGGCCAACACTGCCTC
AGCCCGCCCAAACTATTCGAACGGGAGGAAAACATAATTCATTGTGCTTGGATTTGGCAATT
GATTACAAGATCACGTAGTATCCCGTTTTCTATCTCGTCGTGTGTCTACTACCAGGCCGCGCA
TTGATATTAGATATCGAAGTTAAATGCCGATTTTTTTTAAATAAATACCTTTTTTTATTGCGA
TGAAGGGGTAAATACCATTTTTTTCTGCGGTGGGCGGAAAATACAGCCAAAAGTGGTATAAT
AAACCGCGACGGTGGATTTGAAAAACCGGCGGCGCTCTGAGCCCACAATTCAGAGCGGAAA
AACCCCCCCAATGTGTAAAGTGTATTCCTCCGAGCATCTATAAAAGGCCATCTACCTAGGAA
ACAAATATTCACCAACACCTAACAAAGATTTGCTTTTGTAGAATACTTTACAATACTTCCGC
GATGGTTGGAGAGGTTGGCAAGTGGGGTAGTTCCTTCAAACATTCGTGGGCTTTAATCCCAC
TGGTATGAAAACATTCATAGTAGTTGGCTTCTTTTTATGAAACTCATGGTTAATACTTGTTCT
TCTATATGAACTTCATGGCTATTCTCTTCCTTCAAAAAAAACTCCATGGCTTTTCTCATTTCTT
GGTTCTTGTCCTTCCACCTACAACTATTGCTTACCTGAGCGGAAACTAGATCTGCGAGGTTAT
CATCTGCTTAAGCTTTTTTTGAGGGATTCATCTTGCCTAAGCTTAGTGCAACAAAGACATCCT
TCCGCATATGTGGAGATGTGATCTCGAAAATAGATCTATGTAGTGGTACGGTATTCTATTGG
AAAAAGAGGATTACCCCCGTCTCTTGGCATAGGCGTGAGGTTCAAAAGTAATTCTAATGATT
TGTGTCTTACAATTAAATTTTGTTTCGTACGATATAGATATGTTCCTATGCCTGTAAGGCGCA
GATTTGAAAAGTAGATCTAACTGATTTTTATCGTTTCTGTTTCAGTTATGTATATTACAGATA
TCATCCTGCACGTGTCAGGCATGCGAGATCTCTTTCTTTAGATAACAATGAAAACTATATCTT
CATAATTTTTCATCTTGTAATTGAAGATTCTGCTCCGTACAAGCCGGATATCCTCCCACGTGT
GTTTTAGGCATGGATTCAAAAAGTAAATATAATTTTATTTTATCTTGTAATTAGGAATCCGCT
CCGTACAATATTAGTATCCTTTCACTTGTGTAAGACGTGCTCTAAGAATACATCTATGCAGGT
TTTAATATATAATTTGGCTTCTGCTCCATACATCACATATATCTCCCCACATCTGTGAGGCAG
TGGCGAGCCGAAGATCTACCTTTTGTCGTATGCTCCTATATTGTTTTTCTGGACGCCGTGTGT
TAGATCTACCCATGGAATGTCACGACAAATGTTGTCGTTAGATGAGCCCAAAGGCGATCTGT
TAGCAAGATGTCACGACAAATTATATCTTTAGATTAGTGAAAACCCTCGAAATCTTGACTTA
TTACTTGTGGAACAAATGTTACCGTTAGACGAGTTGAAACACGTTGCAACGCCGCTTGTCGA
GGCCTCGTCAGCCTAAAAGACAGTTTAATTTATTAAAAATACACTTTAATTTAATAATTATGC
ATTTTCTATTTTATTTTTTACATGTTGCAAATATATTTTTCTGACGTCACACTTTTATTGCACTT
GCACCCATGCAGGTTGCCCATGGAATCATCGTGATCGTAGTGGCTCTCGCTTACTCTTTCATC
TCGTCGCACATAAATGATGATGCCACGAGCGCCATGGACGCGTCGCTGGCGCACGTCGCCGC
CGGTGTGCAGCCGCTCGTGGAAGCCAACCGCTCCGCCGCCGTCGTCGCACACTCTCTGTTCA
TCCCCAGCAACGAATCATCTTATTTCCGATACGTAATTAACCGAGCACCTTTGCGTTAAATTG
AAATATGTTTCTCTTTTCTCTGTATAACGTGATTAATTTCATCAAGTATATGCATTTCCTTTTT
GAACCACAATTATTTCCTTACTTTAAATAACAAATCTTTTTGGGGGGAATTAAATAACAAAT
CTTAATTACTAGCCGGGCCGACCCAGTAACTAGTTATTGCGTATGATTCTCTTCTGATTTTCG
TAATAATACGAGCATTGATATGAATATTCGCATGCATATACAACAAATAATTTTTGCGTACC
CTTTTTTATGCAGTACACGTCCAAAATAACATATCAACACGTACGTGCAAATATACATCTAA
CATTTACATGTATTCTTGACCTGACAGGTGGGACCGTATATGGTCATGGCGTTGGCCATGCA
GCCGCAGGTGGCCGAGATATCATACGCCAGCGTGGACGGCGCCGCGTTGACGTACTACCG
CGGCGAGAACGGCCAGCCGAGAGCCAAGTTCGTGAGCGAGAGCAGCGAATGGTACACCCA
GGACGTTGATCCTGTGAACGGCCGTCCCACCGGCCGCCCCGACCCGGCGGCTCAGCCGGAG
CACCTACCCAACGCGACGCAGGTCCTCGCCGACGCCAAGAGCGGCTCGCCCGCCGCCCTC
GGGGCCGGGTGGGTCAGCTCCAACGTCCAGATGGTGGTCTTCTCCGCGCCTGTCAGTGAC
ACTGCCGGCGTGGTCTCCGCCGCGGTCCCCGTCGACGTCCTCGCGATCGCCAACCAGGGCGA
TGCCGCCGCCGATCCCGTCGCGCGGACGTACTACGCGATCACCGACAAGCGTGACGGCGG
CGCCCCGCCAGTTTACAAGCCTTTGGACGCCGGGAAGCCCGGCCAGCACGACGCGAAGCTG
ATGAAGGCCTTTTCCTCGGAGACCAAATGCACCGCGTCCGCCATTGGCGCGCCCAGCAGCAA
GCTCGTGCTCCGCACCGTCGGGGCGGACCAAGTCGCGTGCACGAGCTTCGACCTCTCCGGAG
TAAACCTGGTAACGTGTCCATCTAGCATCAATCACAGGCCATCCATATATGCATACGTACAC
GAACGTGCACACACCCTATATATCTAACATAATTGCTCTGCATTTTGGTCAAGACTTAATCCC
AGCATGTAATATTTCTTCCAAGTATGCTGTTTATACATTCAAGCAACTGACAATGAAAAAAG
GTTAGATGAGCTAAGGGGACTTCGGCAAAAAAAACTAATAAAACGTTTTTTGTTTTGGCGAC
CTCAATAAACATCCTTCCGACTTGGAGTGAGGAAAGAAAATCAGGGAACGCTCCGCTATGG
AAAGTGGACGTACATGATCTTTCTTCCTTCCTTCCTTTGGCATGTAAAAAACTAAAACCTTTC
CAAATCAAGGTGTCCCAAAATTAGGAAGAAAATCTTAATGGAGAGTACAAAGTCTTCTTCTT
CCTCTTCTTCCCCAAAGCAAGATTTCTCCTTCTGTTCTTCCCCAAACGGAGCTCTGGCACAAA
ACTGCGGTGAGCTCGATCGTCTCCTACGTACTTTTCTTGCATCTGCTAGTGCCTTGCATGCAT
ATCACCGATTGTCGTTCATGTATATCTCTCCTCAGTTCTTTTGCAATTTATTTACAGCGTAGA
GAGCAGTTTAGACTACGTAGTAAATCACCATTGAAACTGATCTTGCCATCCGTCGCCTATGC
AACGAGCGCAGGGGCTGCGCTAGCTTGATAATTCTCCGACCAGGCCGGGCGGCGCCTCGCG
CGCCATGGAAACAGTCTTTTTTTTTTTTGATAAAGGCCATGGAAACAACCTGGCGTACAGCC
TCTTGATAAAAAAAAATATTTTCTTCGTAGTATAAGCACGCATTTGCATGTTTGAGAATTTTT
ATTGGGACGGCTAACAAGTATCTCCGGTTGTATTTCTTCTTGTTTTTCAGGGTGTTCGTCTTGT
GGTCAGCGACTGGAGCGGGGCAGCCGAGGTCCGGCGGATGGGGGTGGCCATGGTGAGCGTC
GTGTGCGTGGCCGTGGCGGTCGCGACGCTGGTGTCCATCCTTATGGCACGGGCGCTGTGGCG
GGCCGGGGCACGGGAGGCGGCTCTAGAGGCTGACCTCGTGAGGCAGAAGGAGGCGCTCCAG
CAAGCGGAGCGCAAGAGCATGAACAAGAGCAATGCCTTCGCCCGCGCCAGCCACGACATCC
GCTCCTCACTCGCTGTCGTCGTTGGACTCATCGACGTTTCCCGGATAGAGGCCGAGAGCAAC
CCCAACCTCAGCTATAACCTCGACCAGATGAACATTGGCACCAACAAGCTCTTCGGTCAGTC
TACCATGCATGGCAATACCATGAATAGCTTGTGCTACCTTTTAGTAGATCTATCCGTACTTGG
TAATTTAGCAATGTGATCATAGCATTATAAATTTGCATGTCATAGAAGTAAAATTTTCGTAA
ATAATTTAATTACTGTTTTAGGTGTAGAAGTGTGCAATAGCCCACCTGTTATACATTTAAGGT
ATCAAATTTGCTCATAAAATTTAAAATATGCAAGAAGTCAATCTCTGTTTGGTAAAGAAATA
GCATTTTATTTGTAAAAATTAAAGTTTAGGATAGTTCAAATAGAATTGTCAGATATCACTAC
GTTAGCAACCAGTAAACTTATAAAGTTTCAATATTTCTATCGATTTTGCTGTTGGGCAAATTT
TGTCACATTTACGCATCATATTGTTTGTGCGTGTCTTCGAGTGCATAAAGCAAAAAAGTTTAT
TATTTCCCAAAAATGAACTTTATACCATCTTTGTGTTCTCAGTTGAGCATATGTTGGCTCATC
CTCCATGTTTATACATGTGTATGTGTACATGCAGATATACTTAACACGATACTGGACATGGG
CAAGGTGGAGTCCGGGAAGATGCAGCTAGAGGAGGTGGAGTTCAGGATGGCAGACGTCCTT
GAGGAATCCATGGACCTGGCGAACGTCGTCGGCATGTCAAGAGGCGTCGAAGTGATCTGGG
ACCCTTGTGACTTCTCCGTGTTGCGGTGCACCACCACCTTGGGCGACTGCAAGCGTATCAAA
CAGATCCTTGACAACCTACTTGGCAACGCCATCAAGTTCACACACGAAGGCCACGTCATGCT
TCGGGCATGGGCCAACCGCCCCATCATGAGGAGCTCCGTGGTCAGCACCCCATCGAGGTTCA
CCCCCCGTCGCCCCGCGGGTGGGATCTTTCGGCGGCTGCTTGGAAGGAGGGAGAACCGTTCT
GAACAGAATAGCCGAATGTCCTTACAAAATGATCCGAATTCGGTTGAGTTTTACTTTGAGGT
GGTTGACACTGGTGTGGGGATACCCCAGGAAAAGAGGGAGTCCGTGTTTGAGAACTACGTT
CAAGTGAAGGAAGGGCATGGTGGCACCGGGCTCGGACTTGGAATTGTGCAATCCTTTGTAA
GTGATCTCGTCTTTTTTCGTGCATGATAAAATCTTGTCAACTGCATCAAAGAAAAGTACTATC
TCCATTCCAGACGTTTGAATGGAGGCAGTATATTTTTCACTAATGTTTTCGTTTTTTCTTGTTT
ACTTAGTTTTGTTTGCTGTTGTTGTTGATGTAAATAAAGGTTCGTTTGATGGGAGGAGAAATC
AGCATCAAGGACAAGGAGCCAGGAGAAGCGGGGACGTGCTTTGGCTTCAACATCTTCCTCA
AGGTCAGCGAGGCGTCAGAGGTGGAAGAGGACCTCGAGCAAGGGAGGACGCCGCCGTCGC
TGTTCAGGGAGCCCGCCTGCTTCAAGGGCGGGCACTGCGTCCTCCTCGCCCACGGCGACGAG
ACACGCCGGATCCTGTATACATGGATGGAGAGCCTCGGGATGAAGGTCTGGCCCGTCACGC
GCGCCGAGTTCCTCATCCCGACCCTCGAGAAGGCGCGCTCCGCCGCCGGCGCCTCGCCGTTG
AGGTCGGCGTCGACGTCGTCGCTGCATGGCGTTGGGAGCGCCGACTCCAACATTACGACGG
ACCGGTGCTTCAGCTCCAAGGAGATGGTCAGCCACCTGCGGAACAGCAGCGGCATGGCCGG
CAGCCACGGCGGGCACCTCCACCCCTTCGGCCTGCTCGTCATCGTCGACGTCTCCGGCGGGA
GGCTCGACGAGGTTGCCCCCGAGGCGGCGAGCTTGGCAAGGATCAAGCAGCAGGCGCCGTG
CAGGATCGTCTGCCTGACGGACATCAAGACCCCCTCCGAGGATATGAGGAGGTTCAGTGAG
GCGGCAAGCATCGACCTCAACCTGCGCAAGCCCATCCATGGCTCCCGGCTGCACCAACTCCT
CCAGGTCATGAGAGACCTCCAGGCCAACCCGTTTACACAGCAGCAACCACATCAGTCCGGC
ACGGCCATGAAAGAACTGCCGGCTGCTGATGAGACCTCTGCGGCGGAGGCGTCTTCTGAAA
TCACGCCCGCGGCGGAGGCGTCTTCTGAAATCACTCCCGCGGCGGAGGCGTCTTCTGAAATC
ACTCCCGCGGCGGAGGCGTCTTCTGAAATCACTCCCGCGGCGGAGGCGTCTGAAATCATGCC
GGCAGCACCGGCGCCAACTCCCCAGGGACCGGCCAATGCTGGAGAAGGCAAGCCGCTGGAG
GGGATGCGCATGCTGCTGGTCGACGACACCACGCTGCTGCAGGTAGTCCAGAAGCAGATAC
TGACCAATTACGGGGCAACCGTGGAGGTCGCCACGGACGGCTCCATGGCCGTGGCCATGTTT
ACAAAGGCTCTTGAGAGCGCAAATGGCGTCTCAGAGAGCCATGTGGACACAGTGGCCATGC
CCTACGATGTCATCTTCATGGATTGCCAGGTACATTTTTCTCCAGCAAACAACGTGCCAAGC
ACATCGTCTTCTTCCTGAAGATGATTTAATCTGACGTTGCTGACAATTCGATCTTCTTGTTTC
AGATGCCAGTGATGAATGGCTACGATGCTACGAGGCGCATCCGCGAGGAAGAAAGCCGCTA
CGGCATCCGCACCCCGATCATCGCGCTGACCGCGCATTCCGCGGAGGAGGGGCTGCAGGAG
TCCATGGAAGCAGGGATGGATCTTCACCTGACGAAGCCCATACCCAAGCCGGCAATCGCAC
AGATTGTTCTAGACCTCTGCAACCAAGTTAATAACTGATCGCCGAGATCCATCGTTCCCCAC
TCCCCCGCCGCATGATCAAAAATCGAGATAGGTGTAGGTGGTTTTTCAGCGAGCGAATGCGG
TTATCATCCTAGTCACTGAAAAACCACCTACATCTCTGAGTTTCGATCATGCGACCCGGGGC
ATCATCGTAGTTTGTAGCATTTAGCAGCAGCAGCTGAACTTTGTTGTTGTATCAAAATCAGGT
CAGGTTTATTTCCAGAATTGCTCTTGGACAATGTATTGTCAATTTTGAATTTCCAGAAACAAT
TATGGTTAAGTTTTGAGTTCCAGAGTTTGTGTGTCAGAGTTCTTTTTTCCCAGAGTTTGTGTTT
TCAGAGCTTGTGATTAGACGCCATATATTTGCTTCCATTGCCCATCCCAGGAGAAAGAAAGT
ACAGGTTGCTACAAGCCATGAAATCCCTAGCTAGCTACCCCAGATATCTAGTAGTGTACACA
AGCATAGCTGATAAATACCCATAGGAATAACTGTACCATCTTCTCTAGGTATGTAGTGGACT
AGCCTAAATATTACTAGGACTAGCTCTCATATGCAGGCACCAAGTGGGAAAAGTTCACACCC
GAGGTCATTTTCCGTGAACGGCACTTCGTCGCTCTCCTGCGTTGAAATCGGAAAAGGGGTTC
AGAAAAATCACCATCAAAATTCTAAGAACATAGGTTTAATAAATAACGATTTTAAAGGAAA
TATGACTGTCCTAAATAGTGAAATTTTAAGCATGACTGATGTCCTTACCACTTTTATCATGGA
ATTGAGAAGACCATCAAAGTTGCAGCTGCTAGAATTGAATCCACCCTGCAGCTTTGGAACTC
CCAAATCGTACATATCGGTCTGCTCAGGCACAATGGCACCACCATCATAGCTAAAACCTCCA
CTGTTCAGTCTTTGGTCTTGTCTCATTCCACCATCCATTCTATGTCTAGAGTTGCTTGCAACCA
CAAACTGCAAGTATTTTGAATGTTTCTCGGTATCATGAGGAGGGAGCAGCATTGTACTACCA
CAAGAACTAGCTCCACATTTGGCATTGCTGGCTACTATAAGGCTCTCAGAAGGACAAACAGT
AACAGCCATTCTTTCCGAAAATATCCCCTTTTGGTCCATCTCTTGTTCTCCAGCCACAAAGCT
AGCATCAAGTTTTGTCGTGCCGGTACCATCGAACGGGATGTGCAACGGTAACTTGTCGACAG
AAAACCCATCACTGATTGGAAGAGCACACTGCTGAGATATACAAGAATCCCGCAAATCGGC
GGAAACTCCGACAGACCTTTCAAGCAGCCCTGAACTCCCAGTTGGTATTCCTATTGATGGAT
GGGCTCCAACTTTGATGTGTGCAGTGCACTCCAAAAGATCTTCTGGTGGCAGTGAACTGCTT
GTAACTCTTTGAAGTGTGCCAGACATAGTGTTAGCCAGAGCACCCCCGGAAAAGACAGAGC
ACAAGTCACTGGTTTCTTGATGAATCCACTTCTGATGGAGCTGGGGCTGCCCAAGCGACGAG
GTCAAGCCTTGCGATAAATCTGCCTGTTGGTTGTCTTGTAAGCTCACAATGTGGAACTTCTCC
GTATCGCCGGCGCAATGACTTACTGCGCCGTTGCTGCTAGAACACTGGTTTGCCTTGGGAGA
AGCAAGGTCCTGAAGCCCAAATGCTGCTGCACCAGCACCACTCAGCAGTCCATGTGGATTGA
GAGATGGAAGAGCAGCAGGAGGGGCAAAGGGTTGATGATAACTATGAAGTCCTTCAAATGC
TCCCATGTGCAAGAAGGGGTCTCTGCCTCCAAAAGCAGCAGCAATGCTGGCTTGCTGTGATG
CCACGGCACTTAGCCGTCTGAGGTATAGCCTGTACTTCTGCATTGGCATAGAAAGTAACCTG
ATTAGACATGGAGGAAATTATGTAATCATCAGAATTCTGAAACATGAGATATACAGTCTTAA
GATAACTGCTTCCTAGTTTGGTATGGAGATTCTTGCTGAACTGGTACATAAAAGCACTCCAG
GATATAGAAAAATCTGATGAATTTTCTTAACCCTTTAATGTAGGCTTGTTAACAAAATTTCTA
TTTGTCAATTCATTGACATGCAAAGCTTTTGCATTTCTCTAAAAAAATATTTTAACCAAAAGG
TGTACTACTACCTCCGTCTCAAAATATAAGACAGAGGTAGTACAACGCAGTTTACATATGTA
GCTTTGTAATCTGTACAGGGTGGTGTCAGTGTCTTCCTTCTAGCAGTTTTATAGAAGTGAAAC
AATGTAGCCAAAATACTACATTAAACTCAGCAGTACATACCAAAACATCACATTAAACTTGT
ACAGCAGTACATACTGAAATACAACATTAACTTGTACAGCAGTGCATACCAAAATACCACA
GTTAACTTGTACAGCAGTATTACCTAATTACTCTGCATTAAACCTGTACAACATTACATACTA
ATTTGAGCATTTTATACCTGTGTAGTTAGATCTAATCTACTAGTCTACATGGTTGGGGGAATA
ATCAGATATTTGTATAAGATATCTCCTTCTTCAGGTTTTCTGAAAGATTCAGTATAACTGAAC
TGAATGTTTCTTTGTTTTCAGACCAGCTGAACATATTCAACTTGTCAAGAAAAATGAAGAAA
TGTGAAGGTGAAACTATGTAACCGCAAAATTAAACTACTTAACTCATTTTGCACGAACACCA
GGAACATGTTAACATATTTGTTAAAAGAGTTCTGCTGTGCAACACCATAATATCGAATTAAG
CTCTGCTCTGCAACTAATAGGTGTGTCGGTACCAAATACAGTAAGGGGATAAGGACTCTGTA
TACGAATATCTTCTTTTTTAAAATTGTTCAAAAGTTGGCCCTCTTAATTCGACACAGGAAAAT
AAGGCCACTTTGCACATAATTTTACCAAAAGTTTCTGTAAAAGTTTTTCTTCCTGTGCACCAC
ATGCTGATCAACGAAAGAGATAACACAGGTAAAGGCGATGATGGAAGTAAATGCCACGTCG
CCATACCTGCAGATGGCTCGCAACATTTTCCCTGGTGAGCTTCTCCACGTTCATAAGCTCGAG
TATTCGTTTGGGTACAGCCTCTGCGAACAGCAGCAAGAGAAAGGACACAAGCGTAAATGGG
TTTGCTGCAGCCTGCAAGAACATTTAACATAAAGCAAAAGGCAACGTGGGGAGGTGTTTTTT
CTTACTGTCGATCCCGAGCTGGTTGACGGCGGCGACGAACTTCCGGTGCAGCTCCACTGACC
ACACCACCCTCGGCTTCTTCGACCCCGCGGCGTCGTCGTTGTCCTCGCCTTCGTCTTCCTCCT
CGCTGTGCGGCTCCTTCCGCTTCTTGCCGGCCCTGTTGCTCTGGTCGGACGACATGCCGCACG
TCGCCTGGCCGAGCCGGTGGTACGAGTCCGCGCTCGGCGGCTTGCTGATCTCCTTGCAGAGG
TCATGGTTGTTGTTCGGCTCACGGTTGCTGAACTTCCTCCTAACGACGTGCTGCCACACGTTC
CTGAGCTCTTCGATCCGAACGGGCTTTAGGAGGTAGTCGCAGGCGCCATGGGTTATCCCCTT
GAGCACAGATTTTGTCTCTCCGTTCACCGATAACACTGCACGAGTTGAATGGTGCCTCATTA
ACATC
SEQ ID NO: 20 OV1-D CDS
ATGGCTGGAGAGGTTGGCAAGTGGGGTAGTTCCTTCAAACATTCTTGGGCTTTAATCCCACT
GGTTGCCCATGGAATCATCGTGGTCGTAGTGGCTCTCGCTTACTCTTTCATCTCGTCGCACGT
AAATGATGATGCCGTGAGCGCCATGGACGCGTCGCTGGCGCACGTCGCCGCCGGCGTGCAG
CCTCTAATGGAAGCCAACCGCTCCGCCGCCGTCGTCGCGCACTCTCTGCAGATCCCCAGCAA
CGAATCATCTTATTTCCGATACGTGGGACCATACATGGTCATGGCGTTGGCCATGCAGCCGA
AGCTGGCCGAGATATCATACACCAGCGTGGACGGCGCCGCGTTGACGTACTACCGCGGCGA
GAACGGCCAGCCGAGAGCCAAGTTCGGGAGCCAGAGCGGCGAATGGCACACCCAGGCCGTT
GATCCGGTGAACGGCCGTCCCACCGGCCGCCCCGACCCAGCGGCGAGGCCGGAGCACCTAC
CCAACGCGACGCAGGTCCTCGCCGACGCCAAGAGCGGCTCGCCCGCGGCCCTCGGGGCCGG
GTGGGTCAGCTCCAACGTCCAGATGGTGGTCTTCTCCGCGCCTGTCGGTGACACTGCCGGCG
TGGTCTCCGCCGCGGTCCCCGTCGACGTCCTGGCGATCGCCAGCCAGGGCGACGCCGCCGCC
GATCCCGTCGCGCGGACGTACTACGCGATCACTGACAAGCACGACGGCGGGGCCCCGCCGG
TCTACAAGCCTTTGGACGCCGGGAAGCCCAACCAGCACGACGCGAAGCTGATGAGGGCCTT
TTCCTCGGAGACCAAATGCACCGCGTCCGCCATTGGCGCGCCCGGCAAGCTCGTGCTCCGCG
CCGTCGGGGCGGACCAAGTCGCGTGCACGAGCTTCGACCTCTCCGGAGTGAACCTGGGAGTT
CGTCTTGTGGTCAGCGACTGGGGCGGGGCAGCCGAGGTCCGGCGAATGGGGGTGGCCATGG
TGAGCGTCGTGTGCGTGGTCGTGGCGGTCGCGACGCTGGTGTGCATCCTTATGGCACGGGCG
TTGTGGCGGGCCGGGGCGCGGGAGGCCGCTCTAGAGGCTGACCTGGTGAGGCAGAAGGAGG
CGCTCCAGCAAGCGGAGCGCAAGAGCATGAACAAGAGCAATGCCTTCGCCCGCGCCAGTCA
TGACATCCGCTCCTCACTCGCTGCCGTCGTTGGACTCATCGATGTTTCCCGGGTAGAGGCCG
AGAGCAACTCCAACCTCACCTATAACCTCGACCAGATGAACATTGGCACAAACAAACTCTTG
GATATACTTAACACGATACTGGACATGGGCAAGGTGGAGTCCGGGAAGATGCAGCTAGAGG
AGGTGGAGTTCAGGATGGCAGACGTCCTTGAGGAATCCATGGACCTGGCAAACGTCGTCGG
CATGTCAAGAGGCGTCGAAGTGATCTGGGACCCTTGCGACTTCTCCGTGCTCCGGTGCACCA
CCACCATGGGCGATTGCAAGCGTATCAAACAAATTCTTGACAACCTACTCGGCAACGCCATC
AAGTTCACACACGACGGCCACGTCATGCTTCGAGCATGGGCCAACCGTCCCATCATGAGGA
GCTCCATAATCAGCACCCCGTCGAGGTTCACCCCCCGTCGCCGCACGGGTGGGATCTTTCGG
CGGCTGCTTGGAAGGAAGGAGAACCGTTCGGAACAAAATAGCCGAATGTCATTACAAAATG
ATCCTAATTCGGTTGAGTTTTACTTTGAGGTGGTTGACACTGGTGTGGGCATACCCCAGGAA
AAGAGGGAGTCTGTGTTTGAGAACTACGTTCAGGTGAAGGAAGGGCATGGTGGCACCGGGC
TCGGGCTTGGAATTGTGCAATCCTTTGTTCGTTTGATGGGAGGAGAAATCAGCATTAAGGAC
AAGGAGCCAGGAGAAGCGGGGACGTGCTTCGGCTTCAACATCTTCCTCAAGGTCAGCGAGG
CGTCAGAGGTGGAAGAGGACCTCGAGCAAGGGAGGACGCCGCCGTCGCTGTTCAGGGAGCC
CGCCTGCTTCAAGGGCGGGCACTGCGTCCTCCTCGCCCACGGCGACGAGACCCGCCGGATCC
TGTACACGTGGATGGAGAGCCTCGGGATGAAGGTCTGGCCCGTCACGCGCGCCGAGTTCCTC
GCCCCGACCCTCGAGAAGGCGCGCTCCGCCGCCGGCGCCTCGCCGTTGAGGTCAGCGTCGAC
GTCGTCGCTGCATGGCATCGGGAGCGGCGACTCCAACACTACGACGGACAGGTGCTTCAGCT
CCAAGGAGATGGTCAGCCACCTGCGGAACAGCAGCGGCATGGCCGGCAGCCACGGCGGGCA
CCTCCACCTCTTCGGCCTGCTCGTCATTGTCGACGTCTCTGGCGGGAGGCTCGACGAGGTCG
CCCCCGAGGCGGCGAGCTTGGCGAGGATCAAGCAGCAGGCGCCGTGCAGGATCGTCTGCCT
CACGGACCTCAAGACCCCCTCCGAGGATCTGAGGAGGTTCAGTGAGGCGGCGAGCATCGAC
CTCAACCTGCGCAAGCCCATCCACGGCTCCCGGCTGCACAAGCTCCTCCAGGTCATGAGAGA
CCTCCATGCCAACCCGTTTACGCAGCAGCAGCCGCAGCAGCTCGGTACAGCCATGAAAGAA
CTGCCGGCGGCTGATGAGACCTCTGCGGCGGAGGCGTCTTCTGAAATCACGCCCGCGGCAG
AGGCGTCTTCTGAAATCACGGCCGCTGCGGAGGCGTCTGAGATCATGCCGGCGGCGCCGGC
GCCGGCTCCCCAGGGACCGGCCAATGCAGGAGAAGGCAAGCCGCTGGAGGGGATGCGCATG
CTGCTGGTGGACGACACCACGCTGCTGCAGGTAGTCCAGAAGCAGATACTGGCCAATTACG
GAGCAACGGTGGAGGTCGCCACGGATGGCTCCATGGCCGTGGCCATGTTTACAAAGGCTCTT
GAGAGCGCAAATGGCGTCTCAGAGAGCCATGTGGACACAGTGGCCATGCCCTACGATGTCA
TCTTCATGGATTGCCAGATGCCAGTGATGAATGGCTATGATGCGACGAGGCGCATCCGGGAG
GAAGAAAGCCGCTACGGCATCCGCACCCCGATCATCGCGCTGACCGCGCATTCCGCAGAGG
AGGGGCTGCAGGAGTCCATGGAGGCAGGGATGGATCTTCACCTGACCAAGCCGATACCCAA
GCCGACAATCGCACAGATTGTTCTAGACCTCTGCAACCAAGTTAATAACTGA
SEQ ID NO: 21 OV1-D polypeptide sequence
MAGEVGKWGSSFKHSWALIPLVAHGIIVVVVALAYSFISSHVNDDAVSAMDASLAHVAAGVQP
LMEANRSAAVVAHSLQIPSNESSYFRYVGPYMVMALAMQPKLAEISYTSVDGAALTYYRGENG
QPRAKFGSQSGEWHTQAVDPVNGRPTGRPDPAARPEHLPNATQVLADAKSGSPAALGAGWVSS
NVQMVVFSAPVGDTAGVVSAAVPVDVLAIASQGDAAADPVARTYYAITDKHDGGAPPVYKPL
DAGKPNQHDAKLMRAFSSETKCTASAIGAPGKLVLRAVGADQVACTSFDLSGVNLGVRLVVSD
WGGAAEVRRMGVAMVSVVCVVVAVATLVCILMARALWRAGAREAALEADLVRQKEALQQA
ERKSMNKSNAFARASHDIRSSLAAVVGLIDVSRVEAESNSNLTYNLDQMNIGTNKLLDILNTILD
MGKVESGKMQLEEVEFRMADVLEESMDLANVVGMSRGVEVIWDPCDFSVLRCTTTMGDCKRI
KQILDNLLGNAIKFTHDGHVMLRAWANRPIMRSSIISTPSRFTPRRRTGGIFRRLLGRKENRSEQN
SRMSLQNDPNSVEFYFEVVDTGVGIPQEKRESVFENYVQVKEGHGGTGLGLGIVQSFVRLMGGE
ISIKDKEPGEAGTCFGFNIFLKVSEASEVEEDLEQGRTPPSLFREPACFKGGHCVLLAHGDETRRIL
YTWMESLGMKVWPVTRAEFLAPTLEKARSAAGASPLRSASTSSLHGIGSGDSNTTTDRCFSSKE
MVSHLRNSSGMAGSHGGHLHLFGLLVIVDVSGGRLDEVAPEAASLARIKQQAPCRIVCLTDLKT
PSEDLRRFSEAASIDLNLRKPIHGSRLHKLLQVMRDLHANPFTQQQPQQLGTAMKELPAADETSA
AEASSEITPAAEASSEITAAAEASEIMPAAPAPAPQGPANAGEGKPLEGMRMLLVDDTTLLQVVQ
KQILANYGATVEVATDGSMAVAMFTKALESANGVSESHVDTVAMPYDVIFMDCQMPVMNGY
DATRRIREEESRYGIRTPIIALTAHSAEEGLQESMEAGMDLHLTKPIPKPTIAQIVLDLCNQVNN
SEQ ID NO: 22 OV1-D genomic sequence. Start codon at 3,112-3,114. Stop
codon at 9,974-9,976
GTGTATGTTGTGATCTGGGAAAAGCTTCCCTGCCCTAATTGGGTCCACTACTTCGTTAACGTT
TACCGCTTCAATTAAACGAGTTCAATAACGATACGCTTTTGTACAAATGTACCAACCTTTATG
GTTTATTTATGTAACCAATCATGACATATTCACCCAAGTACATTCTGATATTTATGTTGAATG
TGCACATTGTCTATTAATCGTGGGGTAGTGTATATAGTCACTAGGGTGCTCATGTGCTTAAGT
CGCGTCCCCACAATTGTTTATATTTACTGCAAAACAAAGATAACCGGATCAACAAACCAATA
AATTGGCGAGTGGTCCTTTCATGCTATCCACCATATGGGGCAATTGTTTTTAAGTTATAGATT
TCGTAGGCTTCCTTTTCATGTTATTTTTCTTTTCGTTTAGTCAGATGGTGTGTGTTGTGATCTG
CTGGGAAAAAGCTCCCCCCTCCATTGGTGGCAATAAACATAAAAAGGGCCGGCTCTCACGTC
GTACAAAAAGAAAAGAAAACAGATTTGAATATAAATCAATATAATTTACAATAACACACGA
GACCATCCCATTACCATAGTAGGAAACGCCACACCCTTTCCCATTTTTGGAAATTGACCACC
ACTAGTGGCTCATCCTGTAAACCTTCCCTTCAACTTCTCGGTTGTTCTCATCTACTTCTAACTT
AATTATTTAAGGGACATGCATAGAAGGTGACTACCAGTGATGAGCACGGTGTCATGGGAGC
CTATGAACAACTTTCAACCATATATGACACACCTTTTATAAAGGGAAACCCATATTTCTAAC
TAAACTTCAACAATATTCATAAAAAAAACCATGTGATGCCACTTTCGACCAAAATTTAGTAT
CCAAAAATCTACAATTTTTTGAATCAATTGTTTAATTTTGTACAAAATTCAACTGGCTTAAAT
AAACATTCATGCATTTCTAACTAAAATTTCTCACGAAAATTCTTCCAACTTTCAACTCAAGGG
AAACCGAAAGATTTGGCCAATCCTACTATAGAGATGGCTTTATCAAGGCATGATCATGATAA
TGGGACAAGTATAGCCTCCCAAGGGTTGTTAAAAAACGTGCATGTTGAAATTACCATCATCA
TAAGATCCATGCCCCCCCCCACACACACACACATACGTACATGATCCAATCACAAGAGATTT
GGTGGGGCAGGGTATTATATTTTCTTGGGTGTGGTTTGAAAAATTCAAGCCAACCTTGTCTAT
TATGTCTTAATTAAAAGTTGTGTCGTGCAATCGTTTAAATGAAGTTTCATTTTTCTCCAGAAA
GACAAATGACCAAAACAACACCTTCATTTACCATCGCTACCTTTTACCCATATCCATTCCCTG
CTCCCATCCGTCCTCGGACACAGCTCCTACACCATGAGACAGAGGGAGGGGATCCTCATCTC
TCATTTGATGTGTAGGAAAAGTTCGTTGGTTAGTTTTTGGTGTCACGGTGACATCACAGTGAC
AGTGACGACTCCAATACTGTCTCGACGGTGTTTGATGAGGTGTTGCCAAGGAGATTGTGAGT
ATTTTTTGTTCTTGTCGGCCTTTTTGGACGACGTTGTGGTTCTTGTTGCTATTTTGGCGAGGCA
TCATTGACTGGTGTCGTCTTCTTCAACAACCCTTTCCTTCGAGCCTTTGCGAGCAATGTCAGT
GGCCTAGTTTGGCAATATGAGTGTGGTTGCCTTCCTAGATCCCCCACGCCAAATGTTCTTGGC
ATGATTGCTAATACCTGTTATACACGACTGTCTACCATTGCGACCATTCAAGATGTGGTCCTC
AAGAGGTCTTCACTGAGTAAAATTCTCAATGTTATTCTCCGCCGGCGAGAGCTAGGTGGGGC
AACGACACGAGTTTGGTTACGGTTTGGCTTGAATTTTGCGGCCTACGGTGTTGGTTATAATTC
ACCATCTGTTTATGGTTATTTCTATGCTTGTGGGTTTAGTTACCTCGTTATTTGAATGTATTCG
CCTTTTTCTTTGTGATATATGATAGAACTGATTAAAAAAATTGTGCTAGTCATAGTGAGGCA
AATAAGCTACATATGTACATTAAAAACAATATGACATGTCCACTAACTATAATACGCACAAA
AGAATTGTGTATGAGTCTTGTATTTTTTTTTCATCGTTTATTTTCAATGGAATATAGGTGACAT
GTTGTACAGTCTCACACGAACAGCTCTACATATTGCCCACTCGGCACACAAGAGGAACGCTC
GAACTTGTCGTGTGTGTGCGGACAAAATAAATAGAACTTTTCAATTTCCGCGTTCGAGATTTT
CAGCGGATGATTAACGCATTAGACAGAGCATCAAACGAATCGATCGAGTTTGAATAAGTAA
GTCACAGGCTCAAAAAAAGCAAGACACAGTTCATCTTTTTTTTCAGGAAAGGACCTGGTTCA
TGTTCATTTTATTTTTTGAGGAAAAGGGCCTGGTTCATCCTAGCTGTCCTGGTAACGGAGCTA
GTTCAACATAAGTCGAGCCTGTGCTTCTATATTTAGCATCAAACGTAACGAAGAGTTAACTT
AACAAAACTAATGATAATGCTATCTTAAGACAGGAAGCTAATTGATCGGTGTTCACTTGCAC
AACAGGATGGCCTTATGGCGATCGTGCGGCTGCCAACACTGCCTCACGCCCGCCCAAACTAT
TCGAACGGGAGGAAAACATAATTCATTGTGCTCAGATTTGGCAATTGATTACAAGATCGCGT
AGTATCCCTTTTTCTATCTCGTCGTGTGTCTACTACCGGGCCGCGCATTGATATTAGATATCA
AAGTTAAATGCTGATTTTTAAGAATAAATACCTCTTTTTTATTCCGGTGCAGGGGTAAATACC
GATTTTTTTTCTGCGGTGCAGGGGAAATACTGCCAAAAGTGGTATAATAAACCGCGATGGCG
GATTTGAAAAACCAGCGGCGCTCCGAGCCCATAATTCAGAGCGGAAAAACCCCCCAATGTG
TAAAGTGTATTCCTCCGAGCATCTATAAAAGGCCGTATACCTAGGAAACAAATACTCACCAA
CACCTAACAAAGATTTGCTTCTGTAGAGTACTTTACAACACTTCCACGATGGCTGGAGAGGT
TGGCAAGTGGGGTAGTTCCTTCAAACATTCTTGGGCTTTAATCCCACTGGTATGAAAATATTC
ATAGTACTTGGCTTCTTTTTGTGAAACTCATGGTTAATACTTGTTCTTCTATATGAACTTCATG
GCTATTCTTTTAAAAAAAACTCCACGACTTTTCTCATTTCTTGGTTCTTGTCCTTCCACCTACA
ATTATTGCTTACCTGAGCGAAAACTAGATCTGCGAGGTTATCATCTGCTGAAGCTTTTTTTGA
GGGATTCATCTTACTTAAGCTTAGTGCAACAAAGACATCCTTCCGCATATGTAGGTGTGTGA
TCTCGACAATAGATCTATGTAGTGGTACGGTATTCTTTTGGAAGAGGAGGATTACCCCTGGT
CTCTTAGCATAGGCGTGAGTTTCAAAAGTAATTCTAATGATTTTTGTCTTACAATTAAATTCT
GTTTCGTACGATATAGATATCTTCCTATGCCTGTAAGGTGCAGATTTGAAAAGTAGATCTAA
CTGATTTTTATCGTTTATGTTTCAGTTCTGTATATTACAGATATCATCCTGCACGTGTCAGGC
ATGCGAGATCTATTTCTTTAGATAACAATGAAAACTAGATCTTCATAATTTTTCATCTTGTAA
TTGAAGATTCTGCTCCGTACAAGACGGATATCCTCCCACGTGTGTTTTAGGCGTGAATTCAA
AAAGTAAATATAATTTTATTTTATCTTGTAATTAGGAATCCGCTCCGTACAATATTAGTGTCC
TTTCACCTGTGTAAGGTGTGCTCTAAAAATACATCTATGTAGGTTTTAATATATAATTTGGCT
TCTGCTCCGTACATCACAGATATCTCCCCACATGTGTGAGGCAGTGGCGAGTCGAAGAACTC
CCTTTTGTTGTATGCTCCTATATTGTTTCTCTGCTCACCGTGTGTTAGATCTACCCATGGAATG
TCATGACAAATGTTGTCGTTAGATGAGCCCAAAGGCGATCTATTAGCGGGATGCCACGGCAA
ATTATATCTTTAGATTAGTGAAAACCCTCGAAATCTTGACTTGTTACTTGCGCAACAAATCTT
ACCGTTAGACGAGTTGAAACTCGTTGCAATGCCGCTTGTCAAGGCCTCGTCAGCCTAAAAGA
CAGTTTAATTTGTCTACAAAAAAAGACACTTTAATTTAGTAATTATGCATTTTCTATTTTACT
TTTTACATGTTGCAAATATATATTTTTTCTGACGTCACACTTTTATTGCACTTGCACGCATGCA
GGTTGCCCATGGAATCATCGTGGTCGTAGTGGCTCTCGCTTACTCTTTCATCTCGTCGCACGT
AAATGATGATGCCGTGAGCGCCATGGACGCGTCGCTGGCGCACGTCGCCGCCGGCGTGCAG
CCTCTAATGGAAGCCAACCGCTCCGCCGCCGTCGTCGCGCACTCTCTGCAGATCCCCAGCAA
CGAATCATCTTATTTCCGATACGTAATTAACCGAGAACCTTTGGGTTAATTAAATTATGCCTT
TTTTTCTCTGTATAACGTGATTAGTTTCATCACGTATATGCACTTCCTTTTTGAACCACAATTA
TTTCCTTACTTTAAATAACAAATCTTAATTACTAGCCGGGCCGACGCGGTAACTGGTTATTGT
GTATGATTCTGTTCTGATTTTCGTAGTAATGCGAGCATTGATATGAATATACGCATGCATATA
CAAACAAATCATTTTTGCATACATTTTTTTATGTAGGACACGTCCAAGATAACATAGCAACA
CGTACGTGCAAATATACATCTAACATTTACGTATATATGCTTGACCTGACAGGTGGGACCAT
ACATGGTCATGGCGTTGGCCATGCAGCCGAAGCTGGCCGAGATATCATACACCAGCGTGGA
CGGCGCCGCGTTGACGTACTACCGCGGCGAGAACGGCCAGCCGAGAGCCAAGTTCGGGAG
CCAGAGCGGCGAATGGCACACCCAGGCCGTTGATCCGGTGAACGGCCGTCCCACCGGCCGC
CCCGACCCAGCGGCGAGGCCGGAGCACCTACCCAACGCGACGCAGGTCCTCGCCGACGCC
AAGAGCGGCTCGCCCGCGGCCCTCGGGGCCGGGTGGGTCAGCTCCAACGTCCAGATGGTG
GTCTTCTCCGCGCCTGTCGGTGACACTGCCGGCGTGGTCTCCGCCGCGGTCCCCGTCGACGTC
CTGGCGATCGCCAGCCAGGGCGACGCCGCCGCCGATCCCGTCGCGCGGACGTACTACGCG
ATCACTGACAAGCACGACGGCGGGGCCCCGCCGGTCTACAAGCCTTTGGACGCCGGGAAGC
CCAACCAGCACGACGCGAAGCTGATGAGGGCCTTTTCCTCGGAGACCAAATGCACCGCGTC
CGCCATTGGCGCGCCCGGCAAGCTCGTGCTCCGCGCCGTCGGGGCGGACCAAGTCGCGTGC
ACGAGCTTCGACCTCTCCGGAGTGAACCTGGTAACGTGTCCATCTAGCATCGATCACAGGCC
ATCCATATATGCATACGTACACGAACGTGCACACACCCTATATATCTAACATAATTGCTCTG
CATTTTTGTCAAGAATTCATCCCAGCATGTAATATTTCTTCCAAGTTTGCTGTTTATACATTCA
AGCAACGGACAATGAAAAAAGGTTAGATGAGGTAAGGGCATCTACAATGCTAGGAGCTTAC
ATAGGCGCTTATAGACAAAATAATAAATAAATAAAAATCTGAAACACACCTAAGCGCCTCA
TCCCTCAACGCTAGACGCTAATTAACTAAACAACGTGGACGCTAAGTACTGGAAGGAAAAT
GACCAAGCGCCGGGTGCATGGTTTGGCGTCGATGCCAGGGTTGACACCAGGATTACACCCG
GCAACCGCTAATCTGCCGAGATTATGAACCTGGCGCCTGGCCTAAACGCCCAGCAATGGAG
ATGCCCTAAGGGGACTTCGGCCAAAAAAAAGGGGAACTAATAAAACGTTTTTTTGTTTTGAC
GACCTCAATAAACATCATTCCGACTTGGAGTGAGGAAAAAAGGGAAACAGGGAACGCTCCT
AGCTATTGACAGTACGTACATAATCTTGCTTCCTTCCTTCCGCGTGTAAAAAAACTGAAAAC
TTTCCAAATCAAGGGATCCCAAAATTAGGAAGAAAATCTTAATAGAGAGTACAAAGTCTTCT
TCTTCCTCTTCTTCCCCAAAGCAAGATTTCTCCTTCTGTTCTTCCCCAAACGGAGCTCTGGCA
CAAAACTGCGGTGAGCTCGATCGTCTCGTACTTTTCTTGCATCTGCTAGCTAGGGTCTTGCAT
GCATATCACCGGTTGTCGTTCATGGATATCTCCCATCAGTTCTTTTGCAATTTATTTACAGCG
TAGAGAGCAGTATACTATGTACGCAGTAAATCACTATATAAACTGATCTTGACATCCGTCAC
CTATGCAACGAGCGCACGGCAAAGGGGCTGCGCTGGTCCGTTTGATAATTCAACGGCCAGG
CCGGGCGGCGCCTCGCGCTCCATGGAAACACCCTTTTTTCAGATAAAGGCCATGGAAACAAC
CTGACGTACAGTGCCGATCGCAATACCATAAAGGACCCAGTCATAAAAAAAAATTCCCAGA
GATTAGCCTCTTGATACAAAAAATATATTTTCTTTGTAGTTTAGCACGCATATGCATGTTTGA
GAATTCCCTTTTTTTGGGGACGGCTGACAAGTATCTTCGGTTGTATTTCTTCTTGTTTTCCAGG
GAGTTCGTCTTGTGGTCAGCGACTGGGGCGGGGCAGCCGAGGTCCGGCGAATGGGGGTGGC
CATGGTGAGCGTCGTGTGCGTGGTCGTGGCGGTCGCGACGCTGGTGTGCATCCTTATGGCAC
GGGCGTTGTGGCGGGCCGGGGCGCGGGAGGCCGCTCTAGAGGCTGACCTGGTGAGGCAGAA
GGAGGCGCTCCAGCAAGCGGAGCGCAAGAGCATGAACAAGAGCAATGCCTTCGCCCGCGCC
AGTCATGACATCCGCTCCTCACTCGCTGCCGTCGTTGGACTCATCGATGTTTCCCGGGTAGAG
GCCGAGAGCAACTCCAACCTCACCTATAACCTCGACCAGATGAACATTGGCACAAACAAAC
TCTTGGGTTAGTCCGCATCCATGCCCTACGTACCATGCATGGCAATACCAGCTTGCTCTACGT
TTTAGTAGATCTATCCGTACTTGGCAATTTAGCTAATGTGATCATAGCATTATAAATTTGCAT
GCCATAGAAGTAAAGTTTTCCTAAATAATTTAATTACAATCTTAGGTGTAGAGTGTGCAATA
GTCCAGCTGTTATACATTTAAGGTATCGAATTTGCTCATAAAATTTAAAATATGCAAGAAAT
CAATCTCTGTTTTGGAAAGAAATAGCATTTTATTTGAAAAAAAAAGTTTAGGATAGTTCAAA
TAGAATTGTCAGATCTCACTATGTTAGCAGCTCGTAAACTTATAAAGTTTCAACATTTCTATC
TATTTTGCTGTTGGGGAAATTTTGTCACATTTATGCATCATATTGTTTGTACGCGTGTTCGAG
TGCATAAAGCAAAAATTTTATTATTTCCGAAAAAATGAACTTTATACCATCTTTGTGTTCTCA
GCTCAGCCTATGTTGACTCATCATCCATGTTTATACATGTGTATGTGTACATGCAGATATACT
TAACACGATACTGGACATGGGCAAGGTGGAGTCCGGGAAGATGCAGCTAGAGGAGGTGGA
GTTCAGGATGGCAGACGTCCTTGAGGAATCCATGGACCTGGCAAACGTCGTCGGCATGTCAA
GAGGCGTCGAAGTGATCTGGGACCCTTGCGACTTCTCCGTGCTCCGGTGCACCACCACCATG
GGCGATTGCAAGCGTATCAAACAAATTCTTGACAACCTACTCGGCAACGCCATCAAGTTCAC
ACACGACGGCCACGTCATGCTTCGAGCATGGGCCAACCGTCCCATCATGAGGAGCTCCATAA
TCAGCACCCCGTCGAGGTTCACCCCCCGTCGCCGCACGGGTGGGATCTTTCGGCGGCTGCTT
GGAAGGAAGGAGAACCGTTCGGAACAAAATAGCCGAATGTCATTACAAAATGATCCTAATT
CGGTTGAGTTTTACTTTGAGGTGGTTGACACTGGTGTGGGCATACCCCAGGAAAAGAGGGAG
TCTGTGTTTGAGAACTACGTTCAGGTGAAGGAAGGGCATGGTGGCACCGGGCTCGGGCTTGG
AATTGTGCAATCCTTTGTAAGTGATCTCGTCTTYTTCATGCATGTTAAAATCTTGTCAACTGC
ATCAACGACAACTAGCCGTAAATGTATTTCGTTTTTTCTTGTTTACTTATAGTTTTGTTTGGTG
TTGTTGTTGTTGATGTAAATATAGGTTCGTTTGATGGGAGGAGAAATCAGCATTAAGGACAA
GGAGCCAGGAGAAGCGGGGACGTGCTTCGGCTTCAACATCTTCCTCAAGGTCAGCGAGGCG
TCAGAGGTGGAAGAGGACCTCGAGCAAGGGAGGACGCCGCCGTCGCTGTTCAGGGAGCCCG
CCTGCTTCAAGGGCGGGCACTGCGTCCTCCTCGCCCACGGCGACGAGACCCGCCGGATCCTG
TACACGTGGATGGAGAGCCTCGGGATGAAGGTCTGGCCCGTCACGCGCGCCGAGTTCCTCGC
CCCGACCCTCGAGAAGGCGCGCTCCGCCGCCGGCGCCTCGCCGTTGAGGTCAGCGTCGACGT
CGTCGCTGCATGGCATCGGGAGCGGCGACTCCAACACTACGACGGACAGGTGCTTCAGCTCC
AAGGAGATGGTCAGCCACCTGCGGAACAGCAGCGGCATGGCCGGCAGCCACGGCGGGCACC
TCCACCTCTTCGGCCTGCTCGTCATTGTCGACGTCTCTGGCGGGAGGCTCGACGAGGTCGCC
CCCGAGGCGGCGAGCTTGGCGAGGATCAAGCAGCAGGCGCCGTGCAGGATCGTCTGCCTCA
CGGACCTCAAGACCCCCTCCGAGGATCTGAGGAGGTTCAGTGAGGCGGCGAGCATCGACCT
CAACCTGCGCAAGCCCATCCACGGCTCCCGGCTGCACAAGCTCCTCCAGGTCATGAGAGACC
TCCATGCCAACCCGTTTACGCAGCAGCAGCCGCAGCAGCTCGGTACAGCCATGAAAGAACT
GCCGGCGGCTGATGAGACCTCTGCGGCGGAGGCGTCTTCTGAAATCACGCCCGCGGCAGAG
GCGTCTTCTGAAATCACGGCCGCTGCGGAGGCGTCTGAGATCATGCCGGCGGCGCCGGCGCC
GGCTCCCCAGGGACCGGCCAATGCAGGAGAAGGCAAGCCGCTGGAGGGGATGCGCATGCTG
CTGGTGGACGACACCACGCTGCTGCAGGTAGTCCAGAAGCAGATACTGGCCAATTACGGAG
CAACGGTGGAGGTCGCCACGGATGGCTCCATGGCCGTGGCCATGTTTACAAAGGCTCTTGAG
AGCGCAAATGGCGTCTCAGAGAGCCATGTGGACACAGTGGCCATGCCCTACGATGTCATCTT
CATGGATTGCCAGGTACATTTCTCCAGCAACAGCATGCCAAGCACATCAGCCCCATCCTCCT
GTTCCTGAAGATGATTTAATCTGACGCTGCTGACAATTCGATCTTCTTTGTTTCAGATGCCAG
TGATGAATGGCTATGATGCGACGAGGCGCATCCGGGAGGAAGAAAGCCGCTACGGCATCCG
CACCCCGATCATCGCGCTGACCGCGCATTCCGCAGAGGAGGGGCTGCAGGAGTCCATGGAG
GCAGGGATGGATCTTCACCTGACCAAGCCGATACCCAAGCCGACAATCGCACAGATTGTTCT
AGACCTCTGCAACCAAGTTAATAACTGATCACCGAGACTCTTCGTTCCCCGTTCCGCCGTCG
CATGATCAAAAATCAAGATAGGTGTAGGTGGTTTTTCAGCGAGCGAATGCAGTTATCATCCT
AGTCACTGAAAACCCACCTACACCTCGAGTTTCGATCATGCGACCCGGGGCATTATCGTAGT
TTGTAGCATTTAGCAGCAGCAGCTGAACTTTGTTGTTGTATCAAAATCAGGTCAGGTTTATTT
CCAGAATTATTCTTGGACAATGTATTGTCGATTTTGAATTTCCAGAAACAATTATGGTTAAGT
TTTGAATTTTCAGAGTTTGTGTTTTTAGAGCTCTTTTTTCCCAGAGTTTGTGTTTTCAGAGCAT
GTGATTAGACACCATATATTTGCTTCCACTGCCCATTCACAGGAGAAAGAAAGTACAGATTC
CTACAAGCCATGAAATCCCTAGCTAGCTACCCCAGATATCTAGTAGTGTACACATAGCTGAT
AAATACCCATAGGAATAGCTGTACAATCTCCTCTAGGTCTGTAGTGGACTAGCCTAAATATT
ACTAGGAGTAGCTCTCATATGCAGGCACCAAGTGGGAAAAGTTCACACCCGAGGTCGTTTTC
CGTGAACGGCACTTCGTCGCTCTCCTGCATTGAAATCGGAAAAGGGGTTCAGAAAAATCACC
ATCAAAATTCTAAGAACATAGGTTTACTAAATAACGATTTTAAAGGAAATATGACTGTCATA
AATAGTGAAATTTTAAGCATGACTGATGTCTTTACCACTTTTATTATGGAGTTGAGAAGACC
ATCAAAGTTGCAGCTGCTGGAATTGAACCCACCCTGCAGCTTTGGAACTCCCAAATCGTACA
TATCGGTCTGTTCAGGCACAATGGCACCACCATCATAGCTAAAACCTCCACTGTTCAGTCTTT
GGTCTTGTCTCATTCCATCCATTCTATGTCTGGAGTTGCTTGCAGCCCCAAACTGGAGGTATT
TTGAATGTCTTTCGGTATCACGAGGAAGGGGCAGAATTATACTGCCAGAGGAACTAGCTCTA
CATTTGGCATTGCTGGCTACTATAAGGCTCTCAGAAGGACAAACACTAACAGACATTCTTTC
CAAAAATATCCCCTTTTGGTCCATCTCTTGTTCTCCAGCCACAAAGCTAGCATCAAGTTTTGT
CACGCCAGTACCATCGAACGGGATGTGCAACGGTAACTTGTTAACAGAAAACCCTTCACTGA
TTGGAAGAGCACACTGCTGAGATATACAAGAATCCCGCAAATTGGCGGAAACTCCGACAGA
CCTTTCAAGCAGCCCTGAACTCCCAGTTGGTATTCCTATGGATGGATGAGCTCCAACTTTGAT
GTGTGGAGTGCACTCCAAAAGATCCTCTGGTGGCAGTGAACTGCTTGTAACTCTTTGGAGTG
TGCCAGACATAGTGTTAGCCAGAGCACCCCCGGAAATGACAGAGCACAGGTCATTGGTTTCC
TGATGGATCCACTTCTGCTGGAGCTGAGGCTGCCCAAGCGACGATGCGGTCAAGCCTTGTGA
TAAATCTGCCTGTTGGTTGTCTTGTAAGCTCACAATGTGGAACTTCTCCGTATCGCCGGCGCA
ATGACTTGCTATGCCGTTGCTGCTAGAACACTGGTTTGCCTTGGGAGAAGCAAGGTCCTGAA
GCCCAAATGCTGCTGCACCAGCACTACTCAGCAGTCCATGTGGATTGAAAGATGGAAGAGC
AGCAGAAGGGGCAAAGGGTTGATGATAGCTATGAAGTCCTTCAAATGCTCCCATGTGCAAG
AAGGGGTCTCTGCCTCCAAAAGCAGCAGCAATGCTGGCTTGCTGTGATGCCACAGCACTTAG
CCGTCTGAGGTATAGCCTGTACTTCTGCATTGGCATACAAAGTAACCTGATTAGACATGGAG
GAAATTATGTAATCATCAGAATTCTGAAACATGAGCTATACAGTCTTGAGATAACTGCTTCC
TGGTTTGGTATGGAGTTGGTACATGAAAGCACTCCAGGATATAGTAAAATCTGATGAATTTT
CTTAACCTTTTAATGTAGGCTTGTTAATAAACTTTCTATTTGTCAATTCATTGACATGCAAAG
CTTTTGCATTTCTATAAAAGAATATTTTAACCAAAAGGTGTACTACTACCTCCGTCGCAAAAT
ATAAGACAGAGGTAGTACAACGCAGTTTACATATATGTAGCTTTGTAATCTGGACAGGGTGT
GTCAGTGTCTTCCTTCTGGCAGTTTTATAGAAGTGAAACAATGTAGCCAAAATACTACATTA
AACTCAGCAGTACATACCAAAACACCACATTAAACATGTACAGCAGTACATACCAAAATAC
AACATTAACTTGTACAGCAGTGCATACCAAAATACTACAATAAACTTGTACAGCAGTATTAC
CTAATTACTCTGCATTAAACCTGTACAAGAGTACATACTAATTTGAGAATTTTATACCTGCGT
AGTTAGATCTAATCTACCACTGTAGTCTACATGGTTGAGGGAATGATCAGATATTTGTATTA
GATATCTCCTTCAGGGTTTCTGAAAGATTCAGCATAACTTTTTTTTCGAAAAGGGGGATCTTC
CCGGCCTCTGCATCAGAATGATGCATACGGCCATCTTATTAGCGAAATAAAAGGTTCCAACA
AGGTTCCAAAGTCTCCGACTGAAAAGTAATAAAAAGACAGCTCACATAGAGCTAAAGAGGC
TGGACACACAGACTAGCCAAGATAAGACTCCACAACCGGCTGGCTAAAGATAGATAGGTAA
ACTAATTGCCTATCCATTACATGACCGCCATCCAAACCGGTTGAGATATCCCGAAGATTCAG
TATAACTGAACTGAATGTTTCTTTGTTTTCAGACCCGCTGAACATATTCAACTTGTCAAGAAA
AATGAAGAAATGTGGAGGTGAAACTATGTAACCGCAAAATTAAACTACTTAACTCATTTTGC
ACAAACATCAGGAACATATTAACTTATTTGTTAAAAGAGTTCTGCTCTGCAACTAATAGGTG
TGTCGATACCAAATACAGTAAGGGGATAAGGACTATGTATACGAATATCTTTCTTTTTTTAA
ATTGTTCAAAAGTTGGCCCTCTTAATTCGACACAGGAAAATAAGGCCATTTGCAGATAATTT
TACCAAAAGTTCTGTAAAAGTTTTCCTTCCTGTGCACCACATGCTGATCAATGAAAGAGATA
ACACAGGTAAAGGCGATGATGGAAGACTGGAAGTAAATGCCATGTCGCCATACCTGCAGAT
GGCTCGCAACATTTTCCCTGGTGA
OV/ guides (first, second and fourth guides are in the reverse
direction relative to the coding sequence)
SEQ ID NO: 23 AACGCGGCGCCGTCCACGCTGG
SEQ ID NO: 24 GCGAGGACCTGCGTCGCGTTGG
SEQ ID NO: 25 GTCAGCTCCAACGTCCAGATGG
SEQ ID NO: 26 GCGTAGTACGTCCGCGCGACGG
SEQ ID NO: 27 M s 1 -A CDS
ATGGAGAGATCCCGCCGCCTGCTGCTGGTGGCGGGCCTGCTCGCCGCGCTGCTCCCGGCGGC
GGCGGCCGCCTTCGGGCAGCAGCCGGGGGCGCCGTGCGAGCCCACGCTGCTGGCGACGCAG
GTGGCGCTCTTCTGCGCGCCCGACATGCCCACCGCGCAGTGCTGCGAGCCCGTCGTCGCCGC
CGTCGACCTCGGCGGCGGGGTCCCCTGCCTCTGCCGCGTCGCCGCGGAGCCGCAGCTCGTCA
TGGCGGGCCTCAACGCCACCCACCTCCTCACGCTCTACAGCTCCTGCGGCGGCCTCCGTCCC
GGCGGCGCCCACCTCGCCGCCGCCTGCGAAGGACCCGCTCCCCCGGCCGCCGTCGTCAGCAG
CCCCCCGCCCCCGCCACCGTCGACCGCACCTCGCCGCAAGCAGCCAGCGCACGACGCACCA
CCGCCGCCGCCGCCGTCCAGCGACAAGCCGTCGTCCCCGCCGCCGTCCCAGGAACACGACG
GCGCCGCTCCCCACGCCAAGGCCGCCCCCGCCCAGGCGGCTACCTCCCCGCTCGCGCCCGCT
GCTGCCATCGCCCCGCCGCCCCAGGCGCCACACTCCGCCGCGCCCACGGCGTCATCCAAGGC
GGCCTTCTTCTTCGTCGCCACGGCCATGCTCGGCCTCTACATCATCCTCTGA
SEQ ID NO: 28 Ms1-A AA
MERSRRLLLVAGLLAALLPAAAAAFGQQPGAPCEPTLLATQVALFCAPDMPTAQCCEPVVAAV
DLGGGVPCLCRVAAEPQLVMAGLNATHLLTLYSSCGGLRPGGAHLAAACEGPAPPAAVVSSPPP
PPPSTAPRRKQPAHDAPPPPPPSSDKPSSPPPSQEHDGAAPHAKAAPAQAATSPLAPAAAIAPPPQ
APHSAAPTASSKAAFFFVATAMLGLYIIL
SEQ ID NO: 29 Ms1-A genomic
CGCTTCTGCAAAAATCTCCACTAGCCATTGCATAAGCTCAGGAAAATTACCTTTATGTAGTG
AAACTTCTCTCCATCATGTTCACGAAAATCTAACCCTTGACAAAAAAGGAACCTCGGGCATT
AAAAGGAATATGTCAGGCCAGCTCTATATAAAACCTTGTCTCGTTTGATGGTTGAACAAAAT
GACTCTATGATTGTTGTGTTTGCTGCAATGAAGAAATTGTATTTCTCTTGTGCTTTGTTACGT
GCACACTGCACTATTGATTTCACCGACATGTTTCACAAAACTATCCTTGTGATTCTAATTTCT
AAGTCACCCATTCACCAAAAATCTCCACCAACATGCAAATTATCATTGAAAAGATAACATAC
AAGCATAAAGCACCATCTAGTTCTTTACTATACTCAAGCCAACTATAAGACTTAAACCATTT
AGCTACAAATATTGTTGCACACCTCCGGTGGGGTGTTGTGGAAAAGCATATTTTTTCGGTCA
ACAAGCCCCTTTTGCAATGTATCCTCTTCTAATCCTATTCGGACCATTAACATCATAAGTTGC
GATTGGCATCCTCTTCCTAGGATCAGATTCACTCAATCGAACATCATAAACTGCATCTTCAAT
GTCACCCATTTCCTATATTTTTTCAGATTATTGGCTTGCTTCGTTCGCAATATTAGGTACTGTG
ATTGGACTTCTGTTGATGCCACTAATAATTTGCAGTTGTTGCGGAATATGAACTCAAGGGGA
GCTCATGGTGCTATGAAGTTGATTCGGTGGGAAATTGTTCTACATCCGCACTTGCTGCTCAAC
CTAAATACATGGGTTGGATTTCTTCCCAACTTTAGTACATAAAGTTCTCAAATTAATGTTCTA
CTACATTAAAATTGAAATCCGCAAACATTTTTTAGTACCCAAACATTTTTCTAATATACGGTG
AACATTTTTCATCTACTGATTTTTTGATATATGGTGAAAATTGGTGTAATATATGCTGGCATG
TTTTTAAATACTACATATTGACCATGTAGATAAAAAATTTATAGTATATGATGAACATTTTTG
TAATATAGATGGCCATTTTTAAAATATACATTGCACATTTTATAATATACGATGAGCAGTTTA
TAATACTAGATGAACCTTTTTTGGAGTTCTGAACATTTTTTTGAAAACAGCAGCCATTGTACA
AGAAAAAACCAAAACAAAAGAAATGAGAAACCCAAAAACAAAAACAAAACAAAACAAAA
CAGAGAAACCTACAGAAAAAAACGAAACAGAAAAAGGCAAAGGAAGAACCCGAACTGGGC
CAGCCGGCTCGGCGTGCCCCAGTGGGCCGTCGTGGCGAATGCAACGGCTACATGGGCCGCT
CTTCGTGAAAGAGAAGGAGGTCAGTTCATGGACCGCTACCAGTACACGGGCCTCGCTGTGG
CAACACCCGCCGTGTACTAGTTTTCGCGGGAATCCAATGCCAAAATCGCTCCCCGCGGGAAC
CCGACGTCGGTCTGGTGACTTCTGGAGCCTTCCAGAACACTCCACAAGCTCCCAGAGCCGTC
TGATCAGATCAGCACGAAGCACGAACATTGGCGCGCGAAGATATTTTCCTTCCCGACGACGC
CACACTGCATTTCATTTGAATTTCAAAAATCGAAAACGGAAAACACTTTCTCTCATCCCGAG
GAGAGGCGGTTAGTGCCAGAGGAGCACGAGAGAGGCCACCCCCCCCCCAGCCAGCTCACGT
GCCGTGCCCTCGCACCCTGCGCGGCCGCATCCGGGCCGTCCGCGCGGACAGCTGGCCGCGCC
CCACCCGAACCGACGCCCAGGATCGCGCCCGCCACCCGCTTGCCTTAGCGTCCACGGCTCCT
CCGGCTATATAACCCGCCCCTCACCCGCTCCCCCTCCGGCATTCCATTTCCGTCCCACCACCG
CACCACCACCACTCCACCAAAACCCTAGCGGGCGAGCGAGGGAGAGAGAGACCACCCCGCC
CGACCCCGCCGATGGAGAGATCCCGCCGCCTGCTGCTGGTGGCGGGCCTGCTCGCCGCGCTG
CTCCCGGCGGCGGCGGCCGCCTTCGGGCAGCAGCCGGGGGCGCCGTGCGAGCCCACGCTGC
TGGCGACGCAGGTGGCGCTCTTCTGCGCGCCCGACATGCCCACCGCGCAGTGCTGCGAGCCC
GTCGTCGCCGCCGTCGACCTCGGCGGCGGGGTCCCCTGCCTCTGCCGCGTCGCCGCGGAGCC
GCAGCTCGTCATGGCGGGCCTCAACGCCACCCACCTCCTCACGCTCTACAGCTCCTGCGGCG
GCCTCCGTCCCGGCGGCGCCCACCTCGCCGCCGCCTGCGAAGGTACGCGCACGTTCACCGCC
CTCCGTCCCTCCCTCTCTCTGTCTACGTGCAGATTTTCTGTGCTCTCTTTCCTGCTTGCCTAGT
ACGTAGTGTTCCATGGCCTCTCGGGCCGCTAGCGCTCCGATTTGCGTTGGTTTCCTTGCTGTT
CTGCCGGATCTGTTGGCACGGCGCGCGGCGTCGGGTTCTCGCCGTCTCCCGTGGCGAGCGAC
CTGCGCAGCGCGCGCGGCCTGGCTAGCTTCATACCGCTGTACCTTGAGATACACGGAGCGAT
TTAGGGTCTACTCTGAGTATTTCGTCATCGTAGGATGCATGTGCCGCTCGCGATTGTTTCATC
GATTTGAGATCTGTGCTTGTTCCCGCGAGTTAAGATGGATCTAGCGCCGTACGCAGATGCAG
AGTCTGTTGCTCGAGTTACCTTATCTACCGTCGTTCGACTATGGTATTTGCCTGCTTCCTTTTG
GCTGGGTTTATCGTGCAGTAGTAGTAGACATGTGGACGCGTTCTTCTTATTTTGTGCCGACCA
TCGTCGAGATACTTTTCCTGCTACAGCGTTTCATCGCCTGCACCATCCCGTTCGTGATAGCAC
TTTTGTGTCAAACCGCAACGCAGCTTTGCTTTCTGCGGTATCTTCTGCCTTGTTTGTCGCCTTG
CTTGGTCAAAACTGAGAACTCTTGCTGTTTGATCGACCGAGGGCAGAGGCAGAGCAAGAGC
CTGCCGTGCTTTTGGCTCTGCAGTGCGTCGTCTCTGCCTCCTTTGCCAAACATTTCCATGTTG
ATCCTCTGGGGGCACTGCTTTTTCGCATGCGGTTTCCGTAGCCTTCCTCTTTCATGAAAAAAG
GTTTGGGTCAAATCAAATGGATCGCCTATTGGCAGAGCAGCAGCAGATAGCTGGCTGTCTCA
CAGCTTTGGCAGAATCGGTCTGTTGCCTGCCACCGTGTCTCTTATCTTGCCTGCCACCGTGTC
TCTTTTCTTGTTGCGCACGTCGTCACCTCCTCCTACTTCTTTTCCAGTTTTGTTTACTTTTGATG
AAATACGGACGAACGGCTGGTAATCATTAACTTTGGTTGCTGTTGTTACTGTGGATTTTGGA
CGCAGGACCCGCTCCCCCGGCCGCCGTCGTCAGCAGCCCCCCGCCCCCGCCACCGTCGACCG
CACCTCGCCGCAAGCAGCCAGCGCGTGCGTACCTCTCCCTCTCGCCCGCATCTCGCTCCGTAT
TAACTGATTGTGTCTGCATACTGACGTGTGCTTTGGCTTTGGATCTGTTTCGCAGACGACGCA
CCACCGCCGCCGCCGCCGTCCAGCGACAAGCCGTCGTCCCCGCCGCCGTCCCAGGAACACG
ACGGCGCCGCTCCCCACGCCAAGGCCGCCCCCGCCCAGGCGGCTACCTCCCCGCTCGCGCCC
GCTGCTGCCATCGCCCCGCCGCCCCAGGCGCCACACTCCGCCGCGCCCACGGCGTCATCCAA
GGCGGCCTTCTTCTTCGTCGCCACGGCCATGCTCGGCCTCTACATCATCCTCTGAGTCGCCGA
CCCCGAGGCCATGGTCCGTCCAGTTGCAGTAGAATGCTCGTCGTCTTGTTCCGTTTCATGCTT
GTCGCCGTTCGAGGTTCGTTTCTGCAGTCCGATTGAGAAGAAGACGGTGGGTTTTGATCGCG
TCCCGAGATTTCTGTTGTCGATCGTAGCGTCCTGGTAGTAGTAGTGTCTGGTAGCAGCAGTAT
GTTCATGTGTCCTCGGTCGCCTAGTTTTGGTCTCAAGTAGTACTGTCTGTCCACCGTGTTTGC
GTGGTCGCGGAGAACATCATTGGGTTTTGCGATTCCTCTGGTCAGATGAACCACTGCTATGT
GATCGATCGATATGATCTGAATGGAATGGATCAAGTTTTGCGTTCTGCTGATGACGTGATGC
TTCTTCAGTTATATTCATGCTCGATCTATTTCTGTTTCCCCCATTTGAATTTGTGGAGCAGCAG
TTTGGCTTTCTTTTGTTCTGCTATGGATGAATGCTTCTTGCATGCATCTTGTCTTTGCTTAATT
TGAACTGTAGAACGGATGCAGTTCTGGTTTCTGCTAATGATGTGATGATTCTTCATATGCATA
TGCTTTACATGTTCATCTCTTCAAATTTGTGCAGCAACAGTTTGTAGCTTTCATTCGGCTCTG
AATGAAATGCCTCTTGCATGTTGTCTTTGCTTAATTTGTTTTTCACGGGGAGCCTGCTGCAGC
TTTCTGTTGCCATGTTGTTTTCCACGCCAGGACAAAATAGATGGTGCGGTTTGATTCGATCCC
GGTTAATTGCTTGATGCTAGCTTCTGATCAATCCCTTCATCACGATGTTCCGGAGAGCCACAT
GGAACTGGAGGGGGGAGATTCAAATTCATGCATGCAAATTTGTGTTGGTGTTGGGTCACGTC
AAGCAGTCACTTTTTGCAGTATCACTCTTACCATTTTATCCTTTTGTTGAAACCTCTCTCCTCA
CCCCAAAAGTTGATGCAATAGTGCTATGCCCACCCATGCTTTTTTCATAATCTTTTGAGCCCA
AAGTCCCATTTTACTATCTGTTTGCATATTTGTGTTCCTTGCGGCGAGGGCTATCAAGCAAGG
CCTTTCTTGAATATATTTTGGCAAGTTTTCAAATTTGAATTCTAAAAGATGGTGAAACTCTAT
GAAACAAATCTCAAAGTATATGACCTTATCACCAATCCACCATTCTACAAATATTTCATTCTC
CGGCATCGCCTGCTTCCGACGGCGATGCCGCTGTAGGTCCTCGCCGCCGCCGCAACTCTTCC
GCTAGCTATGTGGTGGAACTGTTGGCACTCCACCCTCATTTCCCGTCTCTTTCTATCGTCTCT
AACCACCGCACAACGTTCCCTACGTGGGGAGGAGCAACAATATCTGCTTCAACTCTTTGGAG
GGTAAATTGCATGGATTTCATTCACAAAGAAAATATTGCATGGATTTTAAGTCAATTTTTGTG
GCTGTGGATCAATCAACCAAACAATTTGGGAGAAAAAATTCAGCTTAGAATCTGTATGAGTG
TGGTTGTGTTTGTGTGACCCTTGCGTGAGGAACAGCAGGGACGCCAAGGAGGGTTGCCATGG
ACGCAACAAGCAGAGGAGCCGACGGGCCTCTGAGGAATGCTGTCGCGGACGGCGGGGGAG
AGTAGCGGGGAGGGAGCGCCCAGTGCTTCCACAAGCAGGAGAGTTGCGACAGCGTCGATGA
CGAACGGACGGAGCACTCATAATTAGAAGAGTGTGGCGCTAGAGAAAAAGGACAAGGGGA
TTTGCATCTAATTGCTTTGAGATTCGTTTTGTCACGTGTCACCCGCTGGAGAAGGTCTCACGA
CCGGAGGCGTGCGACCCGGGTCATATAGGAATTTTTTTCGTGCCATCGATAACAACCGAAAT
TCTTCGTGCCGTTGAAATCTTGAATAGATCTGAATCAGCAGAAGTTACTTTAACCCCACCGTC
GTAAAAAGAAAGGCAGAAATCGACGAACTCAAGGAGGATGGGAACCAAAGACA
SEQ ID NO: 30 Ms1-B CDS
ATGGAGAGATCCCGCGGGCTGCTGCTGGTGGCGGGGCTGCTGGCGGCGCTGCTGCCGGCGG
CGGCGGCGCAGCCGGGGGCGCCGTGCGAGCCCGCGCTGCTGGCGACGCAGGTGGCGCTCTT
CTGCGCGCCCGACATGCCGACGGCCCAGTGCTGCGAGCCCGTCGTCGCCGCCGTCGACCTCG
GCGGCGGGGTGCCCTGCCTCTGCCGCGTCGCCGCCGAGCCGCAGCTCGTCATGGCGGGCCTC
AACGCCACCCACCTCCTCACGCTCTACAGCTCCTGCGGCGGCCTCCGCCCCGGCGGCGCCCA
CCTCGCCGCCGCCTGCGAAGGACCCGCTCCCCCGGCCGCCGTCGTCAGCAGCCCCCCGCCCC
CGCCTCCACCGTCCGCCGCACCTCGCCGCAAGCAGCCAGCGCACGACGCACCACCGCCGCC
ACCGCCGTCGAGCGAGAAGCCGTCGTCCCCGCCGCCGTCCCAGGACCACGACGGCGCCGCC
CCCCGCGCCAAGGCCGCGCCCGCCCAGGCGGCCACCTCCACGCTCGCGCCCGCCGCCGCCGC
CACCGCCCCGCCGCCCCAGGCGCCGCACTCCGCCGCGCCCACGGCGCCGTCCAAGGCGGCCT
TCTTCTTCGTCGCCACGGCCATGCTCGGCCTCTACATCATCCTCTGA
SEQ ID NO: 31 Ms1-B AA
MERSRGLLLVAGLLAALLPAAAAQPGAPCEPALLATQVALFCAPDMPTAQCCEPVVAAVDLGG
GVPCLCRVAAEPQLVMAGLNATHLLTLYSSCGGLRPGGAHLAAACEGPAPPAAVVSSPPPPPPPS
AAPRRKQPAHDAPPPPPPSSEKPSSPPPSQDHDGAAPRAKAAPAQAATSTLAPAAAATAPPPQAP
HSAAPTAPSKAAFFFVATAMLGLYIIL
SEQ ID NO: 32 Ms1-B genomic
AACATATTTATAATAAATGGTGAACATTTTTTTTAATAATTGATGACCATTTTTAAAATGCAT
ATTGAACATTTTATAATATACACTGTACAGTTTTATAATAATCGACGAACATCTTTTGGAGTT
CTGAACATTTTTTTCAAAAACACAAGCCATTTTCCAGGAAGAATACAAATGCAAAAGAAATG
AGATATCCAAAAAGCAAAAAAGAAAAACAAAACAAAACAGAGAAACCTACAGGAAAATCC
AAACAGAAAAGGCAAAGAAAGAACCCGAACTGGGCCAGGCAATGTTTCCAACGGCCTCGCT
CTTCCTGAACAAGAAGGCCAGTCAGCCCATGGGCTGCTCCCAGTACTCGGGCCCCGCTGTGG
CAGCACGCCATGTAATAGTTTTCGCGGGAATCCAACGCCGAAATCGCCCGCAGCGGGAACC
CGACGTCGGTCTGGTGCGTTCTGGCGCCTTCCAGAACTCTCCACAGGCTCCCGCAGCCGTCC
GATCAGATCAGCACGAAGCACGAACATTGGCGCGCGGCGATATTTTCTTTCCTCGCCCGACG
ACGGCCGCACTGCATTTCATTTTGAATTTCAAAATTCGGAAACGGAAAAGCTTTCTCGCATC
CCGAGGCGAGGCGGTTACGGGCGCCAGAGGGGCCACCCCACCCACCCACCCCCGCCCTCAC
GTGCCCCGCGCGGCCGCATCCGGGCCGTCCGCGCGGACAGCTGGCCGCGCCCAGCCCGAAC
CGACGCCCAGGATCGAGCGAGGGCGGCGCGCCCGGGGCTTGGCTTAGCGTCCACGCCACCT
CCGGCTATATAAGCCGCCCCACACCCGCTCCCCCTCCGGCATTCCATTCCGCCACCGCACCA
CCACCACCACCAAACCCTAGCGAGCGAGCGAGGGAGAGAGAGACCGCCCCGCCGCGACGAT
GGAGAGATCCCGCGGGCTGCTGCTGGTGGCGGGGCTGCTGGCGGCGCTGCTGCCGGCGGCG
GCGGCGCAGCCGGGGGCGCCGTGCGAGCCCGCGCTGCTGGCGACGCAGGTGGCGCTCTTCT
GCGCGCCCGACATGCCGACGGCCCAGTGCTGCGAGCCCGTCGTCGCCGCCGTCGACCTCGGC
GGCGGGGTGCCCTGCCTCTGCCGCGTCGCCGCCGAGCCGCAGCTCGTCATGGCGGGCCTCAA
CGCCACCCACCTCCTCACGCTCTACAGCTCCTGCGGCGGCCTCCGCCCCGGCGGCGCCCACC
TCGCCGCCGCCTGCGAAGGTACGTTGTCCGCCTCCTCCCCTCCCTCCCTCCCTCCCTCTCTCTC
TACGTGCTCGCTTTCCTGCTTACCTAGTAGTACGTAGTTTCCCATGCCTTCTTGACTCGCTAG
AAGTGCTCCGGTTTGGGTCTGTTAATTTCCTCGCTGTACTACCGGATCTGTCGTCGGCACGGC
GCGCGGCGTCGGGTCCTCGCCTTCTCCCGTGGCGACCGACCTGCGCAGCGCGCGCGCGGCCT
AGCTAGCTTCATACCGCTGTACCTCGACATACACGGAGCGATCTATGGTCTACTCTGAGTAT
TTCCTCATCGTAGAACGCATGCGCCGCTCGCGATTGTTTCGTCGATTCTAGATCCGTGCTTGT
TCCCGCGAGTTAGTATGCATCTGCGTGCATATGCCGTACGCACGCAGATGCAGAGTCTGTTG
CTCGAGTTATCTACTGTCGTTCGCTCGACCATATTTGCCTGTTAATTTCCTGTTCATCGTGCAT
GCAGTAGTAGTAGCCATGTCCACGCCTTCTTGTTTTGAGGCGATCATCGTCGAGATCCATGG
CTTTGCTTTCTGCACTATCTTCTGCCTTGTTTTGTTCTCCGCAGTACGTACGTCTTGCTTGGTC
AAAACTGAAAAACGCTTTGCTGTTTGTTTGATCGGCAAGAGCTGGCCGTGCTTTTGGCACCG
CAGTGCGTCGCCTCTGCCGCTTTTGCGAAACATTTCCATGTTGATCCTCTGGCGGAACTACTT
TTTCGCGTGCGGTTTGCGTGGCCTTCCTCTCTCGTGAAAAGAGGTCGGGTCAAACCAAATGG
ATCGCCTCTTGGCAGAGCAGCGGCAGCAGATAGCTGGCCGTCTCGCAGCTTTGGCAGAACCG
GTCTGTGGCCATCTGTCGCCGCCTGCCACCGTTTCCCTGATGTTTGTTTCTCTCTCGCCTGCCA
CTGTTTCTTTTCTTGTTGCGCACGTACGTCGTCACCTCCTCCTACTTTTTTGCCAGTTTTGTTTA
CTTTTGATGAAATATACGGATGAATCGGCTGGTGATTAACTTTGGCTGCTGCTGTTAATTACT
GTGGATTTTGGATGCAGGACCCGCTCCCCCGGCCGCCGTCGTCAGCAGCCCCCCGCCCCCGC
CTCCACCGTCCGCCGCACCTCGCCGCAAGCAGCCAGCGCGTAAGAACCTCTCCCTCTCCCTC
TCTCTCTCCCTCTCGCCTGCATCTCGCTATGTTTATCCATGTCCATATGTTGATCAGCCTTGTT
TAGTTACTAACATGTGCACCGGATCGGGTTCTCGCAGACGACGCACCACCGCCGCCACCGCC
GTCGAGCGAGAAGCCGTCGTCCCCGCCGCCGTCCCAGGACCACGACGGCGCCGCCCCCCGC
GCCAAGGCCGCGCCCGCCCAGGCGGCCACCTCCACGCTCGCGCCCGCCGCCGCCGCCACCG
CCCCGCCGCCCCAGGCGCCGCACTCCGCCGCGCCCACGGCGCCGTCCAAGGCGGCCTTCTTC
TTCGTCGCCACGGCCATGCTCGGCCTCTACATCATCCTCTGAGTCGCGCGCCGACCCCGCGA
GAGACCGTGGTCCGTCCAGTCGCAGTAGAGTAGAGCGCTCGTCGTCTCGTTCCGTTTCGTGC
CTGTCGCCGTTCGAGGTTCGTTTCTGCGTGCAGTCCGGTCGAAGAAGCCGGTGGGTTTTGAG
TACTAGTGGTAGTAGTAGCAGCAGCTATCGTTTCTGTCCGCTCGTACGTGTTTGCGTGGTCGC
GGAGAACAATTAATTGGGTGTTTGCGAGTCCTCTGGTTAAGATGAACCACTGATGCTATGTG
ATCGATCGATCGGTATGATCTGAATGGAAATGGATCAAGTTTTGCGTTCTGCTGATGATGTG
ATCCATTTGGATCTGTGTGGGGCAACAGTTTCGCTTGCTTTTGCTCTGCGATGAACGAATGCT
TCTTGCATGCATCTTGTCTTTGCTTAATTTGAACTGTAGAACGGATGCAGTACTGATTTCTGC
TTATGATGTGACGATTCGTCGTACGCATATCATCTCTTCAAATTTGTGTAGCAGCTGTTTGTA
GCTTCCATTCTGCTATGGACGAATGCCTGTTTTTCACGGAGAACCGCGCGCGGGGACCGATG
CGGCTTTGTGTTGCCATGTTGTTTTCCACGCCAGGACAAAATAGATGGTGCGGTTTTGATCCC
CAATCCCACCATCACCATGTTCCGGAGAGCCACATGGAACTCACGTCAAGCGGTCACTTTTT
GCAGAATCACTCTTACCATTTTACCCTTTTGTTGAAACCTCTCTCCTCATCCCCAAAAGTTGA
TGCAACAGTGCTATGCGCGCCCACCCATGCTTTTTCATATGATTGTAAAATTTGGATCGATTT
TATCTTTTGAACCCTAAGTCCGGTTTACAATCTGTTTGCATGTTTATGTTCCTTGCGGCGAGG
ACCATTAAACAAGACTACTATTGGATATATTTCGACAGGCTTTGAAATCCGAATTCTAAAAC
ATGGTGAGACTCTATGAGACACAAGAATGCTCTTTAGAACACGAGGAAACCTAATTAAGAT
TGATAAGAACAGA
SEQ ID NO: 33 Ms1-D CDS
ATGGAGAGATCCCGCGGCCTGCTGCTGGCGGCGGGCCTGCTGGCGGCGCTGCTGCCGGCGG
CGGCGGCCGCGTTCGGGCAGCAGCCGGGGGCGCCGTGCGAGCCCACGCTGCTGGCGACGCA
GGTGGCGCTCTTCTGCGCGCCCGACATGCCCACGGCCCAGTGCTGCGAGCCCGTCGTCGCCG
CCGTCGACCTCGGCGGCGGGGTGCCCTGCCTCTGCCGCGTCGCCGCGGAGCCGCAGCTCGTC
ATGGCGGGCCTCAACGCCACCCACCTCCTCACGCTCTACGGCTCCTGCGGCGGCCTCCGTCC
CGGCGGCGCCCACCTCGCCGCCGCCTGCGAAGGACCCGCTCCCCCGGCCGCCATCGTCAGCA
GCCCCCCGCCCCCGCCACCACCGTCCGCCGCACCTCGCCGCAAGCAGCCAGCGCACGACGC
ACCGCCGCCGCCGCCGCCGTCTAGCGAGAAGCCGTCGTCCCCGCCGCCGTCCCAGGAGCAC
GACGGCGCCGCCCCCCGCGCCAAGGCCGCGCCCGCCCAGGCGACCACCTCCCCGCTCGCGC
CCGCTGCCGCCATCGCCCCGCCGCCCCAGGCGCCACACTCCGCGGCGCCCACGGCGTCGTCC
AAGGCGGCCTTCTTCTTCGTCGCCACGGCCATGCTCGGCCTCTACATCATCCTCTGA
SEQ ID NO: 34 Ms1-D AA
MERSRGLLLAAGLLAALLPAAAAAFGQQPGAPCEPTLLATQVALFCAPDMPTAQCCEPVVAAV
DLGGGVPCLCRVAAEPQLVMAGLNATHLLTLYGSCGGLRPGGAHLAAACEGPAPPAAIVSSPPP
PPPPSAAPRRKQPAHDAPPPPPPSSEKPSSPPPSQEHDGAAPRAKAAPAQATTSPLAPAAAIAPPPQ
APHSAAPTASSKAAFFFVATAMLGLYIIL
SEQ ID NO: 35 Ms1-D genomic
CCAAACAACAGAGTCCACTGTCTCCAAGAGCACCGGGAAGGAAGCAGCAAGACGGTGCCAA
TCTTCCAACTCTACAGGGGACAACACCCTATGGAAACCCAAATCCCAATTCCTACCAGAGAG
CTCCGTGATGGAGATCCCTGGATCTGAGCAATAAGAAAACAAGTTTGGAAAAGAGCCCGAG
AGAGGCCCCTCTCCAACCCACCAATCCAACCGAAAGCAAACAAAACGACCATCTCCCACCA
CGAACTTAACTAGAGACCTGAAATCATCATGAACATGAATCAGCTGGCGCAAGAACCGGGA
GCCCCCAGATCCAGAGGCAAACAATGGGTTGCCAGTGGGGAAATATTTAGCTTTCAGGATAT
CCCACCATAGGGTCCTCGCACTAGTTCTTTACTATACTCAAGCCAACTATAAGACTTAAACC
ATTTAGCTACAAATATCGATGCACACCTCCCGTGGGGTGTTGCGGAAAAGCATGTTTTTTTG
GTCGACAAGCCCCTTTCACAATGTATCCTCTTCTAATTCTATTCAGATCATTAACATCAGCTG
TGATTGACATCCTCTTCCCAAGATCAGATTCACGCAATTGAACATCATAAACCACATCTTCA
ATGTCATCCTCTTCCTATATATTTTTAGATGATTAGCTTGCTTCGTTCTCAATATCAGGTTCTA
TGAATGGACTTGAGTTGATGCCACTAATAATTTGTAGTTGTTGCAAAATGTGAACTGAAGGG
GAGCTATGAATGAACTTGAGTTGATTTGATGGGAAATTGTTCTACACATGCACTTGCTGCTC
AACTTAAATACGTGCCTTGGATTTCTTCCCAACTTTAGTACATAAAGTTCTCCAAGTAATGTT
CTACTACATAAAATTTGAAATCTGCAAACATTTTTTAGTACACGAACATTTTTCTATATACAG
TGAACATTTTTCATCTACTGATTTTATTTTAATATATGGTGAAAATTGGTGTAATATATGCTG
ACATGTTTTTAAGTACATATTGAACATATATATAAAATACATGATGAACATTTTTGTTATATA
TGATGCTCATTTTTTCAATACATATTGAACATTTTATATTATACGATGGACAGTTTTATAATA
ATCAATGAACAACTTTTGGAGTTCTGAACATGCTTTTGAAAACACAAGACATTTTCCAATAA
AAAACAAAACAAAAGAAATGAGAAACCCAAAAACAAAAACAAAACAAAACAGAGAAACCT
ACAGAAAAAACGAAACAGAGAAGGCAAAGAAAGAACCGGAACTGGGCCAGCCAACTCGGC
GTGCCCCAGTGGTCCGTCGTGGCGAATGTTTGCAACGGCTACATGGGCCGCTCCTCGTGAAA
AAGAAGAAGGTCAGTCCATGGGCTGCTACCAGTACACGGGCCTCGCTGTGGCAAACTGGCA
ACACGCCATATTAGTTTTCGCGGGAATCCAATGCCGAAAACCACCCACCGCGGGAACCCGA
CGTCGGTCTGGTGACTTCTGGCGCCTTCCAGAACCCTCCACAAGCTCCCAGAGCCGTCTGAT
CAGATCAGCACGAAGCACGAAGCACGAACATTGGCGCGCGAAGATATTTTCTTTCCCCAGCC
TCCGCCTCGCCCGACGACGCCGCACTGCATTTCATTTGAATTTCAAAAATCGAAAACGGAAA
AACTTTCTCGCATCCCGAGGAGAGGCGGTTACGCGCGCCAGAGGAGCACGAGAGAGGCCAC
CCCACGCACCCAGCCAGCTCACGTGCCGCCCTCGCACCCCCCGCGGCCGCATCCGGGCCGTC
CGATCGCACAGCTGGCCGCGCTCCACCCGAACCGACGCCCAGGATCGCGCCCGCCACCCGCT
TGCCTTCGCGTCCACGGCTCCTCCGGCTATATAACCCGCCCCCCACCCGCTCCCCCTCCGGCA
TTCCACCCCAACACCGCATCACCACCACCACTCCACCAAACCCTAGCGACCGAGCGAGAGA
GGGAGAGACCGCCCCGCCGATGGAGAGATCCCGCGGCCTGCTGCTGGCGGCGGGCCTGCTG
GCGGCGCTGCTGCCGGCGGCGGCGGCCGCGTTCGGGCAGCAGCCGGGGGCGCCGTGCGAGC
CCACGCTGCTGGCGACGCAGGTGGCGCTCTTCTGCGCGCCCGACATGCCCACGGCCCAGTGC
TGCGAGCCCGTCGTCGCCGCCGTCGACCTCGGCGGCGGGGTGCCCTGCCTCTGCCGCGTCGC
CGCGGAGCCGCAGCTCGTCATGGCGGGCCTCAACGCCACCCACCTCCTCACGCTCTACGGCT
CCTGCGGCGGCCTCCGTCCCGGCGGCGCCCACCTCGCCGCCGCCTGCGAAGGTACGTCGCGC
ACGTTCACCGCCTCCCTCCCTCCCTCGCTCTCTCTCTCTCTCTCTCTCTCTCTCTCTACGTGCC
GATTCTCTGTGTTCGCTTCCCTGCTTACCTAGCACGTAGTTTTCCATGGCTTCTCGACTCGCTG
GTCCTCCGATTTGGGTCGGTTAATTTCCTCGCTGTACTACCGGATCTGTCGGCACGGCGCGCG
GCGTCGGGTTCTCGCCGTCTCCCGTGGCGAGCGACCTGCGCAGCGCGCGCGCGGCCTAGCTA
GCTTCATACCGCTGTACCTTCAGATACACGGAGCGATTTAGGGTCTACTCTGAGTATTTCGTC
ATCGTAGGATGCATGTGGCAGTCGCGATTGTTTCATCGATTTTAGATCTGTGCTTGTTCCCGC
GAGTTAAGATGGATCTAGCGCCGTACGCAGACGCAGATGGTCTTGCTGTCTCTGTTGCTCGA
GTTATCTTATCTACTGTCGTTCGAGTATATTTGCCTGCTTCCTTTTGATCTGTGTTTATCGTGC
AGTAGCAGTAGCCATGTCCACGCCTTCTTGTTTCGAGGCGATCATCGTCGAGATAGCGCTTT
GTTTCAAACCGCAACGCAGCCTTTGCTTTCTGCGGTATCTTCTGCCTTGTTTTTGTTCTGTGCA
GTACGTCTTGCTTGGTCAAAAGTAAAAACTCTTGCTGTTCGATCGACCGAGGCCTGATGCAG
AGCAAGAGCTGGCCGTGCTTTTCGCTCTGCAGTGCATCGCCTCTGCCTCTTTGGCCAAACATT
TCCATGTTGATCCTCTGGTGTGGTACTACTTTTTTGCATGCGGTTTGCGTAGCCTTCCTCTTTC
GTGAAAAAAGGTCGGGTCGCCTATTGGCAGAGCAGCAGCAGCAGCAACAGATAGCTGGCTG
TCTCGCAGCTTTGACAGAACCGGTCTGTGGCCATCTGTCGCCGCCTGCCACCGTTTCCCTGAT
GTTTGTTTCTCTCGTCTCATCTCGCCTGCCACTGTTTCTTTTCTTGTTGCGCACGTCGTCACCT
CCTCCTACTTTTTTTTCCAGTTTTGTTTACTTTTGAGATACGGACGAACGGCTGGTAATTACTA
ACTTTGGTTGCTGTTGTTACTGTGGATTTTGGACGCAGGACCCGCTCCCCCGGCCGCCATCGT
CAGCAGCCCCCCGCCCCCGCCACCACCGTCCGCCGCACCTCGCCGCAAGCAGCCAGCGCGTA
CGAACCTCTCCCTCCCTCTCTCTCGCCTGCATCTCGCTCTGTATTAGCTGATTGTGTTTACTTA
CTGACGTGTGCTTTGGCTTTGGATCTGTTTCGCAGACGACGCACCGCCGCCGCCGCCGCCGT
CTAGCGAGAAGCCGTCGTCCCCGCCGCCGTCCCAGGAGCACGACGGCGCCGCCCCCCGCGC
CAAGGCCGCGCCCGCCCAGGCGACCACCTCCCCGCTCGCGCCCGCTGCCGCCATCGCCCCGC
CGCCCCAGGCGCCACACTCCGCGGCGCCCACGGCGTCGTCCAAGGCGGCCTTCTTCTTCGTC
GCCACGGCCATGCTCGGCCTCTACATCATCCTCTGAGTGGCCGACCCCGCAAGACCATGTCC
GTCCAGTTGCAGTAGAGTAGAGTGCTCGTCGTCTTGTTCCGTTTCATGCTTGTCGCCGTTCGA
GGTTCGTCTCTGCATGCAGTCCGATCGAAGAAGACGGTGGATTTTGAGTAGTAGCTGTCGTT
GGCAGGAGTATGGAGTTCATGTGTCCTCGGTCGCCTAGTTTTGGTCTCAAGTAGTGTCTGTCT
GTCCGCCGTGTTTGCGTGGTCGCGGAGAAGTACAATTGGTGGGTGTTTGCGATTCCTCTGGTT
AGATGAACCACTGCTATGTGATCGATCGATATGATCTGAATGGAATGGATCAAGTTTTGCGT
TCCGCTGATGATGATGTGATATGCTTCTTCATGTATATATATTCATGCTCGATCTATTTGTGTT
TCTCCGATTTGAATCTGTGTTAAGCAACAGTTTGTCTTGCTTCTGTTCTGCAGCTTCTGCTATG
GATGGATGCTTCTTGCATGCATCTTGTCTTTGCTTAATTTGTAGTAGAACGGATGCAGTTTTG
ATCTCTGCTGATGATGTGATGATTCTTCATATGCATATGCTCTGTACATGTCTCTTCAAATTT
GTGTAGCAACAGTCTGTAGTTCTCGTTCTGCTCTGAATGAATGCCTCTTGCATGTTGTCTTTG
CTAGCTTTGTGGTAGAAATGTAGAATGCAGACATTGCTTCCGTCCCAAATAATCTGTTCCTTG
CTTCGTATATATATTGACATGTTGTGCATATAATCTGTGAATGAAGTTGTGAACAAGTCTTCT
TTCAGAAAAAAAAGTTGTGAACAAGTGCCTCACCTCACCTACAAGGCTACAAACACAACAA
CAACAGAAGCTGGCCTCTTCACGGAGAACCGCGCGGGGACTGCTGCAGCTTTCTGTTGCCAT
ATTGTTTTTCACGCCAGGACAAAATAGACGGTGCGGTTTGATTCGATCCCGGTTAATTCTCA
ATCCCTTCGTCACTATGTTCCACATGGAACCGGAGGGGGTAGATTCACATTCGTGCATGCAA
AATTTATTGGTATTGCTCGATCCATCAACTCGTGTACCGTCAACTGGGTCACGTTTTGCCATA
AAAGTCTTACCATTTTACCCTAGCGCTATGCCCACCCATGCTTTTTCATATGATTCTGAAGTT
TTAAATCTATTTTATCTTTGAGGCACTAGGTGGTGCGGTTTGATTTGATCCCGGTTAATTCTC
AATCAAATTTTATTGGTGTTGCTCTAGTGGGGGAGCTTGAGCAAAATTTAAGAGGGGGCCAT
GACTCAAGGGGAACAAATTAGTAGGCCTTTAGGGGCTACTCACTTGTTGAAATACTAATTAG
GCCTAAAAGCTAGCACGCTTTTTAATGAATGCCAAAATTAGGAGGGGGGGGGGGGGGGGGC
ATGCCCCCCTTGGTCTACACTAAACTCCGCCAGTGTATCGCCGTCATTTGGGTCATGTCAAGC
AGCCACTTTTTGCCATAACACTCTTACCATTTTACCCTTTTGTTGAAACCTCTCTCCTCACTCC
AAAAGTACCTGACGAGTAATGCTACGCCCACCCATGCATTTTCATAGTATGATTTTAAAGTT
TTAAATCTATCTTATCTTTTGAATTGAAAGTCTGATTTACAATCTGTTTGCATATTTATGTTCC
TTGCGGCAAGGACTTTCAAACAAAAGACCTTTCTTGAATATATTTCGACAAGTTTTAAAATTT
GATTTCTAAAACATGGTGAAACGCTATCAAACATATATAGTGATGCTCTCCCGAACAAGAAA
AAAAATCTACTAATAAAACTTGATAAGAACACACATTAATAACTTGATAAAAACATTTTAGA
TTCGTACGAAGACTGCTTAAAGTGTCATTGTTTACCAAGTTCCACATGCATTGATCGATTTGA
TTAGTTGGAACTGTCGAGGTTGGGTCAACCACGAATAGTTCAAGAACTTGTGTGTCTCTCTA
AGGCGCATCGTCCCAATATTATCTATCTTTCT
SEQ ID NO: 36 Ms26-A CDS
ATGAGCAGCCCCATGGAGGAAGCTCACCATGGCATGCCGTCGACGACGACGGCGTTCTTCCC
GCTGGCAGGGCTCCACAAGTTCATGGCCATCTTCCTCGTGTTCCTCTCGTGGATCTTGGTCCA
CTGGTGGAGCCTGAGGAAGCAGAAGGGGCCGAGGTCATGGCCGGTCATCGGCGCGACGCTG
GAGCAGCTGAGGAACTACTACCGGATGCACGACTGGCTCGTGGAGTACCTGTCCAAGCACC
GGACGGTCACCGTCGACATGCCCTTCACCTCCTACACCTACATCGCCGACCCGGTGAACGTC
GAGCATGTGCTCAAGACCAACTTCAACAATTACCCCAAGGGGGAGGTGTACAGGTCCTACA
TGGACGTGCTGCTCGGCGACGGCATCTTCAACGCCGACGGCGAGCTCTGGAGGAAGCAGAG
GAAGACGGCGAGCTTCGAGTTCGCTTCCAAGAACCTGAGAGACTTTAGCACGATCGTGTTCA
GGGAGTACTCCCTGAAGCTGCGCAGCATCCTGAGCCAGGCTTGCAAGGCCGGCAAAGTCGT
GGACATGCAGGAGCTGTACATGAGGATGACGCTGGACTCGATCTGCAAGGTGGGGTTCGGG
GTCGAGATCGGCACGCTGTCGCCGGAGCTGCCGGAGAACAGCTTCGCGCAGGCGTTCGACG
CCGCCAACATCATCGTGACGCTGCGGTTCATCGACCCGCTGTGGCGCGTGAAGAAGTTCCTG
CACGTCGGCTCGGAGGCGCTGCTGGAGCAGAGCATCAAGCTCGTCGACGAGTTCACCTACA
GCGTCATCCGCCGGCGCAAGGCCGAGATCGTGCAGGCCCGGGCCAGCGGCAAGCAGGAGAA
GATCAAGCACGACATACTGTCGCGGTTCATCGAGCTGGGCGAGGCCGGCGGGGACGACGGC
GGCAGCCTGTTCGGGGACGACAAGGGCCTCCGCGACGTGGTGCTCAACTTCGTGATCGCCGG
GCGGGACACCACGGCCACGACGCTCTCCTGGTTCACCTACATGGCCATGACGCACCCGGCCG
TGGCCGAGAAGCTCCGCCGCGAGCTGGCCGCCTTCGAGGCGGACCGCGCCCGCGAGGATGG
CGTCGCGCTGGTCCCCTGCAGCGACTCAGACGGCGACGGCTCCGACGAGGCCTTCGCCGCCC
GCGTGGCGCAGTTCGCGGGGCTGCTGAGCTACGACGGGCTCGGGAAGCTGGTGTACCTCCA
CGCGTGCGTGACGGAGACGCTGCGCCTGTACCCGGCGGTGCCGCAGGACCCCAAGGGCATC
GCGGAGGACGACGTGCTCCCGGACGGCACCAAGGTGCGCGCCGGCGGGATGGTGACGTACG
TGCCCTACTCCATGGGGCGGATGGAGTACAACTGGGGCCCCGACGCCGCCAGCTTCCGGCCG
GAGCGGTGGATCGGCGACGACGGCGCGTTCCGCAACGCGTCGCCGTTCAAGTTCACGGCGTT
CCAGGCGGGGCCGCGGATCTGCCTCGGCAAGGACTCGGCGTACCTGCAGATGAAGATGGCG
CTGGCCATCCTGTGCAGGTTCTTCAGGTTCGAGCTCGTGGAGGGCCACCCCGTCAAGTACCG
CATGATGACCATCCTCTCCATGGCGCACGGCCTCAAGGTCCGCGTCTCCAGGGCGCCGCTCG
CCTGA
SEQ ID NO: 37 Ms26-A AA
MSSPMEEAFIEIGMPSTTTAFFPLAGLHKFMAIFLVFLSWILVHWWSLRKQKGPRSWPVIGATLEQ
LRNYYRMHDWLVEYLSKHRTVTVDMPFTSYTYIADPVNVEHVLKTNFNNYPKGEVYRSYMDV
LLGDGIFNADGELWRKQRKTASFEFASKNLRDFSTIVFREYSLKLRSILSQACKAGKVVDMQELY
MRMTLDSICKVGFGVEIGTLSPELPENSFAQAFDAANIIVTLRFIDPLWRVKKFLHVGSEALLEQSI
KLVDEFTYSVIRRRKAEIVQARASGKQEKIKHDILSRFIELGEAGGDDGGSLFGDDKGLRDVVLN
FVIAGRDTTATTLSWFTYMAMTHPAVAEKLRRELAAFEADRAREDGVALVPCSDSDGDGSDEA
FAARVAQFAGLLSYDGLGKLVYLHACVTETLRLYPAVPQDPKGIAEDDVLPDGTKVRAGGMVT
YVPYSMGRMEYNWGPDAASFRPERWIGDDGAFRNASPFKFTAFQAGPRICLGKDSAYLQMKM
ALAILCRFFRFELVEGHPVKYRMMTILSMAHGLKVRVSRAPLA
SEQ ID NO: 38 Ms26-A genomic
TCTCATCTGTGGAACATATTTATTTGGCAGCACTAGATGCCTCGGCATATTGCAAGGTTTTTA
ATATTTGCGATCTTTTCTGTTTCAAGCTTCTAATAAATAGAAGGTGACCACTTTCATCAAAAT
TTTCTTCTGTTTAGCTTCTGCTACAAATTTCTAATAAATATAGAAGGGGGAACTTTCAGCAAG
ATTTTTTATATTTGTGATTTTCAGGCTTTTTCCATTTAGGGAGAACATCAGAGCACCCCTTGA
CAGTTGACACCCCTTCATTCGAAATTTCTCAACTTGTTCTGCTTTGACTTCAAAAACTGTTTC
ACTGAAAGATGCACTTTGTATTGGTTAGTGCGGGTTCAATAAAGACCAGATGGACCATAACC
ATGGCTCCATGGCTCCAACTGTGAAGATGACATAATCACAACGCTAACTGTCATCAAACGCA
TCACCTACATCCCCCGCAAAACGAAATAAAAATGCATCAGTGCATCACCTACATTTATAGTA
AAACAGAAGGAAAATGCAGAATCCATGACCTAGCTTAGCACCAAGCACATACTAACATACC
TAGTTATGCATATAAAAATGAGTGTTTTCTTGGTCAGCAGATCACAAAAAGGACACAAACGG
TAGGTTCCATCTAGTCAGGGGGTTAGGTTAGGGACGCCATGTGGATGAGGCAATCTTAATTC
TCGGCCACACCAAGATTGTTTGGTGCTCGGCGCCACTAATGCCCAATATATTACCTAACCGA
GCCATCCAAATGCTACATAGAATTAATCCTCCTGTAGACTGAACCCACTTGATGAGCAGCCC
CATGGAGGAAGCTCACCATGGCATGCCGTCGACGACGACGGCGTTCTTCCCGCTGGCAGGG
CTCCACAAGTTCATGGCCATCTTCCTCGTGTTCCTCTCGTGGATCTTGGTCCACTGGTGGAGC
CTGAGGAAGCAGAAGGGGCCGAGGTCATGGCCGGTCATCGGCGCGACGCTGGAGCAGCTGA
GGAACTACTACCGGATGCACGACTGGCTCGTGGAGTACCTGTCCAAGCACCGGACGGTCAC
CGTCGACATGCCCTTCACCTCCTACACCTACATCGCCGACCCGGTGAACGTCGAGCATGTGC
TCAAGACCAACTTCAACAATTACCCCAAGGTGAAACTGAAAGAACCCCTCAGCCTTGTGAAT
TTTTTTGCCAAGGTTCAGAAGTTTACACTGACACAAATGTCTGAAATTGTACGTGTAGGGGG
AGGTGTACAGGTCCTACATGGACGTGCTGCTCGGCGACGGCATCTTCAACGCCGACGGCGA
GCTCTGGAGGAAGCAGAGGAAGACGGCGAGCTTCGAGTTCGCTTCCAAGAACCTGAGAGAC
TTTAGCACGATCGTGTTCAGGGAGTACTCCCTGAAGCTGCGCAGCATCCTGAGCCAGGCTTG
CAAGGCCGGCAAAGTCGTGGACATGCAGGTAACCGAACTCAGTCCCTTGGTCATCTGAACAT
TGATTTCTTGGACAAAATTTCAAGATTCTGACGCGAGCGAGCGAATTCAGGAGCTGTACATG
AGGATGACGCTGGACTCGATCTGCAAGGTGGGGTTCGGGGTCGAGATCGGCACGCTGTCGC
CGGAGCTGCCGGAGAACAGCTTCGCGCAGGCGTTCGACGCCGCCAACATCATCGTGACGCT
GCGGTTCATCGACCCGCTGTGGCGCGTGAAGAAGTTCCTGCACGTCGGCTCGGAGGCGCTGC
TGGAGCAGAGCATCAAGCTCGTCGACGAGTTCACCTACAGCGTCATCCGCCGGCGCAAGGC
CGAGATCGTGCAGGCCCGGGCCAGCGGCAAGCAGGAGAAGGTGCGTACGTGATCGTCGTCG
TCAAGCTCCGGATCGCTGGTTTGTGTAGGTGCCATTGATCACTGACACACTAGCTGGGTGCG
CAGATCAAGCACGACATACTGTCGCGGTTCATCGAGCTGGGCGAGGCCGGCGGGGACGACG
GCGGCAGCCTGTTCGGGGACGACAAGGGCCTCCGCGACGTGGTGCTCAACTTCGTGATCGCC
GGGCGGGACACCACGGCCACGACGCTCTCCTGGTTCACCTACATGGCCATGACGCACCCGGC
CGTGGCCGAGAAGCTCCGCCGCGAGCTGGCCGCCTTCGAGGCGGACCGCGCCCGCGAGGAT
GGCGTCGCGCTGGTCCCCTGCAGCGACTCAGACGGCGACGGCTCCGACGAGGCCTTCGCCGC
CCGCGTGGCGCAGTTCGCGGGGCTGCTGAGCTACGACGGGCTCGGGAAGCTGGTGTACCTCC
ACGCGTGCGTGACGGAGACGCTGCGCCTGTACCCGGCGGTGCCGCAGGACCCCAAGGGCAT
CGCGGAGGACGACGTGCTCCCGGACGGCACCAAGGTGCGCGCCGGCGGGATGGTGACGTAC
GTGCCCTACTCCATGGGGCGGATGGAGTACAACTGGGGCCCCGACGCCGCCAGCTTCCGGCC
GGAGCGGTGGATCGGCGACGACGGCGCGTTCCGCAACGCGTCGCCGTTCAAGTTCACGGCG
TTCCAGGCGGGGCCGCGGATCTGCCTCGGCAAGGACTCGGCGTACCTGCAGATGAAGATGG
CGCTGGCCATCCTGTGCAGGTTCTTCAGGTTCGAGCTCGTGGAGGGCCACCCCGTCAAGTAC
CGCATGATGACCATCCTCTCCATGGCGCACGGCCTCAAGGTCCGCGTCTCCAGGGCGCCGCT
CGCCTGATCTTGACCTGGTTCCGGCGACGGTGATGGACGCTCCGGTGGCTGGCTGGCCGGAC
GGCCGGCGCGTTATGACAGGCTCGATTTAGCTTGGCAACTGTGATAAACTCGTATATGTAGG
CAGAGTGGAGAGGGTGTTGATCGATTCGCCATGGACGTTGCTCGTCCGTTGTTACCATCGTA
CCATGTTTGTATTGCTTCTAGATCACTTTATAGTTCGTGTTTGTTCTTGAGCCTAAGTATTTAT
TGCACATTTCAAAAGTGACAAATGTATGCAATTGTCTTTTTGGGGTGTTTTCTAAGGGTAGTA
TTTTCGTAGATTTATTTTGTCGACCAAACCCTGGCCGTCACACATGATTCGATCCCTCTTGCC
GCCGCCAGCGTGCGACACCAGCGCGGGGGGGGGGGGGGGGGGGGGGGGGGGGGGGGGGG
GGGGGGGGGGGGGCTAGGGTGCTACACCGGCGGGCGCCGCTGTTGCTTGTGGGGAGTCCAG
TATGGAGGAGGGCGACGACGATGAGTGGTCGGATTACAACTGTCGACAGCTGATGCTCGTT
GGCGGCACTAGTGAAGCCGACATTGGTCGGAGGTTCACATATCCAGTGGGAAGGTCATCAA
CGGCAGCCTGGCTTGGCCCGGACATCGGAGAAGAGGGCGTCGATGTATGGTCCTGGATGGC
GACAAGCTTGATATCAAACTCGGCCCTATCATGCAGCGGCATGTTTTCTTCTTCTTCTTCAGG
TTTACTTTAGGAAGTCCCAGTTTAGGAGTAATGTTTTCCCAGTTTTATTGGTGTGTTTATCGTC
GGCGGAGGACATGTGGAACTGTGTCTTCGATTTTCTTTTAGGATCTACCCGGCTTACATTTTT
CGCTGGATCCATTTGGATTCTTTCGACTTTCATAGTCTACAGAGTTTCTACATGTCCT
SEQ ID NO: 39 Ms26-B CDS
ATGAGCAGCCCCATGGAGGAAGCTCACCTTGGCATGCCGTCGACGACGGCCTTCTTCCCGCT
GGCAGGGCTCCACAAGTTCATGGCCGTCTTCCTCGTGTTCCTCTCGTGGATCCTGGTCCACTG
GTGGAGCCTGAGGAAGCAGAAGGGGCCACGGTCATGGCCGGTCATCGGCGCGACGCTGGAG
CAGCTGAGGAACTACTACCGGATGCACGACTGGCTCGTGGAGTACCTGTCCAAGCACCGGA
CGGTCACCGTCGACATGCCCTTCACCTCCTACACCTACATCGCCGACCCGGTGAACGTCGAG
CACGTGCTCAAGACCAACTTCAACAATTACCCCAAGGGGGAGGTGTACAGGTCCTACATGG
ACGTGCTGCTCGGCGACGGCATATTCAACGCCGACGGCGAGCTCTGGAGGAAGCAGAGGAA
GACGGCGAGCTTCGAGTTCGCTTCCAAGAACTTGAGAGACTTCAGCACGATCGTGTTCAGGG
AGTACTCCCTGAAGCTGTCCAGCATCCTGAGCCAGGCTTGCAAGGCAGGCAAAGTTGTGGAC
ATGCAGGAGCTGTACATGAGGATGACGCTGGACTCGATCTGCAAGGTGGGGTTCGGGGTGG
AGATCGGCACGCTGTCGCCGGAGCTGCCGGAGAACAGCTTCGCGCAGGCCTTCGACGCCGC
CAACATCATCGTGACGCTGCGGTTCATCGACCCGCTGTGGCGCGTGAAGAAATTCCTGCACG
TCGGCTCGGAGGCGCTGCTGGAGCAGAGCATCAAGCTCGTCGACGAGTTCACCTACAGCGTC
ATCCGCCGGCGCAAGGCCGAGATCGTGCAAGCCCGGGCCAGCGGCAAGCAGGAGAAGATC
AAGCACGACATACTGTCGCGGTTCATCGAGCTGGGCGAGGCCGGCGGCGACGACGGCGGCA
GCCTGTTCGGGGACGACAAGGGCCTCCGCGACGTGGTGCTCAACTTCGTCATCGCCGGGCGG
GACACGACGGCCACGACGCTCTCCTGGTTCACCTACATGGCCATGACGCACCCGGCCGTGGC
CGAGAAGCTCCGCCGCGAGCTGGCCGCCTTCGAGTCCGAGCGCGCCCGCGAGGATGGCGTC
GCTCTGGTCCCCTGCAGCGACGGCGAGGGCTCCGACGAGGCCTTCGCCGCCCGCGTGGCGCA
GTTCGCGGGACTCCTGAGCTACGACGGGCTCGGGAAGCTGGTGTACCTCCACGCGTGCGTGA
CGGAGACGCTCCGCCTGTACCCGGCGGTGCCGCAGGACCCCAAGGGCATCGCGGAGGACGA
CGTGCTCCCGGACGGCACCAAGGTGCGCGCCGGCGGGATGGTGACGTACGTGCCCTACTCC
ATGGGGCGGATGGAGTACAACTGGGGCCCCGACGCCGCCAGCTTCCGGCCAGAGCGGTGGA
TCGGCGACGACGGCGCCTTCCGCAACGCGTCGCCGTTCAAGTTCACGGCGTTCCAGGCGGGG
CCGCGGATCTGCCTGGGCAAGGACTCGGCGTACCTGCAGATGAAGATGGCGCTGGCCATCCT
GTGCAGGTTCTTCAGGTTCGAGCTCGTGGAGGGCCACCCCGTCAAGTACCGCATGATGACCA
TCCTCTCCATGGCGCACGGCCTCAAGGTCCGCGTCTCCAGGGTGCCGCTCGCCTGA
SEQ ID NO: 40 Ms26-B AA
MSSPMEEAHLGMPSTTAFFPLAGLHKFMAVFLVFLSWILVHWWSLRKQKGPRSWPVIGATLEQ
LRNYYRMHDWLVEYLSKHRTVTVDMPFTSYTYIADPVNVEHVLKTNFNNYPKGEVYRSYMDV
LLGDGIFNADGELWRKQRKTASFEFASKNLRDFSTIVFREYSLKLSSILSQACKAGKVVDMQELY
MRMTLDSICKVGFGVEIGTLSPELPENSFAQAFDAANIIVTLRFIDPLWRVKKFLHVGSEALLEQSI
KLVDEFTYSVIRRRKAEIVQARASGKQEKIKHDILSRFIELGEAGGDDGGSLFGDDKGLRDVVLN
FVIAGRDTTATTLSWFTYMAMTHPAVAEKLRRELAAFESERAREDGVALVPCSDGEGSDEAFAA
RVAQFAGLLSYDGLGKLVYLHACVTETLRLYPAVPQDPKGIAEDDVLPDGTKVRAGGMVTYVP
YSMGRMEYNWGPDAASFRPERWIGDDGAFRNASPFKFTAFQAGPRICLGKDSAYLQMKMALAI
LCRFFRFELVEGHPVKYRMMTILSMAHGLKVRVSRVPLA
SEQ ID NO: 41 Ms26-B genomic
GCGGGAGCTACATGCACCGGGCTGCCCTTTAGCTTCTGCTAAAAATTTCTAGCAAGTATAGA
AGGGCGGAACTTTCAACAAAGATATGAGAACATCAGAGCACTCCTTGACACCCCTTCATTCC
AAATTTCTCAACTTGCTCTGCTTTGACTTCAAAAACTGTCTCACTGAAAGATGCACTTTGTAT
TGGTTAGTGCGGGTTCATTAAAGATCAGACGGACCATAACCATGGTTCCAACTGTGAAGATG
AGACCATCACAATGCTAACTGTCATCAAATGCATCACCTACATTCCCTGCAAAATAAAAATA
AAAATGCACGACCTACATGTGCAGTAAAACAGAAGGAAAATGCAGAATCCATGACCTAGCT
CAGCATCAAGCACATACAAACATATCTAGTTATATGCATATAAAAATCAGTATTTTCTTGGT
CAGCAGATCACAAAAAGGACACAAACGGTAGGTTCCATCTAGTCAGGGGGTTAGGTTAGGG
ACACCATGTGGATGAGGCAATCTTAATTCTCGGCCACACCAAGATTGTTTGGTGCTCGGCAG
CACTAATGCCCAATATATTACCTAACCGAGCCATCCAAATGCTACATAGAGTTAATCCTCCT
GTAGACCTGAACCCCCTTCATGAGCAGCCCCATGGAGGAAGCTCACCTTGGCATGCCGTCGA
CGACGGCCTTCTTCCCGCTGGCAGGGCTCCACAAGTTCATGGCCGTCTTCCTCGTGTTCCTCT
CGTGGATCCTGGTCCACTGGTGGAGCCTGAGGAAGCAGAAGGGGCCACGGTCATGGCCGGT
CATCGGCGCGACGCTGGAGCAGCTGAGGAACTACTACCGGATGCACGACTGGCTCGTGGAG
TACCTGTCCAAGCACCGGACGGTCACCGTCGACATGCCCTTCACCTCCTACACCTACATCGC
CGACCCGGTGAACGTCGAGCACGTGCTCAAGACCAACTTCAACAATTACCCCAAGGTGAAA
CAATCCTCGAGATGTCAGTCAAGGTTCAGTATAATCGGTACTGACAGTGTTACAAATGTCTG
AAATCTGGAATTGTGTGTGTAGGGGGAGGTGTACAGGTCCTACATGGACGTGCTGCTCGGCG
ACGGCATATTCAACGCCGACGGCGAGCTCTGGAGGAAGCAGAGGAAGACGGCGAGCTTCGA
GTTCGCTTCCAAGAACTTGAGAGACTTCAGCACGATCGTGTTCAGGGAGTACTCCCTGAAGC
TGTCCAGCATCCTGAGCCAGGCTTGCAAGGCAGGCAAAGTTGTGGACATGCAGGTAACTGA
ACTCTTTCCCTTGGTCATATGAACGTTGATTTCTTGGACAAAATCTCAAGATTCTGACGCGAG
CGAGCCAATTCAGGAGCTGTACATGAGGATGACGCTGGACTCGATCTGCAAGGTGGGGTTC
GGGGTGGAGATCGGCACGCTGTCGCCGGAGCTGCCGGAGAACAGCTTCGCGCAGGCCTTCG
ACGCCGCCAACATCATCGTGACGCTGCGGTTCATCGACCCGCTGTGGCGCGTGAAGAAATTC
CTGCACGTCGGCTCGGAGGCGCTGCTGGAGCAGAGCATCAAGCTCGTCGACGAGTTCACCTA
CAGCGTCATCCGCCGGCGCAAGGCCGAGATCGTGCAAGCCCGGGCCAGCGGCAAGCAGGAG
AAGGTGCGTACGTGGTCATCGTCATTCGTCAAGCTCCCGATCGCTGGTTTGTGCAGATGCCA
CTGATCACTGACACATTAACTGGGCGCGCAGATCAAGCACGACATACTGTCGCGGTTCATCG
AGCTGGGCGAGGCCGGCGGCGACGACGGCGGCAGCCTGTTCGGGGACGACAAGGGCCTCCG
CGACGTGGTGCTCAACTTCGTCATCGCCGGGCGGGACACGACGGCCACGACGCTCTCCTGGT
TCACCTACATGGCCATGACGCACCCGGCCGTGGCCGAGAAGCTCCGCCGCGAGCTGGCCGC
CTTCGAGTCCGAGCGCGCCCGCGAGGATGGCGTCGCTCTGGTCCCCTGCAGCGACGGCGAG
GGCTCCGACGAGGCCTTCGCCGCCCGCGTGGCGCAGTTCGCGGGACTCCTGAGCTACGACGG
GCTCGGGAAGCTGGTGTACCTCCACGCGTGCGTGACGGAGACGCTCCGCCTGTACCCGGCGG
TGCCGCAGGACCCCAAGGGCATCGCGGAGGACGACGTGCTCCCGGACGGCACCAAGGTGCG
CGCCGGCGGGATGGTGACGTACGTGCCCTACTCCATGGGGCGGATGGAGTACAACTGGGGC
CCCGACGCCGCCAGCTTCCGGCCAGAGCGGTGGATCGGCGACGACGGCGCCTTCCGCAACG
CGTCGCCGTTCAAGTTCACGGCGTTCCAGGCGGGGCCGCGGATCTGCCTGGGCAAGGACTCG
GCGTACCTGCAGATGAAGATGGCGCTGGCCATCCTGTGCAGGTTCTTCAGGTTCGAGCTCGT
GGAGGGCCACCCCGTCAAGTACCGCATGATGACCATCCTCTCCATGGCGCACGGCCTCAAGG
TCCGCGTCTCCAGGGTGCCGCTCGCCTGATCTTGATCTGGTTCCGGCGACGGTGATGGACGC
TCCGGTGGCTGTCTGGCCAGACGGCCGGCGTGTTATGACAGGCTCGATTTAACTTAGCAATT
GTGATAAACTCGTATATGTAGGCAGAGTGGAGAGTGTGTTGATCGATTTGCCATGGACGTTG
CTCGTCCGTTGTTACCGTCGTACCATGTTTGTATTGCTTCTAGATCATTATAGTTCGTGTTTGT
TCTTGAGCCTAAGTATTTATTGCACATTTCAAAAATGACAAATGTGTGCAATTGTCTTTTTTG
GGTGTTTTCTAAGGGTAGTATTTTCGCAGATTTATTCTGTCGACCAAACCTTAGCCTTTGACC
CCTCTCGCCGTCGTCCGGATGCGACGTGGGCAGGAAGGCTGCTCCTCGTGGGGTGCCAGACA
TGTTGGAGCTGGTGGAATGTTGCAGGACAGCGACGGTGATGAGTGGTCAGATTGCCGTTGTC
GACAGGCGATGCTCGATGGTGGCGCTGGTGAAGGTGACGGTGGTCGGAGGATCACATATCC
AGCACGACGATCTTCAACAGCGGCCCGGCTTGGCTAGGTCATTGGACAAGCAATAATCCTAC
ACCTACGAAAATTGCTACGTAGGCTTACTTAACCTTTCATAAAATTCTCTCCTTCCCCGTGAC
TTTAACCGGGGTGGACCCCAGCTGCTAATCCTGGCCCAATTAGCAACCTCCACATCATCTTTT
ACGTCAGATCTATACGTAACATTACGTATGTGTAGCATTGCTCACAAGCTTGGACAAGAGGG
TATTGATGCATGGTCCTGGATGGTGACGAGCTCGACATCAGACCCAGAGCTATCATGCAACG
ATATGTGTTTTTTC
SEQ ID NO: 42 Ms26-D CDS
ATGAGCAGCCCCATGGAGGAAGCTCACGGCGGCATGCCGTCGACGACGGCCTTCTTCCCGCT
GGCAGGGCTCCACAAGTTCATGGCCATCTTCCTCGTGTTCCTCTCGTGGATCTTGGTCCACTG
GTGGAGCCTGAGGAAGCAGAAGGGGCCGAGGTCATGGCCGGTCATCGGCGCGACGCTGGAG
CAGCTGAGGAACTACTACCGGATGCACGACTGGCTCGTGGAGTACCTGTCCAAGCACCGGA
CGGTGACCGTCGACATGCCCTTCACCTCCTACACCTACATCGCCGACCCGGTGAACGTCGAG
CATGTGCTCAAGACCAACTTCAACAATTACCCCAAGGGGGAGGTGTACAGGTCCTACATGG
ACGTGCTGCTCGGCGACGGCATATTCAACGCCGACGGCGAGCTCTGGAGGAAGCAGAGGAA
GACGGCGAGCTTCGAGTTCGCTTCCAAGAACTTGAGAGACTTCAGCACGATCGTGTTCAGGG
AGTACTCCCTGAAGCTGTCCAGCATACTGAGCCAGGCTTGCAAGGCCGGCAAAGTTGTGGAC
ATGCAGGAGCTGTATATGAGGATGACGCTGGACTCGATCTGCAAAGTGGGGTTCGGAGTCG
AGATCGGCACGCTGTCGCCGGAGCTGCCGGAGAACAGCTTCGCGCAGGCGTTCGACGCCGC
CAACATCATCGTGACGCTGCGGTTCATCGACCCGCTGTGGCGCGTGAAGAAGTTCCTGCACG
TCGGCTCGGAGGCGCTGCTGGAGCAGAGCATCAAGCTCGTCGACGAGTTCACCTACAGCGTC
ATCCGCCGGCGCAAGGCCGAGATCGTGCAGGCCCGGGCCAGCGGCAAGCAGGAGAAGATC
AAGCACGACATACTGTCGCGGTTCATCGAGCTGGGCGAGGCCGGCGGCGACGACGGCGGCA
GTCTGTTCGGGGACGACAAGGGCCTCCGCGACGTGGTGCTCAACTTCGTGATCGCCGGGCGG
GACACCACGGCCACGACGCTGTCCTGGTTCACCTACATGGCCATGACGCACCCGGACGTGGC
CGAGAAGCTCCGCCGCGAGCTGGCCGCCTTCGAGGCGGAGCGCGCCCGCGAGGATGGCGTC
GCTCTGGTCCCCTGCGGCGACGGCGAGGGCTCCGACGAGGCCTTCGCTGCCCGCGTGGCGCA
GTTCGCGGGGTTCCTGAGCTACGACGGCCTCGGGAAGCTGGTGTACCTCCACGCGTGCGTGA
CGGAGACGCTGCGCCTGTACCCGGCGGTGCCGCAGGACCCCAAGGGCATCGCGGAGGACGA
CGTGCTCCCGGACGGCACCAAGGTGCGCGCCGGCGGGATGGTGACGTACGTGCCCTACTCC
ATGGGGCGGATGGAGTACAACTGGGGCCCCGACGCCGCCAGCTTCCGGCCGGAGCGGTGGA
TCGGCGACGACGGCGCCTTCCGCAACGCGTCGCCGTTCAAGTTCACGGCGTTCCAGGCGGGG
CCGCGGATTTGCCTCGGCAAGGACTCGGCGTACCTGCAGATGAAGATGGCGCTGGCAATCCT
GTGCAGGTTCTTCAGGTTCGAGCTCGTGGAGGGCCACCCCGTCAAGTACCGCATGATGACCA
TCCTCTCCATGGCGCACGGCCTCAAGGTCCGCGTCTCCAGGGCGCCGCTCGCCTGA
SEQ ID NO: 43 Ms26-D AA
MSSPMEEAHGGMPSTTAFFPLAGLHKFMAIFLVFLSWILVHWWSLRKQKGPRSWPVIGATLEQL
RNYYRMHDWLVEYLSKHRTVTVDMPFTSYTYIADPVNVEHVLKTNFNNYPKGEVYRSYMDVL
LGDGIFNADGELWRKQRKTASFEFASKNLRDFSTIVFREYSLKLSSILSQACKAGKVVDMQELY
MRMTLDSICKVGFGVEIGTLSPELPENSFAQAFDAANIIVTLRFIDPLWRVKKFLHVGSEALLEQSI
KLVDEFTYSVIRRRKAEIVQARASGKQEKIKHDILSRFIELGEAGGDDGGSLFGDDKGLRDVVLN
FVIAGRDTTATTLSWFTYMAMTHPDVAEKLRRELAAFEAERAREDGVALVPCGDGEGSDEAFA
ARVAQFAGFLSYDGLGKLVYLHACVTETLRLYPAVPQDPKGIAEDDVLPDGTKVRAGGMVTYV
PYSMGRMEYNWGPDAASFRPERWIGDDGAFRNASPFKFTAFQAGPRICLGKDSAYLQMKMALA
ILCRFFRFELVEGHPVKYRMMTILSMAHGLKVRVSRAPLA
SEQ ID NO:44 M26-D genomic
CTTTGTAGAGATTTCACTATGAACCACATACGGATGTATATAAATGCATTTTAGAAGTAGAT
TCACTCATTTTGCTCCATATGTAGTCCATAGTGAAACCTCTACAAAGACTTGTATTTAGGACG
GATGGAGCAATAAATAGAAGGTGATCATTTTCATCAAAAATTTCATTTGTTTGGTCCTGTTA
AAAAATTCTAATTAATATAGAAGGGGGAAACTTTCAACAATATTTTCCATCTTTGTGATTTTC
AGGCTTTTTCCATTTAGGGAGAACATCAGAGCACCCCTTGACACCCCTTCATTCCAAATTTCT
CAACTTGCTCTGCTTTTGACTTCAAAAACTATTGGTTAGTGCGGGTTCATTAAAGATCAGATG
GACCATAACCATGGCTCCAACTGTGAAGATGAGATCATCACAGTGCTAATTGTCAAAAAAAT
GCATCACCTACATCCCCCGCAAAAGAAAATAAAAATGCATCACCTACATGTACAGTATTTTC
TTGGTCAGCAGATCACAAAAAGGACACAAACGGTAGGTTCCATCTAGTCAGGGGGTTAGGT
TAGGGACACCATGTGGATGAGGCAATCTTAATTCTCGGCCACACCAAGATTGTTTGGTGCTC
GGCAGCACTAATGCCCAATATATTACCTAACCGAGCCATCCAAATGCTACATACAGTTAATC
CTCCTGTAGACTGAACCCCCTTCATGAGCAGCCCCATGGAGGAAGCTCACGGCGGCATGCCG
TCGACGACGGCCTTCTTCCCGCTGGCAGGGCTCCACAAGTTCATGGCCATCTTCCTCGTGTTC
CTCTCGTGGATCTTGGTCCACTGGTGGAGCCTGAGGAAGCAGAAGGGGCCGAGGTCATGGC
CGGTCATCGGCGCGACGCTGGAGCAGCTGAGGAACTACTACCGGATGCACGACTGGCTCGT
GGAGTACCTGTCCAAGCACCGGACGGTGACCGTCGACATGCCCTTCACCTCCTACACCTACA
TCGCCGACCCGGTGAACGTCGAGCATGTGCTCAAGACCAACTTCAACAATTACCCCAAGGTG
AAACAATCCTCGAGATGTCAGTAAAGGTTCAGTATAATCGGTACTGACAGTGTTACAAATGT
CTGAAATCTGAAATTGTATGTGTAGGGGGAGGTGTACAGGTCCTACATGGACGTGCTGCTCG
GCGACGGCATATTCAACGCCGACGGCGAGCTCTGGAGGAAGCAGAGGAAGACGGCGAGCTT
CGAGTTCGCTTCCAAGAACTTGAGAGACTTCAGCACGATCGTGTTCAGGGAGTACTCCCTGA
AGCTGTCCAGCATACTGAGCCAGGCTTGCAAGGCCGGCAAAGTTGTGGACATGCAGGTAAC
TGAACTCATTCCCTTGGTCATCTGAACGTTGATTTCTTGGACAAAATTTCAAGATTCTGACGC
GAGCGAGCGAATTCAGGAGCTGTATATGAGGATGACGCTGGACTCGATCTGCAAAGTGGGG
TTCGGAGTCGAGATCGGCACGCTGTCGCCGGAGCTGCCGGAGAACAGCTTCGCGCAGGCGT
TCGACGCCGCCAACATCATCGTGACGCTGCGGTTCATCGACCCGCTGTGGCGCGTGAAGAAG
TTCCTGCACGTCGGCTCGGAGGCGCTGCTGGAGCAGAGCATCAAGCTCGTCGACGAGTTCAC
CTACAGCGTCATCCGCCGGCGCAAGGCCGAGATCGTGCAGGCCCGGGCCAGCGGCAAGCAG
GAGAAGGTGCGTGCGTGGTCATCGTCATTCGTCAAGCTCCCGGTCGCTGGTTTGTGTAGATG
CCATGGATCACTGACACACTAACTGGGCGCGCAGATCAAGCACGACATACTGTCGCGGTTCA
TCGAGCTGGGCGAGGCCGGCGGCGACGACGGCGGCAGTCTGTTCGGGGACGACAAGGGCCT
CCGCGACGTGGTGCTCAACTTCGTGATCGCCGGGCGGGACACCACGGCCACGACGCTGTCCT
GGTTCACCTACATGGCCATGACGCACCCGGACGTGGCCGAGAAGCTCCGCCGCGAGCTGGC
CGCCTTCGAGGCGGAGCGCGCCCGCGAGGATGGCGTCGCTCTGGTCCCCTGCGGCGACGGC
GAGGGCTCCGACGAGGCCTTCGCTGCCCGCGTGGCGCAGTTCGCGGGGTTCCTGAGCTACGA
CGGCCTCGGGAAGCTGGTGTACCTCCACGCGTGCGTGACGGAGACGCTGCGCCTGTACCCGG
CGGTGCCGCAGGACCCCAAGGGCATCGCGGAGGACGACGTGCTCCCGGACGGCACCAAGGT
GCGCGCCGGCGGGATGGTGACGTACGTGCCCTACTCCATGGGGCGGATGGAGTACAACTGG
GGCCCCGACGCCGCCAGCTTCCGGCCGGAGCGGTGGATCGGCGACGACGGCGCCTTCCGCA
ACGCGTCGCCGTTCAAGTTCACGGCGTTCCAGGCGGGGCCGCGGATTTGCCTCGGCAAGGAC
TCGGCGTACCTGCAGATGAAGATGGCGCTGGCAATCCTGTGCAGGTTCTTCAGGTTCGAGCT
CGTGGAGGGCCACCCCGTCAAGTACCGCATGATGACCATCCTCTCCATGGCGCACGGCCTCA
AGGTCCGCGTCTCCAGGGCGCCGCTCGCCTGATCTTGATCTGGTTCCGGCGACGGTGATGGA
CCTGGACGCTCCGGTGGCTGGCTGGCCGGACGGCCGGCGCGTTATGACACGCTCGATTTAAC
TTGGCAACTGTGATAAACTCGTATATGTAGGCAGAGTGGAGAGGGTATTGATCGATTTGCCA
TTGACGTTGCCCTACTCCATGGATGTTTGTATTGCCTCTAGATCATTATAGTTCGTGTTTGTTC
TTGAGCCTAAGTATTTATTGCACATTTCAAAATGACAAATGTATGCAATTGTCTTTTCTGGAT
GTTTTCTAAGGATTTTCGTAGATTTATTTTGTCGATCAAACCCTAGCCGTCACACATGATTCG
ATCCCTCTATGGGAGCTCGACACGGAGGAGCTGGTGAGCTGCTACAGGACGACGACGCTAA
TGAGTGGTCGAATTGCGGTTGTTGGCAGGCGATGCTCGATGGCGGCGTTGGTGAAGCCGGCG
GTGGTCGCAGGGTCACATATCCAGCGCGGCGATCTTCAACAGAGGCCCAACTTGGCCAGATC
ATCGGAGAAGAGGGCATCGATGCATGGTCCTGGATGGCGACGAGCTCGACATCAGACCCGC
ACCTATCATGCAGCGGCATGTTTGTTAGTCCTAATTTAGGAATAAGGTCCCCCTGGTCCGTTC
ATATGTTTATCCCGACGAAGGGCGTGTTGAGCCGTGTCTTTGATTTGTCTTCTGGGATTCGGT
TGGCTTAGATTTCGTGGTGGATTCATCTGGATTCAGACGACTTTCGTAGTCTACGAAATTCCT
ACAGGTCCTTATCGGCATTTTCTTCTCTGGGGCACCGATTTGATTCGTAGATCGTGGCCGCCG
GCATCTTCTAGTCTAGATCAACGACTTCCCTGACGCTGCTTCTACAAGCTTATGAGTTTTAAA
AAAGTTTGCTTC
SEQ ID NO: 45 Ms45-A CDS
ATGGAAGAGAAGAAGCCGCGGCGGCAGGGAGCCGCAGGACGCGATGGCATCGTGCAGTAC
CCGCACCTCTTCATCGCGGCCCTGGCGCTGGCCCTGGTCCTCATGGACCCCTTCCACCTCGGC
CCGCTGGCCGGGATCGACTACCGGCCGGTGAAGCACGAGCTGGCGCCGTACAGGGAGGTCA
TGCAGCGCTGGCCGAGGGACAACGGCAGCCGCCTCAGGCTCGGCAGGCTCGAGTTCGTCAA
CGAGGTGTTCGGGCCAGAGTCCATCGAGTTCGACCGCCAGGGCCGCGGGCCCTACGCCGGG
CTCGCCGACGGCCGCGTCGTGCGGTGGATGGGGGACAAGGCCGGGTGGGAGACGTTCGCCG
TCATGAATCCTGACTGGTCGGAGAAAGTTTGTGCTAACGGAGTGGAGTCGACGACGAAGAA
GCAGCACGGGAAGGAGAAGTGGTGCGGCCGGCCTCTCGGGCTGAGGTTCCACAGGGAGACC
GGCGAGCTCTTCATCGCCGACGCGTACTATGGGCTCATGGCCGTTGGCGAAAGCGGCGGCGT
GGCGACCTCCCTGGCGAGGGAGGCCGGCGGGGACCCGGTCCACTTCGCCAACGACCTCGAC
ATCCACATGAACGGCTCGATATTCTTCACCGACACGAGCACGAGATACAGCAGAAAGGACC
ATTTGAACATTTTGCTGGAAGGAGAAGGCACGGGGAGGCTGCTGAGATATGACCGAGAAAC
CGGTGCCGTTCATGTCGTGCTCAACGGGCTGGTCTTCCCAAACGGCGTGCAGATCTCACAGG
ACCAGCAATTTCTCCTCTTCTCCGAGACAACAAACTGCAGGATCATGAGGTACTGGCTGGAA
GGTCCAAGAGCGGGCCAGGTGGAGGTGTTCGCGAACCTGCCGGGGTTCCCCGACAACGTGC
GCTTGAACAGCAAGGGGCAGTTCTGGGTGGCGATCGACTGCTGCCGGACGCCGACGCAGGA
GGTGTTCGCGCGGTGGCCGTGGCTGCGGACCGCCTACTTCAAGATCCCGGTGTCGATGAAGA
CGCTGGGGAAGATGGTGAGCATGAAGATGTACACGCTTCTCGCGCTCCTCGACGGCGAGGG
GAACGTGGTCGAGGTACTCGAGGACCGGGGCGGCGAGGTGATGAAGCTGGTGAGCGAGGTG
AGGGAGGTGGACCGGAGGCTGTGGATCGGGACCGTTGCGCACAACCACATCGCCACGATCC
CTTATCCGTTGGACTAG
SEQ ID NO: 46 Ms45-A AA
MEEKKPRRQGAAGRDGIVQYPHLFIAALALALVLMDPFHLGPLAGIDYRPVKHELAPYREVMQ
RWPRDNGSRLRLGRLEFVNEVFGPESIEFDRQGRGPYAGLADGRVVRWMGDKAGWETFAVMN
PDWSEKVCANGVESTTKKQHGKEKWCGRPLGLRFHRETGELFIADAYYGLMAVGESGGVATSL
AREAGGDPVHFANDLDIHMNGSIFFTDTSTRYSRKDHLNILLEGEGTGRLLRYDRETGAVHVVL
NGLVFPNGVQISQDQQFLLFSETTNCRIMRYWLEGPRAGQVEVFANLPGFPDNVRLNSKGQFWV
AIDCCRTPTQEVFARWPWLRTAYFKIPVSMKTLGKMVSMKMYTLLALLDGEGNVVEVLEDRG
GEVMKLVSEVREVDRRLWIGTVAHNHIATIPYPLD
SEQ ID NO: 47 Ms45-A genomic
AGGACAGACGCTTAATTAGACGTTTCTCCTGTAGAAATAGGCACAAATGCTTCAAAAAAATC
CGATTTGTTTTTATAAGCACCTAGCATTGTACGAGGCCTTACGTATTTGTTGGGTGCTTAAAA
AGGAAGAGAAAGAAAGAAAGAAAGCGATCTAGAAATTTAAACACTGAAGGGACCCATGTC
GTCACCCTAGGGCCTTCCGAAACGTAGGACCGACCCTACACGCACCGCATTACGCCAATTAT
CTCTCCCTCTAATCCCCTTATAATTACCTCTATAACATCTGTCAATAACTAAATCATTATCAC
GAATGATACCGAATTCTTGACTGCTCCCTTGCTCTTCTGCTTCTTTCTCCTCCAAAGTTTGCTC
TTCTCTCCCTGATCCTGATCCTCACCAGATCAGGTCATGCATGATAATTGGCTCGGTATATCC
TCCTGGATCACTTTATGCTTGCTTTTTTTGAGAATCCACTTTATGCTTGTTGACCTGTACATCT
TGCATCACTATCCAAGCAACGAAGGCATGCAAATCCCAAATTCCAAAAGCGCCATATCCCCT
TAGCTGTTCTGAACCGAAATACACCTACTCCCAAACGATCACACCGACCCATGCAACCTCCG
TGCGTGCCGGGATAATATTGTCACGCTAGCTGACTCATGCAACTCCCGTGCATGTCGGTATA
TATTTTCGGGGCAAATCCATTAAGAATTTAAGATCACATTGCCCGCGCTTTTTTCGTCCGCAT
GCAAACTAGAGCCACTGCCCTCTACCTCCATGGAAGAGAAGAAGCCGCGGCGGCAGGGAGC
CGCAGGACGCGATGGCATCGTGCAGTACCCGCACCTCTTCATCGCGGCCCTGGCGCTGGCCC
TGGTCCTCATGGACCCCTTCCACCTCGGCCCGCTGGCCGGGATCGACTACCGGCCGGTGAAG
CACGAGCTGGCGCCGTACAGGGAGGTCATGCAGCGCTGGCCGAGGGACAACGGCAGCCGCC
TCAGGCTCGGCAGGCTCGAGTTCGTCAACGAGGTGTTCGGGCCAGAGTCCATCGAGTTCGAC
CGCCAGGGCCGCGGGCCCTACGCCGGGCTCGCCGACGGCCGCGTCGTGCGGTGGATGGGGG
ACAAGGCCGGGTGGGAGACGTTCGCCGTCATGAATCCTGACTGGTATTGGCTTACTGCAGAA
AAACCATAGCTTACCTGTGTGTGTGCAAACTAAAATAGTTTTTTCGGAAAAAAAAAGGTCGG
AGAAAGTTTGTGCTAACGGAGTGGAGTCGACGACGAAGAAGCAGCACGGGAAGGAGAAGT
GGTGCGGCCGGCCTCTCGGGCTGAGGTTCCACAGGGAGACCGGCGAGCTCTTCATCGCCGAC
GCGTACTATGGGCTCATGGCCGTTGGCGAAAGCGGCGGCGTGGCGACCTCCCTGGCGAGGG
AGGCCGGCGGGGACCCGGTCCACTTCGCCAACGACCTCGACATCCACATGAACGGCTCGAT
ATTCTTCACCGACACGAGCACGAGATACAGCAGAAAGTGAGCGGAGTACTGCTGCCGATCT
CCTTTTTCTGTTCTTGAGATTTGTGTTTGACAAATGACTGATCATGCAGGGACCATTTGAACA
TTTTGCTGGAAGGAGAAGGCACGGGGAGGCTGCTGAGATATGACCGAGAAACCGGTGCCGT
TCATGTCGTGCTCAACGGGCTGGTCTTCCCAAACGGCGTGCAGATCTCACAGGACCAGCAAT
TTCTCCTCTTCTCCGAGACAACAAACTGCAGGTGAGATAAACTCAGGTTTTCAGTATGATCC
GGCTCGAGAGATCCAGGAACTGATGACGCCTTTATTAATCGGCTCATGCATGCACACTAGGA
TCATGAGGTACTGGCTGGAAGGTCCAAGAGCGGGCCAGGTGGAGGTGTTCGCGAACCTGCC
GGGGTTCCCCGACAACGTGCGCTTGAACAGCAAGGGGCAGTTCTGGGTGGCGATCGACTGC
TGCCGGACGCCGACGCAGGAGGTGTTCGCGCGGTGGCCGTGGCTGCGGACCGCCTACTTCA
AGATCCCGGTGTCGATGAAGACGCTGGGGAAGATGGTGAGCATGAAGATGTACACGCTTCT
CGCGCTCCTCGACGGCGAGGGGAACGTGGTCGAGGTACTCGAGGACCGGGGCGGCGAGGTG
ATGAAGCTGGTGAGCGAGGTGAGGGAGGTGGACCGGAGGCTGTGGATCGGGACCGTTGCGC
ACAACCACATCGCCACGATCCCTTATCCGTTGGACTAGAGTGTGTAGTGTCTCATTTGATTTG
CTGGTTTTATATTAGCAAGGAGGTGTATCAGTTTATGGTTTGCTTGTTTATTGGGTTCGTGTG
ATGATCATGTTGTGAATTTGACGATGGATTCTTTTTCTTTTGTGACAAGAACTCGGATCTTTA
TAAAAGCTCACGAGAAGTACAAGGCATAATAAAAATTACATTGAGATTCTAGAACTGTAAT
GCAATTGTTTGAGTTTTCATGTATATATGAATTGATCATGTTTTTTGATTTGTTTGTACACCAC
CTCGACATACAAGGACCAAAGAGTATAAGGACTTATAGTTCTACGCAACGAGCTCAACCTC
AAACGCATTGTCATCCCTTCTCTCCTTGAAATAAAAAAGCAATATTGATGCAAGCACCGCGC
CAGGGCGTTGGCCCTCTACAGCTTGACATGTGTCATCATCTACTTGGTTGCCACGTACATGCC
AATTTAGAAGTTTTTCTTAACTTTCTTTTTTCTATATTCATTGAGATTTACCGTTGAGGCCATG
GAAATATTCGAATGGGTCTCGGCCTGCCCACTCCAAATCTCCCGCTCCATCCCTTTCTTTGTT
CTTCTAGTCCAAACGGAAATATGAGAGAAGGTTAGAGTCTTGATTGTTGTGCCTAGAAAAAA
ACGATGCCTGAGTGGAGCCTGAGTGGGGGACCTTTTTTGCCTGGCCAGGCAAGCCTAGGCGT
GGGTGTTTGGTTCCTTCTCTAGGTGGTCAGTTTGTCCTTTAGCACTTAGATAAATTTTGTACT
GCGGGCCATACTGTTTATGACTCGCTATCAGCGCTAGGCAGGCAGCTGGCCAGGCAGAACA
AATAGAATGCCTGGGCGAGGCTAACCAGGTTGCCTGGGCCAAGCATGTTTCTTTCTTTTGTTT
TTTAAATCTAGACCAAGTTAATCACGTTGCATGGACTCCCATGCCAGGAAGATGTTTCATTTT
CTAGGACACCATCCAAATAAATA
SEQ ID NO: 48 Ms45-B CDS
ATGGAAGAGAAGAAGCCGCGGCGGCAGGGAGCCGCAGTACGCGATGGCATCGTGCAGTAC
CCGCACCTCTTCATCGCGGCCCTGGCGCTGGCCCTGGTCGTCATGGACCCCTTCCACCTCGGC
CCGCTGGCTGGGATCGACTACCGGCCGGTGAAGCACGAGCTGGCGCCATACAGGGAGGTCA
TGCAGCGCTGGCCGAGGGACAACGGCAGCCGCCTCAGGCTCGGCAGGCTCGAGTTCGTCAA
CGAGGTGTTCGGGCCGGAGTCCATCGAGTTCGACAGCCAGGGCCGCGGGCCCTACGCCGGG
CTCGCCGACGGCCGCGTCGTGCGGTGGATGGGGGACAAGACCGGGTGGGAGACGTTCGCCG
TCATGAATCCTGACTGGTCGGAGAAAGTTTGTGCTAACGGAGTGGAGTCAACGACGAAGAA
GCAGCACGGGAAGGAGAAGTGGTGCGGCCGGCCTCTCGGGCTGAGGTTCCACAGGGAGACC
GGCGAGCTCTTCATCGCCGACGCGTACTATGGGCTCATGGCCGTCGGCGAAAGCGGCGGCGT
GGCGACCTCCCTGGCAAGGGAGGTCGGCGGGGACCCGGTCCACTTCGCCAACGACCTCGAC
ATCCACATGAACGGCTCGATATTCTTCACCGACACGAGCACGAGATACAGCAGAAAGGATC
ATTTGAACATTTTGCTAGAAGGAGAAGGCACGGGGAGGCTGCTGAGATATGACCGAGAAAC
CGGTGCCGTTCATGTCGTGCTCAACGGGCTGGTCTTCCCAAACGGCGTGCAGATTTCACAGG
ACCAGCAATTTCTCCTCTTCTCCGAGACAACCAACTGCAGGATCATGAGGTACTGGCTGGAA
GGTCCAAGAGCGGGCCAGGTGGAGGTGTTCGCGAACCTGCCGGGGTTCCCCGACAACGTGC
GCCTGAACAGCAAGGGGCAGTTCTGGGTGGCGATCGACTGCTGCCGGACGCCGACGCAGGA
GGTGTTCGCGAGGTGGCCGTGGCTGCGGACCGCCTACTTCAAGATCCCGGTGTCGATGAAGA
CGCTGGGGAAGATGGTGAGCATGAAGATGTACACGCTTCTCGCGCTCCTCGACGGCGAGGG
GAACGTGGTGGAGGTGCTCGAGGACCGGGGCGGCGAGGTGATGAAGCTGGTGAGCGAGGT
GAGGGAGGTGGACCGGAGGCTGTGGATCGGGACCGTTGCGCACAACCACATCGCCACGATC
CCTTACCCGCTGGACTAG
SEQ ID NO: 49 Ms45-B AA
MEEKKPRRQGAAVRDGIVQYPHLFIAALALALVVMDPFHLGPLAGIDYRPVKHELAPYREVMQ
RWPRDNGSRLRLGRLEFVNEVFGPESIEFDSQGRGPYAGLADGRVVRWMGDKTGWETFAVMN
PDWSEKVCANGVESTTKKQHGKEKWCGRPLGLRFHRETGELFIADAYYGLMAVGESGGVATSL
AREVGGDPVHFANDLDIHMNGSIFFTDTSTRYSRKDHLNILLEGEGTGRLLRYDRETGAVHVVL
NGLVFPNGVQISQDQQFLLFSETTNCRIMRYWLEGPRAGQVEVFANLPGFPDNVRLNSKGQFWV
AIDCCRTPTQEVFARWPWLRTAYFKIPVSMKTLGKMVSMKMYTLLALLDGEGNVVEVLEDRG
GEVMKLVSEVREVDRRLWIGTVAHNHIATIPYPLD
SEQ ID NO: 50 Ms45-B genomic
TCTGTCACAAGTACGTATTCATCCATCCTAATTTTGTGTGTCCTATTCATGCCTAGGGTTCTC
ATGTATAAATTTCTAATTCTTCGTGTTCTCTTTTCTTCATAATTTTAGGATATTAGCCCGCCTT
ACAATGTTGTCTAAGACCCGTAAAAGAAACAATGTTCTCTAAGAAGCATTTGCCGGGTGCTT
AAAAAAGAAGAAAAGAAAGAAAGAAAGTGATCTGAAAATTCAAACACTGAAGGGGCCCAT
GTCGTCGACCTAGGGCCTTCCGAAACGTAGAACCAAACCTACACGCACCGCATTACGCCAAT
TATCTCTCCCTCTAATCCTCTGACAATTTCCTTTATAATGACTGTCAATAACTAAATCCTTATC
ACGAATGAGACCGAATTTTGCTCTTCTCTCCCTGTATCCTGATCCTCACCAGATCAGGTCATG
CATGATAATTGGCTCGGTATATCCTCCTGGATCACTTTATGCTTGTTGACCTGTACATCTTGC
ATCACTTTCCAAGCAACAAAGGCATGCAAGTCTCAAATTCCAAAAAGGCCATATCCCCTTAG
CTGTTCTGAACCGAAATACACCTACTCCCAAACGATCACACCGACCCATGCAACCTCCGTGC
ATGTCGGGATAATCTTGTGACGCTAGCTAACTCATGCAACTCCCGTGCATGTCGGAATATAT
TTTCGGGGCAAATCCATTAAGAATTTAAGATCACGTTGCCCGCGCTTTTTTCGTCTGCATGCA
AACGAGAACCACTGCCCTCTGCCTCCATGGAAGAGAAGAAGCCGCGGCGGCAGGGAGCCGC
AGTACGCGATGGCATCGTGCAGTACCCGCACCTCTTCATCGCGGCCCTGGCGCTGGCCCTGG
TCGTCATGGACCCCTTCCACCTCGGCCCGCTGGCTGGGATCGACTACCGGCCGGTGAAGCAC
GAGCTGGCGCCATACAGGGAGGTCATGCAGCGCTGGCCGAGGGACAACGGCAGCCGCCTCA
GGCTCGGCAGGCTCGAGTTCGTCAACGAGGTGTTCGGGCCGGAGTCCATCGAGTTCGACAGC
CAGGGCCGCGGGCCCTACGCCGGGCTCGCCGACGGCCGCGTCGTGCGGTGGATGGGGGACA
AGACCGGGTGGGAGACGTTCGCCGTCATGAATCCTGACTGGTAATTGGCTTACTGCAGATAA
ATCCATAGCTTACCTGTGTGTTTGCAAACTAAAATGATTTCTTGGGAAAAAAAAAGGTCGGA
GAAAGTTTGTGCTAACGGAGTGGAGTCAACGACGAAGAAGCAGCACGGGAAGGAGAAGTG
GTGCGGCCGGCCTCTCGGGCTGAGGTTCCACAGGGAGACCGGCGAGCTCTTCATCGCCGACG
CGTACTATGGGCTCATGGCCGTCGGCGAAAGCGGCGGCGTGGCGACCTCCCTGGCAAGGGA
GGTCGGCGGGGACCCGGTCCACTTCGCCAACGACCTCGACATCCACATGAACGGCTCGATAT
TCTTCACCGACACGAGCACGAGATACAGCAGAAAGTGAGCGGAGTACTGTCGCTGATCTCC
ATTTTTGTTCTTGAGATGTTGTGTTTGAGTGTCTGACACCATGACTGATCATGCAGGGATCAT
TTGAACATTTTGCTAGAAGGAGAAGGCACGGGGAGGCTGCTGAGATATGACCGAGAAACCG
GTGCCGTTCATGTCGTGCTCAACGGGCTGGTCTTCCCAAACGGCGTGCAGATTTCACAGGAC
CAGCAATTTCTCCTCTTCTCCGAGACAACCAACTGCAGGTGAGATAAACTCAGGTTTTCAGT
ATGATCCGGCTCGAGAGATCCAGGAACTGATGACGGATCATGCATGCACGCTAGGATCATG
AGGTACTGGCTGGAAGGTCCAAGAGCGGGCCAGGTGGAGGTGTTCGCGAACCTGCCGGGGT
TCCCCGACAACGTGCGCCTGAACAGCAAGGGGCAGTTCTGGGTGGCGATCGACTGCTGCCG
GACGCCGACGCAGGAGGTGTTCGCGAGGTGGCCGTGGCTGCGGACCGCCTACTTCAAGATC
CCGGTGTCGATGAAGACGCTGGGGAAGATGGTGAGCATGAAGATGTACACGCTTCTCGCGC
TCCTCGACGGCGAGGGGAACGTGGTGGAGGTGCTCGAGGACCGGGGCGGCGAGGTGATGAA
GCTGGTGAGCGAGGTGAGGGAGGTGGACCGGAGGCTGTGGATCGGGACCGTTGCGCACAAC
CACATCGCCACGATCCCTTACCCGCTGGACTAGAGGGAGTGTGCAGTGTCCATTTGCTGGTT
TATATTAGCAAGGAGGTGTATCAGTTTATGGTTTGCTTGTTTATTGGGTTCGTGTGATGATCA
TATTGTGAATTTGACGATGGATTCTTTTTCTTTTGTGACAAGAACTCTGATCTTTATAAAGGC
TCACGAGAAGTATATAAGCATAATAAAAATTATATCAAGGTCCTTGAATCGTCGAACAACCA
TTGCCGCCATCAGAACAAGCCGTTGTCGTCGCTTCTGCTGGAGCCGGCCTAATGTTGTAGAT
CAGCGCCTTCTAGTTGCAGTCGTCACCGTCAAAGCCTTGAATCGATCTAAAGAATCCTACAC
CAAATCTTGCCATCGCGTATGCACGACGAGAAACCCTAACCTCACCGCACCGAGAAGCTAG
CGGGAATCAAAGACAGGGCTCCATCTAATCCGCCCCTACTTACGAACTTGAGGAGGATCAA
AACCTATAGAAGAGTAATGATGAGTGGATTTCTCAGTCATTTTCATCCATGTTTAAACCGGA
TATTCTCAGATTTTTTCGAGATAATCACTTCAATTTGCCTACTAATGACTAAAATAATTGCAT
AAGATTGCAAATCACATTGATTATTTTATTTCATGCAAAAATTTGCTATTTTCGGTGATAAAT
TAGGCCATAAAAGGGACATAATGGCTCAAGATCAAACTCAATCAGTCGGAGCCGTGTAGCA
GCTTCCAGAGGAAGAGACAACATGCGGTACAAACATGGCTACTCGTATCGATACTCGTACC
AAGCGCCAACGACCCCATGACGTATCCCTAACGAC
SEQ ID NO: 51 Ms45-D CDS
ATGGAAGAGAAGAAACCGCGGCGGCAGGGAGCCGCAGTACGCGATGGCATCGTGCAGTAC
CCGCACCTCTTCATCGCGGCCCTGGCGCTGGCCCTGGTCCTCATGGACCCGTTCCACCTCGGC
CCGCTGGCCGGGATCGACTACCGACCGGTGAAGCACGAGCTGGCGCCGTACAGGGAGGTCA
TGCAGCGCTGGCCGAGGGACAACGGCAGCCGCCTCAGGCTCGGCAGGCTCGAGTTCGTCAA
CGAGGTGTTCGGGCCGGAGTCCATCGAGTTCGACCGCCAGGGCCGCGGGCCTTACGCCGGG
CTCGCCGACGGCCGCGTCGTGCGGTGGATGGGGGACAAGGCCGGGTGGGAGACGTTCGCCG
TCATGAATCCTGACTGGTCGGAGAAAGTTTGTGCTAACGGAGTGGAGTCGACGACGAAGAA
GCAGCACGGGAAGGAGAAGTGGTGCGGCCGGCCTCTCGGCCTGAGGTTCCACAGGGAGACC
GGCGAGCTCTTCATCGCCGACGCGTACTATGGGCTCATGGCCGTCGGCGAAAGGGGCGGCG
TGGCGACCTCCCTGGCGAGGGAGGCCGGCGGGGACCCGGTCCACTTCGCCAACGACCTTGA
CATCCACATGAACGGCTCGATATTCTTCACCGACACGAGCACGAGATACAGCAGAAAGGAC
CATTTGAACATTTTGCTGGAAGGAGAAGGCACGGGGAGGCTGCTGAGATATGACCGAGAAA
CCGGTGCCGTTCATGTCGTGCTCAACGGGCTGGTCTTCCCAAACGGCGTGCAGATATCACAG
GACCAGCAATTTCTCCTCTTCTCCGAGACAACAAACTGCAGGATCATGAGGTACTGGCTGGA
AGGTCCAAGAGCGGGCCAGGTGGAGGTGTTCGCGAACCTGCCGGGGTTCCCCGACAATGTG
CGCCTGAACAGCAAGGGGCAGTTCTGGGTGGCCATCGACTGCTGCCGTACGCCGACGCAGG
AGGTGTTCGCGCGGTGGCCGTGGCTGCGGACCGCCTACTTCAAGATCCCGGTGTCGATGAAG
ACGCTGGGGAAGATGGTGAGCATGAAGATGTACACGCTTCTCGCGCTCCTCGACGGCGAGG
GGAACGTCGTGGAGGTGCTCGAGGACCGGGGCGGCGAGGTGATGAAGCTGGTGAGCGAGGT
GAGGGAGGTGGACCGGAGGCTGTGGATCGGGACCGTTGCGCACAACCACATCGCCACGATC
CCTTACCCGCTGGACTAG
SEQ ID NO: 52 Ms45-D AA
MEEKKPRRQGAAVRDGIVQYPHLFIAALALALVLMDPFHLGPLAGIDYRPVKHELAPYREVMQ
RWPRDNGSRLRLGRLEFVNEVFGPESIEFDRQGRGPYAGLADGRVVRWMGDKAGWETFAVMN
PDWSEKVCANGVESTTKKQHGKEKWCGRPLGLRFHRETGELFIADAYYGLMAVGERGGVATS
LAREAGGDPVHFANDLDIHMNGSIFFTDTSTRYSRKDHLNILLEGEGTGRLLRYDRETGAVHVV
LNGLVFPNGVQISQDQQFLLFSETTNCRIMRYWLEGPRAGQVEVFANLPGFPDNVRLNSKGQFW
VAIDCCRTPTQEVFARWPWLRTAYFKIPVSMKTLGKMVSMKMYTLLALLDGEGNVVEVLEDR
GGEVMKLVSEVREVDRRLWIGTVAHNHIATIPYPLD
SEQ ID NO: 53 Ms45-D genomic
AGGCTTTCTTTAAGTATCGGTGCTTATTTGTACAGGTCAGACGCTTAATTAGGCGTCTCTCCT
GTAGAAATAGGCACCGATGCTTCAAAAAAAAACCCGCTCTATTTTTCTAAGCACATAACATT
GTACAAGACCTTAAGCATTTGTCGGGTGCTTAAAAGAAAGAAAAAGAAAGAAAGAATGCGA
TCTGAAAATTTAAACACTGAAGGGACCCATGTCGTCGCCCTAGGGCCTTCCTAAACGTAGGA
CCGACCCTGCATGCACCGCATTACGCCAATTATCTCTCCCTCTAATCTTCTTACAATTATCTC
CATAACAACTGCTAATAACTAAATCATTATCACGAATGAGGCTGAATTCTTGACTTCTCCCTT
GCTCTTCTGCTTCTTTCTCCTCCAAAGTTTGCTCTTCTCTCCCTGTATACTGATCCTCACCAGA
TCAGGTCATGCATGAAAATTGGCTCGGTATCCTCCTGGATCACTTTATGCTTGTTGACCTGTA
CATCTTGCATCACTATCCAAGCAACGAAGGCATGCAAGTCCCAAATTCCAAAAGCGCCATAT
CCCCTTAGCTGTTCTGAACCGAAATACACCTACTCCCAAACGATCACACCGACCCATGCAAC
CTCCGTGCGTGTCGGGATAATCTTGTGACGCTAGCTGACTCATGCAACTCCCGTGCGTGTCG
GAATATATTTTCGGAGCAAATCCATTAAGAATTTAAGATCACATTGCCCGCGCTTTTTTCGTC
TGCATGCAAAACAGAGCCACTGCCCTCTACCTCCATGGAAGAGAAGAAACCGCGGCGGCAG
GGAGCCGCAGTACGCGATGGCATCGTGCAGTACCCGCACCTCTTCATCGCGGCCCTGGCGCT
GGCCCTGGTCCTCATGGACCCGTTCCACCTCGGCCCGCTGGCCGGGATCGACTACCGACCGG
TGAAGCACGAGCTGGCGCCGTACAGGGAGGTCATGCAGCGCTGGCCGAGGGACAACGGCAG
CCGCCTCAGGCTCGGCAGGCTCGAGTTCGTCAACGAGGTGTTCGGGCCGGAGTCCATCGAGT
TCGACCGCCAGGGCCGCGGGCCTTACGCCGGGCTCGCCGACGGCCGCGTCGTGCGGTGGAT
GGGGGACAAGGCCGGGTGGGAGACGTTCGCCGTCATGAATCCTGACTGGTACTGGCTTACT
GCAGAAAAACCCATAGCTTACCTGTGTGTGTGCAGACTAAAATAGTTTCTTTCATAAAAAAA
AGGTCGGAGAAAGTTTGTGCTAACGGAGTGGAGTCGACGACGAAGAAGCAGCACGGGAAG
GAGAAGTGGTGCGGCCGGCCTCTCGGCCTGAGGTTCCACAGGGAGACCGGCGAGCTCTTCA
TCGCCGACGCGTACTATGGGCTCATGGCCGTCGGCGAAAGGGGCGGCGTGGCGACCTCCCT
GGCGAGGGAGGCCGGCGGGGACCCGGTCCACTTCGCCAACGACCTTGACATCCACATGAAC
GGCTCGATATTCTTCACCGACACGAGCACGAGATACAGCAGAAAGTGAGCGGAGTACTGCT
GCCGATCTCCTTTTTCTGTTCTTGAGATTTGTGTTTGACAAATGACTGATCATGCAGGGACCA
TTTGAACATTTTGCTGGAAGGAGAAGGCACGGGGAGGCTGCTGAGATATGACCGAGAAACC
GGTGCCGTTCATGTCGTGCTCAACGGGCTGGTCTTCCCAAACGGCGTGCAGATATCACAGGA
CCAGCAATTTCTCCTCTTCTCCGAGACAACAAACTGCAGGTGAGATAAACTCAGGTTTTCAG
TATGATCCGGCTCGAGAGATCCAGGAACTGATGACGGCTCATGCATGCACACTAGGATCATG
AGGTACTGGCTGGAAGGTCCAAGAGCGGGCCAGGTGGAGGTGTTCGCGAACCTGCCGGGGT
TCCCCGACAATGTGCGCCTGAACAGCAAGGGGCAGTTCTGGGTGGCCATCGACTGCTGCCGT
ACGCCGACGCAGGAGGTGTTCGCGCGGTGGCCGTGGCTGCGGACCGCCTACTTCAAGATCCC
GGTGTCGATGAAGACGCTGGGGAAGATGGTGAGCATGAAGATGTACACGCTTCTCGCGCTC
CTCGACGGCGAGGGGAACGTCGTGGAGGTGCTCGAGGACCGGGGCGGCGAGGTGATGAAG
CTGGTGAGCGAGGTGAGGGAGGTGGACCGGAGGCTGTGGATCGGGACCGTTGCGCACAACC
ACATCGCCACGATCCCTTACCCGCTGGACTAGAGGGAGTGTGTAGTGTCCCATTTGATTTGC
TGGTTTTATATTAGCAAGGAGGTGTATCAGTTTATGGTTTGCTTGTTCATTGGGTTCGTGTGA
TGATCATGTTGTGAATTTGACGGTGGATTCTTTTTCTTTTGTGACAAGAACTCGGATCTTTAT
AAATGCTCACGAGAAGTACAAAGCATAATAAAAAATTATATCAAGGTTCTAGAACTGTAAT
GCAATTGTTTGAGTTTTCATGTATATGAATTGATCATGTTTTTTGATCTATTTGTACACCACCT
CGACATACGAGGACCAAAGAGTACAAGGACTTATAGTTCTACGCGACGAGCTCAACCTCAA
ACGCATTGCCATCCCTTCTCTCCTTGAAATAAAAAATTATATATTTTTTGCAGGGAAATAAAA
AAACAATATTGATGTATGCATGGGCACGGCGTGCCGCCACGCCAGGGCGTTGGCCTTCTGCA
GCTTGGCATGTGTCCTCATCTACTTGGTTGCCATGCACAAGTCAATCTAGAAGTTTTTTTAAC
TTTCTTTTTTCTATATTCATTGAGATTTACCGTTTAGGCCATGGAAATATTTGAATGGGGCTC
AACCTGCCCACTCCCAATCTCTCGCTCCTTCGCTTTCTTCGTTCTTCCAGTCCAAACAGAAAG
ATGAGAGAAGGTTAGAGTCCTGAATGTTGTGTCTGGAAAAAAATGATGCTTGAGTGGAGCC
TGAGTGGGGGACCTTTTTTGCCTAGCCAGGCAAGCCTAGGTGTCGGTGTTTGGTTCCTTTCCT
GAGTGGTCGGTTTATCCTTTAGCGTGGGTGTTTGGTTCCTTCCCTGGGTG
SEQ ID NO: 54 TRIAE_CS42_7AS_TGACv1_569364_AA1814330.1 or
TraesCS7A01G014100.1 A genome (first gene following [from the distal end
of the chromosome] PV1-A)
CCACACCTAAGAAGACTAAAAAAACCTAACCTAAACTATTAACCAGACCGAAAGCACCGGG
ATCCCTAACCCCGCCACCAGTCGTCGGAGCGGCAGGTAGAGGGGAGGCGAATCCACGGTCT
CATTGATGAAGCCTGGAGGGGAGATTTACCATAGCCACCAAGGGATAGGGGAGTTCATATC
ACGGAAACCACACAATATTAATATGTCTTGGTGGTAAAAGGAATTGTTGCATCCTACATCTT
TGGAAATTGAAATGGAGAGGGTCTAACTATTGTAACTTTCATGAAATATGAAAGTTAATGAT
GCATGTCTTGATTTTCAGCGCAAAATAAAATGTAACATGTGGTGCTTGAGTATAGCACATAT
CCGAGTCCTAGAGTTTCAACCTGCCAACTACGAAACAAATTGGACTAAAAACTCACATAACC
TTTTATGTTACAAAAAAATGCATTTATGTGCCTTAAGAAAAAAACATAAAGTTGATATTCTG
ATGAGATTTTGAATGTTTGATTTGATTTTATTGGATTGCAGAGGGTGTTGGCATCCAGCAGA
AGAACCCTGGCTCTTTTCCAGATTTCCGCTCTGGTCGACTTCCTCATCCAAAAAAGTGGAAA
CTGCAAAGTTTTCATGGCAGCGGAAGTATTTAAAAAATAAAATCAAAGAGGGCTCAAGAAA
GTGCAAAATGCAAATTCTTTTGGGCAGCAGAAGGATTTTAAAAATAAAATCAAAGGAAAAA
ATGGAGATCCCTTAGCGGGCCTGGCCTGGCACCGGCCCGGTCCGGCGGGAAGCCGAGCCAC
CAATACTCTCACTCGCGCACCCGTTGGTGGCAGCCGCCGGCCCCCGTCGCCTCCTTCCCCTGA
ACCCTACTCCACCATGGCCGCCGCCCGCGCCGCCCTCCTCCGCCGCCACGGCCTCGGCGCCG
CCGCCACCAACCCCGTCCTCTTCTCCGGCCACGGCCTCCGCTACCGCAAGCTCGAGGTCATC
CTCACCACGGTAAGCCACCCCCCCTGCCCCCCTCCCCCCGCGACGCTCCGCTCGGTCCTCCAC
CTCTGCAGGATGCATCCCGCGATCTCGCTAGCCGCTTGCGTTTCCGGTCAGTTCGAGCGGGG
TGCTTTCGATCCGGTGAGGCCGGTGCCGCCTCAGCGACAGTTTGACCGATAAATCTCCAG
CATTTCTGAAACTTTTTAAGCTAGCAGTAGTATTTTTGACCGATAAAGCTTCAGTTTTGGCCT
TTCATTTCAACAATGTCCCTAGAGATTTTAGATTGTGCGGGGAAGTGGAACTAACCTTTGA
CATCACCCATCGCTTGTTCACCTCAGACGATCGACAAGCTGGGGAAGGCGGGGGAGACG
GTGAAGGTGGCGCCGGGGCACTTCCGCAACTACCTCATGCCCAAGATGCTCGCCGTCCCC
AACATCGACAAGTTCGCCATACTCATGCGCGAGCAGAGCAAGGTTAGCTTCCCCCTTCTTTT
CCCCGATAGAAATAAACATGCCGCGATGGCCGTGCAGTTTGGAATGCTCCGTCGCGGCTCAC
AAGCATTTGCTTACTAACTACTAGCTTACTCTGCTGGCTTTTGAGGCTCTGCTTTCCAGAGTT
CGTTTATCAGTTCTTCATCTGCGTATTGAATGAATTTTACGAGCTCTCCAATACTTGTTGCAT
CTTAGTAGCTCTTTGGTAGAATAGGCTTATATACAACTCTATGCTACTTGTGTTCAGTGAGCA
AGTTGGTGCTGAAGTTATTTTGGTTCATTCTAGTTCATCTCCACTGAAGTATTTGCTTCTTGTA
AAGTTCTGTTACCGTAAGAATATTTACATTGTAAACTATTACTGCTTTGCTGGCTTACAAGTC
TCTCATTTGTTTGATCAGCTTTACAAACGCGAAGTGGAGGTGGTTGTCAAAGAAGTCTCAAA
GGAGGAGGATGATGCTCGGGTATGTCGTGCACCGAGTGGTTCTTCCTTTTTCGTGAACTGCA
GTGTAGATGAGAGTTCTTAGTACACAACTAACTGACCTATCCTGTTCCTTTTCTGAAACCCTG
TTTGCCATTCAACAGAACTTTTGCCTACAGCTGTCTTTGGTTGATTTGTGCACAATTTCTGTCT
AACTGACCTCTCCTATTCATCCTCTGAAACTCAGTTTGCTATTTAACACCATATCAGTCCAGA
GCCGTCTTTGGTGGATTTGTTTTGGTTAACGCACTTCGAGTCCACTCTAAACATCTGGTGCTG
AAAATAGTGAATATGTTGCCTTGGAAAAGTCTTCTTGTACCTCTTGGCTTCTGCTCCCTCTGT
TCCTAAATGTTTGTGTTTCTAGAGATTTCAAATGGACTGCCACATATGGATGTATATAGACAT
ATTTTAGAGTGTAGATTCACTCATTTTGCTCTGTATGTAGGCACTTGTTGAAATTTCTAGAAA
GACAAATATTTAGGGACGGAGGAAGTAGATATTATGTTTAGAATGTAGGGCCTCTTAAGTTC
ATTTTTGTCACTGTGGCCAGCTACTCAACACTGTTCCAATACTGTTTTTCAGATACACAAGTA
TAGTAGCTGTCCTCTCAACTGTATCGTCCAGCTCATCCATTTCATACTGTACATAGAAAAGAT
CCTTTTGATGCTTACTTAAAACAACATTTTTTGTAACTGAATCCTGACATTGAAGTTCTTGTTT
TCTCAGTTCGGTTAAATACAGATCTTTTATGTGTTTTCCCAGTCACCGGCCTCTTAAGTTCATT
TCGTTTTAAAATGTTACTCATTTACTTCTCAACTCAACCATTTGAACAGCAAGCGGAAGAGA
AACTGAAGCAGTGTCAAGCAGCAGCAAAACGGCTCGATAATGCTCTCTTGGTTAGTATGGTT
CCACCAAATTTTGGAGTCCTGGTGCAGCATACTTTTGTTGATGTTCCAGATGGATAATCTTGT
CCACTATTAGCAAGTGCTGATCAAAATATTTATGTTTCATAGGTGTTCAGACGGTTCATCTCC
GAAGGGATCGAGTTGCGCTCTCCTGTAACGAAGGATGAAATTGTTTCTGAGGTTCTGCTCCA
GCGCTGTCATCCGCTATTTATTCAAAAACATTAATCTGTAGGTAGTAACATCAAACTTTACCA
ACAGGTGGCGAGGCAACTCAACGTCAACATTTACCCAGACAATTTACACCTGGTGTCACCAT
TGTCATCCCTCGGAGAATTTGAGGTGCCACTTCGCTTACCGAGGGATATACCACGCCCAGAA
GGCAAGCTACAATGGACTCTCAAGGTCAAGATCAGGAGACCCTAAGCACTTTCGGTGGGCG
ATCTTTGCTCTCCTTCCAAGGTGCTGAATGGTACCAAAAGGCCATTTCAGCCTTCGGATGAA
GAGACACGCTGAAATAGACCTTCAAACTCAACCTTTTCCCTTTACAATTTGCTTGGGCCATCG
TCTCGGGCGGGGCGATATGATCGGGCCTTTTGTTCCTACAAAAAAACGTTGAGAAATAGTGA
ATAATTTGCCTGGAGTAGGGGGCCTAGTATTGTTGTTGCTGTTTGACTTTATCATATTCTTGA
CTTGTTAGTGTGCCCAATCCTGGTGTGAAAGGGGGGAGATGGATATAAAGAAAGAAAGGTT
GTGTGTGCAAGGCATCCTTGAAAAGGAGAGGCAGGGAGTGAAAGCTTCCTCAGAAATGCTC
ATTTGGCGTCGTCATCATCATATGAAAAAATGGCCGTCTCCATCGACGTTTTAGTCGGCTTAT
ATTGCTCTACGTGTCGTGTGACGGCCTTTTGCTTTGATGTGGAAATGCTCTTTAATTCGCGCA
CGCATATTTTGTGCTTCCTTATCTCCCCCATTTGACTGAATGGTTATCAGTTGATCCATGGAC
CCCGGGCAATCATTGTCTTGGTCCTATTTTAAGATCTGAGCTGAATTACATTGACACTGACTT
GTCAGTGGAGACCCATTGATCTCTGCGATCTCTGCTTAATCTTGTTTCCCATTTTTTGCCAGG
CATTACTTTGAAAAAATTATTGCGGTAATTACGCGTCGACAAGGGCTATCTTTGCATCCAAA
GTGCTAATACAAAATGTTGAAAGAGAAGGGCACTGGTGCAAAAAATAAGAGTGAAAATCAG
CACTTTGGCAGTCTGATGAACTTTCATGTGGAGCTGGGGTGCCCAGATCCTCACTTTGCTTGA
GCACTGCAAAATACCTTTCCTATGCAGCAAGAGAAAGCTGTAAAGCAGGTGATCTCACCTGC
AAGGCATCAGGGTTGAGAAGCAACAGAGATGCCT
SEQ ID NO: 55 TRIAE_CS42_7DS_TGACv1_622424_AA2039410.1 or
TraesCS7D01G011300.1 D genome (following PV1-D)
GAAAAATTGTTGCATCCTACATCTTTGGAAATTGAAATGGAGAGGGTCTAACTATTGTAACT
TTCCTGAAATAGGAAAATTAATGATACATATCTTGATTTTCAGCGCAAAATAACATGTAACA
TGTGGTGCTTGAATATATCAGATAACCAAGTCCAAGAGTTTTGACTTGCCAACTATGAAACA
AATTGGACTAAAAACTCACATAACCTTTATGTTACGAAAAATAGCATTTATGTGCCTTCAGA
AAAAAGGCCTAAAGTTGATATTTTGATGAGATTTTGAATGTCTGATTTGATTTTTATTGGATT
GCGGAGGGTGCTGGCATCCGGCAGAAGAATCCCTACTCTTTTCCGGATTTCTTTCCTGGTCTA
CTACCTCGCTCAGGAAAGTGCAAATTGCAAAGTTTTCATGGCAGCAGAAGAATTTTGAAAAA
TAAATCAAAGGGACTCGAGATTTTTTTTAGTCTATCAAAGGGACTCAAGAAAATGCAAATGC
AAATTCTTTTGGGCAGCAGAAGTGTTTTAAAAATAAATTCAAAGGGAAAAAATGGAGATCC
CTTAGCGGGCCTGGCCTGGCACCGGCCCGGTCCGGCGGGAAGCCACCAATACTCTCCCTCGC
GCACCCGTTTTGTGGCAGCCGCCGGCCCCCTTCCCCTGAACCCTACTCCACCATGGCCGCCG
CCCGCGCCGCCCTCCTCCGCCGTCCCGGCCTCGGCGCCGCCGCCGCCAACCCCGTCCTCTTCT
CCGGCCACGGCCTCCGCTACCGCAAGCTCGAGGTCATCCTCACCACGGTAAGCCAGCCCCCC
TGCCCCCTCCCTCCCCTTCTCCTCCAAATCTCAACCCGCGACCCTCCGCTCGGTCCTCCACCT
CTGCAGGATGCATCCCGCTTGCGTTTCCGGTCAGTTCGAGCGGGATGTTTCCGATCCGGTGA
GGCCGGTGCCGCCTCAGCGACAGTTTGACCGATAAATCTTCAGCATTTCTGAAACTTTTTAA
GCTAGCAGTAGTATTCTTGACCGGTAAAGCTTCGGTTTTGGCTGTTCATTTCAACAATATCCC
TAGAGATTTTCAATGTGCCGGGAAGTGGAATTATTAACCTTTGCCATCGTCCATCGCTTGTTC
ACTTCAGACGATCGACAAGCTGGGGAAGGCGGGGGAGACGGTGAAGGTGGCGCCGGG
GCACTTCCGCAACTACCTCATGCCCAAGATGCTCGCCGTCCCCAACATCGACAAGTTCGCCA
TACTCATGCGCGAGCAGAGCAAGGTTAGCTTCCTCCTTTTCCCCGATAGAAATAAACATGCT
ACGATGGCCGTGCAGTTTeTGGAATGCTCGGTTGCAGCTCACAAGCATTACTTACTAGCTTAC
TCTTGTTGGCTTTTGAGGCTATGCTTTCCGGAGTTTCGTATATCAGTTCTGCCTCTGTGTATTG
TATGATTTGTACGAGTTCTCCAATAGTTGTTGCACCTTAGCTCTTTGGTAGAATAGCCTTATA
TGCAACTCTATGCTAGTGTTCAGTGAGGAAGTTGTACTGAAGTCATCGTGGTTCATTCTAGTT
CATCTCCACTGAAGTACGCTTCTTGTAGAGTTCAGTCACTGTAAGAATATTTGCAGTGTAAA
CTGTTACTTTTTAGGCTTACAAGTCTCTCATTTGTTTGATCAGCTTTACAAGCGTGAAGAGGA
GGTGGTTGTCAAAGAAGTCTCAAAGGAGGAGGATGATGCTCGGGTATGTCGTGCACTGATT
AGTTCTTCCTTTTTCATGAACTGAAGTGTCGATGAGTTCTTAGCACACAACTAACTGACCTGT
CCTGTTCCTTCTCTGAAACCCTGTTTGCCATTTAACACAATTTCTGCCCAGATCTGTCTTTGGA
GGATCTGTTGTTAACGCACTTCAAGCCCATTCTAAATCTGGTGTTGAAAATATTGAATATTTT
GCCTTGAAAAAGTCTTCTTCTACCTCTCGGCTTCTAGATATTATGTTTAGAATGTAGTGTATC
TTAAGTCCATTTGTCACTGTAGCCGACTACTCAACACTGTTCCAATACTGTTTTTCAGATATA
CAATTATAGTAGCTGTCCTCTGAACTGTAATGTGCATCTCATCCATTCCATACTGTACATATA
AAAGGTCCCTTTGATGCTTACTTAAAACCCATTTTTTTTAACTGAATACTCTGAGATTGAAGT
TATTGTTAAATGATGCTCCTAAAATTATTGGTTCGGTTAACTATAGATCTTGTATGTGTTTTC
CCTGTCATACTTCATTTTGTTTTTAACACCAAATTTCTCTTCCTTTTCTGAAACCCCGTTTGCC
ATTCAACACAATTTCTGTCTAGATCTGTCTTTGGTTGATTTGTACACAAAACTGACTTCTCCT
ATTCATCTTCTGAAACTCCGTTCCATATCAGTCCAGAGCTGTCTTCGCTGGATTTGTTTTGGTT
AACGCACTTCAAGCCCACTCTAAACATATGGTGCTGAAAATAGTGAAGATGTTGCCTTGGAA
AAGTCGTCTTCTATCTGTTGGCTTCTAGATATTATGTTTAGAATATAGTGCTTTTTAAGTTCAT
TTGTCACTGTAGCCAGCTAGTTAACACTGTTCCAATACTGTTTTTCAGATATACAAGTATAGT
ATCAGCTGTCCTCTGAACTGTAACGTCCAGCTCATCCAGTTCATACTGTACATAGAAAAGAT
CCTTTTGATGCTTACTTAAAACAACATTTTTTGTAACTGAATCCTGACATCGAAGTTCTTGTT
TTTGTATGCTTCTCAGTTCAGTTAAATACAGATCTTTATATGTGTTTTCCCAGCCATACTTCAT
TTTGTTTTTAAATGTTACTCATTTACTTCTGAACTCAACCATTTGAACAGCAAGCGGAAGAGA
AACTGAAGCAGTGTCAAGCAGCAGCAAAACGGCTCGATAATGCTCTTTTGGTTAGTATGGTT
CCACCAAATTTTGGAGTCCTGGTGCAGCATACTTTTGTTGATGTTCCAGATGGACAATTTTGT
CCAGTATTAGCACGTGCTGATCAAAATATTTATGTTTCATAGGTGTTCAGACGGTTCATCTCT
GAAGGGATCGAGTTGCGCTCTCCTGTAACAAAGGATGAAATTGTTTCTGAGGTTCTGCTCCA
GCGCTATCATCCGCTATTTATTCAAGAACATTAATCTGTAGGTAGTAACATCAAACTTTACCA
ACAGGTGGCAAGGCAACTCAATGTCAACATTTACCCAGACAATTTGCACCTGGTGTCACCAT
TGTCATCCCTTGGAGAATTCGAGGTGCCACTTCGCTTACCGAGGGCTATACCACGCCCAGAA
GGCAAGCTACAATGGACTCTCAAGGTCAAGATCAGGAGACCCTAAGCACTTTCGGTGGGCG
ATCTTGCTCTCCTTCCAAGGTGCTGAATTGTACCGAAAGACCGTTGCAGCCTTCAGATGAAG
AGACACGCTGAAATAGACCTTCAAACTCAACCTTTTCCCTTTACAATTTGCTTGGGCTATCGT
CTCGAGCGGGGCGATATGATGGGCCTTTCGTTCCTACAAACAAACGTTAAGAAATAGTGAAT
AATTTGCTTGGGGTAGGATGCCTAGCATTGTTGTTGCTGTTTGACTTTATCATATTCTTGACTT
GTTAGTGTGCCCAATCCTGGTGTGAAAGGGGGGAGATGGATATAAAGAAAGAAAGGTTGTG
TGTGCAAGGCATCCTTGAAAAGGAGAGGCCGGGAGTGAAAGCTTCCTCAGAAATGCTCATT
TGGCGTCGTCATCATCATATGAAAAAATGGCTGTCTCCATCGACGTTTTAGTCGGCTTATATT
TCTCTACGTGTCGTGCGACGGCCTTTTGCTTTGATGTGGAAATGCTCTTTAATTCGCGCACGC
ATATTTTGTGCCTCCTTATCTTCCCCATTTGACTGAATGGTTATCAGTTGATCCATGGACCCCT
GGCAATCATTGTCTTGGTCCTATTTTAAGATCTGAGCTGAATTACATTGACACTGACTGGACA
GTGGAGACCCATTGATCTCTGCTTAATCTTGTTTCCCATTTTTGCCAGCCATTACTTTGAAAA
AACTATTGTGGTAATTTACGTGTCGACAAGGGTTATCTTTGCATCCAAAGTAGTAATACAAA
ATGTTCAAAGAGAAGGGCACTGGTACAAAAAAATAAGAGTGAAAATCAGCACTTTGGCAGT
CTGATGAACTTTCATGTGGAGCTGGGGTGCCCAGATCCTCACTTTGCTTGAGCACTGCAAAA
TACCTTTCCTATGCAGCAAGAGAAAGCTGTAAAGCAGTGATCTCACCTGCAAGGCATCAGGG
TTGAGAAGCAACAGAGATGCCTTCTTTTGGGGAGGAAACAGCAGCCCAATTAGTAGCACTTC
ATTGTTAAGGTGCTGTTCAAGCTTCTTATATG
SEQ ID NO: 56 TRIAE_CS42_7AS_TGACv1_569258_AA1811670.1 or
TraesCS7A01G146100 .1 A genome (preceding Mfw 2-A)
CGCTGGCGCCAGAGAAGAGGCCCATCTCTGTTGTGGTTGTGGTGGCTAGGGTTTGCCGGCGA
CGAGGGAGCAAAGGATGGCAGATGTGGACGGCGAGTTTGGACAAGGACGGCCCCGCCGCA
CGGACGGCTTAAAAAGGACGAGCGTCATCGCTGACATGTGGGCCCGTCGTCATAAATTAAG
CTGACAGCGTGGACAACGGGTAGTTGGACGGCCGCCATGTGGGAACACGGCGGACAACAGG
AAGGCGCGCGAAGCGTCCGTTCGGCGTCCGCGCCGACGCATTTGGGGCGCAAATTTGGACC
GCAAATGCGTCGGCGCGGACATGACGCGGATGTGATTTGGGTTTGGGTCGCGCGTTGAGCCG
TCATTTTTGTCCGCGCCGACCCAAACGGGCGCAGGCGGATGAAATGAGTCGACCCTTTGGAG
TTGCTCTTAAGCGATGTTCAAGTGGGAGCTGTAATTTATCCCGCATTCGGAAATTATATTAAA
CCAATGGCAATGACCAAAATAAGATTTTACCAGTAAAACAAAAAGTCGTTCATGGGCAGGC
AAAGCCCAGCACGAATCTTGGCGGCTCGCATCCTCTATTGCGGCGCTGCATCATGGACACGC
CAGCCTGCCAAAGCCAAAGCCAAAGCGCCCCAATGCGATGCCACGAAAAAGCGATCAGCAT
CAGACACAGCCGCGCGACAATCTGCTAAAGAAACCCACATAAAAACGCGCAGCGCCCGGAA
CGCCGCGCGGCGACCACGGTGCCGTGCGGGGGTGTCTGCGTCTCTCTCTCCCCTCCCTCTCTC
CGCCGACGCGGCGCGGGCCGAGGGAATGGCCGCCGCCGCCTCCGCCTCCGCCTCCGCCTCGT
CTTCTTCCTCCACCTCCACCTCGGCCGGGTCCTCCGCGTCCACCTCCACGCCCCGGCCCGCCC
CGCGCCAGGCCGCCGCGGCGCCGTCGTCGTCCCCGGTCTTCCTCAACGTGTACGACGTGACC
CCGGCCAACGGGTACGCGCGGTGGCTGGGGCTCGGCGTGTACCACTCGGGCGTGCAGGTCC
ACGGGGTGGAGTACGCGTACGGCGCGCACGAGGGCGCCGGGAGCGGCATCTTCGAGGTGCC
CCCGCGGCGGTGCCCCGGCTACGCGTTCCGGGAGGCGGTGCTGGTGGGCACCACGGCGCTG
ACCCGCGCCGAGGTGCGCGCCCTCATGGCCGACCTCGCCGCCGACTTCCCGGGCGACGCCTA
CAACCTCGTCTCCCGCAACTGCAACCACTTCTGCGACGCCGCGTGCCGCCGCCTCGTCGCCC
GCGCCCGCATCCCGCGCTGGGTCAACCGCCTCGCCAAGATCGGGGTCGTCTTCACCTGCGTC
ATCCCCAGCAGCAGCAGGCACCAGGTGCGCCGCAAGGGGGAGCCGCAGCTGCCCGCCCCCG
TCAAGAGCCGCTCCGCGCGCCAGCCCGCCGCCCCGCCGCGGCCCAGGACCTTCTTCCGCTC
CCTCTCCGTCGGCGGCGGCAAGAACGTCACGCCCCGCCCGCTCCAGACCCCGCCGGTGGG
GCCGCCCCTGACGTTGACGACGCCGGCACCGACGCCGTTGGCCTCCATGTAACGGCGCCA
TTACTCCTTTTTCGTTTACAGCTCACACCATCCATTTTTTTTCCTTCGACAGTTACCTGAATTT
TGTCCATAGTACTGTACTCTTCGAGATTAAGATTTGTGCTCTGCTAGTGCTGCACTGTCACCA
TGATTAGCAGTAGTAACTGCAGTTCATTAGGCTATTAATTCCCGATTTTGTCTGGCTTTACTA
CCTAGACACACCTGGCTGGCTGTGTCCGCTGCCAAATCGCCATTAATGATTACTAATTTGGG
TCGCTGTTACGCGCTGCATTTACGTTGCGGTTAACGACGCCTATCATGCAATTGTTTTTGTTG
TGTGGCATGGATGCAATTCTATCCGGCGAGCCGTCCAATGGGAATATATTCGCTCCTCCTTTC
GCCCGTTCTTTGGAGTAAACAACCATGGAGCTGAAGCCTTGTTTGGATTTTCAACTATAGAT
AAAAGCTACACACAGGCTATGCACCGATCGGCCGATATGCTTTTGCTGATGCAAAGAATTCC
CCGTGTCTGGACAGTGGACCTGTCATCACTGCCGTTGTCATGGGACACGATTAGATTAGTCC
TCGTGTTGTTGTTTCTTGCATGATTGCGTCCGGCCTCCGTGCCTATCTGGAAATGCGGAGGGC
GGGATAATTTTAACGTGACTTGTCGCGTGAAAGGCGAGCTCGCTTCGACAGAAATCTTGGGG
AGCTCGCCGGTTGCGTGTCCAGCGCGCCTCGCCGTTGACCGGCGACCGGTGTGTCCATGC
CGGTGGCGAAGACGGCGGCGCGGGGTCAGAATTGGGCACCGACGGGAGGAGGGTTCGC
ATTTGTGGAGGACACCGCCACGCAGCACAGTGCACCACATTGGCCTTGACCCGTCCGATCAG
CGATCAGCGATCAGGATGGACGGGCCACTATCGATCCT
SEQ ID NO: 57 TRIAE_CS42_7DS_TGACv 1_622598_AA2042320.1 or
TraesCS7D01G147600.1 D genome (preceding Mfw 2-D)
GAGCCATCATTTCTACGGTCGGGCTGCTTTTGTAGGGTCAGCGTGTTTTCCGCGACAAATTCT
CATTGTGCCTATCCCGTCCCCCTTCGCCCACTAGGCAAGAACTCTCAGTGTCGTGACTAGGTT
TTGACGTGCAGAGAGTACACGACGCGTGATCGTGAGGCCAACACCCAACAGTATCCTAGGC
CCTAACGCATGGAAACCAAGTGACCGAGCGAGAAGAGAATGGAGGCCCAGAATCTTTGGTG
GAAAGAAACGACGTGGTTGTCATGTACTTATGCTGATTACAAAATTGCAAAGTCTGGTCAGA
ACCATCATTTGGTCGGCATTGAGAGTTTTCCTTCTTTTTGAATGGACAGGAATTGAGAGTTGA
TGGTGCATGGTGCCATTTAAGCGATGTTCAAACGGGAGCTGTAAATCATCCCGCATTCGGAA
ATTATTAAACCAATGGCAGTTCATGGGCAGGCAAAGCCCTGCACGAATCTTGGCGGCTCGCA
TCCTCTATTGCGGCGCTGCATCATGGACACGCCAGCCTGCCAAAGCCAAAGCCAAAGCGCCC
CAATGCGATGCCACGAAAAAGCGATCAGCGTCAGACACAGCCGCGCGACAAGCTGCTAAAG
AAACCCACATAAAAACGCGCAGCGCCCGGAACGCCGCGCGGCGACCACGGTGCCGTGCGGG
GGTGTCTGCGTCTCTCTCCCTCCTCTCTCTCTCCGCCGACGAGGCGCGAGGGAGTAAGGACG
CGCGCGCCGGCCGACGGCACGCGGGCCGAGGGAATGGCCGCCGCCGCCACCGCCACCGCCT
CCTCGTCCTCGTCAACCTCCTCCTCGGCCGGCTCCTCCGCGTCCACCTCCACGCCCCGGCCCG
CCCCGCGCCAGGCCGCCGCCGCGCCGTCGTCGTCCCCGGTGTTCCTCAACGTGTACGACGTG
ACCCCCGCCAACGGGTACGCGCGGTGGCTGGGGCTCGGCGTGTACCACTCGGGCGTGCAGG
TCCACGGCGTGGAGTACGCGTACGGCGCGCACGAGGGCGCCGGGAGCGGCATCTTCGAGGT
GCCCCCGCGGCGGTGCCCCGGCTACGCGTTCCGGGAGGCGGTGCTGGTGGGCACCACGGCG
CTGACCCGCGCCGAGGTGCGCGCGCTCATGGCCGACCTCGCCGCCGACTTCCCGGGCGACGC
CTACAACCTCGTCTCCCGCAACTGCAACCACTTCTGCGACGCCGCCTGCCGCCGCCTCGTCG
CCCGCGCCCGCATCCCGCGCTGGGTCAACCGCCTCGCCAAGATCGGGGTCGTCTTCACCTGC
GTCATCCCCAGCAGCAGCAGGCACCAGGTGCGCCGCAAGGGGGAGCAGCAGCTGCCCGCGG
CCGTCAAGAGCCGCTCCGCGCGCCAGGCCGCCGCCCCGCCGCGGCCCAGGACCTTCTTCCG
CTCCCTCTCCGTCGGCGGCGGCAAGAACGTCACGCCCCGCCCGCTCCAGACCCCGCCACC
GACGCCGCCGGTGGCCCCCGCCCTGACGTTGACGACGCCGACACCAACGCCGTTGGCCTC
CATGTAACGGCGCCATTACTCCTTTTTCGTTTACAGCTCACACCTTCCATTTTTTTTCCTTCGA
CAGTTACCTGAATTTTGTCCATAGTACTACTCTTCGAGATTAAGATTTGTGCTCTGCTAGTAG
TAGTACTGCACTGTCACCATGATTACCAGTAGTAACTGCAGTTCATTAGGCTATTAATTTCCG
AATTTGTCTGGCTTTACTACTACCTAGATACACCTGGCTGGCTGTGTGCCCGTGTCACCGTCT
GCTGCCAAATCGCCATTAATGATTACTAATTTGAGTCGCTGTTACGCGCTGCATTTACGTTGC
GGTTAACGACGTCTATCATGCAATTCTTTGTTGTGTGGCGTGGATCCAATTCTATCTGGCGAG
CCATCCAATAGGAATATATTCGCTCCTCCTTTCGCCCATTCTTTGGAATAAACAACCATTGTA
CTAGCTGAAGCCTTGCTTTGGATTTTCAACTAGATAAAGGCTCCAAAGCTAAGCACGGCCGA
TCGATATATGCTTTTGATGACGCAGAGAATTCCCGGTGTCTGGACACTCCACCTGTCATCAC
ACTGGCGTTGTCATGGGACACGATTAGATTAGTCCTCGTGTTGTTGTTTCTTGCATGATTGCG
TCCGGCCTCTGTGCCTATCTGGAAATGCGGAGGGAGGGATGATTTTAACGTGACCTGTCGCA
TGAAAGGCGAGCTTGCTTCGACAGAAATCTTGGGGAGCTCGCCGGTTGCGTGTCGAGCTCGC
CTCGCCGTTGACCGGCGGCGGCGGCGACCGGTGTGTCCATGCCGGTGGCGGAGACGGCGGC
GTAGGGTCAGAAGTGGGCACCGACGGGAGGAGGACTCGCGTTTGTGGAGGACACCAATGTG
CACCACATTGACCTTGACCCGTCCGATCAGCGATCAGGATGGACGGGCCACTATCGATCCTT
GGGCGGGCGTCGCTGGACCCCGGCCGGGCTGGGTTCGGTGCACGGGATGTGACGCCGCAGC
GGCGCCTTTCGATTTCGATCGGCTACAGGAGAGAAGTACGCTCGCTG

Guides to produce large deletions between the genes PV1 and Mfw2 in both A and D genomes (for subsequent selection for deletion in one genome or the other). The sequences are shown below and in bold in SEQ ID NOs 54, 55, 56 and 57.

Guides for proximal side of PV1 sequence—i.e., for the first gene following PV1-A and PV1-D (see SEQ ID NOs: 54 and 55).

SEQ ID NO: 58
ACGATCGACAAGCTGGGGAAGG
SEQ ID NO: 59
GCGGGGGAGACGGTGAAGGTGG
SEQ ID NO: 60
GACGGTGAAGGTGGCGCCGGGG

Guides for distal side of Mfw2 sequence—i.e., for the first gene preceding Mfw2-A and Mfw2-D.

SEQ ID NO: 61
ACGTTCTTGCCGCCGCCGACGG
SEQ ID NO: 62
GCGTCGTCAACGTCAGGGCGGG
SEQ ID NO: 63
GGGAGCGGAAGAAGGTCCTGGG

The reverse complements of SEQ ID Nos; 61-63 are shown in SEQ ID Nos; 64-66 and reflect the sequences as they appear, in the context of SEQ ID Nos: 56 and 57

SEQ ID NO: 64
CCGTCGGCGGCGGCAAGAACGT
SEQ ID NO: 65
CCCGCCCTGACGTTGACGACGC
SEQ ID NO: 66
CCCAGGACCTTCTTCCGCTCCC

Guides to produce a large deletion between the genes PV1 and Mfw2 in the A genome only are provided as SEQ ID NOs: 67-74 and are shown in bold above within the context of SEQ ID NOs 54, 55, 56 and 57.

Guides for the proximal side of PV1 sequence—i.e., for the first gene following PV1 (see also SEQ ID NOs 54 and 55)

SEQ ID NO: 67
GCTTTCGATCCGGTGAGGCCGG
(in SEQ ID NO: 54, first gene following PV1-A)
SEQ ID NO: 68
AGAGATTTTAGATTGTGCGGGG
(in SEQ ID NO: 54, first gene following PV1-A)
SEQ ID NO: 60
GACGGTGAAGGTGGCGCCGGGG
(in SEQ ID NO: 54, first gene following PV1-A
and in SEQ ID NO: 55, first gene following
PV1-D) (this cuts the D genome as well as the A)

Guides for the distal side of Mfw2-A sequence—i.e., for the first gene preceding Mfw2-A.

SEQ ID NO: 69
ATGCGAACCCTCCTCCCGTCGG
(reverse of the relevant forward genomic
sequence in SEQ ID NOs: 72 and 56)
SEQ ID NO: 70
GCGCCGCCGTCTTCGCCACCGG
(reverse of the relevant forward genomic
sequence in SEQ ID NOs: 73 and 56)
SEQ ID NO: 71
GGTCAACGGCGAGGCGCGCTGG
(reverse of the relevant forward genomic
sequence in SEQ ID NOs: 74 and 56)

The reverse complements of SEQ ID NOs 69, 70 and 71 above are shown in SEQ ID NOs; 72, 73 and 74 below and reflect the sequences in the context of the genomic sequence SEQ ID NO: 56, for the gene the distal side of Mfw2-A (where they appear in bold).

SEQ ID NO: 72
CCGACGGGAGGAGGGTTCGCAT
SEQ ID NO: 73
CCGGTGGCGAAGACGGCGGCGC
SEQ ID NO: 74
CCAGCGCGCCTCGCCGTTGACC

### Example 3: PV1 Knocked in at Mfw2 Locus in to Produce a PV1 Knock-in which is Linked to/Part of a Mfw2 Knockout and an OV1 Knocked in to the Neighbouring Gene to Mfw2

To produce plants with targeted insertion of PV1 and OV1 at a Mfw2 site and the gene after Mfw2 (gaMfw2) respectively, a CRISPR CAS system was used to introduce mutations and direct repair in wheat plants to introduce the genes PV1 and OV1. The guide locations for the insertion of PV1 and OV1 were chosen from the previous CRISPR knockout experiments of Mfw2 and the attempt to delete a large portion of chromosome 7A, (see, e.g., International Patent Application PCT/US2017/043009, which is incorporated by reference herein in its entirety).

For the insertion of PV1, a construct was made with PV1 cDNA driven by 1.5 kb of its own promoter with 800 bp of flanking sequence which matches the insertion site around the Mfw2 guide targeted sequence. This gene insertion with Mfw2 flanking sequence and guide sequence targeting GGATGGCCAATGCGAGATGATGG (SEQ ID NO: 75) driven by the TaU6 promoter was synthesized by Genewiz and subsequently cloned into an intermediate vector containing L1 L5r flanking sites for multisite gateway recombination. A second intermediate vector containing a wheat-optimized Cas9 enzyme driven by the maize ubiquitin promoter flanked by L5 and L2 was also produced for multisite gateway recombination. Both intermediary vectors were combined as part of a multisite gateway reaction into the final binary vector. This final vector was introduced into Agrobacterium for transformation into wheat as documented in Example 1.

Plants were then screened for insertion of the gene using a PCR based method where the PCR product was amplified for each homoeologue anchored to the possible insertion and sequenced to verify insertion. Plants were selected which had the PV1 insertion on the same homoeologue as the insertion of OV1 (as follows).

For the insertion of OV1, again an intermediate construct was made with OV1 cDNA driven by 1.5 kb of its own promoter with 800 bp of flanking sequence which matches the insertion site around the gaMFw2 guide targeted sequence. A second intermediate vector containing a wheat-optimized Cas9 enzyme driven by the maize ubiquitin promoter flanked by L5 and L2 was also produced for multisite gateway recombination. Both intermediary vectors were combined as part of a multisite gateway reaction into the final binary vector. This final vector was introduced into Agrobacterium for transformation into wheat as documented in Example 1.

Plants were then screened for insertion of the gene using a PCR based method where the PCR product was amplified for each homoeologue anchored to the possible insertion and sequenced to verify insertion of OV1. Plants were selected which had the OV1 insertion on the same homoeologue as the PV1 insertion above. Plants with an insertion of either PV1 or OV1 were then crossed to combine the inserted sequences in the same plant. This was a plant(s) containing both mfw2:PV1:gaMfw2 on one chromosome of the homologous pair selected and Mfw2:gamfw2:OV1 on the other.

When plants from the above experiment have their endogenous Mfw2, PV1 and OV1 genes knocked out in all loci except Mfw2 on the chromosomes containing the above constructs, this is the basis of the maintainer line. As only the chromosome with Mfw2:gamfw2:OV1 has gaMfw2 knocked out, all other five homoeologous/homologous alleles will express the product.

### Example 4: PV1 and OV1 Knocked-in at Two Homologous/Allelic Mfw2 Loci to Produce, after Appropriate Crossing and Selection, a PV1 Knock-in in One of the Homologous Loci and OV1 in the Other

To produce plants with targeted insertion of PV1 and OV1 at a Mfw2 site, a CRISPR CAS system was used to introduce mutations and direct repair in wheat plants to introduce the genes PV1 and OV1. The guide locations for the insertion of PV1 and OV1 were chosen from the previous CRISPR knockout experiments of Mfw2 and the attempt to delete a large portion of chromosome 7A, (see, e.g., International Patent Application PCT/US2017/043009, which is incorporated by reference herein in its entirety).

For the insertion of PV1, a construct was made with PV1 cDNA driven by 1.5 kb of its own promoter with 800 bp of flanking sequence which matches the insertion site around the Mfw2 guide targeted sequence. This gene insertion with Mfw2 flanking sequence and guide sequence targeting GGATGGCCAATGCGAGATGATGG (SEQ ID NO: 75) driven by the TaU6 promoter was synthesized by Genewiz and subsequently cloned into an intermediate vector containing L1 L5r flanking sites for multisite gateway recombination. A second intermediate vector containing a wheat-optimized Cas9 enzyme driven by the maize ubiquitin promoter flanked by L5 and L2 was also produced for multisite gateway recombination. Both intermediary vectors were combined as part of a multisite gateway reaction into the final binary vector. This final vector was introduced into Agrobacterium for transformation into wheat as documented in Example 1.

Plants were then screened for insertion of the gene using a PCR based method where the PCR product was amplified for each homoeologue anchored to the possible insertion and sequenced to verify insertion. Plants were selected which had the PV1 insertion on the same homoeologue as the insertion of OV1 (as follows).

For the insertion of OV1, again an intermediate construct was made with OV1 cDNA driven by 1.5 kb of its own promoter with 800 bp of flanking sequence which matches the insertion site around the gaMFw2 guide targeted sequence. A second intermediate vector containing a wheat-optimized Cas9 enzyme driven by the maize ubiquitin promoter flanked by L5 and L2 was also produced for multisite gateway recombination. Both intermediary vectors were combined as part of a multisite gateway reaction into the final binary vector. This final vector was introduced into Agrobacterium for transformation into wheat as documented in Example 1.

Plants were then screened for insertion of the gene using a PCR based method where the PCR product was amplified for each homoeologue anchored to the possible insertion and sequenced to verify insertion of OV1. Plants were selected which had the OV1 insertion on the same homoeologue as the PV1 insertion above. Plants with an insertion of either PV1 or OV1 were then crossed to combine the inserted sequences in the same plant. This was a plant(s) containing both mfw2:PV1 on one chromosome of the homologous pair selected and mfw2:OV1 on the other.

When plants from the above experiment have their endogenous PV1 and OV1 genes knocked out in all loci, this is the basis of the maintainer line. As only the chromosomes with the above knock-ins have Mfw2 knocked out, the other four homoeologous alleles will express the product.

1.-104. (canceled)

105. A plant or plant cell comprising a deactivating modification of at least one PV gene.

106. The plant or plant cell of claim 105, comprising deactivating modifications of each of the copy of the PV gene.

107. The plant or plant cell of claim 106, wherein the deactivating modification is identical across each genome of the plant.

108. The plant or plant cell of claim 106, wherein each genome of the plant comprises a different deactivating modification.

109. The plant or plant cell of claim 105, wherein the PV gene is PV1, Ms45, Ms1, Ms26, Mfw1, Mfw4, RPG1, Apv1, or Ipe1.

110. The plant or plant cell of claim 105, wherein the PV gene has at least 95% identity with PV1, Ms45, Ms1, Ms26, Mfw1, Mfw4, RPG1, Apv1, or Ipe1.

111. The plant or plant cell of claim 105, wherein the PV gene has the same activity and at least 95% identity with PV1, Ms45, Ms1, Ms26, Mfw1, Mfw4, RPG1, Apv1, or Ipe1.

112. The plant or plant cell of claim 105, wherein the deactivating modification is a site-directed mutagenic event resulting from the activity of a site-specific nuclease; or
the PV gene is deactivated by site-directed mutagenesis resulting from the activity of a site-specific nuclease.

113. The plant or plant cell of claim 112, wherein the site-specific nuclease is CRISPR-Cas.

114. The plant or plant cell of claim 105, wherein the deactivating modification is excision of at least part of a coding or regulatory sequence; or the PV gene is deactivated by excision of at least part of a coding or regulatory sequence.

115. The plant or plant cell of claim 105, wherein the deactivating modification is insertion of RNAi-encoding sequences; or the PV gene is deactivated by inhibition by expression of RNAi.

116. The plant or plant cell of claim 105, wherein the deactivating modification is non-transgenic mutagenesis; or the PV gene is deactivated by non-transgenic mutagenesis.

117. The plant or plant cell of claim 116, further comprising a deactivating modification of at least one OV or Mf gene.


SEQUENCE LISTING (75 sequences)

SEQ ID NO: 1 - DNA, 2025 bp, Triticum aestivum
atggcggagccggaggacggcggcgaggtcgcccctcctgaggcggcggcggcggcgacgagcgcggccgcccattcgtctccccctgctaaggaggagccggcggcagcggcagaggcaaagccggccagctccggcgaggcggtctccctcaactacgaggaagcgagagctctcttaggaaggctggaatttcagaaaggcaatgtagaagatgcactttgtgtgtttgatggaatagaccttcaagctgccattgagcgcttccagccatcatcctcgaagaaaacaacagaagctactcttgttctcgaagccatttacttgaaagcattgtcccttcagaagctaggaaaatcaatagaggccgctaaacaatgcaaaagcgtcatcgattctgttgaaagtatgttcaagaatggcactcctgacatcgaacagaagctacaagaaactatcaataaatctgtggaacttctcccagaggcctggaaaaaagctggctctcttcaggaaacatttgcttcgtacagacgcgctcttctcagcccgtggaacctcgacgaggaatgcattgcaaggattcaaaagagatttgctgctttcttgttgtatggttgtgtggagtggagtccgcccagctctggttcaccagctgaaggcacttttgttcccaagacaaatattgaggaagccattctactcctcacaacagtattgaagaagttttatcagggaaagacccactgggatccctcggtgatggaacacttgacctacgcattgtcgatttgcagccggccttctcttattgcagatcatctggaggaggttctacctgggatatatcctcggacggagagatggaacacactagcattttgctactatggtgttgctcagaaagaagtcgctctaaatttcctgaggaagtccttgaataagcatgagaacccaaaagatacaatggcattgctgttagccgccaagatatgtagcgaggactgccgtcttgcctccgagggtgtcgagtacgcaagaagagcgattgcaaacacggaatcattagatgttcatctgaagagcactggcctccatttcttggggagttgcctgagtaagaaggccaagattgtttcatccgatcaccaaagagctatgttgcacgcagaaactatgaagtcccttacggagtcgatgtctcttgaccgctacaacccaaacctaatattcgacatgggagttcaatacgctgagcagcggaacatgaacgccgcgctgagatgtgccaaagagtttgtcgacgcgaccggtggagcggtctcgaaaggttggaggtttctagccctagtcctctccgcacagcaaagatactccgaagcagaagtggcgaccaatgccgcgttagacgagaccgcaaagtgggatcaagggtcactgctcaggataaaggctaagctgaaggtcgctcaatcgtcgcccatggaggcggtggaggcataccgggtcctccttgctcttgttcaggcccagaagaattcgcctaaaaaagtggagggagaggctggtggagtaaccgagttcgaaatctggcaaggtcttgcaaatctgtactccggcctctcacacaccagggacgccgaggtatgtttgcagaaagccacagccctgaaatcgtactccgccgcgacactcgaagccgaaggttacatgcacgaggtgcgcaaggagagcaaggaggcgatggcggcctacgtgaacgcctcggcgacggagctggatcacgtgtcgtccaaggtggccatcggggctctgctctccaagcaggggggcaagtacctcccggcggcgagggccttcctctcggacgccctgagggtcgagccgacgaaccggatggcgtggctcaacctggggaaggtgcacaagctcgacgggaggatttccgacgccgccgactgcttccaggcggcggtgatgctcgaggagtcggatcccgtggagagttttaggacgctctcatga

SEQ ID NO: 2 - protein, 674 aa, Triticum aestivum
MAEPEDGGEVAPPEAAAAATSAAAHSSPPAKEEPAAAAEAKPASSGEAVSLNYEEARALLGRLEFQKGNVEDALCVFDGIDLQAAIERFQPSSSKKTTEATLVLEAIYLKALSLQKLGKSIEAAKQCKSVIDSVESMFKNGTPDIEQKLQETINKSVELLPEAWKKAGSLQETFASYRRALLSPWNLDEECIARIQKRFAAFLLYGCVEWSPPSSGSPAEGTFVPKTNIEEAILLLTTVLKKFYQGKTHWDPSVMEHLTYALSICSRPSLIADHLEEVLPGIYPRTERWNTLAFCYYGVAQKEVALNFLRKSLNKHENPKDTMALLLAAKICSEDCRLASEGVEYARRAIANTESLDVHLKSTGLHFLGSCLSKKAKIVSSDHQRAMLHAETMKSLTESMSLDRYNPNLIFDMGVQYAEQRNMNAALRCAKEFVDATGGAVSKGWRFLALVLSAQQRYSEAEVATNAALDETAKWDQGSLLRIKAKLKVAQSSPMEAVEAYRVLLALVQAQKNSPKKVEGEAGGVTEFEIWQGLANLYSGLSHTRDAEVCLQKATALKSYSAATLEAEGYMHEVRKESKEAMAAYVNASATELDHVSSKVAIGALLSKQGGKYLPAARAFLSDALRVEPTNRMAWLNLGKVHKLDGRISDAADCFQAAVMLEESDPVESFRTLS

SEQ ID NO: 3 - DNA, 9524 bp, Triticum aestivum
ctcgaagtgcgttaaccaaaacaaatccaccaaagacggctctggactgatatggtgttaaatagcaaactgagtttcagaggatgaataggagaggtcagttagacagaaattgtgcacaaatcaaccaaagacagctgtaggcaaaagttctgttgaatggcaaacagggtttcagaaaaggaacaggataggtcagttagttgtgtactaagaactctcatctacactgcagttcacgaaaaaggaagaaccactcggtgcacgacatacccgagcatcatcctcctcctttgagacttctttgacaaccacctccacttcgcgtttgtaaagctgatcaaacaaatgagagacttgtaagccagcaaagcagtaatagtttacaatgtaaatattcttacggtaacagaactttacaagaagcaaatacttcagtggagatgaactagaatgaaccaaaataacttcagcaccaacttgctcactgaacacaagtagcatagagttgtatataagcctattctaccaaagagctactaagatgcaacaagtattggagagctcgtaaaattcattcaatacgcagatgaagaactgataaacgaactctggaaagcagagcctcaaaagccagcagagtaagctagtagttagtaagcaaatgcttgtgagccgcgacggagcattccaaactgcacggccatcgcggcatgtttatttctatcggggaaaagaagggggaagctaaccttgctctgctcgcgcatgagtatggcgaacttgtcgatgttggggacggcgagcatcttgggcatgaggtagttgcggaagtgccccggcgccaccttcaccgtctcccccgccttccccagcttgtcgatcgtctgaggtgaacaagcgatgggtgatgtcaaaggttagttccacttccccgcacaatctaaaatctctagggacattgttgaaatgaaaggccaaaactgaagctttatcggtcaaaaatactactgctagcttaaaaagtttcagaaatgctggagatttatcggtcaaactgtcgctgaggcggcaccggcctcaccggatcgaaagcaccccgctcgaactgaccggaaacgcaagcggctagcgagatcgcgggatgcatcctgcagaggtggaggaccgagcggagcgtcgcggggggaggggggcagggggggtggcttaccgtggtgaggatgacctcgagcttgcggtagcggaggccgtggccggagaagaggacggggttggtggcggcggcgccgaggccgtggcggcggaggagggcggcgcgggcggcggccatggtggagtagggttcaggggaaggaggcgacgggggccggcggctgccaccaacgggtgcgcgagtgagagtattggtggctcggcttcccgccggaccgggccggtgccaggccaggcccgctaagggatctccattttttcctttgattttatttttaaaatccttctgctgcccaaaagaatttgcattttgcactttcttgagccctctttgattttattttttaaatacttccgctgccatgaaaactttgcagtttccacttttttggatgaggaagtcgaccagagcggaaatctggaaaagagccagggttcttctgctggatgccaacaccctctgcaatccaataaaatcaaatcaaacattcaaaatctcatcagaatatcaactttatgtttttttcttaaggcacataaatgcatttttttgtaacataaaaggttatgtgagtttttagtccaatttgtttcgtagttggcaggttgaaactctaggactcggatatgtgctatactcaagcaccacatgttacattttattttgcgctgaaaatcaagacatgcatcattaactttcatatttcatgaaagttacaatagttagaccctctccatttcaatttccaaagatgtaggatgcaacaattccttttaccaccaagacatattaatattgtgtggtttccgtgatatgaactcccctatcccttggtggctatggtaaatctcccctccaggcttcatcaatgagaccgtggattcgcctcccctctacctgccgctccgacgactggtggcggggttagggatcccggtgctttcggtctggttaatagtttaggttaggtttttttagtcttcttaggtgtggcgctcagatggatggcagcgctttttctcgagtttgtgtttcggtctccgattctcctcaagttcgttcatctgaacgtaattgaaggacctccgacgtagatttctgtcgtctccttgctacgatgagtttagtgtttctcgtcgtgtgacgagatttgttgtcaggtgcttcagatctattgaagggttcaacggtgacgactacgactctagggcactagtccttacgggcacatgcatgaagacttcccgactgtcatcgtatggtcaagccggctacagtaggggaacaacggtagtggtcattcgatggtgaagaggcgttctttgtgggcaagccaaatgattctgatctattatcaatgttcaccagaaaaaacaaagcaccttgttgtttcaattttgcgaaaaatgattcaaatctattatcaatgttcaccagcaaaaaagaaaaataaagcaccttttgctttgctcttgagcaaaaattcttttgagtggaaaaaatacaccctgttgtttctctttgcagccaaggacaaagcataggttacgatcacatcttggtcaacatatgtggccgttcaagatcatccatgcatgcatttgtattggtggagctagacaatctatttttagcttgtcttagaaaaaaatctatcattagctcgaattttctggaaaaaattgtaaggactcccttaaatttctatcggtattcctgatgaactttccacgtggcaacaagcagtagagagagtagtcgcaaaagagtaaaaatagaagacagaaaaattagtggaaaagggtacgcatgcgaaccgtggaagaagttgccgcctccgctcctctccatcgacgacgaaaccgagcacctcccagctcgacgagatcgggtgcccgtcggtgcgatccctctgctgcagcggcatggcggagccggaggacggcggcgaggtcgcccctcctgaggcggcggcggcggcgacgagcgcggccgcccattcgtctccccctgctaaggaggagccggcggcagcggcagaggcaaagccggccagctccggcgaggcggtctccctcaactacgaggtccgaaatatctgaaactcttttatgaatgtttgtttgatacgtagtacggtgcttgtcctatataatgctgctatgatgtgaacttggtttgcaagaaattgccatgtttgaagtgtttggtcagtgccgccaatgttatgtcaaatttcgtattgccggcgatgatggtgtcaattcaattaagcgatgactttgattgttctcacataaaccgaaaatgtaaagatgccaacgttggtcgtgcgtttttttcaaaaaatattgtttgagaggctttgtgtgggaaatgtgttcctttcttggggatgtcaaatgctgaattgtgattccatttcagttctggttctatttcattgattggtttatccaattgcgaattattcggcaagtttataagacatgcacctttttttgttctttatatatttgggtgagtgaattataacacgatggtgtcaatcaaaatgctttttattgggtgagtgaattgtgaataatcttaatgccagtataggtagcaagattttactgaatgatgtgtaatcatacggagaaagggacattttctttgtccagattatgaagaactgatcatatttctattcccatgaaccatgctattgatctccattgcaattattaatttccaaaaatgaagttcaaacttagcttaatacatggagaattccaaccgtcatgctttctcgggtttattacaccaagttatttttttgcgggtttattacaccaagttcgtttatacatctatcggtaacaggaagcgagagctctcttaggaaggctggaatttcagaaaggcaatgtagaagatgcactttgtgtgtttgatggaatagaccttcaagctgccattgagcgcttccagccatcatcctcgaagaaaacaacagaagctactcttgttctcgaagccatttacttgaaagcattgtcccttcagaagctaggaaaatcaataggtaacaaaattgctttataccgttgtttaagtttaaaacaaattgctttaattgtgttttacaaaaataaattatcatttggaagttgttcttttttttagcttattctttgacttgtaacaaattactgaaatacctgttgaacatgcagaggccgctaaacaatgcaaaagcgtcatcgattctgttgaaagtatgttcaagaatggcactcctgacatcgaacagaagctacaagaaactatcaataaatctgtggaacttctcccagaggcctggaaaaaagctggctctcttcaggaaacatttgcttcgtacagacgcgctcttctcagcccgtggaacctcgacgaggaatgcattgcaaggattcaaaagagatttgctgctttcttgttgtatggttgtgtggagtggagtccgcccagctctggttcaccagctgaaggcacttttgttcccaagacaaatattgaggaagccattctactcctcacaacagtattgaagaagttttatcagggaaagacccactgggatccctcggtgatggaacacttgacctacgcattgtcgatttgcagccggccttctcttattgcagatcatctggaggaggttctacctgggatatatcctcggacggagagatggaacacactagcattttgctactatggtgttgctcagaaagaagtcgctctaaatttcctgaggaagtccttgaataagcatgagaacccaaaagatacaatggcattgctgttagccgccaagatatgtagcgaggactgccgtcttgcctccgagggtgtcgagtacgcaagaagagcgattgcaaacacggaatcattagatgttcatctgaagagcactggcctccatttcttggggagttgcctgagtaagaaggccaagattgtttcatccgatcaccaaagagctatgttgcacgcagaaactatgaagtcccttacggagtcgatgtctcttgaccgctacaacccaaacctaatattcgacatgggagttcaatacgctgagcagcggaacatgaacgccgcgctgagatgtgccaaagagtttgtcgacgcgaccggtggagcggtctcgaaaggttggaggtttctagccctagtcctctccgcacagcaaagatactccgaagcagaagtggcgaccaatgccgcgttagacgagaccgcaaagtgggatcaagggtcactgctcaggataaaggctaagctgaaggtcgctcaatcgtcgcccatggaggcggtggaggcataccgggtcctccttgctcttgttcaggcccagaagaattcgcctaaaaaagtggaggtttgttttcttaatcaaatgcagcaaaaaaaaagtaccatccgtatactatttttctcttggcactttctccattagttcacataccgatgcttcagggagaggctggtggagtaaccgagttcgaaatctggcaaggtcttgcaaatctgtactccggcctctcacacaccagggacgccgaggtatgtttgcagaaagccacagccctgaaatcgtactccgccgcgacactcgaagccgaaggtgagccgaaagctcaggtcaccaaacccttacaaaatttcaccccgatcgatgtacgagtcgatgcaatgcaatgcaggttacatgcacgaggtgcgcaaggagagcaaggaggcgatggcggcctacgtgaacgcctcggcgacggagctggatcacgtgtcgtccaaggtggccatcggggctctgctctccaagcaggggggcaagtacctcccggcggcgagggccttcctctcggacgccctgagggtcgagccgacgaaccggatggcgtggctcaacctggggaaggtgcacaagctcgacgggaggatttccgacgccgccgactgcttccaggcggcggtgatgctcgaggagtcggatcccgtggagagttttaggacgctctcatgagattatcacaacatacaggaactccttactttttctaccctccacattactcctctactctccttgtttctctctcttgttgtagttcaatgcatgtaaagttaaccgatgtgtataggcacaattgttttgcatatttatttattttgccgtgggacctgtatttgctcatggaaagtgtgatgctttcagaaaatgcaagtgtgatggcagctgaactgttacttgaatttcgcttttacttgcactgttttaattttgtatgagaatgatgacgccaaagccttgagttaacagcgctatttaatttacacttcacactgcacactctctatactattgatagcctgggcttattttttgttgccctctatacattctgcatagccatttttttctcttttttttgcgagggtaaagagttttgatagtaattgtggacttatcaggaagctgaacatatatagcaaatgtattgtaaagatggacctgccctatgctgttcttcatcttggcggaggaccggcggtggggagtggtttcggggaggccggggcggcaaggcggcacgggagcgctgccggctgcgggcggcggaggccggcggtttggccagtggtggctggcggcttaagggggtgaaggttgaagaagcactgtaggcctttgatttcacatccaatggctcagaaatcgactgaccacaaatgaaaattttagctgactgatttctagccatttccgtcaacaacacctgggttctgattagtttcttcaggaaagctagaatcagctactgccttcaagaaacaaaaatggtcgacggagggggacaggccagaaccatagaaccattcgtgttatcacccctgatcactgcagttgtgatgcttcgggcgggaacaagaatggacggaggaggacaagctggagatggaggccgagctacaagcagccgggcatcaaacaactaaatttctcaacctaaatggccctgtgcccctgtcctagtgtcgaatttgaatagaatgatgcaatcaattcttctgtaccgctcaaaagagtgataggatacatagttgcatcgcatgctgggacacagatcctctggctaaccctgccttaccctgcctttgggtcgctgacaagtgggccccacgcttggtgggacccatgtgtcagtgtctcaatggcaggttagccaaagtcaggggatcctcgtcccacatgctgatcaccaaaggagtacaatcaatataagtcgaacgtacttgagaacatacacaggcaaaataagacaattcttgtaaattcatcagtcgcaggacatggattttatgcattctaaagatatcaacatgagcttgtagatgcgggggaatgaacaaccagtttcacactattagatttattttagttaagcactcaagtcagcacaagctaaaccatgctataagctgggcataagaacaaccaaacttgagggaaaagggctaaaaaatgaaggcttctgcgataattaaaatgacaagccaccacgcttgctacaaaatagtatgtgtaccagaggattcttgttagaggcacggatgcatattcacaattccattttactcaaaaaattgttataaccactttaaggattctttcatatctattccaccaaggcatgaactgcttaatattgctaagttgcaactgaaacacaagttataacatgtcacaactaagccactagaaaatagaatcacaacgtgtcacaaaactgaaaagattgtgaaataaaaagaaatgggaaaaaagttgcaatctcaaaaaggagagattgtgcagtaaaaaagagaaaagaaacaacttgctatcgccagttaccagatcttgctagatgtatctactacccttatagaaacacctcaacgcctctaagaacacgtgcctgtccacgcggctcctcctcgcccgcctgccgcgtctccttcgccgcgcctcacccgcccatgctagaagaaatcaaacccccactgcggcgcacgaccacgtgccactcgccctgctcaacgcagccctcccagcgtccgtcctcctgggccaccgccgcagccgttgccatgtgtggatcctggacatcctcgtcgtttcatggaactgcttccaacacagtcgccggctgagtcattcacacgccgaagggggccgtcatccccatgctatgaacaatcataagttcattccttttgtcttctggctaaaatcactttgaatccacctctgtatacgagactgtaatctccagagtctcaagatacaagaccaagcttgttatttttccaagttgttcttgcaaggtcaagatatagtggcagtttctttttcgagtgtggtttttgtgcacccacggacattccacccacggtgcacccacgataaaaaacttagcaaaacatttaaaaaaattctgaaattttgtggatgtgattatgaccaaatgttttaggcgcttgcaaaatttggttgcaaaatgacacccatagagctttgtacaaaaaacaaagtttgtgttgaaaacatttgaacagtaaggtaggtgcagagcatcatttgtatttcgtttatatggagatcatttcatatttttcagtgaccaaactttgcaagctcctaaaacattggctcataatcacatccacgaagtttcagaatttttttagtttgtttacatttttttcttcgaatttactgttcactccataggtgcgccgaaggtggatgcatccactacttttctttcctttcctttttctctgtgtattttacatgttcgtacgtttgcaccctgctctgactgctttcttgttccaaggctggtgattctactccagagcttgctacggccatccaggcccagggcgaccatcactcgcggtggcgagaagcacttggtcgaagttgtgaaggttatagatgcgtacaaggtatacggcaagctccgtgttgagaggatgaaccggcaccaattgggagcttggatgaagaaggctacccgtgtggagaaagtggagaagaagtgatgagatgtttatgacagctaattgatgttgttatctaagtttctgaatgtgtgttttggtctgctcggataccttgtttgatatcaaatagccctttcttcccactgttcaaatcagctcttcattgatatgcaaatgttcaaacaatgtagttcaaatagttaagttgttatgccaggaa

SEQ ID NO: 4 - DNA, 2025 bp, Triticum aestivum
atggcggagccggaggacggcggccaggtcgcccctcctgaggcggcggtggcggcgacgagcgcggccgcccattcgtctccccctgctaaggaggagccggcggcagcggcagaggcaaagccggccagctccggcgaggcggtctccctcaactatgaggaagcgagagctctcttgggaaggctggaatttcagaaaggcaatgtagaagatgcactttgtgtgtttgatggaatagaccttcaagctgccattgagcgcttccagccatcatcctcgaagaaaacaacagaagctactcttgttcttgaagccatttacttgaaagcattgtcccttcagaagctaggaaaatcaatggaggccgctaaacaatgcaaaagcgtcatcgattctgttgaaagtatgttcaagaatggcactcctgacatcgaacagaagctacaagaaactatcaataaatctgtggaacttctcccagaggcctggaaaaaagccggttctcttcaggaaacatttgcttcgtacagacgcgctcttctcagcccgtggaacctcgatgaggaatgcatcgcaaggattcaaaagagatttgctgctttcttgttgtatggttgtgtggagtggagtccgcccagctctggttcaccagctgaaggcacttttgttcccaagaccaatattgaggaagctattctactcctcacagtagtattgaagaacttttatcagggaaagacccactgggatccctcggtgatggaacacttgacctacgcattgtcgatttgcagccagccttctcttattgcaaatcatctggaggaggttctacccgggatatatcctcggacggagagatggagcacactagcattttgctactatggtgttggtcagaaagaagtcgctctgaatttcttgaggaagtccttgaataagcatgagaacccaaaagatacaatggcattgctgttagccgccaagatatgcagcgaggactgccgtcttgcttccgagggtgtcgagtatgcacgaagagcgattgcaaacacggaatcgttagatgttcaactgaagagcaccggcctccatttcttggggagttgcctgagtaagaaggctaaggttgtttcatccgatcatcaaagagctatgttgcacgcagaaactatgaagtcgcttacggagtcgatgtctcttgaccgctacaacccaaacctaatattcgacatgggagttcaatacgctgagcagcggaacatgaatgccgcgctgagatgtgccaaagagtttgtcgacgcaaccggtggagcggtctcgaaaggttggaggtttctagcactagtcctctccgcacagcaaagatactccgaagcagaagtggcgaccaatgccgcgttagacgagaccgcaaagtgggatcaagggtcactgctcaggataaaggctaagctgaaggtcgctcaatcatcgcccatggaagcggtggaggcataccgggtccttcttgctcttgttcatgcccagaagaattcgcctaaaaaagtggagggagaggctggtggagtaaccgagttcgaaatctggcaaggtcttgcaaatctgtactccagcctctcacactgcaaggacgccgaggtatgtttgcagaaagccagggccctgaaatcatactccgccgcgacactcgaagccgaaggttacatgcacgaggtgcgcaacgagagcaaggaggcgatggcggcctacgtgaacgcctcggcgacggagctggagcatgtgtcgtccaaggtggccataggggcgctgctctccaagcaggggggcaagtacctcccggcggcgagggccttcctctcagacgccctgagagtcgagccgacgaaccggatggcgtggctcaacctggggaaggtgcacaagctcgacgggaggatttccgacgccgccgactgcttccaggcagcggtgatgctcgaggagtcagatcccgtggagagttttaagacgctctcatga

SEQ ID NO: 5 - protein, 674 aa, Triticum aestivum
MAEPEDGGQVAPPEAAVAATSAAAHSSPPAKEEPAAAAEAKPASSGEAVSLNYEEARALLGRLEFQKGNVEDALCVFDGIDLQAAIERFQPSSSKKTTEATLVLEAIYLKALSLQKLGKSMEAAKQCKSVIDSVESMFKNGTPDIEQKLQETINKSVELLPEAWKKAGSLQETFASYRRALLSPWNLDEECIARIQKRFAAFLLYGCVEWSPPSSGSPAEGTFVPKTNIEEAILLLTVVLKNFYQGKTHWDPSVMEHLTYALSICSQPSLIANHLEEVLPGIYPRTERWSTLAFCYYGVGQKEVALNFLRKSLNKHENPKDTMALLLAAKICSEDCRLASEGVEYARRAIANTESLDVQLKSTGLHFLGSCLSKKAKVVSSDHQRAMLHAETMKSLTESMSLDRYNPNLIFDMGVQYAEQRNMNAALRCAKEFVDATGGAVSKGWRFLALVLSAQQRYSEAEVATNAALDETAKWDQGSLLRIKAKLKVAQSSPMEAVEAYRVLLALVHAQKNSPKKVEGEAGGVTEFEIWQGLANLYSSLSHCKDAEVCLQKARALKSYSAATLEAEGYMHEVRNESKEAMAAYVNASATELEHVSSKVAIGALLSKQGGKYLPAARAFLSDALRVEPTNRMAWLNLGKVHKLDGRISDAADCFQAAVMLEESDPVESFKTLS

SEQ ID NO: 6 - DNA, 9343 bp, Triticum aestivum
tcgctaaaacacctgccccacggtgggcgccaactgtcgtggttctaagtctgacagtagagtgggggggtaggtatggagaggcaaggtcctagctatggagaggttgtaaacacaagagatgtacgagttcaggcccttctcggaggaagtaaaagccctacgtctcggagcccggaggcggtcgagtggattatgtttatatgagttacagggtgccgaacccttctgcctgtggaggggggtggcttatatagggtgcgccaggaccccagccagcccacgtaatgaagggtttaagggtacattaagtccgaggcgttactggtaacgccccacataaagtgtcttaactatcataaagtctacttaattacagaccgttgcagtgcagagtgcctcttgaccttctggtggtcgagtgagacttcgtggtcgagtccttcaattcagtcgagtgagttcctcgtaggtcgactggaaggtgatctcttctaagggtgtccttgggcagggtacttagatcaggtctgtgaccctaccctaggtacatgactccatcagggccggagtgccggaggagtgcgacgaggatcgggaggaagaagaggaggaggaggagccgaacctccttggcacccatggcccgacgcgtcagtgctgcgccggggggtacgccaatggcgggtcgttgccgccctcaaggtacgtgagcacgccctcgagtgtgcggccgggaacgccccaccagaggtgacgcccctcgttgttctgtcggccgccgaacaccggcgcaccgttggtggacgccaatcgctgctgctggcggcgctcgaaatacgccgcccaggccgcgtggttgtcggcggcgtactgggggagggagagttgggcatcggtgagggacgcgcgcacgatctcgacctcctcggcgaagtacttcggcttcgccacggcgtcgggcaacgggggaatgggtactcccccggcgctgagcctccaccccgatggcccggcgcgcatgtccggcggcgccgggatgttcgcctggaacaggagccaggactcctgttcacggagcgaacggcggccgaagccgttggccgccgcctcgtctccggggaagcgttctgccatggcaacggcggggtggggcgggctcgggagaggtagagggaggggccggagggcggcgctcgggagaggcagggagagggaggggttggacggcggcgagggggggactggtctgggcacaggcgagtggaggccgctggcttttatagccgggccgcgcccgtgtgtacgcgtgcgcgggaagggaggcgtcggcgcgccgccccgtgaagcgccgctcgtgaggaatcaatggcaaggctgaccggcggcagccttgccattgattctccgcggaaaaccgaggccgttgggggaagacgaggcgccgagtcgctgacgcggctggcccgcgtctttttcacgccaaaacagctcgccccggcacccccgggcgccccccagcgcgccgggttcgggctaggtccgccggcgctgttttcggcccaagccggcgaaaatcgggctcctgggtgcgcgactgggccgtttttcggcgccggcgcgaaaaaaacgcctggggaggccttcctggggcgcggctggagatgccctaaacttgcgcaccgcacctgggccaacgcaccccctttagtaccgggtcgtagctctaatcggtactaaaggtggggtcttttggttctccgatgatcgtttattctacaattgcccgattttaactagatttgctgctagtccgaagatctacttccgttcatttccatatgtgcatgtgttgcatggatatgagaagccgttgagatacacgggtatggacgcaacaaaatgaggcgtgcccggtcactgcccgcggacgcgaccggatacgtccgcggacgtttgaggggccatatttgtcatatgcggctgtagatgctctaacgtggcagtaacgaccgtgagcagttggcacgtgacggccggccttaatcaacatgtttctccatgccatgggcatctgtcatctgcgccattggtagtgcgaggagatgggacgcgggtgaccctgaggagggaggtaaaacctcctcctgcgcaagcagttgatggatggagcgcccttcaacccaatgctccataatccccaaatatggaggctcgtgggcttgatatgcaacgccttcataaatgataactatcaaagccgtatggctggcgtgtctgatatagtgatttttggtccaaaaggcgttactacgactttgttaaagttgctctaattgcatgcatgaccatccggtcatcttatctgtgccacacaatgaaatcgctcggcatgcaatttctgaaggctcctgagcaatttctacttgtaggcaccacacgaacgttgtgcactttttttgggatcacatcaactggccttcactaaatactactcagaacaagccactacacgttttgtcttgcactgtatatgttttctccaacgtcagactattttgagagagaaaaaacaccttgttgtttctcttttgcagccaagggcaaatcaaaagtaatgggatcgatcacatcttggtcaacataagtggccgttcaagagcaatgtattggcggagctagacaacctatttttacctttctctaaaaaataatctatcattaactcaaattttccggaaaatggcaggactcccttaaatttctctcggtattcctggcgaactttacacgtggcaacaagcagtagagagataggtagagagagtagtcgcaaaagactaaaaatagaagacagaaaaattagtggaaaaaaaggtaaacatgtgaaccgtggaagaagttgccgcctctgtttctctccatcgacgacgaaaccgagcacctccaagctcgacgagatcgggtgcccgtcggtgcgatccctctgctgcattggcatggcggagccggaggacggcggccaggtcgcccctcctgaggcggcggtggcggcgacgagcgcggccgcccattcgtctccccctgctaaggaggagccggcggcagcggcagaggcaaagccggccagctccggcgaggcggtctccctcaactatgaggtccgaaatatctgaaattcttttatgaatgtttgtttgatagtacggtgcttgtcctatataatgctgccatgctgtgaatttggttgacaagaaattgccatgtttgaagtgtttggtcagtgccaccaatgttatgtcaaatctcgtattgccgacgatgatgatgccaattcagtttagccatgactttgattgttctcacatgaaccgaaatgtaaagatgccaacgttggtcgtgcgttttccttgaaaaatattgtttgagaggctttgtgtgggaaatttgttcctttcttggggatgtcaaatgccgaagtgtgatttcatttcagttctggttctatttcattgattggtttatccaattgtgaattattcggcaagcttgtagacatggaccttttttgttctttaaatatttgggtgagtgaattgtgatttgtgaataatcttaatgccagtataggtagcaagattttactgaataatgtgtaatcatatggagaaagggacattttctttgtccagattatgaagaactgaccatatttctattcccacgaaccgtgctattgtatctccattgcaattattaatttccaaaaatgaaattcaaacttagcttaatacatggagaattccgaccgtcatgctttctccggtttattacaccaagttcttttgtttttgcgggtttattacaccaagttcgtttatacatctatcaataacaggaagcgagagctctcttgggaaggctggaatttcagaaaggcaatgtagaagatgcactttgtgtgtttgatggaatagaccttcaagctgccattgagcgcttccagccatcatcctcgaagaaaacaacagaagctactcttgttcttgaagccatttacttgaaagcattgtcccttcagaagctaggaaaatcaatgggtaacaaaattgctttataccgttgtttaaatttaagacaaatttctttaattgtgttttacaaaaataaatcatcatttggaagttgttctgtttttagcatatgtttgacttgtaacaaattattgaaatacctgttgaacatgcagaggccgctaaacaatgcaaaagcgtcatcgattctgttgaaagtatgttcaagaatggcactcctgacatcgaacagaagctacaagaaactatcaataaatctgtggaacttctcccagaggcctggaaaaaagccggttctcttcaggaaacatttgcttcgtacagacgcgctcttctcagcccgtggaacctcgatgaggaatgcatcgcaaggattcaaaagagatttgctgctttcttgttgtatggttgtgtggagtggagtccgcccagctctggttcaccagctgaaggcacttttgttcccaagaccaatattgaggaagctattctactcctcacagtagtattgaagaacttttatcagggaaagacccactgggatccctcggtgatggaacacttgacctacgcattgtcgatttgcagccagccttctcttattgcaaatcatctggaggaggttctacccgggatatatcctcggacggagagatggagcacactagcattttgctactatggtgttggtcagaaagaagtcgctctgaatttcttgaggaagtccttgaataagcatgagaacccaaaagatacaatggcattgctgttagccgccaagatatgcagcgaggactgccgtcttgcttccgagggtgtcgagtatgcacgaagagcgattgcaaacacggaatcgttagatgttcaactgaagagcaccggcctccatttcttggggagttgcctgagtaagaaggctaaggttgtttcatccgatcatcaaagagctatgttgcacgcagaaactatgaagtcgcttacggagtcgatgtctcttgaccgctacaacccaaacctaatattcgacatgggagttcaatacgctgagcagcggaacatgaatgccgcgctgagatgtgccaaagagtttgtcgacgcaaccggtggagcggtctcgaaaggttggaggtttctagcactagtcctctccgcacagcaaagatactccgaagcagaagtggcgaccaatgccgcgttagacgagaccgcaaagtgggatcaagggtcactgctcaggataaaggctaagctgaaggtcgctcaatcatcgcccatggaagcggtggaggcataccgggtccttcttgctcttgttcatgcccagaagaattcgcctaaaaaagtggaggtttgttttcttaatcaaatgcagcaaaaaaaaaagagagagtaccattcgtgtactatttttctcttggcacattctccattagttcacgtactgatgcttcagggagaggctggtggagtaaccgagttcgaaatctggcaaggtcttgcaaatctgtactccagcctctcacactgcaaggacgccgaggtatgtttgcagaaagccagggccctgaaatcatactccgccgcgacactcgaagccgaaggtgagccaaaggttcaggtcaccaaagtcttacaaaatttcacccgatcgatgcacgattcgatgcaatgcaggttacatgcacgaggtgcgcaacgagagcaaggaggcgatggcggcctacgtgaacgcctcggcgacggagctggagcatgtgtcgtccaaggtggccataggggcgctgctctccaagcaggggggcaagtacctcccggcggcgagggccttcctctcagacgccctgagagtcgagccgacgaaccggatggcgtggctcaacctggggaaggtgcacaagctcgacgggaggatttccgacgccgccgactgcttccaggcagcggtgatgctcgaggagtcagatcccgtggagagttttaagacgctctcatgagattatcacaacatacatgaactccttactttttttaccctctacattactcctctactctcatcgtttagcttccctgttgtagttcaatgcatgtaaagttaatcgatgtgtataggcgcaattttttttacgtatttatttattttgccgttggaccctctatacatactattgattgcctgggcttatttcttggtgccctctatatattctgcatagccatttcttagggagatcgtaattgtcgacttatcaagaagttgagcctatatagcaaaatgtattgtatagctggaccagccctatggtgttcctcatcttggcataatggggacaccactacactagcctcttcactgcttcacaaggtgtcaactgtcaaaccagcaaaacaaagagcaaacaaaccaattacttgcatattaaaacacatctccagtttcaggtccatgtgttcttattcatcaattcacgcccgagggactactttggatgggatctcgaccacatacctcctgtccaaggctatatatgattttagaaaccataatgttgagtgaacctgagaggtttttggatcgccagttggacaacaaccaaactgtggttgagttggttagtaggccacactaccggagttcaagtcctatcaggcacaacatatttttacgttccacaagagaaaactgcctccaggaaacctaccccagcccatgttcaagccatcacagaaaacaagaacaatttgatgctgcagctaacaagaacaattaactcactcgttggcatttccactaaactgttccaaagaaaatatgatgcctaaaaaggaatgtcatctccgtattcgtacacacgcttcgaatgcatgtgctactcagcgatatgcggatccagcgccataaccttgtcgaatagggatctaacatacccatatgaccgcatctgcagaatgcataaataaatatatctttacatgagatccattcaacggacactcctgccgtgcatccaactgcaagattgcatccggaattcataaaacaaatactgtactatcatccaggagaatggagtatatatatataacaccaggctgaggaaggaggacacaaattcaaccgaatacggacgtacatggcgggagaaacaactactatgaaagatcttcccgcttattattaattatattatttgactgaggaaataacacaacacaagcaagcaagcaaggaaattaagcgcgggaggaataggtagtacatgcaatcacgcggacggacggacggcgtcctcgcactcgtcaatctcggcgaggctgccagactcaaccagacccacacggaacaacttggagtaggagatgtccccggagacggtgacgtcgacggagttgcagacccagtcctggcgttcccgggtgaggacgaggaggcacggccggatgcatctcgggtccgtgaagctgaattcgtcggtgctgccacggttgaacctggcaccgtcgcggtcgtcttcccagtgtgtcacgacggggctgccgtcctgcaggttgtcgccgtacaaccggaactccacgaatgcctccgtgccggccggaggccagagccccgtcttcaccttcacccggtactcacagtcccccttggtggtgccgctgccggcgaggaaggcggccaccagaatgacaagggcgagcttgggcatgcccatggccacctgcgttagtttagtagctacgatagatagatagatagatagatatatgacgatgacggttgatggatggatggatcagcttcccaccggcatttatatagggtgtttatttgcccagctccagctgcatttatatagggtgtttatttgcccagctccagctgctgcccctaacccatattaataagctagcttattatccctgattcgcatacagccgtgatcgataccagacatcacatgatgagatcagatcaggtcagatcgatcagatggatgataagctttatcaattcccggccggacacgcaagttggtctcccgagaccgaccggcaaatcaagcgcccgatcgcatcacatgcacaacatcaatcttccctttctgggcttaccaataacattaactaactatatacatttccatgcagtgcagagcttcatttaccactaataatggaatggaatacaagtattggatgggatcgggtctggattaattgtatatattttctctctgaaaaaacgatcgatctgacagagttgcgcgccggagctgcagcaacacgacggtgggagtagattgagaactcgggatacgttttctggtatttttttcacgaaatttcacaggggagtagattgagaacccaagttcaatatcccaaaatttcagtttattttttaaaaaaattactatttttttatatttaatattcgtataggggggtggagcacccagaaactcttgtgtatttgtcccttgctcttctttatgcaaattttacaaatgtagtcagtataataggctttttaatacttatgttcctcttaccgcattttattttacctcgcaaggcaaaactgaccaagctaagccaatctgctctctatcacaatcatttatttaggacatggagctaagggcatctccaaggtggacccacaagcctcccacaatcatcccgactgtgctgtccggaccgccgaagccatccaacgcggtctcgtatcggtccgcggggcggcccggacgcgatttctccagcaaaacggagacaaaagtgggggagctttgcaggagtccgaaacacgaaacgtagaagtccaacaccctaggcccacccaaaacccttcccggaccccgcgactccttccttctttctctgttgccgttgccgccactccaccaccccggccgccacgccacacccctgccgaaaatctgcgtctccatcacctccggcgctccagcagggctccccgccgcttctcctccgtctccgttcaaccccctgtcctccaaccgccccgccatatacgatcctctcctgtgcctggcaacacttcaatgaatcgccggagctcagaactgtgtcccttctttttagcaatggattccaatttggagtacatataggagcatctttataatcacctagttacgttgtaggccgtttgtgcaccatgatgcggtgacgtgctgccctgtgcctccttccctcctcggcaccacgtcgtcgccagtccacca

SEQ ID NO: 7 - DNA, 2025 bp, Triticum aestivum
atggcggagccggaggacggcggccaggtcgcccctcctgaggcggcggcggcggcgacgagcgcggccgcccattcgtctccccctgctaaggaggagccggcggcagcggcagaggcaaagccggccagctccggcgaggcggtctccctcaactacgaggaagcgagagctctcttgggaaggctggaatttcagaaaggcaatgtagaagatgcactttgtgtgtttgatggaatagaccttcaagctgccattgagcgcttccagccatcatcctcgaagaaaacaacagaagctactcttgttcttgaagccatttacttgaaagcattgtcccttcagaagctaggaaaatcaatagaggccgctaaacaatgcaaaagcgtcatcgattctgttgaaagtatgttcaagaatggcactcctgacatcgaacaaaagctacaagaaactatcaataaatctgtggaacttctcccagaggcctggaaaaaagctggttctcttcaggaaacatttgcttcatacagacgcgctcttctcagcccatggaacctcgacgaggaatgcatcgcaaggattcaaaagagatttgctgctttcttgttgtatggttgtgtggagtggagtccgcccagctctggttcaccagctgaaggcacttttgttcccaagaccaatattgaggaagctattctactcctcacaacagtattgaagaagttttatcagggaaagacccactgggatccctcggtgatggaacacttgacctacgcattgtcgatttgcagccggccttctcttattgcagatcatctggaggaggtactacctgggatatatcctcggacggagagatggaacacactagcattttgctactatggcgttggtcagaaagaagtctctctgaatttcttgaggaagtccttgaataagcatgagaacccaaaagatacaacggcattgttgttagctgccaagatatgtagcgaggactgccgtcttgcttccgagggtgtcgagtatgcaagaagagcgattgcaaacacggaatcattagatgttcatctgaagagcaccggcctccatttcttggggagttgcctgagtaagaaggccaagattgtttcatccgatcaccaaagagctatgttgcacgcagaaactatgaagtcgcttacggagtcgatgtctcttgaccgctacaacccaaacctaatattcgacatgggagttcaatacgctgagcagcggaacatgaacgccgcgctgagatgtgccaaagagttcatcgacgcaaccggtggagcggtctcgaaaggttggaggtttctagcactagtcctctccgcacaacaaagatactccgaagcagaagtggcgaccaatgccgcgttagacgagaccgcaaagtgggatcaagggtcactgctcaggataaaggctaagttgaaggtcgctcaatcatcgcccatggaggcggtggaggcataccgggtccttcttgctcttgttcaggcccagaagaattcgcctaaaaaagtggagggagaggctggtggagtaaccgagttcgaaatctggcaaggtcttgcaaatctgtactccaacctctcacactgcagggacgccgaggtatgtttgcagaaagccagagccctgaaatcgtactccgccgcgacactcgaagccgaaggttacatgcacgaggtgcgcaacgagagcaaggaggcgatggcggcctacgtgaacgcctcagcgacagagttggagcacgtgtcgtccaaggtggccatcggggcgctgctctccaagcaggggggcaagtacctcccggcggcgagggccttcctctcggacgccctgagggtcgagccgacgaaccggatggcgtggctcaacctggggaaggtgcacaagctcgacgggaggatcgccgatgccgccgactgcttccaggcggcggtgatgctcgaggagtcggatcccgtggagagttttaggacgctctcatga

SEQ ID NO: 8 - protein, 674 aa, Triticum aestivum
MAEPEDGGQVAPPEAAAAATSAAAHSSPPAKEEPAAAAEAKPASSGEAVSLNYEEARALLGRLEFQKGNVEDALCVFDGIDLQAAIERFQPSSSKKTTEATLVLEAIYLKALSLQKLGKSIEAAKQCKSVIDSVESMFKNGTPDIEQKLQETINKSVELLPEAWKKAGSLQETFASYRRALLSPWNLDEECIARIQKRFAAFLLYGCVEWSPPSSGSPAEGTFVPKTNIEEAILLLTTVLKKFYQGKTHWDPSVMEHLTYALSICSRPSLIADHLEEVLPGIYPRTERWNTLAFCYYGVGQKEVSLNFLRKSLNKHENPKDTTALLLAAKICSEDCRLASEGVEYARRAIANTESLDVHLKSTGLHFLGSCLSKKAKIVSSDHQRAMLHAETMKSLTESMSLDRYNPNLIFDMGVQYAEQRNMNAALRCAKEFIDATGGAVSKGWRFLALVLSAQQRYSEAEVATNAALDETAKWDQGSLLRIKAKLKVAQSSPMEAVEAYRVLLALVQAQKNSPKKVEGEAGGVTEFEIWQGLANLYSNLSHCRDAEVCLQKARALKSYSAATLEAEGYMHEVRNESKEAMAAYVNASATELEHVSSKVAIGALLSKQGGKYLPAARAFLSDALRVEPTNRMAWLNLGKVHKLDGRIADAADCFQAAVMLEESDPVESFRTLS

SEQ ID NO: 9 - DNA, 8714 bp, Triticum aestivum
acactacattctaaacataatatctagaagccgagaggtagaagaagactttttcaaggcaaaatattcaatattttcaacaccagatttagaatgggcttgaagtgcgttaacaacagatcctccaaagacagatctgggcagaaattgtgttaaatggcaaacagggtttcagagaaggaacaggacaggtcagttagttgtgtgctaagaactcatcgacacttcagttcatgaaaaaggaagaactaatcagtgcacgacatacccgagcatcatcctcctcctttgagacttctttgacaaccacctcctcttcacgcttgtaaagctgatcaaacaaatgagagacttgtaagcctaaaaagtaacagtttacactgcaaatattcttacagtgactgaactctacaagaagcgtacttcagtggagatgaactagaatgaaccacgatgacttcagtacaacttcctcactgaacactagcatagagttgcatataaggctattctaccaaagagctaaggtgcaacaactattggagaactcgtacaaatcatacaatacacagaggcagaactgatatacgaaactccggaaagcatagcctcaaaagccaacaagagtaagctagtaagtaatgcttgtgagctgcaaccgagcattccaaaactgcacggccatcgtagcatgtttatttctatcggggaaaaggaggaagctaaccttgctctgctcgcgcatgagtatggcgaacttgtcgatgttggggacggcgagcatcttgggcatgaggtagttgcggaagtgccccggcgccaccttcaccgtctcccccgccttccccagcttgtcgatcgtctgaagtgaacaagcgatggacgatggcaaaggttaataattccacttcccggcacattgaaaatctctagggatattgttgaaatgaacagccaaaaccgaagctttaccggtcaagaatactactgctagcttaaaaagtttcagaaatgctgaagatttatcggtcaaactgtcgctgaggcggcaccggcctcaccggatcggaaacatcccgctcgaactgaccggaaacgcaagcgggatgcatcctgcagaggtggaggaccgagcggagggtcgcgggttgagatttggaggagaaggggagggagggggcaggggggctggcttaccgtggtgaggatgacctcgagcttgcggtagcggaggccgtggccggagaagaggacggggttggcggcggcggcgccgaggccgggacggcggaggagggcggcgcgggcggcggccatggtggagtagggttcaggggaagggggccggcggctgccacaaaacgggtgcgcgagggagagtattggtggcttcccgccggaccgggccggtgccaggccaggcccgctaagggatctccattttttccctttgaatttatttttaaaacacttctgctgcccaaaagaatttgcatttgcattttcttgagtccctttgatagactaaaaaaaatctcgagtccctttgatttatttttcaaaattcttctgctgccatgaaaactttgcaatttgcactttcctgagcgaggtagtagaccaggaaagaaatccggaaaagagtagggattcttctgccggatgccagcaccctccgcaatccaataaaaatcaaatcagacattcaaaatctcatcaaaatatcaactttaggccttttttctgaaggcacataaatgctatttttcgtaacataaaggttatgtgagtttttagtccaatttgtttcatagttggcaagtcaaaactcttggacttggttatctgatatattcaagcaccacatgttacatgttattttgcgctgaaaatcaagatatgtatcattaattttcctatttcaggaaagttacaatagttagaccctctccatttcaatttccaaagatgtaggatgcaacaatttttcttaccaccaagatatattaatattgtgtggttttccgtgatatgaactcccctatcccttggcagctatggtaaaatctcccctccaggcttcatcgacgagaccgtggattcgcctcccccttacctgccgatctgacgaccggtggcggggttaggcatcccggtgcttccgctgcggttaatagtttaggttagttttcttttagtcctcttaggtgtggcgctcatatggatggcagcgctttttcttcgagtttgtcttttgggctccgatgctcctcgagttcgtccattagaacgtaattgacggagctccaacgtagattcctaccgtctccttggggcagtgagtttagtgtttctcgtcgtgtgatgagatttgatgtcaggtgcttcagatctattgaagggttcaacaatgacgactgcggctctagggcgctggtccttacaggcggctctagggcgttggtccttacgggcacatgcacgaagccttcccgactgtcatcgataatgtcaagccggctacagtaggggagcggtgacagcgacgtgtcggcagctcgttctgacggcggaattggtcgttcggtggtgaagaggcgttcttcgtgggcaagccaaatgattcagatctattatcaatgttcaccagaaaaattacagcaccttgttgtttcaattttgcgaaaatgattgaaatgtattatcaatgttcaccagtaaaaaaacaaagcaccttaaaaaatttcagaggaaaaaaaacaccctgttggacaaagaataggttacgatcacatcttggtcaacatatgtggccgttcaagatcaatgtgttgacggcgcccacgatccatgcatgcatttgtatcggtggagctagacaatctatttttagcttttctcttagaaaaaaaaaactatcattagctcgaattttctggaaaaaattgtaaggactcccttaaatttctatcggtattcctgatgaactttacacgtggcaacaagcagtatggagatagctagagagagtagtcgcaaaagactaaaaatagaaggcagaaaaattagtggaaaaaggtacgcatgcgaaccgtggaagaagttgccgcctctgtttctctccatcgacgacgaaaccgagcacctcccagctcgacgaaatcgggtgcccgtcggtgcgatccctctgctgcattggcatggcggagccggaggacggcggccaggtcgcccctcctgaggcggcggcggcggcgacgagcgcggccgcccattcgtctccccctgctaaggaggagccggcggcagcggcagaggcaaagccggccagctccggcgaggcggtctccctcaactacgaggtccgaaatatctgaaactcttttatgaatgtttgtatgatagtagggcgcttgtcctatagtataatgctgctatgctgtgaacttggttgacaagaaattgccatgtttgaagtgtttggtcagtgccaccaatgttatgtcaaatttcgtattgccggcgatgatgatgtcaattcaattaagccatgactttgattgttctcacatgaaccgaaaatgtaaagatgccaacgttggtcgtgcgtttttctcgaaaaatattgtttgagaggctttgtgtggaaatttgttcctttcttggggatgtcaaatgccgaagtgtgatttcatgtctgttccggttctatttcattgattggtttatccaattgtgaattattcggcaagcttataagacatgtaccttttatgttctttaaatatttgggtgagtgaattataacacgatggtgtcaatcaaaatgctttttattgggtgagtgaattacgaataatcttaagagtgaattccggtttttacccctaatttagcatttttacactagttacccccattgaacaatttttcatccagattaccccacttagtgacaatcttgactgtttttaccccttttaatttttataagagccttcttgcaaagttcgtgttttactgagagtccagactaagtgcccaagcatatatttatattaaaacaaaaaatccgatcctattatgttaataactggcagtactaattttaaatggacacacatcattggagcccaactgaaaaacacatgtttgacaatagcactacagatctgtaaaatagaatgtgatgtttttatttgatgatttcccaaagcctaaaataaatgcttcatctgatgttttgcatcaaggaagaaaactaaaatatcatcacactaaacctacgaaccacaacccacaatgcaaaattgaatgtactttaccgctcaagataattgttcgtctttcctgcaatgtggtgcacacatacacctgacagccatgagaaaaaaaaaacgagtgcggctaaaccaagtgaccggtttgggcattggaaaaaaaatgccaagaggaatctgatggcagggaattagctgacagatcgctctcaaaagaattactggggtaaaaactggcaaacttttgctaaatggggtaaccaggatgaaaatatatttaatggggtagttagtgtaaaaaatgttaaagtagggggtaaaaactggaattcactctaatcttaatgccagtataggtagcaaaattttaactgaataatgtgtaatcatacggagaaaggggcattttctttgtgcagattacgaagaactgatcatatttctattcccatgaaccgtgctattgtatctccattgcaattattaatttccaaaaaggaagttcaaatttagcttatacatggagaattccaaccgtcatgctttctccggtttattacaccaagttttttttttgtgggtttatgacaccaaattcgtttatacatctatcaataacaggaagcgagagctctcttgggaaggctggaatttcagaaaggcaatgtagaagatgcactttgtgtgtttgatggaatagaccttcaagctgccattgagcgcttccagccatcatcctcgaagaaaacaacagaagctactcttgttcttgaagccatttacttgaaagcattgtcccttcagaagctaggaaaatcaataggtaacaaaattgctttataccgttgtttaagttaaaaaaaatgctttaattgtgttttacaaaaataaattatcatttggaagttgttctgtttgtagcttatgtttgacttgtgacaaattattgaaatacctgttgaacatgcagaggccgctaaacaatgcaaaagcgtcatcgattctgttgaaagtatgttcaagaatggcactcctgacatcgaacaaaagctacaagaaactatcaataaatctgtggaacttctcccagaggcctggaaaaaagctggttctcttcaggaaacatttgcttcatacagacgcgctcttctcagcccatggaacctcgacgaggaatgcatcgcaaggattcaaaagagatttgctgctttcttgttgtatggttgtgtggagtggagtccgcccagctctggttcaccagctgaaggcacttttgttcccaagaccaatattgaggaagctattctactcctcacaacagtattgaagaagttttatcagggaaagacccactgggatccctcggtgatggaacacttgacctacgcattgtcgatttgcagccggccttctcttattgcagatcatctggaggaggtactacctgggatatatcctcggacggagagatggaacacactagcattttgctactatggcgttggtcagaaagaagtctctctgaatttcttgaggaagtccttgaataagcatgagaacccaaaagatacaacggcattgttgttagctgccaagatatgtagcgaggactgccgtcttgcttccgagggtgtcgagtatgcaagaagagcgattgcaaacacggaatcattagatgttcatctgaagagcaccggcctccatttcttggggagttgcctgagtaagaaggccaagattgtttcatccgatcaccaaagagctatgttgcacgcagaaactatgaagtcgcttacggagtcgatgtctcttgaccgctacaacccaaacctaatattcgacatgggagttcaatacgctgagcagcggaacatgaacgccgcgctgagatgtgccaaagagttcatcgacgcaaccggtggagcggtctcgaaaggttggaggtttctagcactagtcctctccgcacaacaaagatactccgaagcagaagtggcgaccaatgccgcgttagacgagaccgcaaagtgggatcaagggtcactgctcaggataaaggctaagttgaaggtcgctcaatcatcgcccatggaggcggtggaggcataccgggtccttcttgctcttgttcaggcccagaagaattcgcctaaaaaagtggaggttagttttcttaatcaaatgcagcaaaaaaagtacgatccgtatactatttttctcttggcactttctccattagttcacgtactgatgcttcagggagaggctggtggagtaaccgagttcgaaatctggcaaggtcttgcaaatctgtactccaacctctcacactgcagggacgccgaggtatgtttgcagaaagccagagccctgaaatcgtactccgccgcgacactcgaagccgaaggtgagccgaaggttcatgtcaccaaaccctcaaaaagtttcacccaattgatgtacgattcgatgcaatgcaggttacatgcacgaggtgcgcaacgagagcaaggaggcgatggcggcctacgtgaacgcctcagcgacagagttggagcacgtgtcgtccaaggtggccatcggggcgctgctctccaagcaggggggcaagtacctcccggcggcgagggccttcctctcggacgccctgagggtcgagccgacgaaccggatggcgtggctcaacctggggaaggtgcacaagctcgacgggaggatcgccgatgccgccgactgcttccaggcggcggtgatgctcgaggagtcggatcccgtggagagttttaggacgctctcatgagattatcacagcatacatgaactccttacttttttttaccctccacattactcctctactctccttgtttatctctcctgttgtagttcaatgcatgtaaagtctttttttcgggaaatcttccgatctattcatcttcaatcatggcagtacaacgaataccaaaaataataaaaattacatccagatccgtagaccacctagcgatgactacaagcactgaagcgagccgaaggatcgccgtcgtcatcgcccctccattgtcagagtcgggcacaacttgttgtagtagacagtcgggaagtcgtcgtgctaaggcctcataggaccagcgcaccagaacagcaatcgcagcagatgaagaataacatagatcggaaggatccaatccgaagacacacgaacgtagacgaacaccaacgagatccgagcaaatccaccaaagttagatccgccggagacacacctccacacgcccaccaacgatgctagacgcaccactggaacgggggctaggcggggagacctttattcctgttggggaacgtagcagaaattcaaaaaatttctacgcatcaccaagatcaatctatggagtactctagcaacgaggggaaggggagtggatctacataccattgtagatcgcgatgcggaagcgttgcaagaacgtggatgagggagtcgtactcgtagtgattcagatcgcggttgattccgatctgagcaccgaagaacggtgcctccgcgttcaacacacgtacagcccggtgacgtctcccacgccttgatccagcaaggagagagggagaggttggggaagactccatccagcagcagcacgatggcgtggtggtgatggaggagcgtggcaatcccgcagggcttcgccaagcaccgcgggagaggaggaggagggagaggggtagggctgcgccgaaagagagacgttctcgtgtctcttgggcagcccaaacctcaactatatataaggggggagggggctgcgccccctctagggttcccaccccaagaggaggcggctagccctagatcccatccaaggggggcggccaaggggaggagagggggggggcgccactagggtgggcctcaaggcccatctggacctagggtttgccccctcccactctcccatgcgcttgggccttggtgggggtgggggcgcaccagcccacctggggctggtcccctcccacacttggcccacgcagccttctggggctggtggccccacttggtggacccccgggaccttcccggtggtcccggtacattaccgatatcacccgaaacttttccggtgaccaaaacaggacttcccatatataaatctttacctccgaaccattccggaactctcgtgacgtccgggatctcatccgggactccgaacaatattcggtaaccacgtacatgctttccctataaccctagcgtcatcgaaccttaagcgtgtagaccctacgggttcgggaactatgtagacatgaccgagacgttctccggtcaataaccaacagcgggatctggatacccatgttggctcccacatgttccacgatgatctcatcggatgaaccacgatgtcggggattcaatcaatcccgtat

SEQ ID NO: 10 - DNA, 22 bp, Artificial Sequence
  Description of Artificial Sequence Synthetic oligonucleotide
gcatggcggagccggaggacgg

SEQ ID NO: 11 - DNA, 22 bp, Artificial Sequence
  Description of Artificial Sequence Synthetic oligonucleotide
gtcgcccctcctgaggcggcgg

SEQ ID NO: 12 - DNA, 22 bp, Artificial Sequence
  Description of Artificial Sequence Synthetic oligonucleotide
aaggaggagccggcggcagcgg

SEQ ID NO: 13 - DNA, 22 bp, Artificial Sequence
  Description of Artificial Sequence Synthetic oligonucleotide
gagaccgcctcgccggagccgg

SEQ ID NO: 14 - DNA, 3246 bp, Triticum aestivum
atggctggagaggttggcaagtggggtagttccttcaaacgttcttgggctttaatcccactggtcgcccatggaatcatcgtggtcgtagtgggtctggcttactctttcatctcgtcgcacataaatgatgatgccgtgagcgccatggacgcgtcgctggcgcacgtcgccgccggcgtgcagcctctaatggaagccaaccgctccgccgccgtcgtcgcgcactctctgcagatccccagcaatgaatcatcttatttccgatacgtgggaccatacatggtcatggcgttggccatgcagccgcagctggccgagatatcatacaccagcgtggacggcgccgcgttgacgtactaccgcggcgagaacggccagccgagagccaagttcgggagccagagcggccaatggcacacccaggccgttgatccggtgaacggccgtcccaccggccgccctgacccaggggcgagtccggagcacctacccaacgcgacgcaggtcctcgccgacgccaagagcggctcgcccgcggccctcgggtccgggtgggtcagctccaacgtccagatggtggtcttctccgcgcctgtcggtgacactgcgggcgtggtcttcgcagcggtccccgtcgacgtcctggcgatcgccagccagggcgacgccgccgccgatcccgtcgcgcggacgtactacgcgatcaccgacaagcgcgacggcggcgccccgccggtttacaagcctttggacggcgggaagcccggccagcacgacgcgaagctgatgaaggcctttccctcggagaccgaatgcaccgcgtccgccattggcgcgcccggcaagctcgtgctccgcgccgtcggggcggaccaagtcgcgtgcacgagcttcgacctctccggagtgaacctgggagttcgtcttgtggtcagcgactggagcggggcagccgaggtccggcgaatgggggtggccatggtgagcgtcgtgtgcgcggtcgtggcgatcgcgacgctggtgtgcatccttatggcacgggcgctgtggcgggccggggcgcgggaggcggctctagaggctgacctggtgaggcagaaggaggcgctccagcaagcggagcgcaagagcatgaacaagagcaatgccttcgcccgcgccagccacgacatccgctcctcactcgctgccgtcgttggactcatcgacgtttcccgggtagaggccgagagcaacgccaatctcacctataacctcgaccagatgaacattggcacaaacaagctcttggatatacttaacacgatactggacatgggcaaggtggagtccgggaagatgcagctagaggaggtggagttcaggatggcagacgtccttgaggaatccatggacctggcgaacgtcgtcggcatgtcaagaggcgtcgaagtgatctgggacccttgtgatttctccgtgctccggtgcaccgccaccatgggcgactacaggcgtatcaaacaaatccttgacaacctactcggcaacgccatcaagttcacacacgacggccacgtcatgcttcgagcatgggccaaccgtcccatcatgaggagctccataatcagcaccccatcgaggttcaccccccgttgccgcacgggtgggatctttcggcggctgcttggaaggaaggagaaccgttcggaacaaaatagccgaatgtcattacaaaatgatcccaattcggtcgagttctacttcgaggtggttgacactggtgtgggcataccccaggaaaagagggagtctgtgtttgagaactacgttcaagtgaaggaagggcatggtggcaccgggctcggacttggaattgtgcaatcctttgttcgtctgatgggaggagaaattagcatcaaggacaaggagccaggagaagcggggacgtgcttcggcttcaacatcttcctcaaggtcagcgaggcatcggaggtggaagaggacctcgagcaagggaggatgccgccgtcgctgttcagggagcccgcctgcttcaagggcgggcactgcgtcctcctcgcccacggcgacgagacccgccggatcctgtacacgtggatggagagcctcgggatgaaggtctggcccgtcacgcgccccgagttcctcgtcccgaccctcgagaaggcgcgctccgccgccggcgcctcgccgttgaggtcggcgtcgacgtcgtcgctgcatggcgtcggcagcggcgactccaacattacgacggaccggtgcttcagctccaaggagatggtcagccacctgcggaacagcagcggcatggccggcagccacggcgggcacctccacctcttcggcctgctcgtcatcgtcgacgtctccggcgggaggctcgacgaggtcgcccccgaggcggccagcttggcgaggatcaagcagcaggcgccgtgcaggatcgtctgcctgacggacctcaagaccccctccgaggatctgaggaggttcagtgaggcggcgagcatcgacctcaacctgcgcaagcccatccacggctcccggctgcacaagctactccaggtcatgagagacctccatgccaacccgtttacgcagcagcagccgcagcagctcggtacagccatgaaagaactgccggctgctgatgagacctctgcggctgaggcgtctgagatcacgcccgcggcggaggcgtcttctgaaatcacgcccgcggcggaggcgtctgaaatcacgccggcagcgccggcgccggcgccccagggagcggccaatgctggagagggcaagccgctggaggggatgcgcatgctgctggtggacgacaccacgctgctgcaggtagtccagaagcagatactgaccaattacggggcaaccgtggaggtcgccacggatggcgccatggccgtggccatgtttacaaaggctcttgagagcgcaaatggcgtctcagagagccatgtggacacagtggccatgccctacgacgtcatcttcatggattgccagatgccagtgatgaatgggtatgatgcgacgaggcgcatccgggaggaagaaagccgctacggcatccgcaccccgatcatcgcgctcaccgctcattccgcggaggaggggctgcaggagtccatggaggcagggatggatcttcacctgaccaagccaatacccaagccgacaatcgcacagattgttcttgacctctgcagccaagttaataactga

SEQ ID NO: 15 - protein, 1081 aa, Triticum aestivum
MAGEVGKWGSSFKRSWALIPLVAHGIIVVVVGLAYSFISSHINDDAVSAMDASLAHVAAGVQPLMEANRSAAVVAHSLQIPSNESSYFRYVGPYMVMALAMQPQLAEISYTSVDGAALTYYRGENGQPRAKFGSQSGQWHTQAVDPVNGRPTGRPDPGASPEHLPNATQVLADAKSGSPAALGSGWVSSNVQMVVFSAPVGDTAGVVFAAVPVDVLAIASQGDAAADPVARTYYAITDKRDGGAPPVYKPLDGGKPGQHDAKLMKAFPSETECTASAIGAPGKLVLRAVGADQVACTSFDLSGVNLGVRLVVSDWSGAAEVRRMGVAMVSVVCAVVAIATLVCILMARALWRAGAREAALEADLVRQKEALQQAERKSMNKSNAFARASHDIRSSLAAVVGLIDVSRVEAESNANLTYNLDQMNIGTNKLLDILNTILDMGKVESGKMQLEEVEFRMADVLEESMDLANVVGMSRGVEVIWDPCDFSVLRCTATMGDYRRIKQILDNLLGNAIKFTHDGHVMLRAWANRPIMRSSIISTPSRFTPRCRTGGIFRRLLGRKENRSEQNSRMSLQNDPNSVEFYFEVVDTGVGIPQEKRESVFENYVQVKEGHGGTGLGLGIVQSFVRLMGGEISIKDKEPGEAGTCFGFNIFLKVSEASEVEEDLEQGRMPPSLFREPACFKGGHCVLLAHGDETRRILYTWMESLGMKVWPVTRPEFLVPTLEKARSAAGASPLRSASTSSLHGVGSGDSNITTDRCFSSKEMVSHLRNSSGMAGSHGGHLHLFGLLVIVDVSGGRLDEVAPEAASLARIKQQAPCRIVCLTDLKTPSEDLRRFSEAASIDLNLRKPIHGSRLHKLLQVMRDLHANPFTQQQPQQLGTAMKELPAADETSAAEASEITPAAEASSEITPAAEASEITPAAPAPAPQGAANAGEGKPLEGMRMLLVDDTTLLQVVQKQILTNYGATVEVATDGAMAVAMFTKALESANGVSESHVDTVAMPYDVIFMDCQMPVMNGYDATRRIREEESRYGIRTPIIALTAHSAEEGLQESMEAGMDLHLTKPIPKPTIAQIVLDLCSQVNN

SEQ ID NO: 16 - DNA, 13255 bp, Triticum aestivum
ttgcttttaagttgtaaatgtcgtaggcttccttctcacgttatttttcttttcttttagtcggagggtgtgtgttgtggtctgctgggaaaagcttccctgccctaattgggtccactacttctttaacgtttaccacttcaattaaacgagttcaataacgaaacgcttttgtacaaatgtaccagcctttatggtttatttatgtaatcaatcatgacgtattcacccaagtacattctgatatttatgttgaatgtgaacattgtctattaatcatggggtagtgtatatactcactagggtgctcatgtgcttaagttgcatccccacaattgtttatatttactacaaaacaaagataactggatcaacgaacgaataaattgacgggtggtcctttcatgctatccaccagatggggcaattgcttttaagttgtagatttcgtaggcttccttttcatgttatttttatttttgattagtcagatggtgtgtgttgtgatctgctgggaaaaatctcccccctccattggtggcaataaacataaaaagggtcagctctcacgtcatacaaaaataaaagaaaacaaatttgaatataaatcaatataatttacaataacacacaagaccgtcccattaccattgtaggaaacgccacaccctttcccatttttggaaattgaccaccaccggtggctcatcctgtaaaccttcccttcaacttcttggttgttctcatctacttctaacttaattatttaagggacatgcatagaaggtgactaccagtgatgagcacggcgtcatgggagcctatgaacaactttcaaccatacatgacacaccttttataaagggaaacccatatttctaactaaacttcaacaatattcataaaaaatagcatgtgatgccactttcaaccaaaatttagtatccaaaaatctacaattttttgaattaattgtttaattttgtacaaaattcagatggcttaaataaacattcatgcatttctaactaaaaattctcacaaaaaattcttccaactttcaactcaagggaaaccgaaagatgtggccaatcctaccttagagatggctttatcaaggcatgatcatgataatgggacaagtatagcctcctaagggttgttaaagaacgtgcatgttgaaattaccatcatcataagatccatgcccccccccccacacacacacatacctacatgatccaatcacaagagatttggtgggacatggtattatattttcttgggtgtggtttgaaaagttcaagccaaccttgtctattatgtcttaattaaaagttgtgtcgtgcaatcgtttaaatgcagttccatttttctccaaaaagacaaatgaccaaaacaacaccttcatttaccatcgctaccttctacccatatccattccctgctcccatccgtcctcggtcacagctcctacaccatgagacagagggaggggatcctcatctctcatttgatgtgtaggaaaagttcgttggttagtttttggtgtcacggacatcacaataacagttacgactccaatactgtcttgacggtgtttggtgaggtgttgccaaggagattgtgagtattttttgttctcgtcggcctttttggacgacgttgtggttcttgttgctattttggcgaggcatcgttgactggtgtcgtcttcttcaacaactctttccttcgagcctttgcgagcaatgtcagtggcctagtttggcaatatgagtgtggctgccttcctagatcccccatgccaaatgttcttggcatgattgctaatacctgttatacacggccgtctaccattgcgaccattcaagatgtggtcctctagaggtcttcactgactaagattctcgatgttgttctccgccggcgagagctaggtggggcaacgacgacgagtttggttacggtttggcttgaattttgcagccgacggtgttggttataattcatcatttgtttatggttatttctatgcttgtgggtttagttacctcgttatttgaatgtattcgcttttttctttgtgatataagatagaactgattaaaaaaattgtgcaagtaatagtgaggcaaataagctacatatgtacattgaaaacaatatgacatatccactaactataatacgcacaaaagaattgcgtatgagtcttgtattttttttttcttattttcaatggaatataggtgacatgttgaacggtctcacacgaacggctctacatattgcccactcggcacacaagaggaacgctcgaacttgtcgtgtgtgtgcggacaaaataaatagaacttttcaatttccgcgctttgagattttcagctgataattaacgcattagacagagcatcgaacgagtcgatcaagtttgaataagtaagtcacaggctaaaaaaagcaagacacagttcatcttttttttaggaaaggacttggttcatgtttattttattttttgaggaaaagggcctggttcatcctagctgtcctggtaacggagctagttcaacataagtcgagcctgtgcttctatatttagcatcaaacgtaacgaagagttaacttaacaaaactaatgataatgctatcttaagacaggaagctaattgatcggtgttcacttgcacaacaggatggccttatggcgatcgtgcggctgccaacactgcctcacgcccacccaaactattcgaacgggaggaaaacataattcattgtgctcagatttggcaattgattacaagatcgcgtagtatccctttttctatctcgtcgtgtgtctactaccgagccgtgcattgatattagatatcgaagttaaatgttgatttttttagaataaatacttcttttttattgcggtgcaggggtaaatatcgattttttttctgcggtacaggagaaatactgccaaaagtggtataataaaccgcgacggcggatttaaaaaaccggcggcgctctgagcccacaattcagagcggaaaaaaccttcaatgtgtaaagtgtattcctccgagcatctataaaaggccgtatacctaggaaacaaatactcacgaacacctaacaaagatttgcttctgtagaatactttacaatacttccgcgatggctggagaggttggcaagtggggtagttccttcaaacgttcttgggctttaatcccactggtatgagaacattcatagtacttggcttctttttgtgaaactcatggttaatacttgttcttctatatgaacttcatggctattctcttaaaaaaaaacttcatggcttttctcatttcttggttcttgtccttccacctacaactattgcttacctgagcggaaactagatctgcgaggttatcatctgcttaagctttttttttttgagggattcatcttacttaagcttagtgcaacaaagacatccttccgcatatgtgggtatgtgatctccacaatagatctatgtagtggtacggtattcttttggaagaggaggattaccccccggtctcttggcataggtgtgagttcaaaagtaattttaatgatttttgtcttacaattaaattctgtttcgtacgatatagatatcttcctatgcctgtaaggcgcagatttgaaaagtagatctaactgatttttattgtttatgtttcatttctgtatattacagatatcatcctgcacgtgtcaggcatgcgagatctctttctttagataacaatgaaaactagatcttcataatttttcatcttgtaattgaagattttgttccgtacaagatggatatcctccgacgtgtgttttaggcatgaattcaaaaagtaaatataattttattttatcttgtaattaggaatccgctccgtacaacattagtgtcctttcacctgtgtaaggtgtgctctaaaaatacatctatgtagattttgatatataatttggcttctgctccgtacatcacagatatctccccacatgtgtgaggcagtggcgagtcaaagaactcccttttgtcgtatgatcctatgttgtttttctggtcaccgtgtgttagatctacccatggaattgtttttctggtcaccgtgtgttagatctacccatggaatgtcacgacaaatgttgtcgttagatgagcccaaaggcgatctgttagcgggatgttcacgacaaattatatctttagattagtgaaaaccctcgaaatcttgacttattacttgtgcaacaaatgttatcgttagacgagttgaaacacaatgcaacgccgcttgtcgaggcctcgtcagcctaaaagacagtttaatttatccgcaaaaaaaagacactttaatttaatatttatgcattttctattttactttttacatgttgcaaaaaaaaatctgacgtcacacttttattgcacttgcacgcatgcaggtcgcccatggaatcatcgtggtcgtagtgggtctggcttactctttcatctcgtcgcacataaatgatgatgccgtgagcgccatggacgcgtcgctggcgcacgtcgccgccggcgtgcagcctctaatggaagccaaccgctccgccgccgtcgtcgcgcactctctgcagatccccagcaatgaatcatcttatttccgatacgtaattaaccaagaacctttgggttaattaaattatgcattttttctatgtaaaacgtgattagtttcatcacgtatatgcacttcctttttgaaccacaattatttccttactttaaataacaaatcttaattactagccgggccgacccggtaactggttattgtgtatgattctgttctgattttcgtagtaatgcgagcattgatatgaatatacgcatgcatatacaagcaaataatttttgcgtgcatttttttttatgtaggacacgtccaagataacatagcaacacgtactacgtgcaaatatgcatctaacatttacgtatatgtttgacctgacaggtgggaccatacatggtcatggcgttggccatgcagccgcagctggccgagatatcatacaccagcgtggacggcgccgcgttgacgtactaccgcggcgagaacggccagccgagagccaagttcgggagccagagcggccaatggcacacccaggccgttgatccggtgaacggccgtcccaccggccgccctgacccaggggcgagtccggagcacctacccaacgcgacgcaggtcctcgccgacgccaagagcggctcgcccgcggccctcgggtccgggtgggtcagctccaacgtccagatggtggtcttctccgcgcctgtcggtgacactgcgggcgtggtcttcgcagcggtccccgtcgacgtcctggcgatcgccagccagggcgacgccgccgccgatcccgtcgcgcggacgtactacgcgatcaccgacaagcgcgacggcggcgccccgccggtttacaagcctttggacggcgggaagcccggccagcacgacgcgaagctgatgaaggcctttccctcggagaccgaatgcaccgcgtccgccattggcgcgcccggcaagctcgtgctccgcgccgtcggggcggaccaagtcgcgtgcacgagcttcgacctctccggagtgaacctggtaacgcgtccatctagcatcgatcacaggccatccatatatgcatacgtacaccaacgtgcacacagcctatctaacgtaattcctgtgcattatttttgtcaagaactaatcccagcatgtaatatttcttccaagtttgctgtttatacattaaaaagcaacggataatgaaaaaaggttagatgagctaaggggactttggcaaaaaaaaaaactaataaaacgtttttttgttttggcgacctcactaaacatccttccgacttggagtgaggaaaaaaaacagggatcgctcctagctattgacagtacgtacacaatcttgcttccttcctttcgcatgtaaaaaaactgaaaactttccaaatcaagggatcccaaaattaggaagaaaatcttaatggagagtacaaagtcttcttcttcctcttcttccccaaagcaagatttctcattctgttcttccccaaacggagctctggcacaaaactgtggtgagctcgatcgtctcctacgtacttttcttgcatctgctagtgtcttgcatgcatatcaccggttgtcgttcatggatatctcccatcagttcttttgcaatttatttacagcgtatgaacgagcgctggtttgaaactgatctcccatctgctagtgcaattaggatatctcccatctgctagtgtacttttattgaaactgatcttgccatccgtcgcctatgaacgagcgctggtttgataattctccgaccaggccgggcggcgcctcgcgcgccatagaaacagtcttttttttcgataaaggccatggaaacaacctgacgtacagcctcttgtaaaaaacaattattttcttcgtagtatagcacgcatatgcatgtttgagaatttttatcgggacggctgacaagtatctccggttgtatttcttcttgtttttcagggagttcgtcttgtggtcagcgactggagcggggcagccgaggtccggcgaatgggggtggccatggtgagcgtcgtgtgcgcggtcgtggcgatcgcgacgctggtgtgcatccttatggcacgggcgctgtggcgggccggggcgcgggaggcggctctagaggctgacctggtgaggcagaaggaggcgctccagcaagcggagcgcaagagcatgaacaagagcaatgccttcgcccgcgccagccacgacatccgctcctcactcgctgccgtcgttggactcatcgacgtttcccgggtagaggccgagagcaacgccaatctcacctataacctcgaccagatgaacattggcacaaacaagctcttgggtcagtctgcatccatgccctacgtaccatgcatgacaataccatgaatagcttgcgctaccttttagtagatctatccgtacttggcaatttagctaatgtcatcatagcattataaaattgcatgtcatagaagtaaagtttctgtaaataatttaattacagtcttaggagtagggtatgcaatatcccagctgttatacatttaaggtatcaaatttgctcataaaatttaaaatatgcaagaaatcaatctctgtttggtaaagaaatagcattttatttgtacaagaaataaagtttaggatagttcaaatcgaattgtcagatatcactatgttagcagccagtaaacttatgaagtttcaatatttctatctattttgctgctgggcaaattttgtcacatttacgcatcatgttttttgtgcgtgtctttgagtgcataaagcaaaaaagtttattatttccgaaaaaatgaactttataccatatttgtgttctcaactgagcatatgttgactcatcatccatgtttatacatgtgtgtgtacatgcagatatacttaacacgatactggacatgggcaaggtggagtccgggaagatgcagctagaggaggtggagttcaggatggcagacgtccttgaggaatccatggacctggcgaacgtcgtcggcatgtcaagaggcgtcgaagtgatctgggacccttgtgatttctccgtgctccggtgcaccgccaccatgggcgactacaggcgtatcaaacaaatccttgacaacctactcggcaacgccatcaagttcacacacgacggccacgtcatgcttcgagcatgggccaaccgtcccatcatgaggagctccataatcagcaccccatcgaggttcaccccccgttgccgcacgggtgggatctttcggcggctgcttggaaggaaggagaaccgttcggaacaaaatagccgaatgtcattacaaaatgatcccaattcggtcgagttctacttcgaggtggttgacactggtgtgggcataccccaggaaaagagggagtctgtgtttgagaactacgttcaagtgaaggaagggcatggtggcaccgggctcggacttggaattgtgcaatcctttgtaagtgatctcatcttttttcatccatgttaaaatcttgtcaagtgcatcaacgttaactagccgtaactgtattcttcatgggtaggatgtgtgtgtgttcgtgtttgtttgtttggaaaagaaaattatatttttcactaacgttttcgttttttcttgtttacttatagttttgtttgctgttgttgttgatgtaaacataggttcgtctgatgggaggagaaattagcatcaaggacaaggagccaggagaagcggggacgtgcttcggcttcaacatcttcctcaaggtcagcgaggcatcggaggtggaagaggacctcgagcaagggaggatgccgccgtcgctgttcagggagcccgcctgcttcaagggcgggcactgcgtcctcctcgcccacggcgacgagacccgccggatcctgtacacgtggatggagagcctcgggatgaaggtctggcccgtcacgcgccccgagttcctcgtcccgaccctcgagaaggcgcgctccgccgccggcgcctcgccgttgaggtcggcgtcgacgtcgtcgctgcatggcgtcggcagcggcgactccaacattacgacggaccggtgcttcagctccaaggagatggtcagccacctgcggaacagcagcggcatggccggcagccacggcgggcacctccacctcttcggcctgctcgtcatcgtcgacgtctccggcgggaggctcgacgaggtcgcccccgaggcggccagcttggcgaggatcaagcagcaggcgccgtgcaggatcgtctgcctgacggacctcaagaccccctccgaggatctgaggaggttcagtgaggcggcgagcatcgacctcaacctgcgcaagcccatccacggctcccggctgcacaagctactccaggtcatgagagacctccatgccaacccgtttacgcagcagcagccgcagcagctcggtacagccatgaaagaactgccggctgctgatgagacctctgcggctgaggcgtctgagatcacgcccgcggcggaggcgtcttctgaaatcacgcccgcggcggaggcgtctgaaatcacgccggcagcgccggcgccggcgccccagggagcggccaatgctggagagggcaagccgctggaggggatgcgcatgctgctggtggacgacaccacgctgctgcaggtagtccagaagcagatactgaccaattacggggcaaccgtggaggtcgccacggatggcgccatggccgtggccatgtttacaaaggctcttgagagcgcaaatggcgtctcagagagccatgtggacacagtggccatgccctacgacgtcatcttcatggattgccaggtacatttctccagcaaacaacgtgccaagcacatcagccccatctctcttgttcctgaagatgatttaatctgacgttgctgacaattcgatcttctttgtttcagatgccagtgatgaatgggtatgatgcgacgaggcgcatccgggaggaagaaagccgctacggcatccgcaccccgatcatcgcgctcaccgctcattccgcggaggaggggctgcaggagtccatggaggcagggatggatcttcacctgaccaagccaatacccaagccgacaatcgcacagattgttcttgacctctgcagccaagttaataactgatcgcggagattcttcgttccctgttccctgttccccggtcacatgatcaaatatcaagataggtgtaggtggtttttcagccagcgaatgcagttgtcatcctagtcactgaaaacccacctacatctcgagttttgatcatgcgaccaggggcattatcgtagtttgtagcatttagcagcagcagctgaactttgttgttgtatcaagatcaggtcaggtttatttccagaattactcttggacaatgtattgtcaattttgaatttccagaaacaattatggttaagttttgagttccagagttggtgttttcagagttcttttttttccggagtttgtgtttgggtctgtctagcacacatctagatgtgacatagttatgtcacatctaacctgataagcactatgtttgtggtctattttttttgtcccagctttttttttatttcttgttgctgtgtagttatttttagaaggttagatgtgacatcactaaaaaacatctagatgtgaattagacaaactgtttgtgttttcagagcatgtgattagacgccatatatttgcttccattgcccattctctggagaaagaaagtacagattcctacaagctatgaaatccctggctagctaccttgtatatctagtagtgtacacaagcatagctgataaatacccataggaataactgtacagtctcctctaggtctgcagtggacttgcctaaatactagtactatctctcatatgcacgcaccaagtgggaaaagttcacacccgaggtcattttcattgaatggcacttcgtcgttctcctgcgttgaaatcagaaaaagggttcagaagaaacaccattgaaaatctaggaacatagggtttacttagcttcgatcagtgcaaacgatttgaaaggaaatatgcctatccgaaatagtgaaattttgaggggggaagagtaaagtcaagcataactgaggttcttaccacttttattatagaattgagaagaccatcaaagttgcagctgctggaattgaacccaccctgcagctttggaactcccaaatcatacatatcggtctgctcaggaacaatggcaccaccatcatagctaaaaccatcattgttcagtctatggtcttgtctcattccatccattctatgtctggagttgcttgcagccccaaactggaggtattttgaatgtctttcggtatcatgaggaaggggcagaattgtactgccagaggaactagctctacatttggcattgctggctactataaggctctcagaaggacaaacactaacacccattctttccgaaaatatccccttttggtccatctcatgttctccagccacaaagctagcgtcaagttttgtcgcgccaactcgatcgaacgggatgtgcaacggtaacttgtcaacagaaaacccatcactgattagaagagcacaccgctgagatatacaagaatcccggaaatcggcggaaactccgacagacctttcaagcagccctgaacttgcagttggtattcctattgatggatgggctccaactttgatgtgtgtagtgcattccaaaagatcttctggtggcaatgaactgcttgtaactctttggagtgtgccagacagagtgttagccagagcacccccggaaaagacagagcacaggtcattggtttcttgatggatccacttctgctggagctgaggctgcccaagcgacgatgaggtcaagccttgcgataaatctgcctgttggttgtcttgtaagctcacaatgtggaacttatcggtatcgccggcgcaatgacttactgcgtcgttgctgctagaacactggtttgccttgggagaagcaaggtcctgaagcccaaatgctgctgcaccagcactactcagcagtccatgtggattgaaagatggaagagcagcagaaggggcaaagggttgatgataactatgaagtccttcaaatgctcccatgtgcaagaaggggtctctgcctccaaaagcagcagcaatgctggcttgctgtgatgccacggcacttagccgtctgaggtatagcctgtacttctgcattggcatacaaagtaacctgattagacatggaggaacttatgtaatcatcagaattctgaaacatgagatatacagtcttaagataactgcttcctggtttggcatggagattcttgctaaactggtacataaaagcactccaggatatagaaaaggctgatggattttcttaaccttttaatgtaggcttgttaataaattttctatttgtggattcattgacatgcaaagcttttgcatttctctaaaaaatatttcagtttacatatgtagcgctgtaatctggacagggtggtgtcagtgtcttccttctagcagttttacagaagtgaaacaatgtagccaaaatactacaataaagtcagcagtacataccaaaacaccacattaaacatgtacagcagtacataccaaaatacaacattaacttgtacagcagtgcatgccaaaatactgtagtaaacttgtacagcagtattacctaattactctgcattaaacctgtacaacagtacatactaatttgagcattttatacctgtgtagttagatataatctactagcctacatggttgggagaataatcagatatttgtattagatatctccttcaggttttctgaaagattcagtataactgaactgaatgtttctttgttttcagaccaactgaacatattcaacttgtcaagaaaaaggaagaaatgtgaaggtgaaactatgtagccgcaaaattaaactacttaactcattttgcacgaacaccaggaacatgttaacttatttgttgaaagagttctgctctgcaacaccatgatatcaaattaagctctgctctgcaactaataggtgtgtcgataccaaatacagtaaggggataaggactctgttataagttggccctcttaattcgacacaggaaaataaggccatttgcagataattttaccaaaagttctgtaaaagtttttcttcctgtgaccaacgaaagagataacacaggtaaaggtgacgatggaagtaaatgccatgtcgctatacctgcagatggctcgcaacattttccctggtgagcttctccacgttcataagctcgagtattcttttgggcacagcctctgcaaacagcagcaagagaaaggacacgagcataaatgggtatgctgtagcctgcaagaacatttaacataaagcaaaaggcaacgtggggaggtgttttttttttctttcttactgtcgatcccgagctggttgacggcggcgacgaacttccggtgcagctccactgaccacaccaccctcggcctcttcga

SEQ ID NO: 17 - DNA, 3312 bp, Triticum aestivum
atggttggagaggttggcaagtggggtagttccttcaaacattcgtgggctttaatcccactggttgcccatggaatcatcgtgatcgtagtggctctcgcttactctttcatctcgtcgcacataaatgatgatgccacgagcgccatggacgcgtcgctggcgcacgtcgccgccggtgtgcagccgctcgtggaagccaaccgctccgccgccgtcgtcgcacactctctgttcatccccagcaacgaatcatcttatttccgatacgtgggaccgtatatggtcatggcgttggccatgcagccgcaggtggccgagatatcatacgccagcgtggacggcgccgcgttgacgtactaccgcggcgagaacggccagccgagagccaagttcgtgagcgagagcagcgaatggtacacccaggacgttgatcctgtgaacggccgtcccaccggccgccccgacccggcggctcagccggagcacctacccaacgcgacgcaggtcctcgccgacgccaagagcggctcgcccgccgccctcggggccgggtgggtcagctccaacgtccagatggtggtcttctccgcgcctgtcagtgacactgccggcgtggtctccgccgcggtccccgtcgacgtcctcgcgatcgccaaccagggcgatgccgccgccgatcccgtcgcgcggacgtactacgcgatcaccgacaagcgtgacggcggcgccccgccagtttacaagcctttggacgccgggaagcccggccagcacgacgcgaagctgatgaaggccttttcctcggagaccaaatgcaccgcgtccgccattggcgcgcccagcagcaagctcgtgctccgcaccgtcggggcggaccaagtcgcgtgcacgagcttcgacctctccggagtaaacctgggtgttcgtcttgtggtcagcgactggagcggggcagccgaggtccggcggatgggggtggccatggtgagcgtcgtgtgcgtggccgtggcggtcgcgacgctggtgtccatccttatggcacgggcgctgtggcgggccggggcacgggaggcggctctagaggctgacctcgtgaggcagaaggaggcgctccagcaagcggagcgcaagagcatgaacaagagcaatgccttcgcccgcgccagccacgacatccgctcctcactcgctgtcgtcgttggactcatcgacgtttcccggatagaggccgagagcaaccccaacctcagctataacctcgaccagatgaacattggcaccaacaagctcttcgatatacttaacacgatactggacatgggcaaggtggagtccgggaagatgcagctagaggaggtggagttcaggatggcagacgtccttgaggaatccatggacctggcgaacgtcgtcggcatgtcaagaggcgtcgaagtgatctgggacccttgtgacttctccgtgttgcggtgcaccaccaccttgggcgactgcaagcgtatcaaacagatccttgacaacctacttggcaacgccatcaagttcacacacgaaggccacgtcatgcttcgggcatgggccaaccgccccatcatgaggagctccgtggtcagcaccccatcgaggttcaccccccgtcgccccgcgggtgggatctttcggcggctgcttggaaggagggagaaccgttctgaacagaatagccgaatgtccttacaaaatgatccgaattcggttgagttttactttgaggtggttgacactggtgtggggataccccaggaaaagagggagtccgtgtttgagaactacgttcaagtgaaggaagggcatggtggcaccgggctcggacttggaattgtgcaatcctttgttcgtttgatgggaggagaaatcagcatcaaggacaaggagccaggagaagcggggacgtgctttggcttcaacatcttcctcaaggtcagcgaggcgtcagaggtggaagaggacctcgagcaagggaggacgccgccgtcgctgttcagggagcccgcctgcttcaagggcgggcactgcgtcctcctcgcccacggcgacgagacacgccggatcctgtatacatggatggagagcctcgggatgaaggtctggcccgtcacgcgcgccgagttcctcatcccgaccctcgagaaggcgcgctccgccgccggcgcctcgccgttgaggtcggcgtcgacgtcgtcgctgcatggcgttgggagcgccgactccaacattacgacggaccggtgcttcagctccaaggagatggtcagccacctgcggaacagcagcggcatggccggcagccacggcgggcacctccaccccttcggcctgctcgtcatcgtcgacgtctccggcgggaggctcgacgaggttgcccccgaggcggcgagcttggcaaggatcaagcagcaggcgccgtgcaggatcgtctgcctgacggacatcaagaccccctccgaggatatgaggaggttcagtgaggcggcaagcatcgacctcaacctgcgcaagcccatccatggctcccggctgcaccaactcctccaggtcatgagagacctccaggccaacccgtttacacagcagcaaccacatcagtccggcacggccatgaaagaactgccggctgctgatgagacctctgcggcggaggcgtcttctgaaatcacgcccgcggcggaggcgtcttctgaaatcactcccgcggcggaggcgtcttctgaaatcactcccgcggcggaggcgtcttctgaaatcactcccgcggcggaggcgtctgaaatcatgccggcagcaccggcgccaactccccagggaccggccaatgctggagaaggcaagccgctggaggggatgcgcatgctgctggtcgacgacaccacgctgctgcaggtagtccagaagcagatactgaccaattacggggcaaccgtggaggtcgccacggacggctccatggccgtggccatgtttacaaaggctcttgagagcgcaaatggcgtctcagagagccatgtggacacagtggccatgccctacgatgtcatcttcatggattgccagatgccagtgatgaatggctacgatgctacgaggcgcatccgcgaggaagaaagccgctacggcatccgcaccccgatcatcgcgctgaccgcgcattccgcggaggaggggctgcaggagtccatggaagcagggatggatcttcacctgacgaagcccatacccaagccggcaatcgcacagattgttctagacctctgcaaccaagttaataactga

SEQ ID NO: 18 - protein, 1103 aa, Triticum aestivum
MVGEVGKWGSSFKHSWALIPLVAHGIIVIVVALAYSFISSHINDDATSAMDASLAHVAAGVQPLVEANRSAAVVAHSLFIPSNESSYFRYVGPYMVMALAMQPQVAEISYASVDGAALTYYRGENGQPRAKFVSESSEWYTQDVDPVNGRPTGRPDPAAQPEHLPNATQVLADAKSGSPAALGAGWVSSNVQMVVFSAPVSDTAGVVSAAVPVDVLAIANQGDAAADPVARTYYAITDKRDGGAPPVYKPLDAGKPGQHDAKLMKAFSSETKCTASAIGAPSSKLVLRTVGADQVACTSFDLSGVNLGVRLVVSDWSGAAEVRRMGVAMVSVVCVAVAVATLVSILMARALWRAGAREAALEADLVRQKEALQQAERKSMNKSNAFARASHDIRSSLAVVVGLIDVSRIEAESNPNLSYNLDQMNIGTNKLFDILNTILDMGKVESGKMQLEEVEFRMADVLEESMDLANVVGMSRGVEVIWDPCDFSVLRCTTTLGDCKRIKQILDNLLGNAIKFTHEGHVMLRAWANRPIMRSSVVSTPSRFTPRRPAGGIFRRLLGRRENRSEQNSRMSLQNDPNSVEFYFEVVDTGVGIPQEKRESVFENYVQVKEGHGGTGLGLGIVQSFVRLMGGEISIKDKEPGEAGTCFGFNIFLKVSEASEVEEDLEQGRTPPSLFREPACFKGGHCVLLAHGDETRRILYTWMESLGMKVWPVTRAEFLIPTLEKARSAAGASPLRSASTSSLHGVGSADSNITTDRCFSSKEMVSHLRNSSGMAGSHGGHLHPFGLLVIVDVSGGRLDEVAPEAASLARIKQQAPCRIVCLTDIKTPSEDMRRFSEAASIDLNLRKPIHGSRLHQLLQVMRDLQANPFTQQQPHQSGTAMKELPAADETSAAEASSEITPAAEASSEITPAAEASSEITPAAEASSEITPAAEASEIMPAAPAPTPQGPANAGEGKPLEGMRMLLVDDTTLLQVVQKQILTNYGATVEVATDGSMAVAMFTKALESANGVSESHVDTVAMPYDVIFMDCQMPVMNGYDATRRIREEESRYGIRTPIIALTAHSAEEGLQESMEAGMDLHLTKPIPKPAIAQIVLDLCNQVNN

SEQ ID NO: 19 - DNA, 13305 bp, Triticum aestivum
gttatatactcactggggtgctcatgtgcttaagtcgtgtccccacaactgttcatatttactgcaaaactaagatagccggatcaacaaacgaataaattgacgggtggtccttccatgctatccaccatatggcgtaattgcttttaagttgtagacttcgtaggcttccttttcacgttattttttcatttagtcggatggtgtgtgttgtgatctgctggaaaaaagacccccattagtggaaataaacatagaaatggcaggctctcatgtcgtacaaaaataaaaaaacaattttgaataaaaatcaatataattcacaataacacacaagaccatcccattaccatagtaggaaatgccacatccatttcccttttttggaaattgaccaccaatggtggctcatcttgtaaactttcccttcaattctcaattgttctcatctacgtacttctaacttaattattttagggacatgcatacaaggtgactagcagtaatgagcacaacgtcatgggagcctgtgaatcactttcaaccataaagggggaaccatatttctaactaaacttcaaaaatattcgtaaaaaaccaatgtgatgccactttcaaccaaaatttagtaaccaaaaatctacaaaaaaatttgactcggtcatttaattttgtataaagttcagatggcttaaaaaacattcatgcatttctaactgaaatttgtcacaaaaattcttacggctttcaactgaagaaaaggaaaccgaaagatgtggccaatcctaccttagagatggctttagcaaggcatgctcatggtaatgggacaagtatagcctcccaaggttggtaaagagtgcatgttgaaattaccatcatcataagaaccatggacgcgaccccccaccccccaccccccacctacatgatcccatattactagagaggaggggacatcatcaaggagacatggaagatgttggtgtgtcgatgaccttgttgtgggtggcacaaatacgtgacatgatagacacgagagaattcgtaagatggtgaaaatcactagagatttggtgggacaaggtattagattttcttaggtgtggtttgaaaatttcaaaccaagcttgtctattatgtcttaattaaacattgtgtcatgcaatcgtttaagcgaagttccatttttctccaaaaatacaactggccaaaacaacgacttcatttaccatcgctaccctctacccatatccaatccttgatcccatccatcctcggatcacagctcctacactgtgatggaggggaagggatcctcatctctcatctgatgtgtagggaatgttatttggttagtttttggtgtcatggtgacatcacaatgatggtgacaactccaatactgtctcggtggtgtttggtcaggtgttgccaaggacattgtgattattttttgttctcgttgacctttttggacgacattgtggttctttctgctatattttcgtgaggcatcattgaccgcggtcatcttcttcgacaaccctttccgtcgagcctttgcgagcaatgtcagtggcctagtttggcaatatgagtgtcgttgcctttctagatcccccatgctaatgttcttggcatgattgctaatatttgttatacacgaccgaccgcctaccattgcgaacattcaagatgtgtttctgtagaggtcttcactgactcagattctcgatgttgttctccgcttgcgagatctaggtggggcaacgacaacgagtttggttacggtttggcttgaattttacggcctacagtgttggatagaattccatttgtttatggttaattctatgcttgtgggtttagttacctcgttatttgaatgtattcgctattttctttgtgatataagatagaactaattttaaaaagaattatgctagtcaatagtgatgaggcaattaagctacatatgtacatcaaaaacagtacgacagatccactaactataatatgcacagaagaattgcgtatgagtcttgtatcttctttcatcatttattttccatggaatataggtgacatgttgtacggtctcacatgaacagtcctacatattgcccactcagcgcacaagaggaacgctcgaacttgtcgtgtgtgtgcgaacaaaataaatataacttttcaatttccgtgcttcaagattttcacatgatgatcgagtttgaataagtatgacacactacaaaaaaatcagctgatgatcgagtttgcataagtaagaaacaggctacaaaaagaaagtaagacacaattcatcgtttttttttcaggaaaggacctggttcatgttcattttatttttttgagggaaaggacctggttcatcagtcttagctgtcctggcaacggagctagttcaacataagtcgagcctgtgcttctatatttagcatcaaacgtaaccaagagttaacttataacaaaactaatgatcatgctatctaagacacgaagctaatggatcggtgtacacttgcacaacaggatggccttatgacgatcgtgcggctggcaacactgcccccatgcaaggccaacactgcctcagcccgcccaaactattcgaacgggaggaaaacataattcattgtgcttggatttggcaattgattacaagatcacgtagtatcccgttttctatctcgtcgtgtgtctactaccaggccgcgcattgatattagatatcgaagttaaatgccgattttttttaaataaatacctttttttattgcgatgaaggggtaaataccatttttttctgcggtgggcggaaaatacagccaaaagtggtataataaaccgcgacggtggatttgaaaaaccggcggcgctctgagcccacaattcagagcggaaaaacccccccaatgtgtaaagtgtattcctccgagcatctataaaaggccatctacctaggaaacaaatattcaccaacacctaacaaagatttgcttttgtagaatactttacaatacttccgcgatggttggagaggttggcaagtggggtagttccttcaaacattcgtgggctttaatcccactggtatgaaaacattcatagtagttggcttctttttatgaaactcatggttaatacttgttcttctatatgaacttcatggctattctcttccttcaaaaaaaactccatggcttttctcatttcttggttcttgtccttccacctacaactattgcttacctgagcggaaactagatctgcgaggttatcatctgcttaagctttttttgagggattcatcttgcctaagcttagtgcaacaaagacatccttccgcatatgtggagatgtgatctcgaaaatagatctatgtagtggtacggtattctattggaaaaagaggattacccccgtctcttggcataggcgtgaggttcaaaagtaattctaatgatttgtgtcttacaattaaattttgtttcgtacgatatagatatgttcctatgcctgtaaggcgcagatttgaaaagtagatctaactgatttttatcgtttctgtttcagttatgtatattacagatatcatcctgcacgtgtcaggcatgcgagatctctttctttagataacaatgaaaactatatcttcataatttttcatcttgtaattgaagattctgctccgtacaagccggatatcctcccacgtgtgttttaggcatggattcaaaaagtaaatataattttattttatcttgtaattaggaatccgctccgtacaatattagtatcctttcacttgtgtaagacgtgctctaagaatacatctatgcaggttttaatatataatttggcttctgctccatacatcacatatatctccccacatctgtgaggcagtggcgagccgaagatctaccttttgtcgtatgctcctatattgtttttctggacgccgtgtgttagatctacccatggaatgtcacgacaaatgttgtcgttagatgagcccaaaggcgatctgttagcaagatgtcacgacaaattatatctttagattagtgaaaaccctcgaaatcttgacttattacttgtggaacaaatgttaccgttagacgagttgaaacacgttgcaacgccgcttgtcgaggcctcgtcagcctaaaagacagtttaatttattaaaaatacactttaatttaataattatgcattttctattttattttttacatgttgcaaatatatttttctgacgtcacacttttattgcacttgcacccatgcaggttgcccatggaatcatcgtgatcgtagtggctctcgcttactctttcatctcgtcgcacataaatgatgatgccacgagcgccatggacgcgtcgctggcgcacgtcgccgccggtgtgcagccgctcgtggaagccaaccgctccgccgccgtcgtcgcacactctctgttcatccccagcaacgaatcatcttatttccgatacgtaattaaccgagcacctttgcgttaaattgaaatatgtttctcttttctctgtataacgtgattaatttcatcaagtatatgcatttcctttttgaaccacaattatttccttactttaaataacaaatctttttggggggaattaaataacaaatcttaattactagccgggccgacccagtaactagttattgcgtatgattctcttctgattttcgtaataatacgagcattgatatgaatattcgcatgcatatacaacaaataatttttgcgtacccttttttatgcagtacacgtccaaaataacatatcaacacgtacgtgcaaatatacatctaacatttacatgtattcttgacctgacaggtgggaccgtatatggtcatggcgttggccatgcagccgcaggtggccgagatatcatacgccagcgtggacggcgccgcgttgacgtactaccgcggcgagaacggccagccgagagccaagttcgtgagcgagagcagcgaatggtacacccaggacgttgatcctgtgaacggccgtcccaccggccgccccgacccggcggctcagccggagcacctacccaacgcgacgcaggtcctcgccgacgccaagagcggctcgcccgccgccctcggggccgggtgggtcagctccaacgtccagatggtggtcttctccgcgcctgtcagtgacactgccggcgtggtctccgccgcggtccccgtcgacgtcctcgcgatcgccaaccagggcgatgccgccgccgatcccgtcgcgcggacgtactacgcgatcaccgacaagcgtgacggcggcgccccgccagtttacaagcctttggacgccgggaagcccggccagcacgacgcgaagctgatgaaggccttttcctcggagaccaaatgcaccgcgtccgccattggcgcgcccagcagcaagctcgtgctccgcaccgtcggggcggaccaagtcgcgtgcacgagcttcgacctctccggagtaaacctggtaacgtgtccatctagcatcaatcacaggccatccatatatgcatacgtacacgaacgtgcacacaccctatatatctaacataattgctctgcattttggtcaagacttaatcccagcatgtaatatttcttccaagtatgctgtttatacattcaagcaactgacaatgaaaaaaggttagatgagctaaggggacttcggcaaaaaaaactaataaaacgttttttgttttggcgacctcaataaacatccttccgacttggagtgaggaaagaaaatcagggaacgctccgctatggaaagtggacgtacatgatctttcttccttccttcctttggcatgtaaaaaactaaaacctttccaaatcaaggtgtcccaaaattaggaagaaaatcttaatggagagtacaaagtcttcttcttcctcttcttccccaaagcaagatttctccttctgttcttccccaaacggagctctggcacaaaactgcggtgagctcgatcgtctcctacgtacttttcttgcatctgctagtgccttgcatgcatatcaccgattgtcgttcatgtatatctctcctcagttcttttgcaatttatttacagcgtagagagcagtttagactacgtagtaaatcaccattgaaactgatcttgccatccgtcgcctatgcaacgagcgcaggggctgcgctagcttgataattctccgaccaggccgggcggcgcctcgcgcgccatggaaacagtcttttttttttttgataaaggccatggaaacaacctggcgtacagcctcttgataaaaaaaaatattttcttcgtagtataagcacgcatttgcatgtttgagaatttttattgggacggctaacaagtatctccggttgtatttcttcttgtttttcagggtgttcgtcttgtggtcagcgactggagcggggcagccgaggtccggcggatgggggtggccatggtgagcgtcgtgtgcgtggccgtggcggtcgcgacgctggtgtccatccttatggcacgggcgctgtggcgggccggggcacgggaggcggctctagaggctgacctcgtgaggcagaaggaggcgctccagcaagcggagcgcaagagcatgaacaagagcaatgccttcgcccgcgccagccacgacatccgctcctcactcgctgtcgtcgttggactcatcgacgtttcccggatagaggccgagagcaaccccaacctcagctataacctcgaccagatgaacattggcaccaacaagctcttcggtcagtctaccatgcatggcaataccatgaatagcttgtgctaccttttagtagatctatccgtacttggtaatttagcaatgtgatcatagcattataaatttgcatgtcatagaagtaaaattttcgtaaataatttaattactgttttaggtgtagaagtgtgcaatagcccacctgttatacatttaaggtatcaaatttgctcataaaatttaaaatatgcaagaagtcaatctctgtttggtaaagaaatagcattttatttgtaaaaattaaagtttaggatagttcaaatagaattgtcagatatcactacgttagcaaccagtaaacttataaagtttcaatatttctatcgattttgctgttgggcaaattttgtcacatttacgcatcatattgtttgtgcgtgtcttcgagtgcataaagcaaaaaagtttattatttcccaaaaatgaactttataccatctttgtgttctcagttgagcatatgttggctcatcctccatgtttatacatgtgtatgtgtacatgcagatatacttaacacgatactggacatgggcaaggtggagtccgggaagatgcagctagaggaggtggagttcaggatggcagacgtccttgaggaatccatggacctggcgaacgtcgtcggcatgtcaagaggcgtcgaagtgatctgggacccttgtgacttctccgtgttgcggtgcaccaccaccttgggcgactgcaagcgtatcaaacagatccttgacaacctacttggcaacgccatcaagttcacacacgaaggccacgtcatgcttcgggcatgggccaaccgccccatcatgaggagctccgtggtcagcaccccatcgaggttcaccccccgtcgccccgcgggtgggatctttcggcggctgcttggaaggagggagaaccgttctgaacagaatagccgaatgtccttacaaaatgatccgaattcggttgagttttactttgaggtggttgacactggtgtggggataccccaggaaaagagggagtccgtgtttgagaactacgttcaagtgaaggaagggcatggtggcaccgggctcggacttggaattgtgcaatcctttgtaagtgatctcgtcttttttcgtgcatgataaaatcttgtcaactgcatcaaagaaaagtactatctccattccagacgtttgaatggaggcagtatatttttcactaatgttttcgttttttcttgtttacttagttttgtttgctgttgttgttgatgtaaataaaggttcgtttgatgggaggagaaatcagcatcaaggacaaggagccaggagaagcggggacgtgctttggcttcaacatcttcctcaaggtcagcgaggcgtcagaggtggaagaggacctcgagcaagggaggacgccgccgtcgctgttcagggagcccgcctgcttcaagggcgggcactgcgtcctcctcgcccacggcgacgagacacgccggatcctgtatacatggatggagagcctcgggatgaaggtctggcccgtcacgcgcgccgagttcctcatcccgaccctcgagaaggcgcgctccgccgccggcgcctcgccgttgaggtcggcgtcgacgtcgtcgctgcatggcgttgggagcgccgactccaacattacgacggaccggtgcttcagctccaaggagatggtcagccacctgcggaacagcagcggcatggccggcagccacggcgggcacctccaccccttcggcctgctcgtcatcgtcgacgtctccggcgggaggctcgacgaggttgcccccgaggcggcgagcttggcaaggatcaagcagcaggcgccgtgcaggatcgtctgcctgacggacatcaagaccccctccgaggatatgaggaggttcagtgaggcggcaagcatcgacctcaacctgcgcaagcccatccatggctcccggctgcaccaactcctccaggtcatgagagacctccaggccaacccgtttacacagcagcaaccacatcagtccggcacggccatgaaagaactgccggctgctgatgagacctctgcggcggaggcgtcttctgaaatcacgcccgcggcggaggcgtcttctgaaatcactcccgcggcggaggcgtcttctgaaatcactcccgcggcggaggcgtcttctgaaatcactcccgcggcggaggcgtctgaaatcatgccggcagcaccggcgccaactccccagggaccggccaatgctggagaaggcaagccgctggaggggatgcgcatgctgctggtcgacgacaccacgctgctgcaggtagtccagaagcagatactgaccaattacggggcaaccgtggaggtcgccacggacggctccatggccgtggccatgtttacaaaggctcttgagagcgcaaatggcgtctcagagagccatgtggacacagtggccatgccctacgatgtcatcttcatggattgccaggtacatttttctccagcaaacaacgtgccaagcacatcgtcttcttcctgaagatgatttaatctgacgttgctgacaattcgatcttcttgtttcagatgccagtgatgaatggctacgatgctacgaggcgcatccgcgaggaagaaagccgctacggcatccgcaccccgatcatcgcgctgaccgcgcattccgcggaggaggggctgcaggagtccatggaagcagggatggatcttcacctgacgaagcccatacccaagccggcaatcgcacagattgttctagacctctgcaaccaagttaataactgatcgccgagatccatcgttccccactcccccgccgcatgatcaaaaatcgagataggtgtaggtggtttttcagcgagcgaatgcggttatcatcctagtcactgaaaaaccacctacatctctgagtttcgatcatgcgacccggggcatcatcgtagtttgtagcatttagcagcagcagctgaactttgttgttgtatcaaaatcaggtcaggtttatttccagaattgctcttggacaatgtattgtcaattttgaatttccagaaacaattatggttaagttttgagttccagagtttgtgtgtcagagttcttttttcccagagtttgtgttttcagagcttgtgattagacgccatatatttgcttccattgcccatcccaggagaaagaaagtacaggttgctacaagccatgaaatccctagctagctaccccagatatctagtagtgtacacaagcatagctgataaatacccataggaataactgtaccatcttctctaggtatgtagtggactagcctaaatattactaggactagctctcatatgcaggcaccaagtgggaaaagttcacacccgaggtcattttccgtgaacggcacttcgtcgctctcctgcgttgaaatcggaaaaggggttcagaaaaatcaccatcaaaattctaagaacataggtttaataaataacgattttaaaggaaatatgactgtcctaaatagtgaaattttaagcatgactgatgtccttaccacttttatcatggaattgagaagaccatcaaagttgcagctgctagaattgaatccaccctgcagctttggaactcccaaatcgtacatatcggtctgctcaggcacaatggcaccaccatcatagctaaaacctccactgttcagtctttggtcttgtctcattccaccatccattctatgtctagagttgcttgcaaccacaaactgcaagtattttgaatgtttctcggtatcatgaggagggagcagcattgtactaccacaagaactagctccacatttggcattgctggctactataaggctctcagaaggacaaacagtaacagccattctttccgaaaatatccccttttggtccatctcttgttctccagccacaaagctagcatcaagttttgtcgtgccggtaccatcgaacgggatgtgcaacggtaacttgtcgacagaaaacccatcactgattggaagagcacactgctgagatatacaagaatcccgcaaatcggcggaaactccgacagacctttcaagcagccctgaactcccagttggtattcctattgatggatgggctccaactttgatgtgtgcagtgcactccaaaagatcttctggtggcagtgaactgcttgtaactctttgaagtgtgccagacatagtgttagccagagcacccccggaaaagacagagcacaagtcactggtttcttgatgaatccacttctgatggagctggggctgcccaagcgacgaggtcaagccttgcgataaatctgcctgttggttgtcttgtaagctcacaatgtggaacttctccgtatcgccggcgcaatgacttactgcgccgttgctgctagaacactggtttgccttgggagaagcaaggtcctgaagcccaaatgctgctgcaccagcaccactcagcagtccatgtggattgagagatggaagagcagcaggaggggcaaagggttgatgataactatgaagtccttcaaatgctcccatgtgcaagaaggggtctctgcctccaaaagcagcagcaatgctggcttgctgtgatgccacggcacttagccgtctgaggtatagcctgtacttctgcattggcatagaaagtaacctgattagacatggaggaaattatgtaatcatcagaattctgaaacatgagatatacagtcttaagataactgcttcctagtttggtatggagattcttgctgaactggtacataaaagcactccaggatatagaaaaatctgatgaattttcttaaccctttaatgtaggcttgttaacaaaatttctatttgtcaattcattgacatgcaaagcttttgcatttctctaaaaaaatattttaaccaaaaggtgtactactacctccgtctcaaaatataagacagaggtagtacaacgcagtttacatatgtagctttgtaatctgtacagggtggtgtcagtgtcttccttctagcagttttatagaagtgaaacaatgtagccaaaatactacattaaactcagcagtacataccaaaacatcacattaaacttgtacagcagtacatactgaaatacaacattaacttgtacagcagtgcataccaaaataccacagttaacttgtacagcagtattacctaattactctgcattaaacctgtacaacattacatactaatttgagcattttatacctgtgtagttagatctaatctactagtctacatggttgggggaataatcagatatttgtataagatatctccttcttcaggttttctgaaagattcagtataactgaactgaatgtttctttgttttcagaccagctgaacatattcaacttgtcaagaaaaatgaagaaatgtgaaggtgaaactatgtaaccgcaaaattaaactacttaactcattttgcacgaacaccaggaacatgttaacatatttgttaaaagagttctgctgtgcaacaccataatatcgaattaagctctgctctgcaactaataggtgtgtcggtaccaaatacagtaaggggataaggactctgtatacgaatatcttcttttttaaaattgttcaaaagttggccctcttaattcgacacaggaaaataaggccactttgcacataattttaccaaaagtttctgtaaaagtttttcttcctgtgcaccacatgctgatcaacgaaagagataacacaggtaaaggcgatgatggaagtaaatgccacgtcgccatacctgcagatggctcgcaacattttccctggtgagcttctccacgttcataagctcgagtattcgtttgggtacagcctctgcgaacagcagcaagagaaaggacacaagcgtaaatgggtttgctgcagcctgcaagaacatttaacataaagcaaaaggcaacgtggggaggtgttttttcttactgtcgatcccgagctggttgacggcggcgacgaacttccggtgcagctccactgaccacaccaccctcggcttcttcgaccccgcggcgtcgtcgttgtcctcgccttcgtcttcctcctcgctgtgcggctccttccgcttcttgccggccctgttgctctggtcggacgacatgccgcacgtcgcctggccgagccggtggtacgagtccgcgctcggcggcttgctgatctccttgcagaggtcatggttgttgttcggctcacggttgctgaacttcctcctaacgacgtgctgccacacgttcctgagctcttcgatccgaacgggctttaggaggtagtcgcaggcgccatgggttatccccttgagcacagattttgtctctccgttcaccgataacactgcacgagttgaatggtgcctcattaacatc

SEQ ID NO: 20 - DNA, 3249 bp, Triticum aestivum
atggctggagaggttggcaagtggggtagttccttcaaacattcttgggctttaatcccactggttgcccatggaatcatcgtggtcgtagtggctctcgcttactctttcatctcgtcgcacgtaaatgatgatgccgtgagcgccatggacgcgtcgctggcgcacgtcgccgccggcgtgcagcctctaatggaagccaaccgctccgccgccgtcgtcgcgcactctctgcagatccccagcaacgaatcatcttatttccgatacgtgggaccatacatggtcatggcgttggccatgcagccgaagctggccgagatatcatacaccagcgtggacggcgccgcgttgacgtactaccgcggcgagaacggccagccgagagccaagttcgggagccagagcggcgaatggcacacccaggccgttgatccggtgaacggccgtcccaccggccgccccgacccagcggcgaggccggagcacctacccaacgcgacgcaggtcctcgccgacgccaagagcggctcgcccgcggccctcggggccgggtgggtcagctccaacgtccagatggtggtcttctccgcgcctgtcggtgacactgccggcgtggtctccgccgcggtccccgtcgacgtcctggcgatcgccagccagggcgacgccgccgccgatcccgtcgcgcggacgtactacgcgatcactgacaagcacgacggcggggccccgccggtctacaagcctttggacgccgggaagcccaaccagcacgacgcgaagctgatgagggccttttcctcggagaccaaatgcaccgcgtccgccattggcgcgcccggcaagctcgtgctccgcgccgtcggggcggaccaagtcgcgtgcacgagcttcgacctctccggagtgaacctgggagttcgtcttgtggtcagcgactggggcggggcagccgaggtccggcgaatgggggtggccatggtgagcgtcgtgtgcgtggtcgtggcggtcgcgacgctggtgtgcatccttatggcacgggcgttgtggcgggccggggcgcgggaggccgctctagaggctgacctggtgaggcagaaggaggcgctccagcaagcggagcgcaagagcatgaacaagagcaatgccttcgcccgcgccagtcatgacatccgctcctcactcgctgccgtcgttggactcatcgatgtttcccgggtagaggccgagagcaactccaacctcacctataacctcgaccagatgaacattggcacaaacaaactcttggatatacttaacacgatactggacatgggcaaggtggagtccgggaagatgcagctagaggaggtggagttcaggatggcagacgtccttgaggaatccatggacctggcaaacgtcgtcggcatgtcaagaggcgtcgaagtgatctgggacccttgcgacttctccgtgctccggtgcaccaccaccatgggcgattgcaagcgtatcaaacaaattcttgacaacctactcggcaacgccatcaagttcacacacgacggccacgtcatgcttcgagcatgggccaaccgtcccatcatgaggagctccataatcagcaccccgtcgaggttcaccccccgtcgccgcacgggtgggatctttcggcggctgcttggaaggaaggagaaccgttcggaacaaaatagccgaatgtcattacaaaatgatcctaattcggttgagttttactttgaggtggttgacactggtgtgggcataccccaggaaaagagggagtctgtgtttgagaactacgttcaggtgaaggaagggcatggtggcaccgggctcgggcttggaattgtgcaatcctttgttcgtttgatgggaggagaaatcagcattaaggacaaggagccaggagaagcggggacgtgcttcggcttcaacatcttcctcaaggtcagcgaggcgtcagaggtggaagaggacctcgagcaagggaggacgccgccgtcgctgttcagggagcccgcctgcttcaagggcgggcactgcgtcctcctcgcccacggcgacgagacccgccggatcctgtacacgtggatggagagcctcgggatgaaggtctggcccgtcacgcgcgccgagttcctcgccccgaccctcgagaaggcgcgctccgccgccggcgcctcgccgttgaggtcagcgtcgacgtcgtcgctgcatggcatcgggagcggcgactccaacactacgacggacaggtgcttcagctccaaggagatggtcagccacctgcggaacagcagcggcatggccggcagccacggcgggcacctccacctcttcggcctgctcgtcattgtcgacgtctctggcgggaggctcgacgaggtcgcccccgaggcggcgagcttggcgaggatcaagcagcaggcgccgtgcaggatcgtctgcctcacggacctcaagaccccctccgaggatctgaggaggttcagtgaggcggcgagcatcgacctcaacctgcgcaagcccatccacggctcccggctgcacaagctcctccaggtcatgagagacctccatgccaacccgtttacgcagcagcagccgcagcagctcggtacagccatgaaagaactgccggcggctgatgagacctctgcggcggaggcgtcttctgaaatcacgcccgcggcagaggcgtcttctgaaatcacggccgctgcggaggcgtctgagatcatgccggcggcgccggcgccggctccccagggaccggccaatgcaggagaaggcaagccgctggaggggatgcgcatgctgctggtggacgacaccacgctgctgcaggtagtccagaagcagatactggccaattacggagcaacggtggaggtcgccacggatggctccatggccgtggccatgtttacaaaggctcttgagagcgcaaatggcgtctcagagagccatgtggacacagtggccatgccctacgatgtcatcttcatggattgccagatgccagtgatgaatggctatgatgcgacgaggcgcatccgggaggaagaaagccgctacggcatccgcaccccgatcatcgcgctgaccgcgcattccgcagaggaggggctgcaggagtccatggaggcagggatggatcttcacctgaccaagccgatacccaagccgacaatcgcacagattgttctagacctctgcaaccaagttaataactga

SEQ ID NO: 21 - protein, 1082 aa, Triticum aestivum
MAGEVGKWGSSFKHSWALIPLVAHGIIVVVVALAYSFISSHVNDDAVSAMDASLAHVAAGVQPLMEANRSAAVVAHSLQIPSNESSYFRYVGPYMVMALAMQPKLAEISYTSVDGAALTYYRGENGQPRAKFGSQSGEWHTQAVDPVNGRPTGRPDPAARPEHLPNATQVLADAKSGSPAALGAGWVSSNVQMVVFSAPVGDTAGVVSAAVPVDVLAIASQGDAAADPVARTYYAITDKHDGGAPPVYKPLDAGKPNQHDAKLMRAFSSETKCTASAIGAPGKLVLRAVGADQVACTSFDLSGVNLGVRLVVSDWGGAAEVRRMGVAMVSVVCVVVAVATLVCILMARALWRAGAREAALEADLVRQKEALQQAERKSMNKSNAFARASHDIRSSLAAVVGLIDVSRVEAESNSNLTYNLDQMNIGTNKLLDILNTILDMGKVESGKMQLEEVEFRMADVLEESMDLANVVGMSRGVEVIWDPCDFSVLRCTTTMGDCKRIKQILDNLLGNAIKFTHDGHVMLRAWANRPIMRSSIISTPSRFTPRRRTGGIFRRLLGRKENRSEQNSRMSLQNDPNSVEFYFEVVDTGVGIPQEKRESVFENYVQVKEGHGGTGLGLGIVQSFVRLMGGEISIKDKEPGEAGTCFGFNIFLKVSEASEVEEDLEQGRTPPSLFREPACFKGGHCVLLAHGDETRRILYTWMESLGMKVWPVTRAEFLAPTLEKARSAAGASPLRSASTSSLHGIGSGDSNTTTDRCFSSKEMVSHLRNSSGMAGSHGGHLHLFGLLVIVDVSGGRLDEVAPEAASLARIKQQAPCRIVCLTDLKTPSEDLRRFSEAASIDLNLRKPIHGSRLHKLLQVMRDLHANPFTQQQPQQLGTAMKELPAADETSAAEASSEITPAAEASSEITAAAEASEIMPAAPAPAPQGPANAGEGKPLEGMRMLLVDDTTLLQVVQKQILANYGATVEVATDGSMAVAMFTKALESANGVSESHVDTVAMPYDVIFMDCQMPVMNGYDATRRIREEESRYGIRTPIIALTAHSAEEGLQESMEAGMDLHLTKPIPKPTIAQIVLDLCNQVNN

SEQ ID NO: 22 - DNA, 13265 bp, Triticum aestivum
gtgtatgttgtgatctgggaaaagcttccctgccctaattgggtccactacttcgttaacgtttaccgcttcaattaaacgagttcaataacgatacgcttttgtacaaatgtaccaacctttatggtttatttatgtaaccaatcatgacatattcacccaagtacattctgatatttatgttgaatgtgcacattgtctattaatcgtggggtagtgtatatagtcactagggtgctcatgtgcttaagtcgcgtccccacaattgtttatatttactgcaaaacaaagataaccggatcaacaaaccaataaattggcgagtggtcctttcatgctatccaccatatggggcaattgtttttaagttatagatttcgtaggcttccttttcatgttatttttcttttcgtttagtcagatggtgtgtgttgtgatctgctgggaaaaagctcccccctccattggtggcaataaacataaaaagggccggctctcacgtcgtacaaaaagaaaagaaaacagatttgaatataaatcaatataatttacaataacacacgagaccatcccattaccatagtaggaaacgccacaccctttcccatttttggaaattgaccaccactagtggctcatcctgtaaaccttcccttcaacttctcggttgttctcatctacttctaacttaattatttaagggacatgcatagaaggtgactaccagtgatgagcacggtgtcatgggagcctatgaacaactttcaaccatatatgacacaccttttataaagggaaacccatatttctaactaaacttcaacaatattcataaaaaaaaccatgtgatgccactttcgaccaaaatttagtatccaaaaatctacaattttttgaatcaattgtttaattttgtacaaaattcaactggcttaaataaacattcatgcatttctaactaaaatttctcacgaaaattcttccaactttcaactcaagggaaaccgaaagatttggccaatcctactatagagatggctttatcaaggcatgatcatgataatgggacaagtatagcctcccaagggttgttaaaaaacgtgcatgttgaaattaccatcatcataagatccatgcccccccccacacacacacacatacgtacatgatccaatcacaagagatttggtggggcagggtattatattttcttgggtgtggtttgaaaaattcaagccaaccttgtctattatgtcttaattaaaagttgtgtcgtgcaatcgtttaaatgaagtttcatttttctccagaaagacaaatgaccaaaacaacaccttcatttaccatcgctaccttttacccatatccattccctgctcccatccgtcctcggacacagctcctacaccatgagacagagggaggggatcctcatctctcatttgatgtgtaggaaaagttcgttggttagtttttggtgtcacggtgacatcacagtgacagtgacgactccaatactgtctcgacggtgtttgatgaggtgttgccaaggagattgtgagtattttttgttcttgtcggcctttttggacgacgttgtggttcttgttgctattttggcgaggcatcattgactggtgtcgtcttcttcaacaaccctttccttcgagcctttgcgagcaatgtcagtggcctagtttggcaatatgagtgtggttgccttcctagatcccccacgccaaatgttcttggcatgattgctaatacctgttatacacgactgtctaccattgcgaccattcaagatgtggtcctcaagaggtcttcactgagtaaaattctcaatgttattctccgccggcgagagctaggtggggcaacgacacgagtttggttacggtttggcttgaattttgcggcctacggtgttggttataattcaccatctgtttatggttatttctatgcttgtgggtttagttacctcgttatttgaatgtattcgcctttttctttgtgatatatgatagaactgattaaaaaaattgtgctagtcatagtgaggcaaataagctacatatgtacattaaaaacaatatgacatgtccactaactataatacgcacaaaagaattgtgtatgagtcttgtatttttttttcatcgtttattttcaatggaatataggtgacatgttgtacagtctcacacgaacagctctacatattgcccactcggcacacaagaggaacgctcgaacttgtcgtgtgtgtgcggacaaaataaatagaacttttcaatttccgcgttcgagattttcagcggatgattaacgcattagacagagcatcaaacgaatcgatcgagtttgaataagtaagtcacaggctcaaaaaaagcaagacacagttcatcttttttttcaggaaaggacctggttcatgttcattttattttttgaggaaaagggcctggttcatcctagctgtcctggtaacggagctagttcaacataagtcgagcctgtgcttctatatttagcatcaaacgtaacgaagagttaacttaacaaaactaatgataatgctatcttaagacaggaagctaattgatcggtgttcacttgcacaacaggatggccttatggcgatcgtgcggctgccaacactgcctcacgcccgcccaaactattcgaacgggaggaaaacataattcattgtgctcagatttggcaattgattacaagatcgcgtagtatccctttttctatctcgtcgtgtgtctactaccgggccgcgcattgatattagatatcaaagttaaatgctgatttttaagaataaatacctcttttttattccggtgcaggggtaaataccgattttttttctgcggtgcaggggaaatactgccaaaagtggtataataaaccgcgatggcggatttgaaaaaccagcggcgctccgagcccataattcagagcggaaaaaccccccaatgtgtaaagtgtattcctccgagcatctataaaaggccgtatacctaggaaacaaatactcaccaacacctaacaaagatttgcttctgtagagtactttacaacacttccacgatggctggagaggttggcaagtggggtagttccttcaaacattcttgggctttaatcccactggtatgaaaatattcatagtacttggcttctttttgtgaaactcatggttaatacttgttcttctatatgaacttcatggctattcttttaaaaaaaactccacgacttttctcatttcttggttcttgtccttccacctacaattattgcttacctgagcgaaaactagatctgcgaggttatcatctgctgaagctttttttgagggattcatcttacttaagcttagtgcaacaaagacatccttccgcatatgtaggtgtgtgatctcgacaatagatctatgtagtggtacggtattcttttggaagaggaggattacccctggtctcttagcataggcgtgagtttcaaaagtaattctaatgatttttgtcttacaattaaattctgtttcgtacgatatagatatcttcctatgcctgtaaggtgcagatttgaaaagtagatctaactgatttttatcgtttatgtttcagttctgtatattacagatatcatcctgcacgtgtcaggcatgcgagatctatttctttagataacaatgaaaactagatcttcataatttttcatcttgtaattgaagattctgctccgtacaagacggatatcctcccacgtgtgttttaggcgtgaattcaaaaagtaaatataattttattttatcttgtaattaggaatccgctccgtacaatattagtgtcctttcacctgtgtaaggtgtgctctaaaaatacatctatgtaggttttaatatataatttggcttctgctccgtacatcacagatatctccccacatgtgtgaggcagtggcgagtcgaagaactcccttttgttgtatgctcctatattgtttctctgctcaccgtgtgttagatctacccatggaatgtcatgacaaatgttgtcgttagatgagcccaaaggcgatctattagcgggatgccacggcaaattatatctttagattagtgaaaaccctcgaaatcttgacttgttacttgcgcaacaaatcttaccgttagacgagttgaaactcgttgcaatgccgcttgtcaaggcctcgtcagcctaaaagacagtttaatttgtctacaaaaaaagacactttaatttagtaattatgcattttctattttactttttacatgttgcaaatatatattttttctgacgtcacacttttattgcacttgcacgcatgcaggttgcccatggaatcatcgtggtcgtagtggctctcgcttactctttcatctcgtcgcacgtaaatgatgatgccgtgagcgccatggacgcgtcgctggcgcacgtcgccgccggcgtgcagcctctaatggaagccaaccgctccgccgccgtcgtcgcgcactctctgcagatccccagcaacgaatcatcttatttccgatacgtaattaaccgagaacctttgggttaattaaattatgcctttttttctctgtataacgtgattagtttcatcacgtatatgcacttcctttttgaaccacaattatttccttactttaaataacaaatcttaattactagccgggccgacgcggtaactggttattgtgtatgattctgttctgattttcgtagtaatgcgagcattgatatgaatatacgcatgcatatacaaacaaatcatttttgcatacatttttttatgtaggacacgtccaagataacatagcaacacgtacgtgcaaatatacatctaacatttacgtatatatgcttgacctgacaggtgggaccatacatggtcatggcgttggccatgcagccgaagctggccgagatatcatacaccagcgtggacggcgccgcgttgacgtactaccgcggcgagaacggccagccgagagccaagttcgggagccagagcggcgaatggcacacccaggccgttgatccggtgaacggccgtcccaccggccgccccgacccagcggcgaggccggagcacctacccaacgcgacgcaggtcctcgccgacgccaagagcggctcgcccgcggccctcggggccgggtgggtcagctccaacgtccagatggtggtcttctccgcgcctgtcggtgacactgccggcgtggtctccgccgcggtccccgtcgacgtcctggcgatcgccagccagggcgacgccgccgccgatcccgtcgcgcggacgtactacgcgatcactgacaagcacgacggcggggccccgccggtctacaagcctttggacgccgggaagcccaaccagcacgacgcgaagctgatgagggccttttcctcggagaccaaatgcaccgcgtccgccattggcgcgcccggcaagctcgtgctccgcgccgtcggggcggaccaagtcgcgtgcacgagcttcgacctctccggagtgaacctggtaacgtgtccatctagcatcgatcacaggccatccatatatgcatacgtacacgaacgtgcacacaccctatatatctaacataattgctctgcatttttgtcaagaattcatcccagcatgtaatatttcttccaagtttgctgtttatacattcaagcaacggacaatgaaaaaaggttagatgaggtaagggcatctacaatgctaggagcttacataggcgcttatagacaaaataataaataaataaaaatctgaaacacacctaagcgcctcatccctcaacgctagacgctaattaactaaacaacgtggacgctaagtactggaaggaaaatgaccaagcgccgggtgcatggtttggcgtcgatgccagggttgacaccaggattacacccggcaaccgctaatctgccgagattatgaacctggcgcctggcctaaacgcccagcaatggagatgccctaaggggacttcggccaaaaaaaaggggaactaataaaacgtttttttgttttgacgacctcaataaacatcattccgacttggagtgaggaaaaaagggaaacagggaacgctcctagctattgacagtacgtacataatcttgcttccttccttccgcgtgtaaaaaaactgaaaactttccaaatcaagggatcccaaaattaggaagaaaatcttaatagagagtacaaagtcttcttcttcctcttcttccccaaagcaagatttctccttctgttcttccccaaacggagctctggcacaaaactgcggtgagctcgatcgtctcgtacttttcttgcatctgctagctagggtcttgcatgcatatcaccggttgtcgttcatggatatctcccatcagttcttttgcaatttatttacagcgtagagagcagtatactatgtacgcagtaaatcactatataaactgatcttgacatccgtcacctatgcaacgagcgcacggcaaaggggctgcgctggtccgtttgataattcaacggccaggccgggcggcgcctcgcgctccatggaaacacccttttttcagataaaggccatggaaacaacctgacgtacagtgccgatcgcaataccataaaggacccagtcataaaaaaaaattcccagagattagcctcttgatacaaaaaatatattttctttgtagtttagcacgcatatgcatgtttgagaattccctttttttggggacggctgacaagtatcttcggttgtatttcttcttgttttccagggagttcgtcttgtggtcagcgactggggcggggcagccgaggtccggcgaatgggggtggccatggtgagcgtcgtgtgcgtggtcgtggcggtcgcgacgctggtgtgcatccttatggcacgggcgttgtggcgggccggggcgcgggaggccgctctagaggctgacctggtgaggcagaaggaggcgctccagcaagcggagcgcaagagcatgaacaagagcaatgccttcgcccgcgccagtcatgacatccgctcctcactcgctgccgtcgttggactcatcgatgtttcccgggtagaggccgagagcaactccaacctcacctataacctcgaccagatgaacattggcacaaacaaactcttgggttagtccgcatccatgccctacgtaccatgcatggcaataccagcttgctctacgttttagtagatctatccgtacttggcaatttagctaatgtgatcatagcattataaatttgcatgccatagaagtaaagttttcctaaataatttaattacaatcttaggtgtagagtgtgcaatagtccagctgttatacatttaaggtatcgaatttgctcataaaatttaaaatatgcaagaaatcaatctctgttttggaaagaaatagcattttatttgaaaaaaaaagtttaggatagttcaaatagaattgtcagatctcactatgttagcagctcgtaaacttataaagtttcaacatttctatctattttgctgttggggaaattttgtcacatttatgcatcatattgtttgtacgcgtgttcgagtgcataaagcaaaaattttattatttccgaaaaaatgaactttataccatctttgtgttctcagctcagcctatgttgactcatcatccatgtttatacatgtgtatgtgtacatgcagatatacttaacacgatactggacatgggcaaggtggagtccgggaagatgcagctagaggaggtggagttcaggatggcagacgtccttgaggaatccatggacctggcaaacgtcgtcggcatgtcaagaggcgtcgaagtgatctgggacccttgcgacttctccgtgctccggtgcaccaccaccatgggcgattgcaagcgtatcaaacaaattcttgacaacctactcggcaacgccatcaagttcacacacgacggccacgtcatgcttcgagcatgggccaaccgtcccatcatgaggagctccataatcagcaccccgtcgaggttcaccccccgtcgccgcacgggtgggatctttcggcggctgcttggaaggaaggagaaccgttcggaacaaaatagccgaatgtcattacaaaatgatcctaattcggttgagttttactttgaggtggttgacactggtgtgggcataccccaggaaaagagggagtctgtgtttgagaactacgttcaggtgaaggaagggcatggtggcaccgggctcgggcttggaattgtgcaatcctttgtaagtgatctcgtctttttcatgcatgttaaaatcttgtcaactgcatcaacgacaactagccgtaaatgtatttcgttttttcttgtttacttatagttttgtttggtgttgttgttgttgatgtaaatataggttcgtttgatgggaggagaaatcagcattaaggacaaggagccaggagaagcggggacgtgcttcggcttcaacatcttcctcaaggtcagcgaggcgtcagaggtggaagaggacctcgagcaagggaggacgccgccgtcgctgttcagggagcccgcctgcttcaagggcgggcactgcgtcctcctcgcccacggcgacgagacccgccggatcctgtacacgtggatggagagcctcgggatgaaggtctggcccgtcacgcgcgccgagttcctcgccccgaccctcgagaaggcgcgctccgccgccggcgcctcgccgttgaggtcagcgtcgacgtcgtcgctgcatggcatcgggagcggcgactccaacactacgacggacaggtgcttcagctccaaggagatggtcagccacctgcggaacagcagcggcatggccggcagccacggcgggcacctccacctcttcggcctgctcgtcattgtcgacgtctctggcgggaggctcgacgaggtcgcccccgaggcggcgagcttggcgaggatcaagcagcaggcgccgtgcaggatcgtctgcctcacggacctcaagaccccctccgaggatctgaggaggttcagtgaggcggcgagcatcgacctcaacctgcgcaagcccatccacggctcccggctgcacaagctcctccaggtcatgagagacctccatgccaacccgtttacgcagcagcagccgcagcagctcggtacagccatgaaagaactgccggcggctgatgagacctctgcggcggaggcgtcttctgaaatcacgcccgcggcagaggcgtcttctgaaatcacggccgctgcggaggcgtctgagatcatgccggcggcgccggcgccggctccccagggaccggccaatgcaggagaaggcaagccgctggaggggatgcgcatgctgctggtggacgacaccacgctgctgcaggtagtccagaagcagatactggccaattacggagcaacggtggaggtcgccacggatggctccatggccgtggccatgtttacaaaggctcttgagagcgcaaatggcgtctcagagagccatgtggacacagtggccatgccctacgatgtcatcttcatggattgccaggtacatttctccagcaacagcatgccaagcacatcagccccatcctcctgttcctgaagatgatttaatctgacgctgctgacaattcgatcttctttgtttcagatgccagtgatgaatggctatgatgcgacgaggcgcatccgggaggaagaaagccgctacggcatccgcaccccgatcatcgcgctgaccgcgcattccgcagaggaggggctgcaggagtccatggaggcagggatggatcttcacctgaccaagccgatacccaagccgacaatcgcacagattgttctagacctctgcaaccaagttaataactgatcaccgagactcttcgttccccgttccgccgtcgcatgatcaaaaatcaagataggtgtaggtggtttttcagcgagcgaatgcagttatcatcctagtcactgaaaacccacctacacctcgagtttcgatcatgcgacccggggcattatcgtagtttgtagcatttagcagcagcagctgaactttgttgttgtatcaaaatcaggtcaggtttatttccagaattattcttggacaatgtattgtcgattttgaatttccagaaacaattatggttaagttttgaattttcagagtttgtgtttttagagctcttttttcccagagtttgtgttttcagagcatgtgattagacaccatatatttgcttccactgcccattcacaggagaaagaaagtacagattcctacaagccatgaaatccctagctagctaccccagatatctagtagtgtacacatagctgataaatacccataggaatagctgtacaatctcctctaggtctgtagtggactagcctaaatattactaggagtagctctcatatgcaggcaccaagtgggaaaagttcacacccgaggtcgttttccgtgaacggcacttcgtcgctctcctgcattgaaatcggaaaaggggttcagaaaaatcaccatcaaaattctaagaacataggtttactaaataacgattttaaaggaaatatgactgtcataaatagtgaaattttaagcatgactgatgtctttaccacttttattatggagttgagaagaccatcaaagttgcagctgctggaattgaacccaccctgcagctttggaactcccaaatcgtacatatcggtctgttcaggcacaatggcaccaccatcatagctaaaacctccactgttcagtctttggtcttgtctcattccatccattctatgtctggagttgcttgcagccccaaactggaggtattttgaatgtctttcggtatcacgaggaaggggcagaattatactgccagaggaactagctctacatttggcattgctggctactataaggctctcagaaggacaaacactaacagacattctttccaaaaatatccccttttggtccatctcttgttctccagccacaaagctagcatcaagttttgtcacgccagtaccatcgaacgggatgtgcaacggtaacttgttaacagaaaacccttcactgattggaagagcacactgctgagatatacaagaatcccgcaaattggcggaaactccgacagacctttcaagcagccctgaactcccagttggtattcctatggatggatgagctccaactttgatgtgtggagtgcactccaaaagatcctctggtggcagtgaactgcttgtaactctttggagtgtgccagacatagtgttagccagagcacccccggaaatgacagagcacaggtcattggtttcctgatggatccacttctgctggagctgaggctgcccaagcgacgatgcggtcaagccttgtgataaatctgcctgttggttgtcttgtaagctcacaatgtggaacttctccgtatcgccggcgcaatgacttgctatgccgttgctgctagaacactggtttgccttgggagaagcaaggtcctgaagcccaaatgctgctgcaccagcactactcagcagtccatgtggattgaaagatggaagagcagcagaaggggcaaagggttgatgatagctatgaagtccttcaaatgctcccatgtgcaagaaggggtctctgcctccaaaagcagcagcaatgctggcttgctgtgatgccacagcacttagccgtctgaggtatagcctgtacttctgcattggcatacaaagtaacctgattagacatggaggaaattatgtaatcatcagaattctgaaacatgagctatacagtcttgagataactgcttcctggtttggtatggagttggtacatgaaagcactccaggatatagtaaaatctgatgaattttcttaaccttttaatgtaggcttgttaataaactttctatttgtcaattcattgacatgcaaagcttttgcatttctataaaagaatattttaaccaaaaggtgtactactacctccgtcgcaaaatataagacagaggtagtacaacgcagtttacatatatgtagctttgtaatctggacagggtgtgtcagtgtcttccttctggcagttttatagaagtgaaacaatgtagccaaaatactacattaaactcagcagtacataccaaaacaccacattaaacatgtacagcagtacataccaaaatacaacattaacttgtacagcagtgcataccaaaatactacaataaacttgtacagcagtattacctaattactctgcattaaacctgtacaagagtacatactaatttgagaattttatacctgcgtagttagatctaatctaccactgtagtctacatggttgagggaatgatcagatatttgtattagatatctccttcagggtttctgaaagattcagcataacttttttttcgaaaagggggatcttcccggcctctgcatcagaatgatgcatacggccatcttattagcgaaataaaaggttccaacaaggttccaaagtctccgactgaaaagtaataaaaagacagctcacatagagctaaagaggctggacacacagactagccaagataagactccacaaccggctggctaaagatagataggtaaactaattgcctatccattacatgaccgccatccaaaccggttgagatatcccgaagattcagtataactgaactgaatgtttctttgttttcagacccgctgaacatattcaacttgtcaagaaaaatgaagaaatgtggaggtgaaactatgtaaccgcaaaattaaactacttaactcattttgcacaaacatcaggaacatattaacttatttgttaaaagagttctgctctgcaactaataggtgtgtcgataccaaatacagtaaggggataaggactatgtatacgaatatctttctttttttaaattgttcaaaagttggccctcttaattcgacacaggaaaataaggccatttgcagataattttaccaaaagttctgtaaaagttttccttcctgtgcaccacatgctgatcaatgaaagagataacacaggtaaaggcgatgatggaagactggaagtaaatgccatgtcgccatacctgcagatggctcgcaacattttccctggtga

SEQ ID NO: 23 - DNA, 22 bp, Artificial Sequence
  Description of Artificial Sequence Synthetic oligonucleotide
aacgcggcgccgtccacgctgg

SEQ ID NO: 24 - DNA, 22 bp, Artificial Sequence
  Description of Artificial Sequence Synthetic oligonucleotide
gcgaggacctgcgtcgcgttgg

SEQ ID NO: 25 - DNA, 22 bp, Artificial Sequence
  Description of Artificial Sequence Synthetic oligonucleotide
gtcagctccaacgtccagatgg

SEQ ID NO: 26 - DNA, 22 bp, Artificial Sequence
  Description of Artificial Sequence Synthetic oligonucleotide
gcgtagtacgtccgcgcgacgg

SEQ ID NO: 27 - DNA, 669 bp, Triticum aestivum
atggagagatcccgccgcctgctgctggtggcgggcctgctcgccgcgctgctcccggcggcggcggccgccttcgggcagcagccgggggcgccgtgcgagcccacgctgctggcgacgcaggtggcgctcttctgcgcgcccgacatgcccaccgcgcagtgctgcgagcccgtcgtcgccgccgtcgacctcggcggcggggtcccctgcctctgccgcgtcgccgcggagccgcagctcgtcatggcgggcctcaacgccacccacctcctcacgctctacagctcctgcggcggcctccgtcccggcggcgcccacctcgccgccgcctgcgaaggacccgctcccccggccgccgtcgtcagcagccccccgcccccgccaccgtcgaccgcacctcgccgcaagcagccagcgcacgacgcaccaccgccgccgccgccgtccagcgacaagccgtcgtccccgccgccgtcccaggaacacgacggcgccgctccccacgccaaggccgcccccgcccaggcggctacctccccgctcgcgcccgctgctgccatcgccccgccgccccaggcgccacactccgccgcgcccacggcgtcatccaaggcggccttcttcttcgtcgccacggccatgctcggcctctacatcatcctctga

SEQ ID NO: 28 - protein, 222 aa, Triticum aestivum
MERSRRLLLVAGLLAALLPAAAAAFGQQPGAPCEPTLLATQVALFCAPDMPTAQCCEPVVAAVDLGGGVPCLCRVAAEPQLVMAGLNATHLLTLYSSCGGLRPGGAHLAAACEGPAPPAAVVSSPPPPPPSTAPRRKQPAHDAPPPPPPSSDKPSSPPPSQEHDGAAPHAKAAPAQAATSPLAPAAAIAPPPQAPHSAAPTASSKAAFFFVATAMLGLYIIL

SEQ ID NO: 29 - DNA, 5857 bp, Triticum aestivum
cgcttctgcaaaaatctccactagccattgcataagctcaggaaaattacctttatgtagtgaaacttctctccatcatgttcacgaaaatctaacccttgacaaaaaaggaacctcgggcattaaaaggaatatgtcaggccagctctatataaaaccttgtctcgtttgatggttgaacaaaatgactctatgattgttgtgtttgctgcaatgaagaaattgtatttctcttgtgctttgttacgtgcacactgcactattgatttcaccgacatgtttcacaaaactatccttgtgattctaatttctaagtcacccattcaccaaaaatctccaccaacatgcaaattatcattgaaaagataacatacaagcataaagcaccatctagttctttactatactcaagccaactataagacttaaaccatttagctacaaatattgttgcacacctccggtggggtgttgtggaaaagcatattttttcggtcaacaagccccttttgcaatgtatcctcttctaatcctattcggaccattaacatcataagttgcgattggcatcctcttcctaggatcagattcactcaatcgaacatcataaactgcatcttcaatgtcacccatttcctatattttttcagattattggcttgcttcgttcgcaatattaggtactgtgattggacttctgttgatgccactaataatttgcagttgttgcggaatatgaactcaaggggagctcatggtgctatgaagttgattcggtgggaaattgttctacatccgcacttgctgctcaacctaaatacatgggttggatttcttcccaactttagtacataaagttctcaaattaatgttctactacattaaaattgaaatccgcaaacattttttagtacccaaacatttttctaatatacggtgaacatttttcatctactgattttttgatatatggtgaaaattggtgtaatatatgctggcatgtttttaaatactacatattgaccatgtagataaaaaatttatagtatatgatgaacatttttgtaatatagatggccatttttaaaatatacattgcacattttataatatacgatgagcagtttataatactagatgaaccttttttggagttctgaacatttttttgaaaacagcagccattgtacaagaaaaaaccaaaacaaaagaaatgagaaacccaaaaacaaaaacaaaacaaaacaaaacagagaaacctacagaaaaaaacgaaacagaaaaaggcaaaggaagaacccgaactgggccagccggctcggcgtgccccagtgggccgtcgtggcgaatgcaacggctacatgggccgctcttcgtgaaagagaaggaggtcagttcatggaccgctaccagtacacgggcctcgctgtggcaacacccgccgtgtactagttttcgcgggaatccaatgccaaaatcgctccccgcgggaacccgacgtcggtctggtgacttctggagccttccagaacactccacaagctcccagagccgtctgatcagatcagcacgaagcacgaacattggcgcgcgaagatattttccttcccgacgacgccacactgcatttcatttgaatttcaaaaatcgaaaacggaaaacactttctctcatcccgaggagaggcggttagtgccagaggagcacgagagaggccaccccccccccagccagctcacgtgccgtgccctcgcaccctgcgcggccgcatccgggccgtccgcgcggacagctggccgcgccccacccgaaccgacgcccaggatcgcgcccgccacccgcttgccttagcgtccacggctcctccggctatataacccgcccctcacccgctccccctccggcattccatttccgtcccaccaccgcaccaccaccactccaccaaaaccctagcgggcgagcgagggagagagagaccaccccgcccgaccccgccgatggagagatcccgccgcctgctgctggtggcgggcctgctcgccgcgctgctcccggcggcggcggccgccttcgggcagcagccgggggcgccgtgcgagcccacgctgctggcgacgcaggtggcgctcttctgcgcgcccgacatgcccaccgcgcagtgctgcgagcccgtcgtcgccgccgtcgacctcggcggcggggtcccctgcctctgccgcgtcgccgcggagccgcagctcgtcatggcgggcctcaacgccacccacctcctcacgctctacagctcctgcggcggcctccgtcccggcggcgcccacctcgccgccgcctgcgaaggtacgcgcacgttcaccgccctccgtccctccctctctctgtctacgtgcagattttctgtgctctctttcctgcttgcctagtacgtagtgttccatggcctctcgggccgctagcgctccgatttgcgttggtttccttgctgttctgccggatctgttggcacggcgcgcggcgtcgggttctcgccgtctcccgtggcgagcgacctgcgcagcgcgcgcggcctggctagcttcataccgctgtaccttgagatacacggagcgatttagggtctactctgagtatttcgtcatcgtaggatgcatgtgccgctcgcgattgtttcatcgatttgagatctgtgcttgttcccgcgagttaagatggatctagcgccgtacgcagatgcagagtctgttgctcgagttaccttatctaccgtcgttcgactatggtatttgcctgcttccttttggctgggtttatcgtgcagtagtagtagacatgtggacgcgttcttcttattttgtgccgaccatcgtcgagatacttttcctgctacagcgtttcatcgcctgcaccatcccgttcgtgatagcacttttgtgtcaaaccgcaacgcagctttgctttctgcggtatcttctgccttgtttgtcgccttgcttggtcaaaactgagaactcttgctgtttgatcgaccgagggcagaggcagagcaagagcctgccgtgcttttggctctgcagtgcgtcgtctctgcctcctttgccaaacatttccatgttgatcctctgggggcactgctttttcgcatgcggtttccgtagccttcctctttcatgaaaaaaggtttgggtcaaatcaaatggatcgcctattggcagagcagcagcagatagctggctgtctcacagctttggcagaatcggtctgttgcctgccaccgtgtctcttatcttgcctgccaccgtgtctcttttcttgttgcgcacgtcgtcacctcctcctacttcttttccagttttgtttacttttgatgaaatacggacgaacggctggtaatcattaactttggttgctgttgttactgtggattttggacgcaggacccgctcccccggccgccgtcgtcagcagccccccgcccccgccaccgtcgaccgcacctcgccgcaagcagccagcgcgtgcgtacctctccctctcgcccgcatctcgctccgtattaactgattgtgtctgcatactgacgtgtgctttggctttggatctgtttcgcagacgacgcaccaccgccgccgccgccgtccagcgacaagccgtcgtccccgccgccgtcccaggaacacgacggcgccgctccccacgccaaggccgcccccgcccaggcggctacctccccgctcgcgcccgctgctgccatcgccccgccgccccaggcgccacactccgccgcgcccacggcgtcatccaaggcggccttcttcttcgtcgccacggccatgctcggcctctacatcatcctctgagtcgccgaccccgaggccatggtccgtccagttgcagtagaatgctcgtcgtcttgttccgtttcatgcttgtcgccgttcgaggttcgtttctgcagtccgattgagaagaagacggtgggttttgatcgcgtcccgagatttctgttgtcgatcgtagcgtcctggtagtagtagtgtctggtagcagcagtatgttcatgtgtcctcggtcgcctagttttggtctcaagtagtactgtctgtccaccgtgtttgcgtggtcgcggagaacatcattgggttttgcgattcctctggtcagatgaaccactgctatgtgatcgatcgatatgatctgaatggaatggatcaagttttgcgttctgctgatgacgtgatgcttcttcagttatattcatgctcgatctatttctgtttcccccatttgaatttgtggagcagcagtttggctttcttttgttctgctatggatgaatgcttcttgcatgcatcttgtctttgcttaatttgaactgtagaacggatgcagttctggtttctgctaatgatgtgatgattcttcatatgcatatgctttacatgttcatctcttcaaatttgtgcagcaacagtttgtagctttcattcggctctgaatgaaatgcctcttgcatgttgtctttgcttaatttgtttttcacggggagcctgctgcagctttctgttgccatgttgttttccacgccaggacaaaatagatggtgcggtttgattcgatcccggttaattgcttgatgctagcttctgatcaatcccttcatcacgatgttccggagagccacatggaactggaggggggagattcaaattcatgcatgcaaatttgtgttggtgttgggtcacgtcaagcagtcactttttgcagtatcactcttaccattttatccttttgttgaaacctctctcctcaccccaaaagttgatgcaatagtgctatgcccacccatgcttttttcataatcttttgagcccaaagtcccattttactatctgtttgcatatttgtgttccttgcggcgagggctatcaagcaaggcctttcttgaatatattttggcaagttttcaaatttgaattctaaaagatggtgaaactctatgaaacaaatctcaaagtatatgaccttatcaccaatccaccattctacaaatatttcattctccggcatcgcctgcttccgacggcgatgccgctgtaggtcctcgccgccgccgcaactcttccgctagctatgtggtggaactgttggcactccaccctcatttcccgtctctttctatcgtctctaaccaccgcacaacgttccctacgtggggaggagcaacaatatctgcttcaactctttggagggtaaattgcatggatttcattcacaaagaaaatattgcatggattttaagtcaatttttgtggctgtggatcaatcaaccaaacaatttgggagaaaaaattcagcttagaatctgtatgagtgtggttgtgtttgtgtgacccttgcgtgaggaacagcagggacgccaaggagggttgccatggacgcaacaagcagaggagccgacgggcctctgaggaatgctgtcgcggacggcgggggagagtagcggggagggagcgcccagtgcttccacaagcaggagagttgcgacagcgtcgatgacgaacggacggagcactcataattagaagagtgtggcgctagagaaaaaggacaaggggatttgcatctaattgctttgagattcgttttgtcacgtgtcacccgctggagaaggtctcacgaccggaggcgtgcgacccgggtcatataggaatttttttcgtgccatcgataacaaccgaaattcttcgtgccgttgaaatcttgaatagatctgaatcagcagaagttactttaaccccaccgtcgtaaaaagaaaggcagaaatcgacgaactcaaggaggatgggaaccaaagaca

SEQ ID NO: 30 - DNA, 663 bp, Triticum aestivum
atggagagatcccgcgggctgctgctggtggcggggctgctggcggcgctgctgccggcggcggcggcgcagccgggggcgccgtgcgagcccgcgctgctggcgacgcaggtggcgctcttctgcgcgcccgacatgccgacggcccagtgctgcgagcccgtcgtcgccgccgtcgacctcggcggcggggtgccctgcctctgccgcgtcgccgccgagccgcagctcgtcatggcgggcctcaacgccacccacctcctcacgctctacagctcctgcggcggcctccgccccggcggcgcccacctcgccgccgcctgcgaaggacccgctcccccggccgccgtcgtcagcagccccccgcccccgcctccaccgtccgccgcacctcgccgcaagcagccagcgcacgacgcaccaccgccgccaccgccgtcgagcgagaagccgtcgtccccgccgccgtcccaggaccacgacggcgccgccccccgcgccaaggccgcgcccgcccaggcggccacctccacgctcgcgcccgccgccgccgccaccgccccgccgccccaggcgccgcactccgccgcgcccacggcgccgtccaaggcggccttcttcttcgtcgccacggccatgctcggcctctacatcatcctctga

SEQ ID NO: 31 - protein, 220 aa, Triticum aestivum
MERSRGLLLVAGLLAALLPAAAAQPGAPCEPALLATQVALFCAPDMPTAQCCEPVVAAVDLGGGVPCLCRVAAEPQLVMAGLNATHLLTLYSSCGGLRPGGAHLAAACEGPAPPAAVVSSPPPPPPPSAAPRRKQPAHDAPPPPPPSSEKPSSPPPSQDHDGAAPRAKAAPAQAATSTLAPAAAATAPPPQAPHSAAPTAPSKAAFFFVATAMLGLYIIL

SEQ ID NO: 32 - DNA, 3810 bp, Triticum aestivum
aacatatttataataaatggtgaacattttttttaataattgatgaccatttttaaaatgcatattgaacattttataatatacactgtacagttttataataatcgacgaacatcttttggagttctgaacatttttttcaaaaacacaagccattttccaggaagaatacaaatgcaaaagaaatgagatatccaaaaagcaaaaaagaaaaacaaaacaaaacagagaaacctacaggaaaatccaaacagaaaaggcaaagaaagaacccgaactgggccaggcaatgtttccaacggcctcgctcttcctgaacaagaaggccagtcagcccatgggctgctcccagtactcgggccccgctgtggcagcacgccatgtaatagttttcgcgggaatccaacgccgaaatcgcccgcagcgggaacccgacgtcggtctggtgcgttctggcgccttccagaactctccacaggctcccgcagccgtccgatcagatcagcacgaagcacgaacattggcgcgcggcgatattttctttcctcgcccgacgacggccgcactgcatttcattttgaatttcaaaattcggaaacggaaaagctttctcgcatcccgaggcgaggcggttacgggcgccagaggggccaccccacccacccacccccgccctcacgtgccccgcgcggccgcatccgggccgtccgcgcggacagctggccgcgcccagcccgaaccgacgcccaggatcgagcgagggcggcgcgcccggggcttggcttagcgtccacgccacctccggctatataagccgccccacacccgctccccctccggcattccattccgccaccgcaccaccaccaccaccaaaccctagcgagcgagcgagggagagagagaccgccccgccgcgacgatggagagatcccgcgggctgctgctggtggcggggctgctggcggcgctgctgccggcggcggcggcgcagccgggggcgccgtgcgagcccgcgctgctggcgacgcaggtggcgctcttctgcgcgcccgacatgccgacggcccagtgctgcgagcccgtcgtcgccgccgtcgacctcggcggcggggtgccctgcctctgccgcgtcgccgccgagccgcagctcgtcatggcgggcctcaacgccacccacctcctcacgctctacagctcctgcggcggcctccgccccggcggcgcccacctcgccgccgcctgcgaaggtacgttgtccgcctcctcccctccctccctccctccctctctctctacgtgctcgctttcctgcttacctagtagtacgtagtttcccatgccttcttgactcgctagaagtgctccggtttgggtctgttaatttcctcgctgtactaccggatctgtcgtcggcacggcgcgcggcgtcgggtcctcgccttctcccgtggcgaccgacctgcgcagcgcgcgcgcggcctagctagcttcataccgctgtacctcgacatacacggagcgatctatggtctactctgagtatttcctcatcgtagaacgcatgcgccgctcgcgattgtttcgtcgattctagatccgtgcttgttcccgcgagttagtatgcatctgcgtgcatatgccgtacgcacgcagatgcagagtctgttgctcgagttatctactgtcgttcgctcgaccatatttgcctgttaatttcctgttcatcgtgcatgcagtagtagtagccatgtccacgccttcttgttttgaggcgatcatcgtcgagatccatggctttgctttctgcactatcttctgccttgttttgttctccgcagtacgtacgtcttgcttggtcaaaactgaaaaacgctttgctgtttgtttgatcggcaagagctggccgtgcttttggcaccgcagtgcgtcgcctctgccgcttttgcgaaacatttccatgttgatcctctggcggaactactttttcgcgtgcggtttgcgtggccttcctctctcgtgaaaagaggtcgggtcaaaccaaatggatcgcctcttggcagagcagcggcagcagatagctggccgtctcgcagctttggcagaaccggtctgtggccatctgtcgccgcctgccaccgtttccctgatgtttgtttctctctcgcctgccactgtttcttttcttgttgcgcacgtacgtcgtcacctcctcctacttttttgccagttttgtttacttttgatgaaatatacggatgaatcggctggtgattaactttggctgctgctgttaattactgtggattttggatgcaggacccgctcccccggccgccgtcgtcagcagccccccgcccccgcctccaccgtccgccgcacctcgccgcaagcagccagcgcgtaagaacctctccctctccctctctctctccctctcgcctgcatctcgctatgtttatccatgtccatatgttgatcagccttgtttagttactaacatgtgcaccggatcgggttctcgcagacgacgcaccaccgccgccaccgccgtcgagcgagaagccgtcgtccccgccgccgtcccaggaccacgacggcgccgccccccgcgccaaggccgcgcccgcccaggcggccacctccacgctcgcgcccgccgccgccgccaccgccccgccgccccaggcgccgcactccgccgcgcccacggcgccgtccaaggcggccttcttcttcgtcgccacggccatgctcggcctctacatcatcctctgagtcgcgcgccgaccccgcgagagaccgtggtccgtccagtcgcagtagagtagagcgctcgtcgtctcgttccgtttcgtgcctgtcgccgttcgaggttcgtttctgcgtgcagtccggtcgaagaagccggtgggttttgagtactagtggtagtagtagcagcagctatcgtttctgtccgctcgtacgtgtttgcgtggtcgcggagaacaattaattgggtgtttgcgagtcctctggttaagatgaaccactgatgctatgtgatcgatcgatcggtatgatctgaatggaaatggatcaagttttgcgttctgctgatgatgtgatccatttggatctgtgtggggcaacagtttcgcttgcttttgctctgcgatgaacgaatgcttcttgcatgcatcttgtctttgcttaatttgaactgtagaacggatgcagtactgatttctgcttatgatgtgacgattcgtcgtacgcatatcatctcttcaaatttgtgtagcagctgtttgtagcttccattctgctatggacgaatgcctgtttttcacggagaaccgcgcgcggggaccgatgcggctttgtgttgccatgttgttttccacgccaggacaaaatagatggtgcggttttgatccccaatcccaccatcaccatgttccggagagccacatggaactcacgtcaagcggtcactttttgcagaatcactcttaccattttacccttttgttgaaacctctctcctcatccccaaaagttgatgcaacagtgctatgcgcgcccacccatgctttttcatatgattgtaaaatttggatcgattttatcttttgaaccctaagtccggtttacaatctgtttgcatgtttatgttccttgcggcgaggaccattaaacaagactactattggatatatttcgacaggctttgaaatccgaattctaaaacatggtgagactctatgagacacaagaatgctctttagaacacgaggaaacctaattaagattgataagaacaga

SEQ ID NO: 33 - DNA, 672 bp, Triticum aestivum
atggagagatcccgcggcctgctgctggcggcgggcctgctggcggcgctgctgccggcggcggcggccgcgttcgggcagcagccgggggcgccgtgcgagcccacgctgctggcgacgcaggtggcgctcttctgcgcgcccgacatgcccacggcccagtgctgcgagcccgtcgtcgccgccgtcgacctcggcggcggggtgccctgcctctgccgcgtcgccgcggagccgcagctcgtcatggcgggcctcaacgccacccacctcctcacgctctacggctcctgcggcggcctccgtcccggcggcgcccacctcgccgccgcctgcgaaggacccgctcccccggccgccatcgtcagcagccccccgcccccgccaccaccgtccgccgcacctcgccgcaagcagccagcgcacgacgcaccgccgccgccgccgccgtctagcgagaagccgtcgtccccgccgccgtcccaggagcacgacggcgccgccccccgcgccaaggccgcgcccgcccaggcgaccacctccccgctcgcgcccgctgccgccatcgccccgccgccccaggcgccacactccgcggcgcccacggcgtcgtccaaggcggccttcttcttcgtcgccacggccatgctcggcctctacatcatcctctga

SEQ ID NO: 34 - protein, 223 aa, Triticum aestivum
MERSRGLLLAAGLLAALLPAAAAAFGQQPGAPCEPTLLATQVALFCAPDMPTAQCCEPVVAAVDLGGGVPCLCRVAAEPQLVMAGLNATHLLTLYGSCGGLRPGGAHLAAACEGPAPPAAIVSSPPPPPPPSAAPRRKQPAHDAPPPPPPSSEKPSSPPPSQEHDGAAPRAKAAPAQATTSPLAPAAAIAPPPQAPHSAAPTASSKAAFFFVATAMLGLYIIL

SEQ ID NO: 35 - DNA, 5895 bp, Triticum aestivum
ccaaacaacagagtccactgtctccaagagcaccgggaaggaagcagcaagacggtgccaatcttccaactctacaggggacaacaccctatggaaacccaaatcccaattcctaccagagagctccgtgatggagatccctggatctgagcaataagaaaacaagtttggaaaagagcccgagagaggcccctctccaacccaccaatccaaccgaaagcaaacaaaacgaccatctcccaccacgaacttaactagagacctgaaatcatcatgaacatgaatcagctggcgcaagaaccgggagcccccagatccagaggcaaacaatgggttgccagtggggaaatatttagctttcaggatatcccaccatagggtcctcgcactagttctttactatactcaagccaactataagacttaaaccatttagctacaaatatcgatgcacacctcccgtggggtgttgcggaaaagcatgtttttttggtcgacaagcccctttcacaatgtatcctcttctaattctattcagatcattaacatcagctgtgattgacatcctcttcccaagatcagattcacgcaattgaacatcataaaccacatcttcaatgtcatcctcttcctatatatttttagatgattagcttgcttcgttctcaatatcaggttctatgaatggacttgagttgatgccactaataatttgtagttgttgcaaaatgtgaactgaaggggagctatgaatgaacttgagttgatttgatgggaaattgttctacacatgcacttgctgctcaacttaaatacgtgccttggatttcttcccaactttagtacataaagttctccaagtaatgttctactacataaaatttgaaatctgcaaacattttttagtacacgaacatttttctatatacagtgaacatttttcatctactgattttattttaatatatggtgaaaattggtgtaatatatgctgacatgtttttaagtacatattgaacatatatataaaatacatgatgaacatttttgttatatatgatgctcattttttcaatacatattgaacattttatattatacgatggacagttttataataatcaatgaacaacttttggagttctgaacatgcttttgaaaacacaagacattttccaataaaaaacaaaacaaaagaaatgagaaacccaaaaacaaaaacaaaacaaaacagagaaacctacagaaaaaacgaaacagagaaggcaaagaaagaaccggaactgggccagccaactcggcgtgccccagtggtccgtcgtggcgaatgtttgcaacggctacatgggccgctcctcgtgaaaaagaagaaggtcagtccatgggctgctaccagtacacgggcctcgctgtggcaaactggcaacacgccatattagttttcgcgggaatccaatgccgaaaaccacccaccgcgggaacccgacgtcggtctggtgacttctggcgccttccagaaccctccacaagctcccagagccgtctgatcagatcagcacgaagcacgaagcacgaacattggcgcgcgaagatattttctttccccagcctccgcctcgcccgacgacgccgcactgcatttcatttgaatttcaaaaatcgaaaacggaaaaactttctcgcatcccgaggagaggcggttacgcgcgccagaggagcacgagagaggccaccccacgcacccagccagctcacgtgccgccctcgcaccccccgcggccgcatccgggccgtccgatcgcacagctggccgcgctccacccgaaccgacgcccaggatcgcgcccgccacccgcttgccttcgcgtccacggctcctccggctatataacccgccccccacccgctccccctccggcattccaccccaacaccgcatcaccaccaccactccaccaaaccctagcgaccgagcgagagagggagagaccgccccgccgatggagagatcccgcggcctgctgctggcggcgggcctgctggcggcgctgctgccggcggcggcggccgcgttcgggcagcagccgggggcgccgtgcgagcccacgctgctggcgacgcaggtggcgctcttctgcgcgcccgacatgcccacggcccagtgctgcgagcccgtcgtcgccgccgtcgacctcggcggcggggtgccctgcctctgccgcgtcgccgcggagccgcagctcgtcatggcgggcctcaacgccacccacctcctcacgctctacggctcctgcggcggcctccgtcccggcggcgcccacctcgccgccgcctgcgaaggtacgtcgcgcacgttcaccgcctccctccctccctcgctctctctctctctctctctctctctctctacgtgccgattctctgtgttcgcttccctgcttacctagcacgtagttttccatggcttctcgactcgctggtcctccgatttgggtcggttaatttcctcgctgtactaccggatctgtcggcacggcgcgcggcgtcgggttctcgccgtctcccgtggcgagcgacctgcgcagcgcgcgcgcggcctagctagcttcataccgctgtaccttcagatacacggagcgatttagggtctactctgagtatttcgtcatcgtaggatgcatgtggcagtcgcgattgtttcatcgattttagatctgtgcttgttcccgcgagttaagatggatctagcgccgtacgcagacgcagatggtcttgctgtctctgttgctcgagttatcttatctactgtcgttcgagtatatttgcctgcttccttttgatctgtgtttatcgtgcagtagcagtagccatgtccacgccttcttgtttcgaggcgatcatcgtcgagatagcgctttgtttcaaaccgcaacgcagcctttgctttctgcggtatcttctgccttgtttttgttctgtgcagtacgtcttgcttggtcaaaagtaaaaactcttgctgttcgatcgaccgaggcctgatgcagagcaagagctggccgtgcttttcgctctgcagtgcatcgcctctgcctctttggccaaacatttccatgttgatcctctggtgtggtactacttttttgcatgcggtttgcgtagccttcctctttcgtgaaaaaaggtcgggtcgcctattggcagagcagcagcagcagcaacagatagctggctgtctcgcagctttgacagaaccggtctgtggccatctgtcgccgcctgccaccgtttccctgatgtttgtttctctcgtctcatctcgcctgccactgtttcttttcttgttgcgcacgtcgtcacctcctcctacttttttttccagttttgtttacttttgagatacggacgaacggctggtaattactaactttggttgctgttgttactgtggattttggacgcaggacccgctcccccggccgccatcgtcagcagccccccgcccccgccaccaccgtccgccgcacctcgccgcaagcagccagcgcgtacgaacctctccctccctctctctcgcctgcatctcgctctgtattagctgattgtgtttacttactgacgtgtgctttggctttggatctgtttcgcagacgacgcaccgccgccgccgccgccgtctagcgagaagccgtcgtccccgccgccgtcccaggagcacgacggcgccgccccccgcgccaaggccgcgcccgcccaggcgaccacctccccgctcgcgcccgctgccgccatcgccccgccgccccaggcgccacactccgcggcgcccacggcgtcgtccaaggcggccttcttcttcgtcgccacggccatgctcggcctctacatcatcctctgagtggccgaccccgcaagaccatgtccgtccagttgcagtagagtagagtgctcgtcgtcttgttccgtttcatgcttgtcgccgttcgaggttcgtctctgcatgcagtccgatcgaagaagacggtggattttgagtagtagctgtcgttggcaggagtatggagttcatgtgtcctcggtcgcctagttttggtctcaagtagtgtctgtctgtccgccgtgtttgcgtggtcgcggagaagtacaattggtgggtgtttgcgattcctctggttagatgaaccactgctatgtgatcgatcgatatgatctgaatggaatggatcaagttttgcgttccgctgatgatgatgtgatatgcttcttcatgtatatatattcatgctcgatctatttgtgtttctccgatttgaatctgtgttaagcaacagtttgtcttgcttctgttctgcagcttctgctatggatggatgcttcttgcatgcatcttgtctttgcttaatttgtagtagaacggatgcagttttgatctctgctgatgatgtgatgattcttcatatgcatatgctctgtacatgtctcttcaaatttgtgtagcaacagtctgtagttctcgttctgctctgaatgaatgcctcttgcatgttgtctttgctagctttgtggtagaaatgtagaatgcagacattgcttccgtcccaaataatctgttccttgcttcgtatatatattgacatgttgtgcatataatctgtgaatgaagttgtgaacaagtcttctttcagaaaaaaaagttgtgaacaagtgcctcacctcacctacaaggctacaaacacaacaacaacagaagctggcctcttcacggagaaccgcgcggggactgctgcagctttctgttgccatattgtttttcacgccaggacaaaatagacggtgcggtttgattcgatcccggttaattctcaatcccttcgtcactatgttccacatggaaccggagggggtagattcacattcgtgcatgcaaaatttattggtattgctcgatccatcaactcgtgtaccgtcaactgggtcacgttttgccataaaagtcttaccattttaccctagcgctatgcccacccatgctttttcatatgattctgaagttttaaatctattttatctttgaggcactaggtggtgcggtttgatttgatcccggttaattctcaatcaaattttattggtgttgctctagtgggggagcttgagcaaaatttaagagggggccatgactcaaggggaacaaattagtaggcctttaggggctactcacttgttgaaatactaattaggcctaaaagctagcacgctttttaatgaatgccaaaattaggagggggggggggggggggcatgccccccttggtctacactaaactccgccagtgtatcgccgtcatttgggtcatgtcaagcagccactttttgccataacactcttaccattttacccttttgttgaaacctctctcctcactccaaaagtacctgacgagtaatgctacgcccacccatgcattttcatagtatgattttaaagttttaaatctatcttatcttttgaattgaaagtctgatttacaatctgtttgcatatttatgttccttgcggcaaggactttcaaacaaaagacctttcttgaatatatttcgacaagttttaaaatttgatttctaaaacatggtgaaacgctatcaaacatatatagtgatgctctcccgaacaagaaaaaaaatctactaataaaacttgataagaacacacattaataacttgataaaaacattttagattcgtacgaagactgcttaaagtgtcattgtttaccaagttccacatgcattgatcgatttgattagttggaactgtcgaggttgggtcaaccacgaatagttcaagaacttgtgtgtctctctaaggcgcatcgtcccaatattatctatctttct

SEQ ID NO: 36 - DNA, 1665 bp, Triticum aestivum
atgagcagccccatggaggaagctcaccatggcatgccgtcgacgacgacggcgttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcatgtgctcaagaccaacttcaacaattaccccaagggggaggtgtacaggtcctacatggacgtgctgctcggcgacggcatcttcaacgccgacggcgagctctggaggaagcagaggaagacggcgagcttcgagttcgcttccaagaacctgagagactttagcacgatcgtgttcagggagtactccctgaagctgcgcagcatcctgagccaggcttgcaaggccggcaaagtcgtggacatgcaggagctgtacatgaggatgacgctggactcgatctgcaaggtggggttcggggtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaagatcaagcacgacatactgtcgcggttcatcgagctgggcgaggccggcggggacgacggcggcagcctgttcggggacgacaagggcctccgcgacgtggtgctcaacttcgtgatcgccgggcgggacaccacggccacgacgctctcctggttcacctacatggccatgacgcacccggccgtggccgagaagctccgccgcgagctggccgccttcgaggcggaccgcgcccgcgaggatggcgtcgcgctggtcccctgcagcgactcagacggcgacggctccgacgaggccttcgccgcccgcgtggcgcagttcgcggggctgctgagctacgacgggctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgcgttccgcaacgcgtcgccgttcaagttcacggcgttccaggcggggccgcggatctgcctcggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 37 - protein, 554 aa, Triticum aestivum
MSSPMEEAHHGMPSTTTAFFPLAGLHKFMAIFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLRSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPAVAEKLRRELAAFEADRAREDGVALVPCSDSDGDGSDEAFAARVAQFAGLLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRAPLA

SEQ ID NO: 38 - DNA, 3645 bp, Triticum aestivum
tctcatctgtggaacatatttatttggcagcactagatgcctcggcatattgcaaggtttttaatatttgcgatcttttctgtttcaagcttctaataaatagaaggtgaccactttcatcaaaattttcttctgtttagcttctgctacaaatttctaataaatatagaagggggaactttcagcaagattttttatatttgtgattttcaggctttttccatttagggagaacatcagagcaccccttgacagttgacaccccttcattcgaaatttctcaacttgttctgctttgacttcaaaaactgtttcactgaaagatgcactttgtattggttagtgcgggttcaataaagaccagatggaccataaccatggctccatggctccaactgtgaagatgacataatcacaacgctaactgtcatcaaacgcatcacctacatcccccgcaaaacgaaataaaaatgcatcagtgcatcacctacatttatagtaaaacagaaggaaaatgcagaatccatgacctagcttagcaccaagcacatactaacatacctagttatgcatataaaaatgagtgttttcttggtcagcagatcacaaaaaggacacaaacggtaggttccatctagtcagggggttaggttagggacgccatgtggatgaggcaatcttaattctcggccacaccaagattgtttggtgctcggcgccactaatgcccaatatattacctaaccgagccatccaaatgctacatagaattaatcctcctgtagactgaacccacttgatgagcagccccatggaggaagctcaccatggcatgccgtcgacgacgacggcgttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcatgtgctcaagaccaacttcaacaattaccccaaggtgaaactgaaagaacccctcagccttgtgaatttttttgccaaggttcagaagtttacactgacacaaatgtctgaaattgtacgtgtagggggaggtgtacaggtcctacatggacgtgctgctcggcgacggcatcttcaacgccgacggcgagctctggaggaagcagaggaagacggcgagcttcgagttcgcttccaagaacctgagagactttagcacgatcgtgttcagggagtactccctgaagctgcgcagcatcctgagccaggcttgcaaggccggcaaagtcgtggacatgcaggtaaccgaactcagtcccttggtcatctgaacattgatttcttggacaaaatttcaagattctgacgcgagcgagcgaattcaggagctgtacatgaggatgacgctggactcgatctgcaaggtggggttcggggtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtacgtgatcgtcgtcgtcaagctccggatcgctggtttgtgtaggtgccattgatcactgacacactagctgggtgcgcagatcaagcacgacatactgtcgcggttcatcgagctgggcgaggccggcggggacgacggcggcagcctgttcggggacgacaagggcctccgcgacgtggtgctcaacttcgtgatcgccgggcgggacaccacggccacgacgctctcctggttcacctacatggccatgacgcacccggccgtggccgagaagctccgccgcgagctggccgccttcgaggcggaccgcgcccgcgaggatggcgtcgcgctggtcccctgcagcgactcagacggcgacggctccgacgaggccttcgccgcccgcgtggcgcagttcgcggggctgctgagctacgacgggctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgcgttccgcaacgcgtcgccgttcaagttcacggcgttccaggcggggccgcggatctgcctcggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggcgccgctcgcctgatcttgacctggttccggcgacggtgatggacgctccggtggctggctggccggacggccggcgcgttatgacaggctcgatttagcttggcaactgtgataaactcgtatatgtaggcagagtggagagggtgttgatcgattcgccatggacgttgctcgtccgttgttaccatcgtaccatgtttgtattgcttctagatcactttatagttcgtgtttgttcttgagcctaagtatttattgcacatttcaaaagtgacaaatgtatgcaattgtctttttggggtgttttctaagggtagtattttcgtagatttattttgtcgaccaaaccctggccgtcacacatgattcgatccctcttgccgccgccagcgtgcgacaccagcgcggggggggggggggggggggggggggggggggggggggggggggggggctagggtgctacaccggcgggcgccgctgttgcttgtggggagtccagtatggaggagggcgacgacgatgagtggtcggattacaactgtcgacagctgatgctcgttggcggcactagtgaagccgacattggtcggaggttcacatatccagtgggaaggtcatcaacggcagcctggcttggcccggacatcggagaagagggcgtcgatgtatggtcctggatggcgacaagcttgatatcaaactcggccctatcatgcagcggcatgttttcttcttcttcttcaggtttactttaggaagtcccagtttaggagtaatgttttcccagttttattggtgtgtttatcgtcggcggaggacatgtggaactgtgtcttcgattttcttttaggatctacccggcttacatttttcgctggatccatttggattctttcgactttcatagtctacagagtttctacatgtcct

SEQ ID NO: 39 - DNA, 1656 bp, Triticum aestivum
atgagcagccccatggaggaagctcaccttggcatgccgtcgacgacggccttcttcccgctggcagggctccacaagttcatggccgtcttcctcgtgttcctctcgtggatcctggtccactggtggagcctgaggaagcagaaggggccacggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcacgtgctcaagaccaacttcaacaattaccccaagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatcctgagccaggcttgcaaggcaggcaaagttgtggacatgcaggagctgtacatgaggatgacgctggactcgatctgcaaggtggggttcggggtggagatcggcacgctgtcgccggagctgccggagaacagcttcgcgcaggccttcgacgccgccaacatcatcgtgacgctgcggttcatcgacccgctgtggcgcgtgaagaaattcctgcacgtcggctcggaggcgctgctggagcagagcatcaagctcgtcgacgagttcacctacagcgtcatccgccggcgcaaggccgagatcgtgcaagcccgggccagcggcaagcaggagaagatcaagcacgacatactgtcgcggttcatcgagctgggcgaggccggcggcgacgacggcggcagcctgttcggggacgacaagggcctccgcgacgtggtgctcaacttcgtcatcgccgggcgggacacgacggccacgacgctctcctggttcacctacatggccatgacgcacccggccgtggccgagaagctccgccgcgagctggccgccttcgagtccgagcgcgcccgcgaggatggcgtcgctctggtcccctgcagcgacggcgagggctccgacgaggccttcgccgcccgcgtggcgcagttcgcgggactcctgagctacgacgggctcgggaagctggtgtacctccacgcgtgcgtgacggagacgctccgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccagagcggtggatcggcgacgacggcgccttccgcaacgcgtcgccgttcaagttcacggcgttccaggcggggccgcggatctgcctgggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggtgccgctcgcctga

SEQ ID NO: 40 - protein, 551 aa, Triticum aestivum
MSSPMEEAHLGMPSTTAFFPLAGLHKFMAVFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLSSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPAVAEKLRRELAAFESERAREDGVALVPCSDGEGSDEAFAARVAQFAGLLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRVPLA

SEQ ID NO: 41 - DNA, 3416 bp, Triticum aestivum
gcgggagctacatgcaccgggctgccctttagcttctgctaaaaatttctagcaagtatagaagggcggaactttcaacaaagatatgagaacatcagagcactccttgacaccccttcattccaaatttctcaacttgctctgctttgacttcaaaaactgtctcactgaaagatgcactttgtattggttagtgcgggttcattaaagatcagacggaccataaccatggttccaactgtgaagatgagaccatcacaatgctaactgtcatcaaatgcatcacctacattccctgcaaaataaaaataaaaatgcacgacctacatgtgcagtaaaacagaaggaaaatgcagaatccatgacctagctcagcatcaagcacatacaaacatatctagttatatgcatataaaaatcagtattttcttggtcagcagatcacaaaaaggacacaaacggtaggttccatctagtcagggggttaggttagggacaccatgtggatgaggcaatcttaattctcggccacaccaagattgtttggtgctcggcagcactaatgcccaatatattacctaaccgagccatccaaatgctacatagagttaatcctcctgtagacctgaacccccttcatgagcagccccatggaggaagctcaccttggcatgccgtcgacgacggccttcttcccgctggcagggctccacaagttcatggccgtcttcctcgtgttcctctcgtggatcctggtccactggtggagcctgaggaagcagaaggggccacggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcacgtgctcaagaccaacttcaacaattaccccaaggtgaaacaatcctcgagatgtcagtcaaggttcagtataatcggtactgacagtgttacaaatgtctgaaatctggaattgtgtgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatcctgagccaggcttgcaaggcaggcaaagttgtggacatgcaggtaactgaactctttcccttggtcatatgaacgttgatttcttggacaaaatctcaagattctgacgcgagcgagccaattcaggagctgtacatgaggatgacgctggactcgatctgcaaggtggggttcggggtggagatcggcacgctgtcgccggagctgccggagaacagcttcgcgcaggccttcgacgccgccaacatcatcgtgacgctgcggttcatcgacccgctgtggcgcgtgaagaaattcctgcacgtcggctcggaggcgctgctggagcagagcatcaagctcgtcgacgagttcacctacagcgtcatccgccggcgcaaggccgagatcgtgcaagcccgggccagcggcaagcaggagaaggtgcgtacgtggtcatcgtcattcgtcaagctcccgatcgctggtttgtgcagatgccactgatcactgacacattaactgggcgcgcagatcaagcacgacatactgtcgcggttcatcgagctgggcgaggccggcggcgacgacggcggcagcctgttcggggacgacaagggcctccgcgacgtggtgctcaacttcgtcatcgccgggcgggacacgacggccacgacgctctcctggttcacctacatggccatgacgcacccggccgtggccgagaagctccgccgcgagctggccgccttcgagtccgagcgcgcccgcgaggatggcgtcgctctggtcccctgcagcgacggcgagggctccgacgaggccttcgccgcccgcgtggcgcagttcgcgggactcctgagctacgacgggctcgggaagctggtgtacctccacgcgtgcgtgacggagacgctccgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccagagcggtggatcggcgacgacggcgccttccgcaacgcgtcgccgttcaagttcacggcgttccaggcggggccgcggatctgcctgggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggtgccgctcgcctgatcttgatctggttccggcgacggtgatggacgctccggtggctgtctggccagacggccggcgtgttatgacaggctcgatttaacttagcaattgtgataaactcgtatatgtaggcagagtggagagtgtgttgatcgatttgccatggacgttgctcgtccgttgttaccgtcgtaccatgtttgtattgcttctagatcattatagttcgtgtttgttcttgagcctaagtatttattgcacatttcaaaaatgacaaatgtgtgcaattgtcttttttgggtgttttctaagggtagtattttcgcagatttattctgtcgaccaaaccttagcctttgacccctctcgccgtcgtccggatgcgacgtgggcaggaaggctgctcctcgtggggtgccagacatgttggagctggtggaatgttgcaggacagcgacggtgatgagtggtcagattgccgttgtcgacaggcgatgctcgatggtggcgctggtgaaggtgacggtggtcggaggatcacatatccagcacgacgatcttcaacagcggcccggcttggctaggtcattggacaagcaataatcctacacctacgaaaattgctacgtaggcttacttaacctttcataaaattctctccttccccgtgactttaaccggggtggaccccagctgctaatcctggcccaattagcaacctccacatcatcttttacgtcagatctatacgtaacattacgtatgtgtagcattgctcacaagcttggacaagagggtattgatgcatggtcctggatggtgacgagctcgacatcagacccagagctatcatgcaacgatatgtgttttttc

SEQ ID NO: 42 - DNA, 1656 bp, Triticum aestivum
atgagcagccccatggaggaagctcacggcggcatgccgtcgacgacggccttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtgaccgtcgacatgcccttcacctcctacacctacatcgccgacccggtgaacgtcgagcatgtgctcaagaccaacttcaacaattaccccaagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatactgagccaggcttgcaaggccggcaaagttgtggacatgcaggagctgtatatgaggatgacgctggactcgatctgcaaagtggggttcggagtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaagatcaagcacgacatactgtcgcggttcatcgagctgggcgaggccggcggcgacgacggcggcagtctgttcggggacgacaagggcctccgcgacgtggtgctcaacttcgtgatcgccgggcgggacaccacggccacgacgctgtcctggttcacctacatggccatgacgcacccggacgtggccgagaagctccgccgcgagctggccgccttcgaggcggagcgcgcccgcgaggatggcgtcgctctggtcccctgcggcgacggcgagggctccgacgaggccttcgctgcccgcgtggcgcagttcgcggggttcctgagctacgacggcctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgccttccgcaacgcgtcgccgttcaagttcacggcgttccaggcggggccgcggatttgcctcggcaaggactcggcgtacctgcagatgaagatggcgctggcaatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 43 - protein, 551 aa, Triticum aestivum
MSSPMEEAHGGMPSTTAFFPLAGLHKFMAIFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLSSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPDVAEKLRRELAAFEAERAREDGVALVPCGDGEGSDEAFAARVAQFAGFLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRAPLA

SEQ ID NO: 44 - DNA, 3545 bp, Triticum aestivum
ctttgtagagatttcactatgaaccacatacggatgtatataaatgcattttagaagtagattcactcattttgctccatatgtagtccatagtgaaacctctacaaagacttgtatttaggacggatggagcaataaatagaaggtgatcattttcatcaaaaatttcatttgtttggtcctgttaaaaaattctaattaatatagaagggggaaactttcaacaatattttccatctttgtgattttcaggctttttccatttagggagaacatcagagcaccccttgacaccccttcattccaaatttctcaacttgctctgcttttgacttcaaaaactattggttagtgcgggttcattaaagatcagatggaccataaccatggctccaactgtgaagatgagatcatcacagtgctaattgtcaaaaaaatgcatcacctacatcccccgcaaaagaaaataaaaatgcatcacctacatgtacagtattttcttggtcagcagatcacaaaaaggacacaaacggtaggttccatctagtcagggggttaggttagggacaccatgtggatgaggcaatcttaattctcggccacaccaagattgtttggtgctcggcagcactaatgcccaatatattacctaaccgagccatccaaatgctacatacagttaatcctcctgtagactgaacccccttcatgagcagccccatggaggaagctcacggcggcatgccgtcgacgacggccttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtgaccgtcgacatgcccttcacctcctacacctacatcgccgacccggtgaacgtcgagcatgtgctcaagaccaacttcaacaattaccccaaggtgaaacaatcctcgagatgtcagtaaaggttcagtataatcggtactgacagtgttacaaatgtctgaaatctgaaattgtatgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatactgagccaggcttgcaaggccggcaaagttgtggacatgcaggtaactgaactcattcccttggtcatctgaacgttgatttcttggacaaaatttcaagattctgacgcgagcgagcgaattcaggagctgtatatgaggatgacgctggactcgatctgcaaagtggggttcggagtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtgcgtggtcatcgtcattcgtcaagctcccggtcgctggtttgtgtagatgccatggatcactgacacactaactgggcgcgcagatcaagcacgacatactgtcgcggttcatcgagctgggcgaggccggcggcgacgacggcggcagtctgttcggggacgacaagggcctccgcgacgtggtgctcaacttcgtgatcgccgggcgggacaccacggccacgacgctgtcctggttcacctacatggccatgacgcacccggacgtggccgagaagctccgccgcgagctggccgccttcgaggcggagcgcgcccgcgaggatggcgtcgctctggtcccctgcggcgacggcgagggctccgacgaggccttcgctgcccgcgtggcgcagttcgcggggttcctgagctacgacggcctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgccttccgcaacgcgtcgccgttcaagttcacggcgttccaggcggggccgcggatttgcctcggcaaggactcggcgtacctgcagatgaagatggcgctggcaatcctgtgcaggttcttcaggttcgagctcgtggagggccaccccgtcaagtaccgcatgatgaccatcctctccatggcgcacggcctcaaggtccgcgtctccagggcgccgctcgcctgatcttgatctggttccggcgacggtgatggacctggacgctccggtggctggctggccggacggccggcgcgttatgacacgctcgatttaacttggcaactgtgataaactcgtatatgtaggcagagtggagagggtattgatcgatttgccattgacgttgccctactccatggatgtttgtattgcctctagatcattatagttcgtgtttgttcttgagcctaagtatttattgcacatttcaaaatgacaaatgtatgcaattgtcttttctggatgttttctaaggattttcgtagatttattttgtcgatcaaaccctagccgtcacacatgattcgatccctctatgggagctcgacacggaggagctggtgagctgctacaggacgacgacgctaatgagtggtcgaattgcggttgttggcaggcgatgctcgatggcggcgttggtgaagccggcggtggtcgcagggtcacatatccagcgcggcgatcttcaacagaggcccaacttggccagatcatcggagaagagggcatcgatgcatggtcctggatggcgacgagctcgacatcagacccgcacctatcatgcagcggcatgtttgttagtcctaatttaggaataaggtccccctggtccgttcatatgtttatcccgacgaagggcgtgttgagccgtgtctttgatttgtcttctgggattcggttggcttagatttcgtggtggattcatctggattcagacgactttcgtagtctacgaaattcctacaggtccttatcggcattttcttctctggggcaccgatttgattcgtagatcgtggccgccggcatcttctagtctagatcaacgacttccctgacgctgcttctacaagcttatgagttttaaaaaagtttgcttc

SEQ ID NO: 45 - DNA, 1242 bp, Triticum aestivum
atggaagagaagaagccgcggcggcagggagccgcaggacgcgatggcatcgtgcagtacccgcacctcttcatcgcggccctggcgctggccctggtcctcatggaccccttccacctcggcccgctggccgggatcgactaccggccggtgaagcacgagctggcgccgtacagggaggtcatgcagcgctggccgagggacaacggcagccgcctcaggctcggcaggctcgagttcgtcaacgaggtgttcgggccagagtccatcgagttcgaccgccagggccgcgggccctacgccgggctcgccgacggccgcgtcgtgcggtggatgggggacaaggccgggtgggagacgttcgccgtcatgaatcctgactggtcggagaaagtttgtgctaacggagtggagtcgacgacgaagaagcagcacgggaaggagaagtggtgcggccggcctctcgggctgaggttccacagggagaccggcgagctcttcatcgccgacgcgtactatgggctcatggccgttggcgaaagcggcggcgtggcgacctccctggcgagggaggccggcggggacccggtccacttcgccaacgacctcgacatccacatgaacggctcgatattcttcaccgacacgagcacgagatacagcagaaaggaccatttgaacattttgctggaaggagaaggcacggggaggctgctgagatatgaccgagaaaccggtgccgttcatgtcgtgctcaacgggctggtcttcccaaacggcgtgcagatctcacaggaccagcaatttctcctcttctccgagacaacaaactgcaggatcatgaggtactggctggaaggtccaagagcgggccaggtggaggtgttcgcgaacctgccggggttccccgacaacgtgcgcttgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtggtcgaggtactcgaggaccggggcggcgaggtgatgaagctggtgagcgaggtgagggaggtggaccggaggctgtggatcgggaccgttgcgcacaaccacatcgccacgatcccttatccgttggactag

SEQ ID NO: 46 - protein, 413 aa, Triticum aestivum
MEEKKPRRQGAAGRDGIVQYPHLFIAALALALVLMDPFHLGPLAGIDYRPVKHELAPYREVMQRWPRDNGSRLRLGRLEFVNEVFGPESIEFDRQGRGPYAGLADGRVVRWMGDKAGWETFAVMNPDWSEKVCANGVESTTKKQHGKEKWCGRPLGLRFHRETGELFIADAYYGLMAVGESGGVATSLAREAGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 47 - DNA, 3248 bp, Triticum aestivum
aggacagacgcttaattagacgtttctcctgtagaaataggcacaaatgcttcaaaaaaatccgatttgtttttataagcacctagcattgtacgaggccttacgtatttgttgggtgcttaaaaaggaagagaaagaaagaaagaaagcgatctagaaatttaaacactgaagggacccatgtcgtcaccctagggccttccgaaacgtaggaccgaccctacacgcaccgcattacgccaattatctctccctctaatccccttataattacctctataacatctgtcaataactaaatcattatcacgaatgataccgaattcttgactgctcccttgctcttctgcttctttctcctccaaagtttgctcttctctccctgatcctgatcctcaccagatcaggtcatgcatgataattggctcggtatatcctcctggatcactttatgcttgctttttttgagaatccactttatgcttgttgacctgtacatcttgcatcactatccaagcaacgaaggcatgcaaatcccaaattccaaaagcgccatatccccttagctgttctgaaccgaaatacacctactcccaaacgatcacaccgacccatgcaacctccgtgcgtgccgggataatattgtcacgctagctgactcatgcaactcccgtgcatgtcggtatatattttcggggcaaatccattaagaatttaagatcacattgcccgcgcttttttcgtccgcatgcaaactagagccactgccctctacctccatggaagagaagaagccgcggcggcagggagccgcaggacgcgatggcatcgtgcagtacccgcacctcttcatcgcggccctggcgctggccctggtcctcatggaccccttccacctcggcccgctggccgggatcgactaccggccggtgaagcacgagctggcgccgtacagggaggtcatgcagcgctggccgagggacaacggcagccgcctcaggctcggcaggctcgagttcgtcaacgaggtgttcgggccagagtccatcgagttcgaccgccagggccgcgggccctacgccgggctcgccgacggccgcgtcgtgcggtggatgggggacaaggccgggtgggagacgttcgccgtcatgaatcctgactggtattggcttactgcagaaaaaccatagcttacctgtgtgtgtgcaaactaaaatagttttttcggaaaaaaaaaggtcggagaaagtttgtgctaacggagtggagtcgacgacgaagaagcagcacgggaaggagaagtggtgcggccggcctctcgggctgaggttccacagggagaccggcgagctcttcatcgccgacgcgtactatgggctcatggccgttggcgaaagcggcggcgtggcgacctccctggcgagggaggccggcggggacccggtccacttcgccaacgacctcgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatctcacaggaccagcaatttctcctcttctccgagacaacaaactgcaggtgagataaactcaggttttcagtatgatccggctcgagagatccaggaactgatgacgcctttattaatcggctcatgcatgcacactaggatcatgaggtactggctggaaggtccaagagcgggccaggtggaggtgttcgcgaacctgccggggttccccgacaacgtgcgcttgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtggtcgaggtactcgaggaccggggcggcgaggtgatgaagctggtgagcgaggtgagggaggtggaccggaggctgtggatcgggaccgttgcgcacaaccacatcgccacgatcccttatccgttggactagagtgtgtagtgtctcatttgatttgctggttttatattagcaaggaggtgtatcagtttatggtttgcttgtttattgggttcgtgtgatgatcatgttgtgaatttgacgatggattctttttcttttgtgacaagaactcggatctttataaaagctcacgagaagtacaaggcataataaaaattacattgagattctagaactgtaatgcaattgtttgagttttcatgtatatatgaattgatcatgttttttgatttgtttgtacaccacctcgacatacaaggaccaaagagtataaggacttatagttctacgcaacgagctcaacctcaaacgcattgtcatcccttctctccttgaaataaaaaagcaatattgatgcaagcaccgcgccagggcgttggccctctacagcttgacatgtgtcatcatctacttggttgccacgtacatgccaatttagaagtttttcttaactttcttttttctatattcattgagatttaccgttgaggccatggaaatattcgaatgggtctcggcctgcccactccaaatctcccgctccatccctttctttgttcttctagtccaaacggaaatatgagagaaggttagagtcttgattgttgtgcctagaaaaaaacgatgcctgagtggagcctgagtgggggaccttttttgcctggccaggcaagcctaggcgtgggtgtttggttccttctctaggtggtcagtttgtcctttagcacttagataaattttgtactgcgggccatactgtttatgactcgctatcagcgctaggcaggcagctggccaggcagaacaaatagaatgcctgggcgaggctaaccaggttgcctgggccaagcatgtttctttcttttgttttttaaatctagaccaagttaatcacgttgcatggactcccatgccaggaagatgtttcattttctaggacaccatccaaataaata

SEQ ID NO: 48 - DNA, 1242 bp, Triticum aestivum
atggaagagaagaagccgcggcggcagggagccgcagtacgcgatggcatcgtgcagtacccgcacctcttcatcgcggccctggcgctggccctggtcgtcatggaccccttccacctcggcccgctggctgggatcgactaccggccggtgaagcacgagctggcgccatacagggaggtcatgcagcgctggccgagggacaacggcagccgcctcaggctcggcaggctcgagttcgtcaacgaggtgttcgggccggagtccatcgagttcgacagccagggccgcgggccctacgccgggctcgccgacggccgcgtcgtgcggtggatgggggacaagaccgggtgggagacgttcgccgtcatgaatcctgactggtcggagaaagtttgtgctaacggagtggagtcaacgacgaagaagcagcacgggaaggagaagtggtgcggccggcctctcgggctgaggttccacagggagaccggcgagctcttcatcgccgacgcgtactatgggctcatggccgtcggcgaaagcggcggcgtggcgacctccctggcaagggaggtcggcggggacccggtccacttcgccaacgacctcgacatccacatgaacggctcgatattcttcaccgacacgagcacgagatacagcagaaaggatcatttgaacattttgctagaaggagaaggcacggggaggctgctgagatatgaccgagaaaccggtgccgttcatgtcgtgctcaacgggctggtcttcccaaacggcgtgcagatttcacaggaccagcaatttctcctcttctccgagacaaccaactgcaggatcatgaggtactggctggaaggtccaagagcgggccaggtggaggtgttcgcgaacctgccggggttccccgacaacgtgcgcctgaacagcaaggggcagttctgggtggcgatcgactgctgccggacgccgacgcaggaggtgttcgcgaggtggccgtggctgcggaccgcctacttcaagatcccggtgtcgatgaagacgctggggaagatggtgagcatgaagatgtacacgcttctcgcgctcctcgacggcgaggggaacgtggtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 49 - protein, 413 aa, Triticum aestivum
MEEKKPRRQGAAVRDGIVQYPHLFIAALALALVVMDPFHLGPLAGIDYRPVKHELAPYREVMQRWPRDNGSRLRLGRLEFVNEVFGPESIEFDSQGRGPYAGLADGRVVRWMGDKTGWETFAVMNPDWSEKVCANGVESTTKKQHGKEKWCGRPLGLRFHRETGELFIADAYYGLMAVGESGGVATSLAREVGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 50 - DNA, 3065 bp, Triticum aestivum
tctgtcacaagtacgtattcatccatcctaattttgtgtgtcctattcatgcctagggttctcatgtataaatttctaattcttcgtgttctcttttcttcataattttaggatattagcccgccttacaatgttgtctaagacccgtaaaagaaacaatgttctctaagaagcatttgccgggtgcttaaaaaagaagaaaagaaagaaagaaagtgatctgaaaattcaaacactgaaggggcccatgtcgtcgacctagggccttccgaaacgtagaaccaaacctacacgcaccgcattacgccaattatctctccctctaatcctctgacaatttcctttataatgactgtcaataactaaatccttatcacgaatgagaccgaattttgctcttctctccctgtatcctgatcctcaccagatcaggtcatgcatgataattggctcggtatatcctcctggatcactttatgcttgttgacctgtacatcttgcatcactttccaagcaacaaaggcatgcaagtctcaaattccaaaaaggccatatccccttagctgttctgaaccgaaatacacctactcccaaacgatcacaccgacccatgcaacctccgtgcatgtcgggataatcttgtgacgctagctaactcatgcaactcccgtgcatgtcggaatatattttcggggcaaatccattaagaatttaagatcacgttgcccgcgcttttttcgtctgcatgcaaacgagaaccactgccctctgcctccatggaagagaagaagccgcggcggcagggagccgcagtacgcgatggcatcgtgcagtacccgcacctcttcatcgcggccctggcgctggccctggtcgtcatggaccccttccacctcggcccgctggctgggatcgactaccggccggtgaagcacgagctggcgccatacagggaggtcatgcagcgctggccgagggacaacggcagccgcctcaggctcggcaggctcgagttcgtcaacgaggtgttcgggccggagtccatcgagttcgacagccagggccgcgggccctacgccgggctcgccgacggccgcgtcgtgcggtggatgggggacaagaccgggtgggagacgttcgccgtcatgaatcctgactggtaattggcttactgcagataaatccatagcttacctgtgtgtttgcaaactaaaatgatttcttgggaaaaaaaaaggtcggagaaagtttgtgctaacggagtggagtcaacgacgaagaagcagcacgggaaggagaagtggtgcggccggcctctcgggctgaggttccacagggagaccggcgagctcttcatcgccgacgcgtactatgggctcatggccgtcggcgaaagcggcggcgtggcgacctccctggcaagggaggtcggcggggacccggtccacttcgccaacgacctcgacatccacatgaacggctcgatattcttcaccgacacgagcacgagatacagcagaaagtgagcggagtactgtcgctgatctccatttttgttcttgagatgttgtgtttgagtgtctgacaccatgactgatcatgcagggatcatttgaacattttgctagaaggagaaggcacggggaggctgctgagatatgaccgagaaaccggtgccgttcatgtcgtgctcaacgggctggtcttcccaaacggcgtgcagatttcacaggaccagcaatttctcctcttctccgagacaaccaactgcaggtgagataaactcaggttttcagtatgatccggctcgagagatccaggaactgatgacggatcatgcatgcacgctaggatcatgaggtactggctggaaggtccaagagcgggccaggtggaggtgttcgcgaacctgccggggttccccgacaacgtgcgcctgaacagcaaggggcagttctgggtggcgatcgactgctgccggacgccgacgcaggaggtgttcgcgaggtggccgtggctgcggaccgcctacttcaagatcccggtgtcgatgaagacgctggggaagatggtgagcatgaagatgtacacgcttctcgcgctcctcgacggcgaggggaacgtggtggaggtgctcgaggaccggggcggcgaggtgatgaagctggtgagcgaggtgagggaggtggaccggaggctgtggatcgggaccgttgcgcacaaccacatcgccacgatcccttacccgctggactagagggagtgtgcagtgtccatttgctggtttatattagcaaggaggtgtatcagtttatggtttgcttgtttattgggttcgtgtgatgatcatattgtgaatttgacgatggattctttttcttttgtgacaagaactctgatctttataaaggctcacgagaagtatataagcataataaaaattatatcaaggtccttgaatcgtcgaacaaccattgccgccatcagaacaagccgttgtcgtcgcttctgctggagccggcctaatgttgtagatcagcgccttctagttgcagtcgtcaccgtcaaagccttgaatcgatctaaagaatcctacaccaaatcttgccatcgcgtatgcacgacgagaaaccctaacctcaccgcaccgagaagctagcgggaatcaaagacagggctccatctaatccgcccctacttacgaacttgaggaggatcaaaacctatagaagagtaatgatgagtggatttctcagtcattttcatccatgtttaaaccggatattctcagattttttcgagataatcacttcaatttgcctactaatgactaaaataattgcataagattgcaaatcacattgattattttatttcatgcaaaaatttgctattttcggtgataaattaggccataaaagggacataatggctcaagatcaaactcaatcagtcggagccgtgtagcagcttccagaggaagagacaacatgcggtacaaacatggctactcgtatcgatactcgtaccaagcgccaacgaccccatgacgtatccctaacgac

SEQ ID NO: 51 - DNA, 1242 bp, Triticum aestivum
atggaagagaagaaaccgcggcggcagggagccgcagtacgcgatggcatcgtgcagtacccgcacctcttcatcgcggccctggcgctggccctggtcctcatggacccgttccacctcggcccgctggccgggatcgactaccgaccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccttacgccgggctcgccgacggccgcgtcgtgcggtggatgggggacaaggccgggtgggagacgttcgccgtcatgaatcctgactggtcggagaaagtttgtgctaacggagtggagtcgacgacgaagaagcagcacgggaaggagaagtggtgcggccggcctctcggcctgaggttccacagggagaccggcgagctcttcatcgccgacgcgtactatgggctcatggccgtcggcgaaaggggcggcgtggcgacctccctggcgagggaggccggcggggacccggtccacttcgccaacgaccttgacatccacatgaacggctcgatattcttcaccgacacgagcacgagatacagcagaaaggaccatttgaacattttgctggaaggagaaggcacggggaggctgctgagatatgaccgagaaaccggtgccgttcatgtcgtgctcaacgggctggtcttcccaaacggcgtgcagatatcacaggaccagcaatttctcctcttctccgagacaacaaactgcaggatcatgaggtactggctggaaggtccaagagcgggccaggtggaggtgttcgcgaacctgccggggttccccgacaatgtgcgcctgaacagcaaggggcagttctgggtggccatcgactgctgccgtacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 52 - protein, 413 aa, Triticum aestivum
MEEKKPRRQGAAVRDGIVQYPHLFIAALALALVLMDPFHLGPLAGIDYRPVKHELAPYREVMQRWPRDNGSRLRLGRLEFVNEVFGPESIEFDRQGRGPYAGLADGRVVRWMGDKAGWETFAVMNPDWSEKVCANGVESTTKKQHGKEKWCGRPLGLRFHRETGELFIADAYYGLMAVGERGGVATSLAREAGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 53 - DNA, 3085 bp, Triticum aestivum
aggctttctttaagtatcggtgcttatttgtacaggtcagacgcttaattaggcgtctctcctgtagaaataggcaccgatgcttcaaaaaaaaacccgctctatttttctaagcacataacattgtacaagaccttaagcatttgtcgggtgcttaaaagaaagaaaaagaaagaaagaatgcgatctgaaaatttaaacactgaagggacccatgtcgtcgccctagggccttcctaaacgtaggaccgaccctgcatgcaccgcattacgccaattatctctccctctaatcttcttacaattatctccataacaactgctaataactaaatcattatcacgaatgaggctgaattcttgacttctcccttgctcttctgcttctttctcctccaaagtttgctcttctctccctgtatactgatcctcaccagatcaggtcatgcatgaaaattggctcggtatcctcctggatcactttatgcttgttgacctgtacatcttgcatcactatccaagcaacgaaggcatgcaagtcccaaattccaaaagcgccatatccccttagctgttctgaaccgaaatacacctactcccaaacgatcacaccgacccatgcaacctccgtgcgtgtcgggataatcttgtgacgctagctgactcatgcaactcccgtgcgtgtcggaatatattttcggagcaaatccattaagaatttaagatcacattgcccgcgcttttttcgtctgcatgcaaaacagagccactgccctctacctccatggaagagaagaaaccgcggcggcagggagccgcagtacgcgatggcatcgtgcagtacccgcacctcttcatcgcggccctggcgctggccctggtcctcatggacccgttccacctcggcccgctggccgggatcgactaccgaccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccttacgccgggctcgccgacggccgcgtcgtgcggtggatgggggacaaggccgggtgggagacgttcgccgtcatgaatcctgactggtactggcttactgcagaaaaacccatagcttacctgtgtgtgtgcagactaaaatagtttctttcataaaaaaaaggtcggagaaagtttgtgctaacggagtggagtcgacgacgaagaagcagcacgggaaggagaagtggtgcggccggcctctcggcctgaggttccacagggagaccggcgagctcttcatcgccgacgcgtactatgggctcatggccgtcggcgaaaggggcggcgtggcgacctccctggcgagggaggccggcggggacccggtccacttcgccaacgaccttgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatatcacaggaccagcaatttctcctcttctccgagacaacaaactgcaggtgagataaactcaggttttcagtatgatccggctcgagagatccaggaactgatgacggctcatgcatgcacactaggatcatgaggtactggctggaaggtccaagagcgggccaggtggaggtgttcgcgaacctgccggggttccccgacaatgtgcgcctgaacagcaaggggcagttctgggtggccatcgactgctgccgtacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactagagggagtgtgtagtgtcccatttgatttgctggttttatattagcaaggaggtgtatcagtttatggtttgcttgttcattgggttcgtgtgatgatcatgttgtgaatttgacggtggattctttttcttttgtgacaagaactcggatctttataaatgctcacgagaagtacaaagcataataaaaaattatatcaaggttctagaactgtaatgcaattgtttgagttttcatgtatatgaattgatcatgttttttgatctatttgtacaccacctcgacatacgaggaccaaagagtacaaggacttatagttctacgcgacgagctcaacctcaaacgcattgccatcccttctctccttgaaataaaaaattatatattttttgcagggaaataaaaaaacaatattgatgtatgcatgggcacggcgtgccgccacgccagggcgttggccttctgcagcttggcatgtgtcctcatctacttggttgccatgcacaagtcaatctagaagtttttttaactttcttttttctatattcattgagatttaccgtttaggccatggaaatatttgaatggggctcaacctgcccactcccaatctctcgctccttcgctttcttcgttcttccagtccaaacagaaagatgagagaaggttagagtcctgaatgttgtgtctggaaaaaaatgatgcttgagtggagcctgagtgggggaccttttttgcctagccaggcaagcctaggtgtcggtgtttggttcctttcctgagtggtcggtttatcctttagcgtgggtgtttggttccttccctgggtg

SEQ ID NO: 54 - DNA, 4266 bp, Triticum aestivum
ccacacctaagaagactaaaaaaacctaacctaaactattaaccagaccgaaagcaccgggatccctaaccccgccaccagtcgtcggagcggcaggtagaggggaggcgaatccacggtctcattgatgaagcctggaggggagatttaccatagccaccaagggataggggagttcatatcacggaaaccacacaatattaatatgtcttggtggtaaaaggaattgttgcatcctacatctttggaaattgaaatggagagggtctaactattgtaactttcatgaaatatgaaagttaatgatgcatgtcttgattttcagcgcaaaataaaatgtaacatgtggtgcttgagtatagcacatatccgagtcctagagtttcaacctgccaactacgaaacaaattggactaaaaactcacataaccttttatgttacaaaaaaatgcatttatgtgccttaagaaaaaaacataaagttgatattctgatgagattttgaatgtttgatttgattttattggattgcagagggtgttggcatccagcagaagaaccctggctcttttccagatttccgctctggtcgacttcctcatccaaaaaagtggaaactgcaaagttttcatggcagcggaagtatttaaaaaataaaatcaaagagggctcaagaaagtgcaaaatgcaaattcttttgggcagcagaaggattttaaaaataaaatcaaaggaaaaaatggagatcccttagcgggcctggcctggcaccggcccggtccggcgggaagccgagccaccaatactctcactcgcgcacccgttggtggcagccgccggcccccgtcgcctccttcccctgaaccctactccaccatggccgccgcccgcgccgccctcctccgccgccacggcctcggcgccgccgccaccaaccccgtcctcttctccggccacggcctccgctaccgcaagctcgaggtcatcctcaccacggtaagccaccccccctgcccccctccccccgcgacgctccgctcggtcctccacctctgcaggatgcatcccgcgatctcgctagccgcttgcgtttccggtcagttcgagcggggtgctttcgatccggtgaggccggtgccgcctcagcgacagtttgaccgataaatctccagcatttctgaaactttttaagctagcagtagtatttttgaccgataaagcttcagttttggcctttcatttcaacaatgtccctagagattttagattgtgcggggaagtggaactaacctttgacatcacccatcgcttgttcacctcagacgatcgacaagctggggaaggcgggggagacggtgaaggtggcgccggggcacttccgcaactacctcatgcccaagatgctcgccgtccccaacatcgacaagttcgccatactcatgcgcgagcagagcaaggttagcttcccccttcttttccccgatagaaataaacatgccgcgatggccgtgcagtttggaatgctccgtcgcggctcacaagcatttgcttactaactactagcttactctgctggcttttgaggctctgctttccagagttcgtttatcagttcttcatctgcgtattgaatgaattttacgagctctccaatacttgttgcatcttagtagctctttggtagaataggcttatatacaactctatgctacttgtgttcagtgagcaagttggtgctgaagttattttggttcattctagttcatctccactgaagtatttgcttcttgtaaagttctgttaccgtaagaatatttacattgtaaactattactgctttgctggcttacaagtctctcatttgtttgatcagctttacaaacgcgaagtggaggtggttgtcaaagaagtctcaaaggaggaggatgatgctcgggtatgtcgtgcaccgagtggttcttcctttttcgtgaactgcagtgtagatgagagttcttagtacacaactaactgacctatcctgttccttttctgaaaccctgtttgccattcaacagaacttttgcctacagctgtctttggttgatttgtgcacaatttctgtctaactgacctctcctattcatcctctgaaactcagtttgctatttaacaccatatcagtccagagccgtctttggtggatttgttttggttaacgcacttcgagtccactctaaacatctggtgctgaaaatagtgaatatgttgccttggaaaagtcttcttgtacctcttggcttctgctccctctgttcctaaatgtttgtgtttctagagatttcaaatggactgccacatatggatgtatatagacatattttagagtgtagattcactcattttgctctgtatgtaggcacttgttgaaatttctagaaagacaaatatttagggacggaggaagtagatattatgtttagaatgtagggcctcttaagttcatttttgtcactgtggccagctactcaacactgttccaatactgtttttcagatacacaagtatagtagctgtcctctcaactgtatcgtccagctcatccatttcatactgtacatagaaaagatccttttgatgcttacttaaaacaacattttttgtaactgaatcctgacattgaagttcttgttttctcagttcggttaaatacagatcttttatgtgttttcccagtcaccggcctcttaagttcatttcgttttaaaatgttactcatttacttctcaactcaaccatttgaacagcaagcggaagagaaactgaagcagtgtcaagcagcagcaaaacggctcgataatgctctcttggttagtatggttccaccaaattttggagtcctggtgcagcatacttttgttgatgttccagatggataatcttgtccactattagcaagtgctgatcaaaatatttatgtttcataggtgttcagacggttcatctccgaagggatcgagttgcgctctcctgtaacgaaggatgaaattgtttctgaggttctgctccagcgctgtcatccgctatttattcaaaaacattaatctgtaggtagtaacatcaaactttaccaacaggtggcgaggcaactcaacgtcaacatttacccagacaatttacacctggtgtcaccattgtcatccctcggagaatttgaggtgccacttcgcttaccgagggatataccacgcccagaaggcaagctacaatggactctcaaggtcaagatcaggagaccctaagcactttcggtgggcgatctttgctctccttccaaggtgctgaatggtaccaaaaggccatttcagccttcggatgaagagacacgctgaaatagaccttcaaactcaaccttttccctttacaatttgcttgggccatcgtctcgggcggggcgatatgatcgggccttttgttcctacaaaaaaacgttgagaaatagtgaataatttgcctggagtagggggcctagtattgttgttgctgtttgactttatcatattcttgacttgttagtgtgcccaatcctggtgtgaaaggggggagatggatataaagaaagaaaggttgtgtgtgcaaggcatccttgaaaaggagaggcagggagtgaaagcttcctcagaaatgctcatttggcgtcgtcatcatcatatgaaaaaatggccgtctccatcgacgttttagtcggcttatattgctctacgtgtcgtgtgacggccttttgctttgatgtggaaatgctctttaattcgcgcacgcatattttgtgcttccttatctcccccatttgactgaatggttatcagttgatccatggaccccgggcaatcattgtcttggtcctattttaagatctgagctgaattacattgacactgacttgtcagtggagacccattgatctctgcgatctctgcttaatcttgtttcccattttttgccaggcattactttgaaaaaattattgcggtaattacgcgtcgacaagggctatctttgcatccaaagtgctaatacaaaatgttgaaagagaagggcactggtgcaaaaaataagagtgaaaatcagcactttggcagtctgatgaactttcatgtggagctggggtgcccagatcctcactttgcttgagcactgcaaaatacctttcctatgcagcaagagaaagctgtaaagcaggtgatctcacctgcaaggcatcagggttgagaagcaacagagatgcct

SEQ ID NO: 55 - DNA, 4406 bp, Triticum aestivum
  modified_base (1321)..(1321): a, c, t or g
gaaaaattgttgcatcctacatctttggaaattgaaatggagagggtctaactattgtaactttcctgaaataggaaaattaatgatacatatcttgattttcagcgcaaaataacatgtaacatgtggtgcttgaatatatcagataaccaagtccaagagttttgacttgccaactatgaaacaaattggactaaaaactcacataacctttatgttacgaaaaatagcatttatgtgccttcagaaaaaaggcctaaagttgatattttgatgagattttgaatgtctgatttgatttttattggattgcggagggtgctggcatccggcagaagaatccctactcttttccggatttctttcctggtctactacctcgctcaggaaagtgcaaattgcaaagttttcatggcagcagaagaattttgaaaaataaatcaaagggactcgagattttttttagtctatcaaagggactcaagaaaatgcaaatgcaaattcttttgggcagcagaagtgttttaaaaataaattcaaagggaaaaaatggagatcccttagcgggcctggcctggcaccggcccggtccggcgggaagccaccaatactctccctcgcgcacccgttttgtggcagccgccggcccccttcccctgaaccctactccaccatggccgccgcccgcgccgccctcctccgccgtcccggcctcggcgccgccgccgccaaccccgtcctcttctccggccacggcctccgctaccgcaagctcgaggtcatcctcaccacggtaagccagcccccctgccccctccctccccttctcctccaaatctcaacccgcgaccctccgctcggtcctccacctctgcaggatgcatcccgcttgcgtttccggtcagttcgagcgggatgtttccgatccggtgaggccggtgccgcctcagcgacagtttgaccgataaatcttcagcatttctgaaactttttaagctagcagtagtattcttgaccggtaaagcttcggttttggctgttcatttcaacaatatccctagagattttcaatgtgccgggaagtggaattattaacctttgccatcgtccatcgcttgttcacttcagacgatcgacaagctggggaaggcgggggagacggtgaaggtggcgccggggcacttccgcaactacctcatgcccaagatgctcgccgtccccaacatcgacaagttcgccatactcatgcgcgagcagagcaaggttagcttcctccttttccccgatagaaataaacatgctacgatggccgtgcagtttntggaatgctcggttgcagctcacaagcattacttactagcttactcttgttggcttttgaggctatgctttccggagtttcgtatatcagttctgcctctgtgtattgtatgatttgtacgagttctccaatagttgttgcaccttagctctttggtagaatagccttatatgcaactctatgctagtgttcagtgaggaagttgtactgaagtcatcgtggttcattctagttcatctccactgaagtacgcttcttgtagagttcagtcactgtaagaatatttgcagtgtaaactgttactttttaggcttacaagtctctcatttgtttgatcagctttacaagcgtgaagaggaggtggttgtcaaagaagtctcaaaggaggaggatgatgctcgggtatgtcgtgcactgattagttcttcctttttcatgaactgaagtgtcgatgagttcttagcacacaactaactgacctgtcctgttccttctctgaaaccctgtttgccatttaacacaatttctgcccagatctgtctttggaggatctgttgttaacgcacttcaagcccattctaaatctggtgttgaaaatattgaatattttgccttgaaaaagtcttcttctacctctcggcttctagatattatgtttagaatgtagtgtatcttaagtccatttgtcactgtagccgactactcaacactgttccaatactgtttttcagatatacaattatagtagctgtcctctgaactgtaatgtgcatctcatccattccatactgtacatataaaaggtccctttgatgcttacttaaaacccattttttttaactgaatactctgagattgaagttattgttaaatgatgctcctaaaattattggttcggttaactatagatcttgtatgtgttttccctgtcatacttcattttgtttttaacaccaaatttctcttccttttctgaaaccccgtttgccattcaacacaatttctgtctagatctgtctttggttgatttgtacacaaaactgacttctcctattcatcttctgaaactccgttccatatcagtccagagctgtcttcgctggatttgttttggttaacgcacttcaagcccactctaaacatatggtgctgaaaatagtgaagatgttgccttggaaaagtcgtcttctatctgttggcttctagatattatgtttagaatatagtgctttttaagttcatttgtcactgtagccagctagttaacactgttccaatactgtttttcagatatacaagtatagtatcagctgtcctctgaactgtaacgtccagctcatccagttcatactgtacatagaaaagatccttttgatgcttacttaaaacaacattttttgtaactgaatcctgacatcgaagttcttgtttttgtatgcttctcagttcagttaaatacagatctttatatgtgttttcccagccatacttcattttgtttttaaatgttactcatttacttctgaactcaaccatttgaacagcaagcggaagagaaactgaagcagtgtcaagcagcagcaaaacggctcgataatgctcttttggttagtatggttccaccaaattttggagtcctggtgcagcatacttttgttgatgttccagatggacaattttgtccagtattagcacgtgctgatcaaaatatttatgtttcataggtgttcagacggttcatctctgaagggatcgagttgcgctctcctgtaacaaaggatgaaattgtttctgaggttctgctccagcgctatcatccgctatttattcaagaacattaatctgtaggtagtaacatcaaactttaccaacaggtggcaaggcaactcaatgtcaacatttacccagacaatttgcacctggtgtcaccattgtcatcccttggagaattcgaggtgccacttcgcttaccgagggctataccacgcccagaaggcaagctacaatggactctcaaggtcaagatcaggagaccctaagcactttcggtgggcgatcttgctctccttccaaggtgctgaattgtaccgaaagaccgttgcagccttcagatgaagagacacgctgaaatagaccttcaaactcaaccttttccctttacaatttgcttgggctatcgtctcgagcggggcgatatgatgggcctttcgttcctacaaacaaacgttaagaaatagtgaataatttgcttggggtaggatgcctagcattgttgttgctgtttgactttatcatattcttgacttgttagtgtgcccaatcctggtgtgaaaggggggagatggatataaagaaagaaaggttgtgtgtgcaaggcatccttgaaaaggagaggccgggagtgaaagcttcctcagaaatgctcatttggcgtcgtcatcatcatatgaaaaaatggctgtctccatcgacgttttagtcggcttatatttctctacgtgtcgtgcgacggccttttgctttgatgtggaaatgctctttaattcgcgcacgcatattttgtgcctccttatcttccccatttgactgaatggttatcagttgatccatggacccctggcaatcattgtcttggtcctattttaagatctgagctgaattacattgacactgactggacagtggagacccattgatctctgcttaatcttgtttcccatttttgccagccattactttgaaaaaactattgtggtaatttacgtgtcgacaagggttatctttgcatccaaagtagtaatacaaaatgttcaaagagaagggcactggtacaaaaaaataagagtgaaaatcagcactttggcagtctgatgaactttcatgtggagctggggtgcccagatcctcactttgcttgagcactgcaaaatacctttcctatgcagcaagagaaagctgtaaagcagtgatctcacctgcaaggcatcagggttgagaagcaacagagatgccttcttttggggaggaaacagcagcccaattagtagcacttcattgttaaggtgctgttcaagcttcttatatg

SEQ ID NO: 56 - DNA, 2505 bp, Triticum aestivum
cgctggcgccagagaagaggcccatctctgttgtggttgtggtggctagggtttgccggcgacgagggagcaaaggatggcagatgtggacggcgagtttggacaaggacggccccgccgcacggacggcttaaaaaggacgagcgtcatcgctgacatgtgggcccgtcgtcataaattaagctgacagcgtggacaacgggtagttggacggccgccatgtgggaacacggcggacaacaggaaggcgcgcgaagcgtccgttcggcgtccgcgccgacgcatttggggcgcaaatttggaccgcaaatgcgtcggcgcggacatgacgcggatgtgatttgggtttgggtcgcgcgttgagccgtcatttttgtccgcgccgacccaaacgggcgcaggcggatgaaatgagtcgaccctttggagttgctcttaagcgatgttcaagtgggagctgtaatttatcccgcattcggaaattatattaaaccaatggcaatgaccaaaataagattttaccagtaaaacaaaaagtcgttcatgggcaggcaaagcccagcacgaatcttggcggctcgcatcctctattgcggcgctgcatcatggacacgccagcctgccaaagccaaagccaaagcgccccaatgcgatgccacgaaaaagcgatcagcatcagacacagccgcgcgacaatctgctaaagaaacccacataaaaacgcgcagcgcccggaacgccgcgcggcgaccacggtgccgtgcgggggtgtctgcgtctctctctcccctccctctctccgccgacgcggcgcgggccgagggaatggccgccgccgcctccgcctccgcctccgcctcgtcttcttcctccacctccacctcggccgggtcctccgcgtccacctccacgccccggcccgccccgcgccaggccgccgcggcgccgtcgtcgtccccggtcttcctcaacgtgtacgacgtgaccccggccaacgggtacgcgcggtggctggggctcggcgtgtaccactcgggcgtgcaggtccacggggtggagtacgcgtacggcgcgcacgagggcgccgggagcggcatcttcgaggtgcccccgcggcggtgccccggctacgcgttccgggaggcggtgctggtgggcaccacggcgctgacccgcgccgaggtgcgcgccctcatggccgacctcgccgccgacttcccgggcgacgcctacaacctcgtctcccgcaactgcaaccacttctgcgacgccgcgtgccgccgcctcgtcgcccgcgcccgcatcccgcgctgggtcaaccgcctcgccaagatcggggtcgtcttcacctgcgtcatccccagcagcagcaggcaccaggtgcgccgcaagggggagccgcagctgcccgcccccgtcaagagccgctccgcgcgccagcccgccgccccgccgcggcccaggaccttcttccgctccctctccgtcggcggcggcaagaacgtcacgccccgcccgctccagaccccgccggtggggccgcccctgacgttgacgacgccggcaccgacgccgttggcctccatgtaacggcgccattactcctttttcgtttacagctcacaccatccattttttttccttcgacagttacctgaattttgtccatagtactgtactcttcgagattaagatttgtgctctgctagtgctgcactgtcaccatgattagcagtagtaactgcagttcattaggctattaattcccgattttgtctggctttactacctagacacacctggctggctgtgtccgctgccaaatcgccattaatgattactaatttgggtcgctgttacgcgctgcatttacgttgcggttaacgacgcctatcatgcaattgtttttgttgtgtggcatggatgcaattctatccggcgagccgtccaatgggaatatattcgctcctcctttcgcccgttctttggagtaaacaaccatggagctgaagccttgtttggattttcaactatagataaaagctacacacaggctatgcaccgatcggccgatatgcttttgctgatgcaaagaattccccgtgtctggacagtggacctgtcatcactgccgttgtcatgggacacgattagattagtcctcgtgttgttgtttcttgcatgattgcgtccggcctccgtgcctatctggaaatgcggagggcgggataattttaacgtgacttgtcgcgtgaaaggcgagctcgcttcgacagaaatcttggggagctcgccggttgcgtgtccagcgcgcctcgccgttgaccggcgaccggtgtgtccatgccggtggcgaagacggcggcgcggggtcagaattgggcaccgacgggaggagggttcgcatttgtggaggacaccgccacgcagcacagtgcaccacattggccttgacccgtccgatcagcgatcagcgatcaggatggacgggccactatcgatcct

SEQ ID NO: 57 - DNA, 2585 bp, Triticum aestivum
gagccatcatttctacggtcgggctgcttttgtagggtcagcgtgttttccgcgacaaattctcattgtgcctatcccgtcccccttcgcccactaggcaagaactctcagtgtcgtgactaggttttgacgtgcagagagtacacgacgcgtgatcgtgaggccaacacccaacagtatcctaggccctaacgcatggaaaccaagtgaccgagcgagaagagaatggaggcccagaatctttggtggaaagaaacgacgtggttgtcatgtacttatgctgattacaaaattgcaaagtctggtcagaaccatcatttggtcggcattgagagttttccttctttttgaatggacaggaattgagagttgatggtgcatggtgccatttaagcgatgttcaaacgggagctgtaaatcatcccgcattcggaaattattaaaccaatggcagttcatgggcaggcaaagccctgcacgaatcttggcggctcgcatcctctattgcggcgctgcatcatggacacgccagcctgccaaagccaaagccaaagcgccccaatgcgatgccacgaaaaagcgatcagcgtcagacacagccgcgcgacaagctgctaaagaaacccacataaaaacgcgcagcgcccggaacgccgcgcggcgaccacggtgccgtgcgggggtgtctgcgtctctctccctcctctctctctccgccgacgaggcgcgagggagtaaggacgcgcgcgccggccgacggcacgcgggccgagggaatggccgccgccgccaccgccaccgcctcctcgtcctcgtcaacctcctcctcggccggctcctccgcgtccacctccacgccccggcccgccccgcgccaggccgccgccgcgccgtcgtcgtccccggtgttcctcaacgtgtacgacgtgacccccgccaacgggtacgcgcggtggctggggctcggcgtgtaccactcgggcgtgcaggtccacggcgtggagtacgcgtacggcgcgcacgagggcgccgggagcggcatcttcgaggtgcccccgcggcggtgccccggctacgcgttccgggaggcggtgctggtgggcaccacggcgctgacccgcgccgaggtgcgcgcgctcatggccgacctcgccgccgacttcccgggcgacgcctacaacctcgtctcccgcaactgcaaccacttctgcgacgccgcctgccgccgcctcgtcgcccgcgcccgcatcccgcgctgggtcaaccgcctcgccaagatcggggtcgtcttcacctgcgtcatccccagcagcagcaggcaccaggtgcgccgcaagggggagcagcagctgcccgcggccgtcaagagccgctccgcgcgccaggccgccgccccgccgcggcccaggaccttcttccgctccctctccgtcggcggcggcaagaacgtcacgccccgcccgctccagaccccgccaccgacgccgccggtggcccccgccctgacgttgacgacgccgacaccaacgccgttggcctccatgtaacggcgccattactcctttttcgtttacagctcacaccttccattttttttccttcgacagttacctgaattttgtccatagtactactcttcgagattaagatttgtgctctgctagtagtagtactgcactgtcaccatgattaccagtagtaactgcagttcattaggctattaatttccgaatttgtctggctttactactacctagatacacctggctggctgtgtgcccgtgtcaccgtctgctgccaaatcgccattaatgattactaatttgagtcgctgttacgcgctgcatttacgttgcggttaacgacgtctatcatgcaattctttgttgtgtggcgtggatccaattctatctggcgagccatccaataggaatatattcgctcctcctttcgcccattctttggaataaacaaccattgtactagctgaagccttgctttggattttcaactagataaaggctccaaagctaagcacggccgatcgatatatgcttttgatgacgcagagaattcccggtgtctggacactccacctgtcatcacactggcgttgtcatgggacacgattagattagtcctcgtgttgttgtttcttgcatgattgcgtccggcctctgtgcctatctggaaatgcggagggagggatgattttaacgtgacctgtcgcatgaaaggcgagcttgcttcgacagaaatcttggggagctcgccggttgcgtgtcgagctcgcctcgccgttgaccggcggcggcggcgaccggtgtgtccatgccggtggcggagacggcggcgtagggtcagaagtgggcaccgacgggaggaggactcgcgtttgtggaggacaccaatgtgcaccacattgaccttgacccgtccgatcagcgatcaggatggacgggccactatcgatccttgggcgggcgtcgctggaccccggccgggctgggttcggtgcacgggatgtgacgccgcagcggcgcctttcgatttcgatcggctacaggagagaagtacgctcgctg

SEQ ID NO: 58 - DNA, 22 bp, Artificial Sequence
  Description of Artificial Sequence Synthetic oligonucleotide
acgatcgacaagctggggaagg

SEQ ID NO: 59 - DNA, 22 bp, Artificial Sequence
  Description of Artificial Sequence Synthetic oligonucleotide
gcgggggagacggtgaaggtgg

SEQ ID NO: 60 - DNA, 22 bp, Artificial Sequence
  Description of Artificial Sequence Synthetic oligonucleotide
gacggtgaaggtggcgccgggg

SEQ ID NO: 61 - DNA, 22 bp, Artificial Sequence
  Description of Artificial Sequence Synthetic oligonucleotide
acgttcttgccgccgccgacgg

SEQ ID NO: 62 - DNA, 22 bp, Artificial Sequence
  Description of Artificial Sequence Synthetic oligonucleotide
gcgtcgtcaacgtcagggcggg

SEQ ID NO: 63 - DNA, 22 bp, Artificial Sequence
  Description of Artificial Sequence Synthetic oligonucleotide
gggagcggaagaaggtcctggg

SEQ ID NO: 64 - DNA, 22 bp, Artificial Sequence
  Description of Artificial Sequence Synthetic oligonucleotide
ccgtcggcggcggcaagaacgt

SEQ ID NO: 65 - DNA, 22 bp, Artificial Sequence
  Description of Artificial Sequence Synthetic oligonucleotide
cccgccctgacgttgacgacgc

SEQ ID NO: 66 - DNA, 22 bp, Artificial Sequence
  Description of Artificial Sequence Synthetic oligonucleotide
cccaggaccttcttccgctccc

SEQ ID NO: 67 - DNA, 22 bp, Artificial Sequence
  Description of Artificial Sequence Synthetic oligonucleotide
gctttcgatccggtgaggccgg

SEQ ID NO: 68 - DNA, 22 bp, Artificial Sequence
  Description of Artificial Sequence Synthetic oligonucleotide
agagattttagattgtgcgggg

SEQ ID NO: 69 - DNA, 22 bp, Artificial Sequence
  Description of Artificial Sequence Synthetic oligonucleotide
atgcgaaccctcctcccgtcgg

SEQ ID NO: 70 - DNA, 22 bp, Artificial Sequence
  Description of Artificial Sequence Synthetic oligonucleotide
gcgccgccgtcttcgccaccgg

SEQ ID NO: 71 - DNA, 22 bp, Artificial Sequence
  Description of Artificial Sequence Synthetic oligonucleotide
ggtcaacggcgaggcgcgctgg

SEQ ID NO: 72 - DNA, 22 bp, Artificial Sequence
  Description of Artificial Sequence Synthetic oligonucleotide
ccgacgggaggagggttcgcat

SEQ ID NO: 73 - DNA, 22 bp, Artificial Sequence
  Description of Artificial Sequence Synthetic oligonucleotide
ccggtggcgaagacggcggcgc

SEQ ID NO: 74 - DNA, 22 bp, Artificial Sequence
  Description of Artificial Sequence Synthetic oligonucleotide
ccagcgcgcctcgccgttgacc

SEQ ID NO: 75 - DNA, 23 bp, Triticum aestivum
ggatggccaatgcgagatgatgg